(19) United States
(12) Patent Application Publication
(10) Pub. No.: US 2008/0004273 A1
(43) Pub. Date: Jan. 3, 2008

(54) Inhibitors of protein tyrosine kinase activity

(76) Inventors: Stephane Raeppel, St. Lazare (CA); Stephen William Claridge, Montreal (CA); Oscar Mario Saavedra, Montreal (CA); Arkadii Vaisburg, Kirkland (CA); Robert Deziel, Mount-Royal (CA); Lijie Zhan, Montreal (CA); Michael Mannion, Montreal (CA); Frederic Gaudette, Verdun (CA); Nancy Z. Zhou, Kirkland (CA); Ljubomir Isakovic, Beaconsfield (CA)

Correspondence Address: KEOWN & ZUCCHERO, LLP, 500 WEST CUMMINGS PARK, SUITE 1200, WOBURN, MA, 01801, US

(21) Appl. No.: 11/807,907

(22) Filed: May 30, 2007

Related U.S. Application Data
(60) Provisional application No. 60/803,412, filed on May 30, 2006.

(51) Int. Cl. A61K 31/47; A61K 31/517; A61K 31/5375; A61P 37/00; C07D 215/00; C07D 239/72; C07D 265/30; C12N 5/00; C12N 9/99

(52) U.S. Cl. 514/235.2; 435/184; 435/375; 514/266.1; 514/312; 544/128; 544/283; 546/153

(57) ABSTRACT

This invention relates to compounds that inhibit protein tyrosine kinase activity. In particular the invention relates to compounds that inhibit the protein tyrosine kinase activity of growth factor receptors, resulting in the inhibition of receptor signaling, for example, the inhibition of VEGF receptor signaling and HGF receptor signaling. More particularly, the invention relates to compounds, compositions and methods for the inhibition of VEGF receptor signaling and HGF receptor signaling. The invention also provides compositions and methods for treating cell proliferative diseases and conditions.

### RELATED APPLICATIONS

This application claims the benefit of prior U.S. Provisional Application Ser. No. 60/803,412, filed on May 30, 2006, the entire teachings of which are incorporated herein by reference.

### BACKGROUND OF THE INVENTION

1. Field of the Invention

This invention relates to compounds that inhibit protein tyrosine kinase activity. In particular the invention relates to compounds that inhibit the protein tyrosine kinase activity of growth factor receptors, resulting in the inhibition of receptor signaling, for example, the inhibition of VEGF receptor signaling and HGF receptor signaling. More particularly, the invention relates to compounds, compositions and methods for the inhibition of VEGF receptor signaling and HGF receptor signaling.

2. Summary of the Related Art

Tyrosine kinases may be classified as growth factor receptor (e.g. EGFR, PDGFR, FGFR and erbB2) or non-receptor (e.g. c-src and bcr-abl) kinases. The receptor type tyrosine kinases make up about 20 different subfamilies. The non-receptor type tyrosine kinases make up numerous subfamilies. These tyrosine kinases have diverse biological activity. Receptor tyrosine kinases are large enzymes that span the cell membrane and possess an extracellular binding domain for growth factors, a transmembrane domain, and an intracellular portion that functions as a kinase to phosphorylate a specific tyrosine residue in proteins and hence to influence cell proliferation. Aberrant or inappropriate protein kinase activity can contribute to the rise of disease states associated with such aberrant kinase activity.

Angiogenesis is an important component of certain normal physiological processes such as embryogenesis and wound healing, but aberrant angiogenesis contributes to some pathological disorders and in particular to tumor growth.^{1,2} VEGF-A (vascular endothelial growth factor A) is a key factor promoting neovascularization (angiogenesis) of tumors.³⁻⁷ VEGF induces endothelial cell proliferation and migration by signaling through two high affinity receptors, the fins-like tyrosine kinase receptor, Flt-1, and the kinase insert domain-containing receptor, KDR.^{8,9,10} These signaling responses are critically dependent upon receptor dimerization and activation of intrinsic receptor tyrosine kinase (RTK) activity. The binding of VEGF as a disulfide-linked homodimer stimulates receptor dimerization and activation of the RTK domain¹¹. The kinase activity autophosphorylates cytoplasmic receptor tyrosine residues, which then serve as binding sites for molecules involved in the propagation of a signaling cascade. Although multiple pathways are likely to be elucidated for both receptors, KDR signaling is most extensively studied, with a mitogenic response suggested to involve ERK-1 and ERK-2 mitogen-activated protein kinases¹².

Disruption of VEGF receptor signaling is a highly attractive therapeutic target in cancer, as angiogenesis is a prerequisite for all solid tumor growth, and that the mature endothelium remains relatively quiescent (with the exception of the female reproductive system and wound healing). A number of experimental approaches to inhibiting VEGF signaling have been examined, including use of neutralizing antibodies^{13,14,15}, receptor antagonists¹⁶, soluble receptors¹⁷, antisense constructs and dominant-negative strategies¹⁹.

Despite the attractiveness of anti-angiogenic therapy by VEGF inhibition alone, several issues may limit this approach. VEGF expression levels can themselves be elevated by numerous diverse stimuli and perhaps most importantly, the hypoxic state of tumors resulting from VEGFr inhibition, can lead to the induction of factors that themselves promote tumor invasion and metastasis thus, potentially undermining the impact of VEGF inhibitors as cancer therapeutics²⁰.

The HGF (hepatocyte growth factor) and the HGF receptor, c-met, are implicated in the ability of tumor cells to undermine the activity of VEGF inhibition²⁰. HGF derived from either stromal fibroblasts surrounding tumor cells or expressed from the tumor itself has been suggested to play a critical role in tumor angiogenesis, invasion and metastasis^{21,22}. For example, invasive growth of certain cancer cells is drastically enhanced by tumor-stromal interactions involving the HGF/c-Met (HGF receptor) pathway^{23,24,25}. HGF, which was originally identified as a potent mitogen for hepatocytes^{26,27} is primarily secreted from stromal cells, and the secreted HGF can promote motility and invasion of various cancer cells that express c-Met in a paracrine manner^{28,29,30}. Binding of HGF to c-Met leads to receptor phosphorylation and activation of Ras/mitogen-activated protein kinase (MAPK) signaling pathway, thereby enhancing malignant behaviors of cancer cells^{30,31}. Moreover, stimulation of the HGF/c-met pathway itself can lead to the induction of VEGF expression, itself contributing directly to angiogenic activity³².

Thus, anti-tumor anti-angiogenic strategies or approaches that target both VEGF/VEGFr signaling and HGF/c-met signaling may circumvent the ability of tumor cells to overcome VEGF inhibition alone and may represent improved cancer therapeutics.

Here we describe small molecules that are potent inhibitors of protein tyrosine kinase activity, such as that of, for example, both the VEGF receptor KDR and the HGF receptor c-met, among others.

### BRIEF SUMMARY OF THE INVENTION

The present invention provides new compounds and methods for treating cell proliferative diseases. The compounds of the invention are inhibitors of protein tyrosine kinase activity. Preferably, the compounds of the invention are dual function inhibitors, capable of inhibiting both VEGF and HGF receptor signaling. Accordingly, the invention provides new inhibitors of protein tyrosine kinase receptor signaling, such as for example, VEGF receptor signaling and HGF receptor signaling, including the VEGF receptor KDR and the HGF receptor c-met.

In a first aspect, the invention provides compounds of formulas I, I-A and I-B that are useful as kinase inhibitors and, therefore, are useful research tools for the study of the role of kinases in both normal and disease states. Preferrably, the invention provides compounds of Formula I that are useful as inhibitors of VEGF receptor signaling and HGF receptor signaling and, therefore, are useful research tools for the study of the role of VEGF and HGF in both normal and disease states.

In a second aspect, the invention provides compounds of formula II that are useful as kinase inhibitors and, therefore, are useful research tools for the study of the role of kinases in both normal and disease states. Preferrably, the invention provides compounds of Formula II that are useful as inhibitors of VEGF receptor signaling and HGF receptor signaling and, therefore, are useful research tools for the study of the role of VEGF and HGF in both normal and disease states.

In a third aspect, the invention provides compounds of formula III that are useful as kinase inhibitors and, therefore, are useful research tools for the study of the role of kinases in both normal and disease states. Preferrably, the invention provides compounds of Formula III that are useful as inhibitors of VEGF receptor signaling and HGF receptor signaling and, therefore, are useful research tools for the study of the role of VEGF and HGF in both normal and disease states.

In a fourth aspect, the invention provides compounds of formulas IV and IV-A that are useful as kinase inhibitors and, therefore, are useful research tools for the study of the role of kinases in both normal and disease states. Preferrably, the invention provides compounds of Formula IV that are useful as inhibitors of VEGF receptor signaling and HGF receptor signaling and, therefore, are useful research tools for the study of the role of VEGF and HGF in both normal and disease states.

In a fifth aspect, the invention provides compounds of formulas V and V-A that are useful as kinase inhibitors and, therefore, are useful research tools for the study of the role of kinases in both normal and disease states. Preferrably, the invention provides compounds of Formula IV that are useful as inhibitors of VEGF receptor signaling and HGF receptor signaling and, therefore, are useful research tools for the study of the role of VEGF and HGF in both normal and disease states.

In a sixth aspect, the invention provides compositions comprising a compound that is an inhibitor of protein tyrosine kinase, or a pharmaceutically acceptable salt thereof, and a pharmaceutically acceptable carrier, excipient or diluent. Preferably, the invention provides compositions comprising a compound that is an inhibitor of VEGF receptor signaling and HGF receptor signaling, or a pharmaceutically acceptable salt thereof, and a pharmaceutically acceptable carrier, excipient, or diluent. In a preferred embodiment, the composition further comprises an additional therapeutic agent.

In a seventh aspect, the invention provides a method of inhibiting protein tyrosine kinase, the method comprising contacting the kinase with a compound according to the present invention, or with a composition according to the present invention. Preferably the invention provides a method of inhibiting VEGF receptor signaling and HGF receptor signaling, the method comprising contacting the receptor with a compound according to the present invention, or with a composition according to the present invention. Inhibition of receptor protein kinase activity, preferably VEGF and HGF receptor signaling, can be in a cell or a multicellular organism. If in a multicellular organism, the method according to this aspect of the invention comprises administering to the organism a compound according to the present invention, or a composition according to the present invention. Preferably the organism is a mammal, more preferably a human. In a preferred embodiment, the method further comprises contacting the kinase with an additional therapeutic agent.

In an eighth aspect, the invention provides a method of inhibiting proliferative activity of a cell, the method comprising contacting the cell with an effective proliferative inhibiting amount of a compound according to the present invention or a composition thereof. In a preferred embodiment, the method further comprises contacting the cell with an additional therapeutic agent.

In a ninth aspect, the invention provides a method of treating a cell proliferative disease in a patient, the method comprising administering to the patient in need of such treatment an effective therapeutical amount of a compound according to the present invention or a composition thereof. In a preferred embodiment, the method further comprises administering an additional therapeutic agent.

In a tenth aspect, the invention provides a method of inhibiting tumor growth in a patient, the method comprising administering to the patient in need thereof an effective therapeutical amount of a compound according to the present invention or a composition thereof. In a preferred embodiment, the method further comprises administering an additional therapeutic agent.

The foregoing merely summarizes certain aspects of the invention and is not intended to be limiting in nature. These aspects and other aspects and embodiments are described more fully below.

### DETAILED DESCRIPTION OF THE PREFERRED EMBODIMENTS

The invention provides compounds and methods for inhibiting protein tyrosine kinase, preferably the VEGF receptor KDR and the HGF receptor c-met. The invention also provides compositions and methods for treating cell proliferative diseases and conditions. The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

For purposes of the present invention, the following definitions will be used (unless expressly stated otherwise):

The terms “inhibitor of VEGF receptor signaling” and “inhibitor of HGF receptor signaling” are used to identify a compound having a structure as defined herein, which is capable of interacting with a HGF receptor and a VEGF receptor and inhibiting the activity of the HGF receptor and the VEGF receptor. In some preferred embodiments, such reduction of activity is at least about 50%, more preferably at least about 75%, and still more preferably at least about 90%.

Reference to “a compound of the formula (I), formula (II), etc.,” (or equivalently, “a compound according to the first aspect”, or “a compound of the present invention”, and the like), herein is understood to include reference to N-oxides, hydrates, solvates, pharmaceutically acceptable salts, prodrugs and complexes thereof, and racemic mixtures, diastereomers, enantiomers and tautomers thereof and unless otherwise indicated.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—. Also, a number of moieties disclosed herein exist in multiple tautomeric forms, all of which are intended to be encompassed by any given tautomeric structure.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperazinyl and piperidinyl (C₆); C₆-heteroaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃ hydrocarbyl” includes a covalent bond, methyl, ethyl, ethenyl, ethynyl, propyl, propenyl, propynyl, and cyclopropyl.

In particular, hydrocarbyl groups include alkyl, alkenyl, alkynyl, aryl, aralkyl, cycloalkyl, cycloalkenyl or cycloalkynyl.

The term “aliphatic” is intended to mean both saturated and unsaturated, straight chain or branched aliphatic hydrocarbons. As will be appreciated by one of ordinary skill in the art, “aliphatic” is intended herein to include, but is not limited to, alkyl, alkenyl, or alkynyl moieties.

The term “alkyl” is intended to mean a straight chain or branched aliphatic group having from 1 to 12 carbon atoms, preferably 1-8 carbon atoms, and more preferably 1-6 carbon atoms. Other preferred alkyl groups have from 2 to 12 carbon atoms, preferably 2-8 carbon atoms and more preferably 2-6 carbon atoms. Preferred alkyl groups include, without limitation, methyl, ethyl, propyl, isopropyl, butyl, isobutyl, sec-butyl, tert-butyl, pentyl, hexyl and the like. A “C₀” alkyl (as in “C₀-C₃alkyl”) is a covalent bond.

The term “alkenyl” is intended to mean an unsaturated straight chain or branched aliphatic group with one or more carbon-carbon double bonds, having from 2 to 12 carbon atoms, preferably 2-8 carbon atoms, and more preferably 2-6 carbon atoms. Preferred alkenyl groups include, without limitation, ethenyl, propenyl, butenyl, pentenyl, and hexenyl.

The term “alkynyl” is intended to mean an unsaturated straight chain or branched aliphatic group with one or more carbon-carbon triple bonds, having from 2 to 12 carbon atoms, preferably 2-8 carbon atoms, and more preferably 2-6 carbon atoms. Preferred alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “azolyl” as employed herein is intended to mean a five-membered saturated or unsaturated heterocyclic group containing two or more hetero-atoms, as ring atoms, selected from the group consisting of nitrogen, sulfur and oxygen, wherein at least one of the hetero-atoms is a nitrogen atom. Preferred azolyl groups include, but are not limited to, optionally substituted imidazolyl, oxazolyl, thiazolyl, pyrazolyl, isoxazolyl, isothiazolyl, 1,3,4-thiadiazolyl, 1,2,4-thiadiazolyl, 1,2,4-oxadiazolyl, and 1,3,4-oxadiazolyl.

The term “carbocycle” as employed herein is intended to mean a cycloalkyl or aryl moiety. The term “carbocycle” also includes a cycloalkenyl moiety having at least one carbon-carbon double bond.

The term “cycloalkyl” is intended to mean a saturated or unsaturated mono-, bi-, tri- or poly-cyclic hydrocarbon group having about 3 to 15 carbons, preferably having 3 to 12 carbons, preferably 3 to 8 carbons, more preferably 3 to 6 carbons, and more preferably still 5 or 6 carbons. In certain preferred embodiments, the cycloalkyl group is fused to an aryl, heteroaryl or heterocyclic group. Preferred cycloalkyl groups include, without limitation, cyclopenten-2-enone, cyclopenten-2-enol, cyclohex-2-enone, cyclohex-2-enol, cyclopropyl, cyclobutyl, cyclobutenyl, cyclopentyl, cyclopentenyl, cyclohexyl, cyclohexenyl, cycloheptyl, cyclooctyl, etc.

The term “heteroalkyl” is intended to mean a saturated or unsaturated, straight chain or branched aliphatic group, wherein one or more carbon atoms in the group are independently replaced by a moiety selected from the group consisting of O, S, N,N-alkyl, —S(O)—, —S(O)₂—, —S(O)₂NH—, or —NHS(O)₂—.

The term “aryl” is intended to mean a mono-, bi-, tri- or polycyclic aromatic moiety, preferably a C₆-C₁₄aromatic moiety, preferably comprising one to three aromatic rings. Preferably, the aryl group is a C₆-C₁₀aryl group, more preferably a C₆aryl group. Preferred aryl groups include, without limitation, phenyl, naphthyl, anthracenyl, and fluorenyl.

The terms “aralkyl” or “arylalkyl” is intended to mean a group comprising an aryl group covalently linked to an alkyl group. If an aralkyl group is described as “optionally substituted”, it is intended that either or both of the aryl and alkyl moieties may independently be optionally substituted or unsubstituted. Preferably, the aralkyl group is (C₁-C₆)alk(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is a mono-, bi-, or polycyclic structure having from about 3 to about 14 atoms, wherein one or more atoms are independently selected from the group consisting of N, O, and S. The ring structure may be saturated, unsaturated or partially unsaturated. In certain preferred embodiments, the heterocyclic group is non-aromatic, in which case the group is also known as a heterocycloalkyl. In certain preferred embodiments, the heterocyclic group is a bridged heterocyclic group (for example, a bicyclic moiety with a methylene, ethylene or propylene bridge). In a bicyclic or polycyclic structure, one or more rings may be aromatic; for example one ring of a bicyclic heterocycle or one or two rings of a tricyclic heterocycle may be aromatic, as in indan and 9,10-dihydro anthracene. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” is intended to mean a mono-, bi-, tri- or polycyclic group having 5 to 14 ring atoms, preferably 5, 6, 9, or 10 ring atoms; having 6, 10, or 14 pi electrons shared in a cyclic array; and having, in addition to carbon atoms, between one or more heteroatoms independently selected from the group consisting of N, O, and S. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

A heteroalicyclic group refers specifically to a non-aromatic heterocyclyl radical. A heteroalicyclic may contain unsaturation, but is not aromatic.

A heterocyclylalkyl group refers to a residue in which a heterocyclyl is attached to a parent structure via one of an alkylene, alkylidene, or alkylidyne radical. Examples include (4-methylpiperazin-1-yl)methyl, (morpholin-4-yl)methyl, (pyridine-4-yl)methyl,2-(oxazolin-2-yl) ethyl, 4-(4-methylpiperazin-1-yl)-2-butenyl, and the like. If a heterocyclylalkyl is described as “optionally substituted” it is meant that both the heterocyclyl and the corresponding alkylene, alkylidene, or alkylidyne radical portion of a heterocyclylalkyl group may be optionally substituted. A “lower heterocyclylalkyl” refers to a heterocyclylalkyl where the “alkyl” portion of the group has one to six carbons.

A heteroalicyclylalkyl group refers specifically to a heterocyclylalkyl where the heterocyclyl portion of the group is non-aromatic.

Preferred heterocyclyls and heteroaryls include, but are not limited to, azepinyl, azetidinyl, acridinyl, azocinyl, benzidolyl, benzimidazolyl, benzofuranyl, benzofurazanyl, benzofuryl, benzothiofuranyl, benzothiophenyl, benzoxazolyl, benzothiazolyl, benzothienyl, benztriazolyl, benztetrazolyl, benzisoxazolyl, benzisothiazolyl, benzimidazolinyl, benzoxazolyl, benzoxadiazolyl, benzopyranyl, carbazolyl, 4aH-carbazolyl, carbolinyl, chromanyl, chromenyl, cinnolinyl, coumarinyl, decahydroquinolinyl, 1,3-dioxolane, 2H,6H-1,5,2-dithiazinyl, dihydrofuro[2,3-b]tetrahydrofuran, dihydroisoindolyl, dihydroquinazolinyl (such as 3,4-dihydro-4-oxo-quinazolinyl), furanyl, furopyridinyl (such as fuor[2,3-c]pyridinyl, furo[3,2-b]pyridinyl or furo[2,3-b]pyridinyl), furyl, furazanyl, hexahydrodiazepinyl, imidazolidinyl, imidazolinyl, imidazolyl, indazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolidinyl, isothiazolyl, isoxazolinyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, oxetanyl, 2-oxoazepinyl, 2-oxopiperazinyl, 2-oxopiperidinyl, 2-oxopyrrolodinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolopyridyl, 2H-pyrrolyl, pyrrolyl, quinazolinyl, quinolinyl, 4H-quinolizinyl, quinoxalinyl, quinuclidinyl, tetrahydro-1,1-dioxothienyl, tetrahydrofuranyl, tetrahydrofuryl, tetrahydroisoquinolinyl, tetrahydroquinolinyl, tetrahydropyranyl, tetrazolyl, thiazolidinyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thiamorpholinyl, thiamorpholinyl sulfoxide, thiamorpholuiyl sulfone, thianthrenyl, thiazolyl, thienyl, thienothiazolyl, thienooxazolyl, thienoimidazolyl, thiophenyl, triazinyl, triazinylazepinyl, triazolyl (e.g., 1,2,3-triazolyl, 1,2,4-triazolyl, 1,2,5-triazolyl, 1,3,4-triazolyl), and xanthenyl.

A “halohydrocarbyl” as employed herein is a hydrocarbyl moiety, in which from one to all hydrogens have been replaced with one or more halo.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, independently selected non-hydrogen substituents. Suitable substituents include, without limitation, halo, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, hydroxy, cyano, oxo, carboxy, formyl, nitro, amino, amidino, guanidino,
(b) C₁-C₅alkyl or alkenyl or arylalkyl imino, carbamoyl, azido, carboxamido, mercapto, hydroxy, hydroxyalkyl, alkylaryl, arylalkyl, C₁-C₈alkyl, C₁-C₈alkenyl, C₁-C₈alkoxy, C₁-C₈alkyamino, C₁-C₈alkoxycarbonyl, aryloxycarbonyl, C₂-C₈acyl, C₂-C₈acylamino, C₁-C₈alkylthio, arylalkylthio, arylthio, C₁-C₈alkylsulfinyl, arylalkylsulfinyl, arylsulfinyl, C₁-C₈alkylsulfonyl, arylalkylsulfonyl, arylsulfonyl, C₀-C₆N-alkyl carbamoyl, C₂-C₁₅N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇heterocycle, C₅-C₁₅heteroaryl or any of these rings fused or spiro-fused to a cycloalkyl, heterocyclyl, or aryl, wherein each of the foregoing is further optionally substituted with one more moieties listed in (a), above; and
(c) —(CR³²R³³)ₛ—NR³⁰R³¹, wherein s is from 0 (in which case the nitrogen is directly bonded to the moiety that is substituted) to 6, R³² and R³³ are each independently hydrogen, halo, hydroxyl or C₁-C₄alkyl, and R³⁰ and R³¹ are each independently hydrogen, cyano, oxo, hydroxyl, C₁-C₈alkyl, C₁-C₈heteroalkyl, C₁-C₈alkenyl, carboxamido, C₁-C₃alkyl-carboxamido, carboxamido-C₁-C₃alkyl, amidino, C₂-C₈hydroxyalkyl, C₁-C₃alkylaryl, aryl-C₁-C₃alkyl, C₁-C₃alkylheteroaryl, heteroaryl-C₁-C₃alkyl, C₁-C₃alkylheterocyclyl, heterocyclyl-C₁-C₃alkyl C₁-C₃alkylcycloalkyl, cycloalkyl-C₁-C₃alkyl, C₂-C₈alkoxy, C₂-C₈alkoxy-C₁-C₄alkyl, C₁-C₈alkoxycarbonyl, aryloxycarbonyl, aryl-C₁-C₃alkoxycarbonyl, heteroaryloxycarbonyl, heteroaryl-C₁-C₃alkoxycarbonyl, C₁-C₈acyl, C₀-C₈alkyl-carbonyl, aryl-C₀-C₈alkyl-carbonyl, heteroaryl-C₀-C₈alkyl-carbonyl, cycloalkyl-C₀-C₈alkyl-carbonyl, C₀-C₈alkyl-NH-carbonyl, aryl-C₀-C₈alkyl-NH-carbonyl, heteroaryl-C₀-C₈alkyl-NH-carbonyl, cycloalkyl-C₀-C₈alkyl-NH-carbonyl, C₀-C₈alkyl-O-carbonyl, aryl-C₀-C₈alkyl-O-carbonyl, heteroaryl-C₀-C₈alkyl-O-carbonyl, cycloalkyl-C₀-C₈alkyl-O-carbonyl, C₁-C₈alkylsulfonyl, arylalkylsulfonyl, arylsulfonyl, heteroarylalkylsulfonyl, heteroarylsulfonyl, C₁-C₈alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, or protecting group, wherein each of the foregoing is further optionally substituted with one more moieties listed in (a), above; or R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰—Y³¹—), wherein said heterocyclyl may also be bridged (forming a bicyclic moiety with a methylene, ethylene or propylene bridge); wherein
   X³⁰ is selected from the group consisting of C₁-C₈alkyl, C₂-C₈alkenyl-, C₂-C₈alkynyl-, —C₀-C₃alkyl-C₂-C₈alkenyl-C₀-C₃alkyl, C₀-C₃alkyl-C₂-C₈alkynyl-C₀-C₃alkyl, C₀-C₃alkyl-O—C₀-C₃alkyl-, HO—C₀-C₃alkyl-, C₀-C₄alkyl-N(R³⁰)—C₀-C₃alkyl-, N(R³⁰)(R³¹)—C₀-C₃alkyl-, N(R³⁰)(R³¹)—C₀-C₃alkenyl-, N(R³⁰)(R³¹)—C₀-C₃alkynyl-, (N(R³⁰)(R³¹))₂—C═N—, C₀-C₃alkyl-S(O)₀₋₂—C₀-C₃alkyl-, CF₃-C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocycyl are optionally substituted with from 1 to 3 substituents from (a); and Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)—N(R³¹)—, —N(R³⁰)—C(NR³⁰)—N(R³¹)—, —N(R³⁰)—C(NR³¹)—, —C(NR³¹)—N(R³⁰)—, —N(R³⁰)—C(S)—N(R³¹)—, —N(R³⁰)—C(O)—, —O—C(O)—N(R³¹)—, —N(R³⁰)—C(S)—O—, —O—C(S)—N(R³¹)—, —S(O)₀₋₂—, —SO₂N(R³¹)—, —N(R³¹)—SO₂— and —N(R³⁰)—SO₂N(R³¹)—.

A moiety that is substituted is one in which one or more (preferably one to four, preferably from one to three and more preferably one or two), hydrogens have been independently replaced with another chemical substituent. As a non-limiting example, substituted phenyls include 2-fluorophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl —CO—.

When there are two optional substituents bonded to adjacent atoms of a ring structure, such as for example a phenyl, thiophenyl, or pyridinyl, the substituents, together with the atoms to which they are bonded, optionally form a 5- or 6-membered cycloalkyl or heterocycle having 1, 2, or 3 annular heteroatoms.

In a preferred embodiment, a hydrocarbyl, heteroalkyl, heterocyclic and/or aryl group is unsubstituted.

In other preferred embodiments, a hydrocarbyl, heteroalkyl, heterocyclic and/or aryl group is substituted with from 1 to 3 independently selected substituents.

Preferred substituents on alkyl groups include, but are not limited to, hydroxyl, halogen (e.g., a single halogen substituent or multiple halo substituents; in the latter case, groups such as CF₃ or an alkyl group bearing Cl₃), oxo, cyano, nitro, alkyl, cycloalkyl, alkenyl, cycloalkenyl, alkynyl, heterocycle, aryl, —OR^{a}, —SR^{a}, —S(═O)R^{c}, —S(═O)₂R^{e}, P(═O)₂R^{e}, —S(═O)₂OR^{e}, P(═O)₂OR^{e}, —NR^{b}R^{c}, NR^{b}S(═O)₂R^{e}, —NR^{b}P(═O)₂R^{e}, —S(═O)₂NR^{b}R^{c}, P(═O)₂NR^{b}R^{c}, —C(═O)OR^{e}, —C(═O)R^{a}, —C(═O)NR^{b}R^{c}, —OC(═O)R^{a}, —OC(═O)NR^{b}R^{c}, —NR^{b}C(═O)OR^{e}, —NR^{d}C(═O)NR^{b}R^{c}, —NR^{d}S(═O)₂NR^{b}R^{c}, —NR^{d}P(═O)₂NR^{b}R^{c}, —NR^{b}C(═O)R^{a} or —NR^{b}P(═O)₂R^{e}, wherein R^{a} is hydrogen, alkyl, cycloalkyl, alkenyl, cycloalkenyl, alkynyl, heterocycle or aryl; R^{b}, R^{c} and R^{d} are independently hydrogen, alkyl, cycloalkyl, heterocycle or aryl, or said R^{b} and R^{c} together with the N to which they are bonded optionally form a heterocycle; and R^{e} is alkyl, cycloalkyl, alkenyl, cycloalkenyl, alkynyl, heterocycle or aryl. In the aforementioned exemplary substituents, groups such as alkyl, cycloalkyl, alkenyl, alkynyl, cycloalkenyl, heterocycle and aryl can themselves be optionally substituted.

Preferred substituents on alkenyl and alkynyl groups include, but are not limited to, alkyl or substituted alkyl, as well as those groups recited as preferred alkyl substituents.

Preferred substituents on cycloalkyl groups include, but are not limited to, nitro, cyano, alkyl or substituted alkyl, as well as those groups recited about as preferred alkyl substituents. Other preferred substituents include, but are not limited to, spiro-attached or fused cyclic substituents, preferably spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on cycloalkenyl groups include, but are not limited to, nitro, cyano, alkyl or substituted alkyl, as well as those groups recited as preferred alkyl substituents. Other preferred substituents include, but are not limited to, spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on aryl groups include, but are not limited to, nitro, cycloalkyl or substituted cycloalkyl, cycloalkenyl or substituted cycloalkenyl, cyano, alkyl or substituted alkyl, as well as those groups recited above as preferred alkyl substituents. Other preferred substituents include, but are not limited to, fused cyclic groups, especially fused cycloalkyl, fused cycloalkenyl, fused heterocycle, or fused aryl, where the aforementioned cycloalkyl, cylcoalkenyl, heterocycle and aryl substituents can themselves be optionally substituted. Still other preferred substituents on aryl groups (phenyl, as a non-limiting example) include, but are not limited to, haloalkyl and those groups recited as preferred alkyl substituents.

Preferred substituents on heterocylic groups include, but are not limited to, cycloalkyl, substituted cycloalkyl, cycloalkenyl, substituted cycloalkenyl, nitro, oxo (i.e., ═O), cyano, alkyl, substituted alkyl, as well as those groups recited as preferred alkyl substituents. Other preferred substituents on heterocyclic groups include, but are not limited to, spiro-attached or fused cylic substituents at any available point or points of attachment, more preferably spiro-attached cycloalkyl, spiro-attached cycloalkenyl, spiro-attached heterocycle (excluding heteroaryl), fused cycloalkyl, fused cycloakenyl, fused heterocycle and fused aryl, where the aforementioned cycloalkyl, cycloalkenyl, heterocycle and aryl substituents can themselves be optionally substituted.

In certain preferred embodiments, a heterocyclic group is substituted on carbon, nitrogen and/or sulfur at one or more positions. Preferred substituents on nitrogen include, but are not limited to alkyl, aryl, aralkyl, alkylcarbonyl, alkylsulfonyl, arylcarbonyl, arylsulfonyl, alkoxycarbonyl, or aralkoxycarbonyl. Preferred substituents on sulfur include, but are not limited to, oxo and C₁₋₆alkyl. In certain preferred embodiments, nitrogen and sulfur heteroatoms may independently be optionally oxidized and nitrogen heteroatoms may independently be optionally quaternized.

Especially preferred substituents on ring groups, such as aryl, heteroaryl, cycloalkyl and heterocyclyl, include halogen, alkoxy and alkyl.

Especially preferred substituents on alkyl groups include halogen and hydroxy.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—C—NH—). The term “carbamoyl” refers to an amide group attached at the carbonyl carbon atom (i.e., NH₂—C—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

The term “radical” as used herein means a chemical moiety comprising one or more unpaired electrons.

Where optional substituents are chosen from “one or more” groups it is to be understood that this definition includes all substituents being chosen from one of the specified groups or the substituents being chosen from two or more of the specified groups. In addition, substituents on cyclic moieties (i.e., cycloalkyl, heterocyclyl, aryl, heteroaryl) include 5- to 6-membered mono- and 9- to 14-membered bi-cyclic moieties fused to the parent cyclic moiety to form a bi- or tri-cyclic fused ring system. Substituents on cyclic moieties also include 5- to 6-membered mono- and 9- to 14-membered bi-cyclic moieties attached to the parent cyclic moiety by a covalent bond to form a bi- or tri-cyclic bi-ring system. For example, an optionally substituted phenyl includes, but is not limited to, the following:

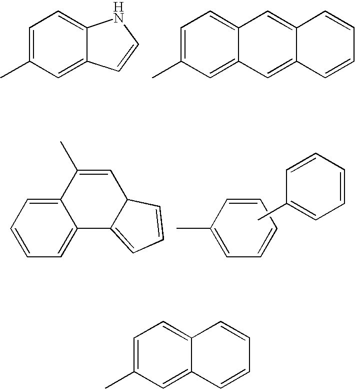

An “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, “unsubstituted aryl” does not include phenyl substituted with any of the optional substituents for which the definition of the moiety (above) otherwise provides.

A saturated or unsaturated three- to eight-membered carbocyclic ring is preferably a four- to seven-membered, more preferably five- or six-membered, saturated or unsaturated carbocyclic ring. Examples of saturated or unsaturated three- to eight-membered carbocyclic rings include phenyl, cyclopropyl, cyclobutyl, cyclopentyl, cyclohexyl, and cycloheptyl.

A saturated or unsaturated three- to eight-membered heterocyclic ring contains at least one heteroatom selected from oxygen, nitrogen, and sulfur atoms. The saturated or unsaturated three- to eight-membered heterocyclic ring preferably contains one or two heteroatoms with the remaining ring-constituting atoms being carbon atoms. The saturated or unsaturated three- to eight-membered heterocyclic ring is preferably a saturated or unsaturated four- to seven-membered heterocyclic ring, more preferably a saturated or unsaturated five- or six-membered heterocyclic ring. Examples of saturated or unsaturated three- to eight-membered heterocyclic groups include thienyl, pyridyl, 1,2,3-triazolyl, imidazolyl, isoxazolyl, pyrazolyl, piperazinyl, piperazino, piperidyl, piperidino, morpholinyl, morpholino, homopiperazinyl, homopiperazino, thiomorpholinyl, thiomorpholino, tetrahydropyrrolyl, and azepanyl.

A saturated or unsaturated carboxylic and heterocyclic group may condense with another saturated or heterocyclic group to form a bicyclic group, preferably a saturated or unsaturated nine- to twelve-membered bicyclic carbocyclic or heterocyclic group. Bicyclic groups include naphthyl, quinolyl, 1,2,3,4-tetrahydroquinolyl, 1,4-benzoxanyl, indanyl, indolyl, and 1,2,3,4-tetrahydronaphthyl.

When a carbocyclic or heterocyclic group is substituted by two C₁₋₆ alkyl groups, the two alkyl groups may combine together to form an alkylene chain, preferably a C₁₋₃ alkylene chain. Carbocyclic or heterocyclic groups having this crosslinked structure include bicyclo[2.2.2]octanyl and norbornanyl.

The terms “kinase inhibitor” and “inhibitor of kinase activity”, and the like, are used to identify a compound which is capable of interacting with a kinase and inhibiting its enzymatic activity.

The term “inhibiting kinase enzymatic activity” is used to mean reducing the ability of a kinase to transfer a phosphate group from a donor molecule, such as ATP, to a specific target molecule (substrate). For example, the inhibition of kinase activity may be at least about 10%. In some preferred embodiments of the invention, such reduction of kinase activity is at least about 50%, more preferably at least about 75%, and still more preferably at least about 90%. In other preferred embodiments, kinase activity is reduced by at least 95% and even more preferably by at least 99%. The IC₅₀ value is the concentration of kinase inhibitor which reduces the activity of a kinase to 50% of the uninhibited enzyme.

The term “inhibiting effective amount” is meant to denote a dosage sufficient to cause inhibition of kinase activity. The kinase may be in a cell, which in turn may be in a multicellular organism. The multicellular organism may be, for example, a plant, a fungus or an animal, preferably a mammal and more preferably a human. The fungus may be infecting a plant or a mammal, preferably a human, and could therefore be located in and/or on the plant or mammal. If the kinase is in a multicellular organism, the method according to this aspect of the invention comprises the step of administering to the organism a compound or composition according to the present invention. Administration may be by any route, including, without limitation, parenteral, oral, sublingual, transdermal, topical, intranasal, intratracheal, or intrarectal. In certain particularly preferred embodiments, compounds of the invention are administered intravenously in a hospital setting. In certain other preferred embodiments, administration may preferably be by the oral route.

Preferably, such inhibition is specific, i.e., the kinase inhibitor reduces the ability of a kinase to transfer a phosphate group from a donor molecule, such as ATP, to a specific target molecule (substrate) at a concentration that is lower than the concentration of the inhibitor that is required to produce another, unrelated biological effect. Preferably, the concentration of the inhibitor required for kinase inhibitory activity is at least 2-fold lower, more preferably at least 5-fold lower, even more preferably at least 10-fold lower, and most preferably at least 20-fold lower than the concentration required to produce an unrelated biological effect.

The term “therapeutically effective amount” as employed herein is an amount of a compound of the invention, that when administered to a patient, treats the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art.

The term “patient” as employed herein for the purposes of the present invention includes humans and other animals, particularly mammals, and other organisms. Thus the compounds, compositions and methods of the present invention are applicable to both human therapy and veterinary applications. In a preferred embodiment the patient is a mammal, and in a most preferred embodiment the patient is human.

The terms “treating”, “treatment”, or the like, as used herein covers the treatment of a disease-state in an animal and includes at least one of: (i) preventing the disease-state from occurring, in particular, when such animal is predisposed to the disease-state but has not yet been diagnosed as having it; (ii) inhibiting the disease-state, i.e., partially or completely arresting its development; (iii) relieving the disease-state, i.e., causing regression of symptoms of the disease-state, or ameliorating a symptom of the disease; and (iv) reversal or regression of the disease-state, preferably eliminating or curing of the disease. In a preferred embodiment of the present invention the animal is a mammal, preferably a primate, more preferably a human. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

The present invention also includes prodrugs of compounds of the invention. The term “prodrug” is intended to represent covalently bonded carriers, which are capable of releasing the active ingredient of the prodrug when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the present invention include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of the invention, amides (e.g., trifluoroacetylamino, acetylamino, and the like), and the like.

The compounds of the invention may be administered in the form of an in vivo hydrolyzable ester or in vivo hydrolyzable amide. An in vivo hydrolyzable ester of a compound of the invention containing carboxy or hydroxy group is, for example, a pharmaceutically acceptable ester which is hydrolyzed in the human or animal body to produce the parent acid or alcohol. Suitable pharmaceutically acceptable esters for carboxy include C₁₋₆-alkoxymethyl esters (e.g., methoxymethyl), C₁₋₆-alkanoyloxymethyl esters (e.g., for example pivaloyloxymethyl), phthalidyl esters, C₃₋₈-cycloalkoxycarbonyloxyC₁₋₆-alkyl esters (e.g., 1-cyclohexylcarbonyloxyethyl); 1,3-dioxolen-2-onylmethyl esters (e.g., 5-methyl-1,3-dioxolen-2-onylmethyl; and C₁₋₆-alkoxycarbonyloxyethyl esters (e.g., 1-methoxycarbonyloxyethyl) and may be formed at any carboxy group in the compounds of this invention

An in vivo hydrolyzable ester of a compound of the invention containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N—(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N—C₁-C₆alkyl or N,N-di-C₁-C₆alkyl amide such as N-methyl, N-ethyl, N-propyl, N,N-dimethyl, N-ethyl-N-methyl or N,N-diethyl amide.

Upon administration to a subject, the prodrug undergoes chemical conversion by metabolic or chemical processes to yield a compound of the present invention, or a salt and/or solvate thereof. Solvates of the compounds of the present invention include, for example, hydrates.

Another aspect of the invention provides compositions including a compound, N-oxide, hydrate, solvate, pharmaceutically acceptable salt, complex or prodrug of a compound according to the present invention as described herein, or a racemic mixture, diastereomer, enantiomer or tautomer thereof. For example, in one embodiment of the invention, a composition comprises a compound, N-oxide, hydrate, solvate, pharmaceutically acceptable salt, complex or prodrug of a compound according to the present invention as described herein present in at least about 30% enantiomeric or diastereomeric excess. In certain desirable embodiments of the invention, the compound, N-oxide, hydrates, solvate, pharmaceutically acceptable salt, complex or prodrug is present in at least about 50%, at least about 80%, or even at least about 90% enantiomeric or diastereomeric excess. In certain other desirable embodiments of the invention, the compound, N-oxide, hydrate, solvate, pharmaceutically acceptable salt, complex or prodrug is present in at least about 95%, more preferably at least about 98% and even more preferably at least about 99% enantiomeric or diastereomeric excess. In other embodiments of the invention, a compound, N-oxide, hydrate, solvate, pharmaceutically acceptable salt, complex or prodrug is present as a substantially racemic mixture.

Some compounds of the invention may have chiral centers and/or geometric isomeric centers (E- and Z-isomers), and it is to be understood that the invention encompasses all such optical, enantiomeric, diastereoisomeric and geometric isomers. The invention also comprises all tautomeric forms of the compounds disclosed herein. Where compounds of the invention include chiral centers, the invention encompasses the enantiomerically and/or diasteromerically pure isomers of such compounds, the enantiomerically and/or diastereomerically enriched mixtures of such compounds, and the racemic and scalemic mixtures of such compounds. For example, a composition may include a mixture of enantiomers or diastereomers of a compound of formula (1) in at least about 30% diastereomeric or enantiomeric excess. In certain embodiments of the invention, the compound is present in at least about 50% enantiomeric or diastereomeric excess, in at least about 80% enantiomeric or diastereomeric excess, or even in at least about 90% enantiomeric or diastereomeric excess. In certain more preferred embodiments of the invention, the compound is present in at least about 95%, even more preferably in at least about 98% enantiomeric or diastereomeric excess, and most preferably in at least about 99% enantiomeric or diastereomeric excess.

The chiral centers of the present invention may have the S or R configuration. The racemic forms can be resolved by physical methods, such as, for example, fractional crystallization, separation or crystallization of diastereomeric derivates or separation by chiral column chromatography. The individual optical isomers can be obtained either starting from chiral precursors/intermediates or from the racemates by any suitable method, including without limitation, conventional methods, such as, for example, salt formation with an optically active acid followed by crystallization.

The term “functional group” is intended to mean a reactive substituent such as nitro, ddd eee cc ddd ece ddd eee cyano, halogen, oxo, ═CR^{ddd}R^{eee}, C(O)₁₋₂R^{ccc}, OR^{ccc}, S(O)₀₋₃R^{ccc}, NR^{ddd}R^{eee}, C(O)NR^{ddd}R^{eee}, OC(O)NR^{ddd}R^{eee}, ═NOR^{ccc}, —NR^{ccc}C(O)₁₋₂R^{ddd}, NR^{ccc}C(O)NR^{ddd}R^{eee}, —N═CR^{ddd}R^{eee}, S(O)₀₋₃NR^{ddd}R^{eee} or —NR^{ccc}S(O)₀₋₃R^{ddd}, wherein R^{ccc}, R^{ddd} and R^{eee} are independently selected from the group consisting of H, optionally substituted hydrocarbyl, optionally substituted heterocyclyl and optionally substituted alkoxy, or R^{ddd} and R^{eee} together form an optionally substituted ring wihc optionally contains further heteroatoms such as oxygen, nitrogen, S, S(O) or S(O)₂.

Suitable optional substituents for hydrocarbyl, heterocyclyl or alkoxy groups R^{ccc}, R^{ddd} and R^{eee} as well as rings formed by R^{ddd} and R^{eee} include halogen, perhaloalky such as trifluoromethyl, mercapto, thioalkyl, hydroxyl, carboxy, alkoxy, heteroaryl, heteroaryloxy, cycloalkyl, cycloalkenyl, cycloalkynyl, alkenyloxy, alkynyloxy, alkoxyalkoxy, aryloxy (where the aryl group may be substituted by halo, nitro, or hydroxyl), cyano, nitro, amino, mono- or di-alkyl amino, oximino or S(O)₀₋₃R^{fff}, wherein R^{fff} is a hydrocarbyl group such as alkyl.

Throughout the specification, preferred embodiments of one or more chemical substituents are identified. Also preferred are combinations of preferred embodiments. For example, the invention describes preferred embodiments of R⁷ in the compounds and describes preferred embodiments of group W. Thus, as an example, also contemplated as within the scope of the invention are compounds in which preferred examples of R⁷ are as described and in which preferred examples of group W are as described.

### Compounds

According to one embodiment, the invention provides compounds of Formula (I) and racemic mixtures, diastereomers and enantiomers thereof: 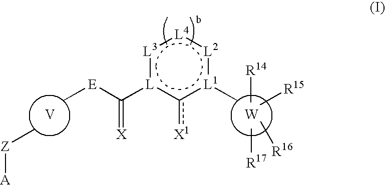
and N-oxides, hydrates, solvates, pharmaceutically acceptable salts, prodrugs and complexes thereof, wherein, 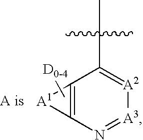
wherein
A¹ represents a fused 6-membered aryl or heteroaryl group;
A2 and A³ are independently selected from N and CR¹⁰⁷;
R¹⁰⁷ is selected from the group consisting of hydrogen, halogen, C₁-C₁₂alkyl, C₂-C₁₂alkenyl, C₂-C₁₂alkynyl, C₃-C₁₂cycloalkyl, C₆-C₁₂aryl, 3-12 membered heteroalicyclic, 5-12 membered heteroaryl, —S(O)₀₋₂R¹⁰⁸, —SO₂NR¹⁰⁸R¹⁰⁹, —S(O)₂OR¹⁰⁸, —NO₂, —NR¹⁰⁸R¹⁰⁹, —(CR¹¹⁰R¹¹¹)₀₋₄OR¹⁰⁸, —CN, —C(O)R¹⁰⁸, —OC(O)R¹⁰⁸, —O(CR110R111)₀₋₄R¹⁰⁸, —NR¹⁰⁸C(O)R¹⁰⁹, —(CR¹¹⁰R¹¹¹)₀₋₄C(O)OR¹⁰⁸, —(CR¹¹⁰R¹¹¹)₀₋₄NR¹⁰⁸R¹⁰⁹, —C(═NR¹¹⁰)NR¹⁰⁸R¹⁰⁹NR¹⁰⁸C(O)NR¹⁰⁹R¹¹⁰, —NR¹⁰⁸S(O)₁₋₂R¹⁰⁹ and —C(O)NR¹⁰⁸R¹⁰⁹, wherein each hydrogen of which is optionally substituted by an R¹¹⁷ group;
each R¹⁰⁸, R¹⁰⁹, R¹¹⁰ and R¹¹¹, which may be the same or different, is independently selected from hydrogen, halogen, C₁-C₁₂alkyl, C₂-C₁₂alkenyl, C₂-C₁₂alkynyl, C₃-C₁₂cycloalkyl, C6-C12aryl, 3-12 membered heteroalicyclic and 5-12 membered heteroaryl, or any two of R¹⁰⁸, R¹⁰⁹, R¹¹⁰ and R¹¹¹ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from N, O and S, or any two of R¹⁰⁸, R¹⁰⁹, R¹⁰⁹ and R¹¹¹ bound to the same carbon atom may be combined to form a C₃-C₁₂ cycloalkyl, C₆-C₁₂ aryl, 3-12 membered heteroalicyclic or 5-12 membered heteroaryl group, and each hydrogen of R¹⁰⁸, R¹⁰⁹, R¹¹ and R¹¹ is optionally substituted by from 1 to 6 R¹¹⁷ groups;
each R¹¹⁷, which may be the same or different, is independently selected from halogen, C₁-C₁₂ alkyl, C₂-C₁₂ alkenyl, C₂-C₁₂ alkynyl, C₃-C₁₂ cycloalkyl, C₆-C₁₂ aryl, 3-12 membered heteroalicyclic, 5-12 membered heteroaryl, —CN, —O—C₁-C₁₂ alkyl, —O—(CH₂)₀₋₄C₃-C₁₂ cycloalkyl, —O—(CH₂)₀₋₄C₆-C₁₂ aryl, —O—(CH₂)₀₋₄(3-12 membered heteroalicyclic) and —O—(CH₂)₀₋₄(5 to 12 membered heteroaryl), —C(O)R¹¹⁹, —C(O)OR¹¹⁹ and —C(O)NR¹¹⁹R¹²⁰, and each hydrogen in R¹¹⁷ is optionally substituted by an R¹¹⁸ group;
each R¹¹⁸, which may be the same or different, is independently selected from hydrogen, halogen, C₁-C₁₂ alkyl, C₁-C₁₂ alkoxy, C₃-C₁₂ cycloalkyl, C₆-C₁₂ aryl, 3-12 membered heteroalicyclic, 5-12 membered heteroaryl, —O—C₁-C₁₂ alkyl, —O—(CH₂)₀₋₄C₃-C₁₂ cycloalkyl, —O—(CH₂)₀₋₄C₆-C₁₂ aryl, —O—(CH₂)₀₋₄(3-12 membered heteroalicyclic), —O—(CH₂)₀₋₄(5-12 membered heteroaryl) and —CN, and each hydrogen in R¹⁸ is optionally substituted by a group selected from halogen, —OH, —CN, —C₁-C₁₂alkyl which may be partially or fully halogenated, —O—C₁-C₁₂ alkyl which may be partially or fully halogenated, —CO, —SO, —SO₂ and —SO₃H;
each R¹¹⁹ and R¹²⁰, which may be the same or different, is independently selected from hydrogen, halogen, C₁-C₁₂ alkyl, C₁-C₁₂ alkoxy, C₃-C₁₂ cycloalkyl, C₆-C₁₂ aryl, 3-12 membered heteroalicyclic and 5-12 membered heteroaryl, and each R¹¹⁹ and R¹²⁰ is optionally substituted by a group selected from halogen, —OH, —CN, —C₁-C₁₂ alkyl which may be partially or fully halogenated, —O—C₁-C₁₂ alkyl which may be partially or fully halogenated and SO₃H, or R¹¹⁹ and R¹²⁰, taken together with the nitrogen atom to which they are attached, may form a 3-12 membered heteroalicyclic ring optionally substituted by from 1 to 6 R¹¹⁸ groups;
each D is independently selected from the group consisting of R²⁵⁹, R⁰⁷⁷, R⁷, R¹ and R²¹, wherein
R²⁵⁹ is selected from the group consisting of H, halogen, cyano, nitro, C₁-C₃alkylsulphanyl, —N(OH)H, —N(OH)C₁-C₃alkyl, trifluoromethyl, C₁₋₆alkyl, —NR^{b}R^{c} (wherein R^{b} and R^{c}, which may be the same or different, each represent hydrogen or C₁₋₆alkyl), and —X²R^{d};
wherein X² is selected from the group consisting of a direct bond, —O—, —CH₂—, —OC(O)—, carbonyl, —S—, —SO—, —SO₂—, —NR^{e}C(O)—, —C(O)NR—, —SO₂NR^{g}—, NR^{h}SO₂— and —NRⁱ—;
wherein R^{e}, R^{f}, R^{g}, R^{h} and Rⁱ are independently selected from the group consisting of H, C₁₋₆alkyl, hydroxyC₁₋₄alkyl, and C₁₋₃alkoxyC₂₋₃alkyl; and
R^{d} is selected from the group consisting of H, optionally substituted hydrocarbyl, optionally substituted heterocyclyl and optionally substituted alkoxy;
R⁰⁷⁷ is —O-M⁴-M³-M²-M¹, wherein
M¹ is H, C₁-C₈alkyl-L²⁰²-L²⁰¹- optionally substituted by Y², G²⁰⁰(CH₂)₀₋₃—, and R²⁵³(R²⁵⁴)N(CH₂)₀₋₃—, wherein
G²⁰⁰ is a saturated five- to seven-membered heterocyclyl or heteroaryl containing one or two annular heteroatoms and optionally substituted with between one and three Y² substitutents;
L²⁰¹ is —C(O)— or —SO₂—;
L²⁰² is a direct bond, —O— or —NH—;
R²⁵³ and R²⁵⁴ are independently C₁-C₃alkyl optionally substituted with between one and three Y² substituents;
M² is a saturated or mono- or poly-unsaturated C₃-C₁₄ mono- or fused-polycyclic hydrocarbyl optionally containing one or two or three annular heteroatoms per ring and optionally substituted with between zero and four Y² substituents;
M³ is —NH—, —N(optionally substituted lower alkyl)-, —O—, or absent;
M⁴ is —CH₂—, —CH₂—CH₂—, —CH₂CH₂CH₂—, or absent;
R⁷ is selected from the group consisting of —H, halogen, nitro, azido, C₁-C₆ alkyl, trifluoromethyl, C₃-C₁₀ cycloalkyl, (C₁-C₆)alkoxy, —C(O)NR⁴²R⁴³, —Y—NR⁴²R⁴³, —NR⁴²C(═O)R⁴³, —SO₂R⁴², —SO₂NR⁴²R⁴³, —NR³⁷SO₂R⁴², —NR³⁷SO₂NR⁴²R⁴³, —C(═N—OR⁴²)R⁴³, —C(═NR⁴²)R⁴³, —NR³⁷C(═NR⁴²)R⁴³, —C(═NR⁴²)NR³⁷R⁴³, —NR³⁷C(═NR⁴²)NR³⁷R⁴³, —C(O)R⁴, —CO₂R⁴², —C(O)(heterocyclyl), —C(O)(C₆-C₁₀ aryl), —C(O)(heteroaryl), —Y—(C₆-C₁₀ aryl), —Y-(heteroaryl), —Y-(5-10 membered heterocyclyl), —NR^{6a}R^{6b}, —NR^{6a}SO₂R^{6b}, —NR^{6a}C(O)R^{6b}, —OC(O)R^{6b}, —NR^{6a}C(O)OR^{6b}, —OC(O)NR^{6a}R^{6b}, —OR^{6a}, —SR^{6a}, —S(O)R^{6a}, —SO₂R^{6a}, —SO₃R^{6a}, —SO₂NR^{6a}R^{6b}, —SO₂NR⁴²R⁴³, —COR^{6a}, —CO₂R^{6a}, —CONR^{6a}R^{6b}, —(C₁-C₄)fluoroalkyl, —(C₁-C₄)fluoroalkoxy, —(CZ³Z⁴)ₐCN, wherein a is an integer ranging from 0 to 6, and the aforementioned R⁷ groups other than —H and halogen are optionally substituted, or R⁷ is a moiety selected from the group consisting of —(CZ³Z⁴)ₐ-aryl, —(CZ³Z⁴)ₐ-heterocycle, (C₂-C₆)alkynyl, —(CZ³Z⁴)ₐ-(C₃-C₆)cycloalkyl, —(CZ³Z⁴)ₐ-(C₅-C₆)cycloalkenyl, (C₂-C₆) alkenyl and (C₁-C₆)alkyl, wherein said moiety is optionally substituted with 1 to 3 independently selected Y² groups, where a is 0, 1, 2, or 3, and wherein when a is 2 or 3, the CZ³Z⁴ units may be the same or different; wherein
each R⁴² and R⁴³ is independently selected from the group consisting of H, C₁-C₆ alkyl, —Y⁴—(C₃-C₁₀ cycloalkyl), —Y⁴—(C₆-C₁₀ aryl), —Y⁴—(C₆-C₁₀ heteroaryl), —Y⁴-(5-10 membered heterocyclyl), —Y⁴—O—Y¹—OR³⁷, —Y₁—C₂—R³⁷, and —Y⁴—OR³⁷, wherein the alkyl, cycloalkyl, aryl, heteroaryl and heterocyclyl moieties of the foregoing R⁴² and R⁴³ groups are optionally substituted by 1 or more substituents independently selected from R⁴⁴; or
R⁴² and R⁴³ taken together with the nitrogen to which they are attached form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are optionally substituted by 1 to 5 R⁴⁴ substituents, with the proviso that R⁴² and R⁴³ are not both bonded to the nitrogen directly through an oxygen;
Y is a bond or —(C(R^{y})(H))ₜ—, wherein t is an integer from 1 to 6; and
R^{y} at each occurrence is independently selected from the group consisting of H and C₁-C₆ alkyl, wherein the C₁-C₆ alkyl is optionally substituted;
Y⁴ is a bond or is —(C(R³⁷)(H))ₙ, wherein n is an integer ranging from 1 to 6;
R³⁷ is selected from H, OR³⁶, C₁-C₆ alkyl and C₃-C₁₀ cycloalkyl;
Y¹ is —(C(R³⁷)(H)) i₆;
each R⁴⁴ is independently selected from the group consisting of halo, cyano, nitro, trifluoromethoxy, trifluoromethyl, azido, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —NR³⁶R³⁹, —OR³⁷, —SO₂NR³⁶R³⁹, —SO₂R³⁶, —NR³⁶SO₂R³⁹, —NR³⁶SO₂NR³⁷R⁴¹, C₁-C₆ alkyl, C₂-C₆ alkenyl, C₂-C₆ alkynyl, C₃-C₁₀ cycloalkyl, —C₁-C₆ alkylamino, —(CH₂)ⱼO—(CH₂)ᵢNR³⁶R³⁹, —(CH₂)ₙO—(CH₂)ᵢOR³⁷, —(CH₂)ₙOR³⁷, —S(O)ⱼ(C₁-C₆ alkyl), —(CH₂)ₙ(C₆-C₁₀ aryl), —(CH₂)ₙ(5-10 membered heterocyclyl), —C(O)(CH₂)ₙ(C₆-C₁₀ aryl), —(CH₂)ₙO—(CH₂)j(C₆-C₁₀ aryl), —(CH₂)ₙO—(CH₂)ᵢ(5 to 10 membered heterocyclyl), —C(O)(CH₂)ₙ(5 to 10 membered heterocyclyl), —(CH₂)ⱼNR³⁹(CH₂)ᵢNR³⁶R³⁹, (CH₂)ⱼNR³⁹CH₂C(O)NR³⁶R³⁹—(CH₂)ⱼNR³⁹(CH₂)ᵢNR³⁷C(O)R⁴⁰, (CH₂)ⱼNR³⁹(CH₂)ₙO—(CH₂)ᵢOR³⁷, —(CH₂)ⱼNR³⁹(CH₂)ᵢS(O)ⱼ(C₁-C₆ alkyl), —(CH₂)ⱼNR³⁹(CH₂)ₙR³⁶, —SO₂(CH₂)ₙ(C₆-C₁₀ aryl), and —SO₂(CH₂)ₙ(5 to 10 membered heterocyclyl) wherein, j is an integer from 0 to 2, n is an integer from 0 to 6 and i is an integer ranging from 2 to 6, the —(CH₂)ᵢ— and —(CH₂)ₙ₁— moieties of the foregoing R⁴⁴ groups optionally include a carbon-carbon double or triple bond wherein n is an integer from 2 to 6, and the alkyl, aryl and heterocyclyl moieties of the foregoing R⁴⁴ groups are optionally substituted by 1 or more substituents independently selected from the group consisting of halo, cyano, nitro, trifluoromethyl, azido, —OH, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —(CH₂)ₙNR³⁶R³⁹, —SO₂R³⁶, —SO₂NR³⁶R³⁹, C₁-C₆ alkyl, C₃-C₁₀ cycloalkyl, —(CH₂)ₙ(C₆-C₁₀ aryl), —(CH₂)ₙ(5 to 10 membered heterocyclyl), —(CH₂)ₙO(CH₂)ᵢOR³⁷ and —(CH₂)ₙOR³⁷, wherein n is an integer from 0 to 6 and i is an integer from 2 to 6;
each R³⁶ and R³⁹ is independently selected from the group consisting of H, —OH, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙO(CH₂)ᵢOR³⁷, —(CH₂)ₙCN(CH₂)ₙOR³⁷, —(CH₂)ₙCN(CH₂)ₙR³⁷, and —(CH₂)ₙOR³⁷, wherein n is an integer ranging from 0 to 6 and i is an integer ranging from 2 to 6, and the alkyl, aryl and heterocyclyl moieties of the foregoing R³⁶ and R³⁹ groups are optionally substituted by one or more substituents independently selected from —OH, halo, cyano, nitro, trifluoromethyl, azido, —C(O)R⁴⁰, —C(O)OR⁴⁰, —CO(O)R⁴⁰, —OC(O)OR⁴⁰, —NR³⁷C(O)R⁴¹, —C(O)NR³⁷R⁴¹, —NR³⁷R⁴¹, —C₁-C₆ alkyl, —(CH₂)ₙ(C₆-C₁₀ aryl), —(CH₂)ₙ(5 to 10 membered heterocyclyl), —(CH₂)ₙO(CH₂)ᵢOR³⁷, and —(CH₂)ₙOR³⁷, wherein n is an integer ranging from 0 to 6 and i is an integer ranging from 2 to 6, with the proviso that when R³⁶ and R³⁹ are both attached to the same nitrogen, then R³⁶ and R³⁹ are not both bonded to the nitrogen directly through an oxygen;
each R⁴⁰ is independently selected from H, C₁-C₁₀ alkyl, —(CH₂)ₙ(C₆-C₁₀ aryl), C₃-C₁₀ cycloalkyl, and —(CH₂)ₙ(5-10 membered heterocyclyl), wherein n is an integer ranging from 0 to 6;
each R³⁷ and R⁴¹ is independently selected from H, OR³⁶, C₁-C₆ alkyl and C₃-C₁₀ cycloalkyl;
each R^{6a} and R^{6b} is independently selected from the group consisting of hydrogen and a moiety selected from the group consisting of —(CZ⁵Z⁶)ᵤ-(C₃-C₆)cycloalkyl, —(CZ⁵Z⁶)ᵤ-(C₅-C₆)cycloalkenyl, —(CZ⁵Z⁶)ᵤ-aryl, —(CZ⁵Z⁶)ᵤ-heterocycle, alkoxy, (C₂-C₆)alkenyl, and (C₁-C₆)alkyl, wherein said moiety is optionally substituted with 1 to 3 independently selected Y³ groups, where u is 0, 1, 2, or 3, and wherein when u is 2 or 3, the CZ⁵Z⁶ units may be the same or different, or
R^{6a} and R^{6b} taken together with adjacent atoms form a heterocycle;
each Z³, Z⁴, Z⁵ and Z⁶ is independently selected from the group consisting of H, F and (C₁-C₆)alkyl, or
each Z³ and Z⁴, or Z⁵ and Z⁶ are selected together to form a carbocycle, or
two Z³ groups on adjacent carbon atoms are selected together to optionally form a carbocycle;
each Y² and Y³ is independently selected from the group consisting of H, halogen, trihalomethyl, cyano, nitro, tetrazolyl, guanidino, amidino, methylguanidino, azido, alkoxy, —C(O)Z⁷, —OC(O)NH₂, —OC(O)NHZ⁷, —OC(O)NZ⁷Z⁸, —N(Z⁷)C(O)Z⁷, —N(Z⁷)CO₂Z⁷, —NHC(O)NH₂, —NHC(O)NHZ⁷, —NHC(O)NZ⁷Z⁸, —C(O)OH, —C(O)OZ⁷, —C(O)NH₂, —C(O)NHZ⁷, —C(O)NZ⁷Z⁸, —P(O)₃H₂, —P(O)₃(Z⁷)₂, —S(O)₃H, —SZ⁷, —S(O)Z⁷, —S(O)₂Z⁷, —S(O)₃Z⁷, —S(O)₂—NZ⁷Z⁸, —N(Z⁷)SO₂Z⁸, -Z⁷, —OZ⁷, —OH, —NH₂, —NHZ⁷, —NZ⁷Z⁸, —C(═NR²²⁵)N(Z⁷)Z⁸, —C(═NR²²⁵)Z⁸, C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxy, —(CZ⁹Z¹⁰)ᵣNH₂, —(CZ⁹Z¹⁰)ᵣNHZ³, —(CZ⁹Z¹⁰)ᵣNZ⁷Z⁸, —X⁶(CZ⁹Z¹⁰)ᵣ-(C₃-C₈)cycloalkyl, —X⁶(CZ⁹Z¹⁰)ᵣ-(C₅-C₈)cycloalkenyl, —X⁶(CZ⁹Z¹⁰)ᵣ-aryl and —X⁶(CZ⁹Z¹⁰)ᵣ-heterocycle, each of which is optionally substituted, wherein
R²²⁵ is selected from the group consisting of H, CN, NO₂, —OZ⁷, —S(O)O-2Z⁸, —CO₂Z⁷, optionally substituted lower alkyl, optionally substituted lower alkenyl and optionally substituted lower alkynyl;
r is 1, 2, 3 or 4;
X⁶ is selected from the group consisting of O, S, NH, —C(O)—, —C(O)NH—, —C(O)O—, —S(O)—, —S(O)₂— and —S(O)₃—;
Z⁷ and Z⁸ are independently selected from the group consisting of H, an alkyl of 1 to 12 carbon atoms, an alkenyl of 2 to 12 carbon atoms, an alkynyl of 2 to 12 carbon atoms, a cycloalkyl of 3 to 8 carbon atoms, a heterocycloalkyl of 3 to 8 carbon atoms, a cycloalkenyl of 5 to 8 carbon atoms, an aryl of 6 to 14 carbon atoms, a heterocycle of 5 to 14 ring atoms, an aralkyl of 7 to 15 carbon atoms, and a heteroaralkyl of 5 to 14 ring atoms, each of which is optionally substituted, or
Z⁷ and Z⁸ together may optionally form a heterocycle;
Z⁹ and Z¹⁰ are independently selected from the group consisting of H, F, a (C₁-C₁₂)alkyl, a (C₆-C₁₄)aryl, a (C₅-C₁₄)heteroaryl, a (C₇-C₁₅)aralkyl and a (C₅-C₁₄)heteroaralkyl, or
Z⁹ and Z¹⁰ are taken together form a carbocycle, or
two Z⁹ groups on adjacent carbon atoms are taken together to form a carbocycle; or
any two Y² or Y³ groups attached to adjacent carbon atoms may be taken together to be —O[C(Z⁹)(Z¹⁰)]ᵣO or —O[C(Z⁹)(Z¹⁰)]ᵣ₊₁, or
any two Y² or Y³ groups attached to the same or adjacent carbon atoms may be selected together to form a carbocycle or heterocycle; and wherein
any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C₁-C₄)alkyl, (C₁-C₄)alkoxy and an —N[(C₁-C₄)alkyl][(C₁-C₄)alkyl];
R¹ is —C≡CH or —C≡C—(CR⁴⁵R⁴⁵)ₙ—R⁴⁶; n is an integer from 0 to 6;
each R⁴⁵ is independently selected from the group consisting of H, a (C₁-C₆)alkyl and a (C₃-C₈)cycloalkyl;
R⁴⁶ is selected from the group consisting of heterocyclyl, —N(R⁴⁷)—C(O)—N(R⁴⁷)(R⁴⁸), —N(R⁴⁷)—C(S)—N(R⁴⁷)(R⁴⁸), —N(R⁴⁷)—C(O)—OR⁴⁸, —N(R⁴⁷)—C(O)—(CH₂)ₙ—R⁴⁸, —N(R⁴⁷)—SO₂R⁴⁷, —(CH₂)ₙNR⁴⁷R⁴⁸, —(CH₂)ₙOR⁴⁸, —(CH₂)ₙSR⁴⁹, —(CH₂)ₙS(O)R⁴⁹, —(CH₂)ₙS(O)₂R⁴⁹, —OC(O)R⁴⁹, —OC(O)OR⁴⁹, —C(O)NR⁴⁷R⁴⁸, heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, —(CH₂)ₙOR⁵¹, —(CH₂)ₙC(O)R⁴⁹, —C(O)₂R⁴⁹, (CH₂)ₙSR⁴⁹, —(CH₂)ₙS(O)R⁴⁹, —(CH₂)ₙS(O)₂R⁴⁹, —(CH₂)ₙR⁴⁹, —(CH₂)ₙCN, aryl optionally substituted with one or more substituents selected from the group consisting of halo, —CF₃, (C₁-C₆)alkoxy, —NO₂, (C₁-C₆)alkyl, —CN, —(CH₂)OR⁴⁹, —(CH₂)heterocyclyl, (CH₂)ₙheteroaryl, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, or
R⁴⁷ and R⁴⁸, together with the atom to which they are attached, form a 3-8 membered carbo- or hetero-cyclic ring;
R⁴⁹ is selected from the group consisting of (C₁-C₆)alkyl, (C₃-C₈)cycloalkyl, heterocyclyl(C₁-C₆)alkylene, aryl(C₁-C₆)alkylene wherein the aryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, heteroaryl(C₁-C₆)alkylene wherein the heteroaryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, aryl optionally substituted with one or more substituents selected from the group consisting of halo, —CF₃, (C₁-C₆)alkoxy, —NO₂, (C₁-C₆)alkyl, —CN, —SO₂R⁵¹ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁵⁰ and R⁵¹ are independently selected from the group consisting of H, (C₁-C₆)alkyl, (C₃-C₈)cycloalkyl and —C(O)R⁴⁵, or
R⁵⁰ and R⁵¹, together with the atom to which they are attached, form a 3-8 membered carbo- or hetero-cyclic ring; and
R²¹ is the group defined by -(Z¹¹)-(Z¹²)ₘ-(Z¹³)ₘ₁, wherein
Z¹¹ is heterocyclyl, when m and m1 are 0, or heterocyclylene, when either m or m1 are 1,
Z¹² is selected from the group consisting of OC(O), OC(S) and C(O);
Z¹³ is selected from the group consisting of heterocyclyl, aralkyl, N(H)R⁵², (C₁-C₃)alkyl, —OR⁵², halo, S(O)₂R⁵, (C₁-C₃)hydroxyalkyl and (C₁-C₃)haloalkyl;
m is 0 or 1;
m1 is 0 or 1;
R⁵² is selected from the group consisting of H, —(CH₂)_{q}S(O)₂R⁵⁴, —(C₁-C₆) alkyl-NR⁵³R⁵³ (C₁-C₃)alkyl, —(CH₂)_{q}OR⁵³, —C(O)R⁵⁴ and —C(O)OR⁵³;
q is 0, 1, 2, 3 or 4;
each R⁵³ is independently (C₁-C₃)alkyl;
R⁵⁴ is (C₁-C₃)alkyl or N(H)R⁵³;
R⁵⁶ is selected from the group consisting of NH₂, (C₁-C₃)alkyl and OR⁵²;
V is a 5 to 7 membered cycloalkyl, aryl, heterocylic or heteroaryl ring system, any of which is optionally substituted with 0 to 4 R² groups;
R² at each occurrence is independently selected from the group consisting of R¹⁰⁷, —H, halogen, trihalomethyl, —O-trihalomethyl, —CN, —NO₂, —NH₂, —OR³, —NR³R⁴, —S(O)O₀₋₂R³, S(O)₂NR³R³, —C(O)OR³, —C(O)NR³R³, —N(R³)SO₂R³, —N(R³)C(O)R³, —N(R³)CO₂R³, C(O)R³, C₁-C₄ alkoxy, C₁-C₄ alkylthio, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, alkylamino, —CH₂(CH₂)₀₋₄-T², wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted;
each R³ is independently selected from the group consisting of —H and R⁴;
R⁴ is selected from the group consisting of a (C₁-C₆)alkyl, an aryl, a lower arylalkyl, a heterocyclyl and a lower heterocyclylalkyl, each of which is optionally substituted, or
R³ and R⁴, taken together with a common nitrogen to which they are attached, form an optionally substituted five- to seven-membered heterocyclyl, the optionally substituted five- to seven-membered heterocyclyl optionally containing at least one additional annular heteroatom selected from the group consisting of N, O, S and P;
Z is selected from the group consisting of —O—, —S—, —S(O)—, S(O)₂—, —CH₂—, NBn, —NR⁵—, —OCH₂—, and —N(R⁵)CH₂—, wherein R⁵ is selected from the group consisting of H, C₁-C₆ alkyl, an optionally substituted (C₁-C₅)acyl and C₁-C₆ alkyl-O—C(O), wherein C₁-C₆ alkyl is optionally substituted;
E is selected from the group consisting of —O—, —N(R¹³)—, —N(H)—, —N(C₁-C₆alkyl)-, —CH₂N(H)— and —N(H)CH₂—;
X is selected from the group consisting of O, S, NH, N-alkyl, N—OH, N—O-alkyl, and NCN; [symbol] is a single or double bond;
X¹ is selected from the group consisting of O, S, CH₂, N—CN, N—O-alkyl, NH and N(C₁-C₆alkyl) when [symbol] is a double bond, wherein any alkyl is optionally substituted, or
X¹ is selected from the group consisting of H, halogen, alkyl, alkenyl, alkynyl, CN, alkoxy, NH₂, trihalomethyl, NH(alkyl) and alkyl-thio, when is a single bond, wherein any said alkyl, alkenyl, alkynyl or alkoxy is optionally substituted;
L and L¹ are independently selected from the group consisting of CH, C, N, C(halogen) and C(C₁-C₆alkyl);
   or
L¹ is 0 and W is absent;
L² and L³ are independently selected from the group consisting of CH, CH₂, N, NH, O, S, —C(O)—, —C(S)—, —C(NH)— and —C(N—C₁-C₆alkyl)-;
L⁴ is selected from the group consisting of absent, CH, CH₂, N, NH, O, S, —C(O)—, —C(S)—, —C(NH)— and —C(N—C₁-C₆alkyl)-;
   the group 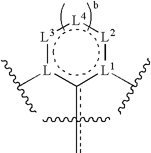
   is aromatic or non-aromatic, provided that two 0 are not adjacent to each other; and wherein any group 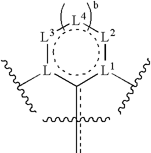
   which comprises a CH₂ group which is attached to 2 carbon atoms or a CH₃ group which is attached to a carbon atom may optionally bear on each said CH₂ or CH₃ group a substituent selected from the group consisting of hydroxy, amino, C₁₋₆alkoxy, N—C₁₋₆alkylamino, N,N—(C₁₋₆alkyl)₂-amino and heterocyclyl;
b is 0-5, preferably 0-4, more preferably 0-1, more preferably 0; and
W is a five- to ten-membered cycloalkyl, aryl, heterocylic or heteroaryl ring system, which is optionally substituted;
R¹³ is selected from the group consisting of H, C₁-C₆alkyl, substituted CN—C₆alkyl, cycloalkyl, substituted cycloalkyl, OH, unsubstituted —O—(C₁-C₆alkyl) and substituted —O—(C₁-C₆alkyl); and
R¹⁴, R¹⁵, R¹⁶ and R¹⁷ are independently selected from the group consisting of —H, halogen, trihalomethyl, —O-trihalomethyl, —CN, —NO₂, —NH₂, —OR³, —OCF₃, —NR³R⁴, —S(O)₀₋₂R³, —S(O)₂NR³R³, —C(O)OR³, —C(O)NR³R³, —N(R³)SO₂R³, —N(R³)C(O)R³, —N(R³)C(O)OR³, —C(O)R³, —C(O)SR³, C₁-C₆ alkoxy, C₁-C₄ alkylthio, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², carboxy, C₁₋₆alkoxycarbonyl, carbamoyl, N—C₁₋₆alkylcarbamoyl, N—(C₁₋₆alkyl)₂-carbamoyl, C₂₋₆alkanoyl, amino, N—C₁₋₆alkylamino, N,N—(C₁₋₆alkyl)₂-amino, an optionally substituted C₁₋₄ alkylcarbonyl, C₁₋₄ alkoxy, an amino optionally substituted by C₁₋₄ alkyl optionally substituted by C₁₋₄ alkoxy and a saturated or unsaturated three- to seven-membered carboxyclic or heterocyclic group, wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted;
   with the proviso that Formula (I) excludes those compounds wherein
   A is 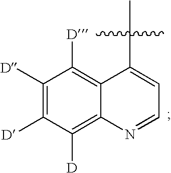
   D, D′, D″ and D′″ are independently selected from R²⁵⁹;
   Z is NH;
   V is an optionally substituted 6-membered aromatic ring containing at least one nitrogen atom,
   E is —N(H)— or —CH₂N(H)—;
   X is O;
   X¹ is selected from the group consisting of halogen, alkyl, alkoxy, amino, alkylamino and one or two oxo, thioxo;
   the group 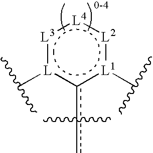
is aryl, heteroaryl or heterocyclyl, optionally substituted with one or more groups selected from halogen and C₁-C₆alkyl, and wherein said heterocyclyl is optionally substituted with one or two oxo or thioxo substituents, and wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituent selected from the group consisting of hydroxy, amino, C₁₋₆alkoxy, N—C₁₋₆alkylamino, N,N—(C₁₋₆alkyl)₂amino and heterocyclyl; and
W is selected from the group consisting of aryl, heteroaryl and heterocyclyl, each optionally substituted with one or more groups selected from hydroxy, halo, C₁₋₆alkyl, C₁₋₆alkoxy, carboxy, C₁₋₆alkoxycarbonyl, carbamoyl, N—C₁₋₆alkylcarbamoyl, N—(C₁₋₆alkyl)₂-carbamoyl, C₂₋₆alkanoyl, amino, N—C₁₋₆alkylamino and N,N—(C₁₋₆alkyl)₂amino, and wherein said heterocyclyl is optionally substituted with one or two oxo or thioxo substituents, wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituents selected from hydroxyl, amino, C₁₋₆alkoxy, N—C1-6alkylamino, N,N—(C₁₋₆alkyl)₂amino and heterocyclyl;
   with the proviso that Formula (I) excludes those compounds wherein
   A is 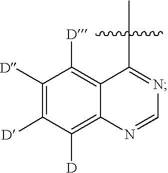
   D, D′, D″ and D′″ are independently selected from R²⁵⁹;
   Z is selected from the group consisting of O, S, S(O), S(O)₂, NH and N(C₁₋₆alkyl);
   V is a group selected from: 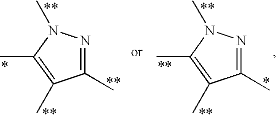
   wherein * indicates the point of attachment to the group Z of Formula I and ** indicates optional points of attachment to the group E of Formula I;
   E is —N(H)— or —CH₂N(H)—;
   X is O or NCN;
   X¹ is selected from the group consisting of halogen, alkyl, alkoxy, amino, alkylamino and one or two oxo, thioxo;
   the group 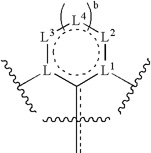
is aryl, heteroaryl or heterocyclyl, optionally substituted with one or more groups selected from halogen and C₁-C₆alkyl, and wherein said heterocyclyl is optionally substituted with one or two oxo or thioxo substituents, and wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituent selected from the group consisting of hydroxy, amino, C₁₋₆alkoxy, N—C₁₋₆alkylamino, N,N—(C₁₋₆alkyl)₂amino and heterocyclyl; and
W is selected from the group consisting of aryl, heteroaryl and heterocyclyl, each optionally substituted with one or more groups selected from hydroxy, halo, C₁₋₆alkyl, C₁₋₆alkoxy, carboxy, C₁₋₆alkoxycarbonyl, carbamoyl, N—C₁₋₆alkylcarbamoyl, N—(C₁₋₆alkyl)₂carbamoyl, C₂₋₆alkanoyl, amino, N—C₁₋₆alkylamino and N,N—(C₁₋₆alkyl)₂amino, and wherein said heterocyclyl is optionally substituted with one or two oxo or thioxo substituents, wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituents selected from hydroxyl, amino, C₁₋₆alkoxy, N—C1-6alkylamino, N,N—(C₁₋₆alkyl)₂amino and heterocycly;
   with the proviso that Formula (I) excludes those compounds wherein
   A is 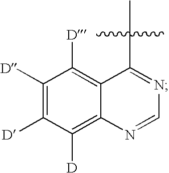
   D, D′, D″ and D′″ are independently selected from R²⁵⁹;
   Z is selected from the group consisting of O, S, S(O), S(O)₂, NH and N(C₁₋₆alkyl);
   V is an optionally substituted 5-membered heteroaromatic ring;
   E is —N(H)— or —CH₂N(H)—;
   X is O or NCN;
   the group 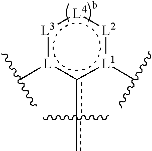
is aryl, heteroaryl or heterocyclyl, optionally substituted with one or more groups selected from halogen and C₁-C₆alkyl, and wherein said heterocyclyl is optionally substituted with one or two oxo or thioxo substituents, and wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituent selected from the group consisting of hydroxy, amino, C₁₋₆alkoxy, N—C₁₋₆alkylamino, N,N—(C₁₋₆alkyl)₂amino and heterocyclyl; and
W is selected from the group consisting of aryl, heteroaryl and heterocyclyl, each optionally substituted with one or more groups selected from hydroxy, halo, C₁₋₆alkyl, C₁₋₆alkoxy, carboxy, C₁₋₆alkoxycarbonyl, carbamoyl, N—C₁₋₆alkylcarbamoyl, N—(C₁₋₆alkyl)₂carbamoyl, C₂₋₆alkanoyl, amino, N—C₁₋₆alkylamino and N,N—(C₁₋₆alkyl)₂amino, and wherein said heterocyclyl is optionally substituted with one or two oxo or thioxo substituents, wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituents selected from hydroxyl, amino, C₁₋₆alkoxy, N—C1-6alkylamino, N,N—(C₁₋₆alkyl)₂amino and heterocyclyl;
   with the proviso that Formula (I) excludes those compounds wherein
   A is 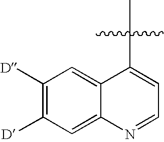
   D′ and D″ are each independently OH or —O—C₁-C₅ alkyl, wherein D′ and D″ optionally together form C₁-C₃alkylene;
   Z is selected from the group consisting of O and S;
   V is a phenyl group;
   E is —N(H)— or —N(C₁-C₄alkyl)-;
   X is selected from the group consisting of O, S, NH, NCN, N(C₁-C₅alkyl) and N—O—C₁-C₅alkyl; the group 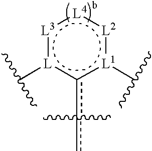
is C₃-C₁₀cycloalkyl, phenyl, furyl, thienyl, 5- or 6-membered heteroaryl having 1 or 2 nitrogen atoms and optionally having another heteroatom selected from the group consisting of nitrogen, oxygen and sulfur atoms, wherein each of said C₃-C₁₀cycloalkyl, phenyl, furyl, thienyl and 5- or 6-membered heteroaryl is optionally substituted by halogen or C₁-C₅alkyl; and
W is selected from the group consisting of C₃-C₁₀cycloalkyl, phenyl, substituted phenyl, phenoxy, substituted phenoxy, phenylthio, substituted phenylthio, phenyl(C₁-Calkyl), substituted phenyl(C₁-C₄alkyl), pyridyl, pyrazinyl, pryimidinyl, pyridazinyl, pyrrolidinyl, piperidinyl, piperazinyl, homopyperazinyl, morpholinyl, quinolyl, quinazolinyl, benzoyl and substituted benzoly;
   with the proviso that Formula (I) excludes those compounds wherein
   A is 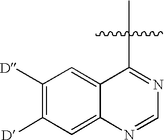
   D′ and D″ are each independently OH or —O—C₁-C₅ alkyl, wherein D′ and D″ optionally together form C₁-C₃alkylene;
   Z is selected from the group consisting of O, S and CH₂;
   V is a phenyl group;
   E is —N(H)— or —N(C₁-C₄alkyl)-;
   X is selected from the group consisting of O, S, NH, NCN, N(C₁-C₅alkyl) and N—O—C₁-C₅alkyl;
   the group 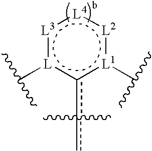
is C₃-C₁₀cycloalkyl, phenyl, furyl, thienyl, 5- or 6-membered heteroaryl having 1 or 2 nitrogen atoms and optionally having another heteroatom selected from the group consisting of nitrogen, oxygen and sulfur atoms, wherein each of said C₃-C₁₀cycloalkyl, phenyl, furyl, thienyl and 5- or 6-membered heteroaryl is optionally substituted by halogen or C₁-C₅alkyl; and
W is selected from the group consisting of C₃-C₁₀cycloalkyl, phenyl, substituted phenyl, phenoxy, substituted phenoxy, phenylthio, substituted phenylthio, phenyl(C1-Calkyl), substituted phenyl(C₁-C₄alkyl), pyridyl, pyrazinyl, pryimidinyl, pyridazinyl, pyrrolidinyl, piperidinyl, piperazinyl, homopyperazinyl, morpholinyl, quinolyl, quinazolinyl, benzoyl and substituted benzoly;
   with the proviso that Formula (I) excludes those compounds wherein
   A is 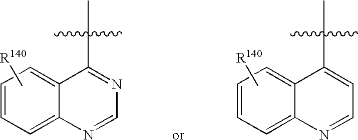
   R¹⁴⁰ is selected from the group consisting of H, halogen, —OR^{140a}, —NO₂, —NH₂, —NR^{140a}R^{104b}, and optionally substituted lower alkyl, alkenyl, alkynyl or cycloalkyl, wherein
   R^{140a} is H or R^{140b},
   R^{140b} is selected from the group consisting of optionally substituted lower alkyl, alkenyl, alkynyl or cycloalkyl, optionally substituted aryl, optionally substituted lower arylalkyl, optionally substituted heterocyclyl, optionally substituted heteroaryl, optionally substituted heterocyclylalkyl and optionally substituted lower heteroarylalkyl, or
   R^{140a} and R^{140b}, when taken together with a common nitrogen to which they are attached, form an optionally substituted five- to seven-membered heterocyclyl or optionally substituted five- to seven-membered heteroaryl, said optionally substituted five- to seven-membered heterocyclyl or optionally substituted five- to seven-membered heteroaryl optionally containing at least one additional annular heteroatom selected from N, O and S;
   Z is selected from the group consisting of —S(O)₀₋₂, —O— and —N(H)— and —N(lower alkyl, alkenyl, alkynyl or cycloalkyl)-;
   V is selected from the group consisting of 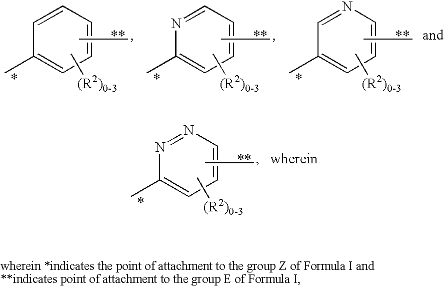
   R² is selected from the group consisting of H, halogen, trihalomethyl, —CN, —NO₂, —NH₂, —OR^{140a}, —NR^{140a}R^{140b}, —S(O)₀₋₂R^{140a}, —SO₂NR^{140a}R^{140b}, —CO₂R^{140a}, —C(O)NR^{140a}R^{140b}, —N(R^{140a})SO₂R^{140a}, —N(R^{140a})C(O)R^{140a}, —N(R^{140a})CO₂R^{140a}, —C(O)R^{140a} and optionally substituted lower alkyl, alkenyl, alkynyl or cycloalkyl;
   E is selected from the group consisting of —N(H)—, —N(lower alkyl, alkenyl, alkynyl or cycloalkyl)-;
   X is O;
   the group 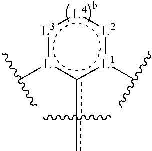
is an optionally substituted five- to seven-membered heterocyclyl or heteroaryl, said optionally substituted five- to seven-membered heterocyclyl or heteroaryl optionally containing at least one additional annular heteroatom selected from N, O, S; and
X¹ is O, S and CH₂;
   with the proviso that Formula (I) excludes those compounds wherein 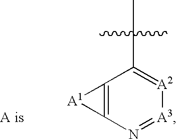
   wherein
   wherein each hydrogen in A¹ is optionally substituted by an R¹⁶⁰ group, wherein
R¹⁰⁶ is selected from the group consisting of halogen, C₁-C₁₂alkyl, C₂-C₁₂alkenyl, C₂-C₁₂alkynyl, C₃-C₁₂cycloalkyl, C₆-C₁₂aryl, 3-12 membered heteroalicyclic, 5-12 membered heteroaryl, —S(O)₀₋₂R¹⁰⁸, —SO₂NR¹⁰⁸R¹⁰⁹, —S(O)₂OR¹⁰⁸, —NO₂, —NR¹⁰⁸R¹⁰⁹, —(CR¹¹⁰R¹¹¹)₀₋₄R¹⁰⁸, —CN, —C(O)R¹⁰⁸, —OC(O)R¹⁰⁸, —O(CR¹¹⁰R¹¹¹)₀₋₄R¹⁰⁸, —NR¹⁰⁸C(O)R¹⁰⁹, —(CR¹¹R¹¹¹)₀₋₄C(O)OR¹⁰⁸, —(CR¹¹⁰R¹¹¹)₀₋₄NR¹⁰⁸R¹⁰⁹, C(═NR¹¹⁰)NR¹⁰⁸R¹⁰⁹, —NR¹⁰⁸C(O)NR¹⁰⁹R¹¹⁰, —NR¹⁰⁸S(O)₁₋₂R¹⁰⁹ and —C(O)NR¹⁰⁸R¹⁰⁹, and each hydrogen in R¹⁰⁶ is optionally substituted by an R¹¹⁷ group;
   Z is O; 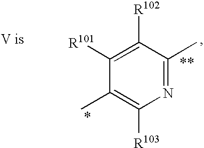
      wherein * indicates the point of attachment to the group Z of Formula I and ** indicates point of attachment to the group E of Formula I,
      R^{10l}, R¹⁰² and R¹⁰³, which may be the same or different, are each independently selected from R¹⁰⁷;
      R¹⁰⁷;
      E is —N(H)—;
      X is O or S;
      X¹ is selected from the group consisting of alkyl, alkenyl, alkynyl, CN, alkoxy and halogen; and
      R¹⁴, R¹⁵, R¹⁶ and R¹⁷ are independently selected from the group consisting of R¹¹⁸; with the proviso that Formula (I) excludes those compounds wherein 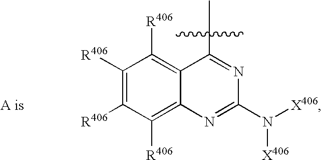
      wherein
each R⁴⁰⁶ is independently selected from the group consisting of hydrogen, —F, —Cl, —Br, —I, —OH, —SH, —NO₂, —CN, —OR^{406r}, —SR^{406d}, —S(O)R^{406d}, —S(O)₂R^{406d}, —NR^{406b}R^{406c}, —C(O)R^{406a}, —C(O)OR^{406e} and an optionally substituted aliphatic, alicyclic, heteroaliphatic, heterocyclic, aromatic, heteroaromatic or acyl moiety, and any two R⁴⁰⁶, together with the carbons to which they are bound, may represent a fused 5-9 membered alicyclic, heterocyclic, aromatic or heteroaromatic ring;
R^{406r} is selected from the group consisting of an optionally substituted aliphatic, alicyclic heteroaliphatic, heterocyclic, aromatic, heteroaromatic or acyl moiety;
R^{406a} is selected from the group consisting of hydrogen or an optionally substituted aliphatic, alicyclic, heteroaliphatic, heterocyclic, aromatic or heteroaromatic moiety;
R^{406b} is selected from the group consisting of hydrogen, —OH, —SO₂R^{406d}, or an optionally substituted aliphatic, alicyclic, heteroaliphatic, heterocyclic, aromatic, heteroaromatic or acyl moiety;
R^{406c} is selected from the group consisting of hydrogen, —OH, —SO₂R⁴, or an optionally substituted aliphatic, alicyclic, heteroaliphatic, heterocyclic, aromatic, heteroaromatic or acyl moiety;
R^{406d} is selected from the group consisting of hydrogen, —N(R^{406e})₂, or an optionally substituted aliphatic, alicyclic, heteroaliphatic, heterocyclic, aromatic or heteroaromatic moiety; and
R^{406e} is hydrogen or an optionally substituted aliphatic moiety;
Z is NH, N (optionally substituted Bn), N (optionally substituted alkyl) or N (optionally substituted acyl);
E is —N(H)— or —N(optionally substituted alkyl);
X is O or S; and
X¹ is selected from the group consisting of alkyl, alkenyl, alkynyl, alkoxy, halogen, cyano, trifluoromethyl, and alkyl-thio;
   with the proviso that Formula (I) excludes those compounds wherein
   A-Z-V- is 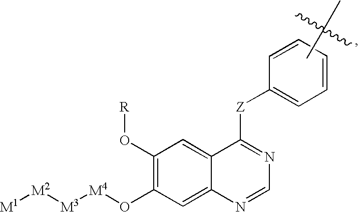
   wherein
M¹, M², M³ and M⁴ are as defined above;
R is C₁-C₃alkyl optionally substituted with between one and three groups independently selected from the group consisting of H, halogen, trihalomethyl, —OZ⁷, —NZ⁷Z⁸, —SZ⁷, —S(O)Z⁷, —S(O)₂Z, —S(O)₂—NZ⁷Z⁸, —C(O)OZ⁷, —C(O)NZ⁷Z⁸, —C(═NR²²⁵)N(Z⁷)Z⁸, —C(═NR²²⁵)Z⁸, N(Z⁷)SO₂Z⁸, —N(Z⁷)C(O)Z⁷, —N(Z⁷)CO₂Z⁷, —C(O)Z⁷, optionally substituted alkoxy, optionally substituted lower alkyl, optionally substituted aryl, optionally substituted lowe arylalkyl, optionally substituted heterocyclyl, and optionally substituted lower heterocyclylalkyl; or each of said groups, when taken together on the same carbon are oxo; or two of said groups, when taken together with a common carbon to which they are attached, form an optionally substituted three- to seven-membered spirocyclyl, said optionally substituted three- to seven-membered spirocyclyl containing at least one additional heteroatom selected from N, O, S and P;
Z is selected from the group consisting of —OCH₂—, —O—, —S(O)₀₋₂—, —N(H)CH₂—, —N(C₁-C₆ alkyl)CH₂—, —NH— and —N(C₁-C₆ alkyl)-;
R¹³ is selected from the group consisting of H, optionally substituted C₁-C₆alkyl;
X is O; and
   the group 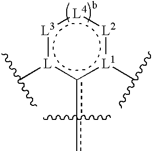
   is an optionally substituted aryl, an optionally substituted lower arylalkyl, optionally substituted heterocyclyl and an optionally substituted lower heterocyclylalkyl; with the proviso that Formula (I) excludes those compounds wherein
   A-Z-V- is 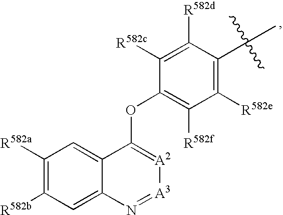
   wherein
   A² and A³ are independently selected from CH and N;
   R^{582a} and R^{582b} are independently selected from the group consisting of H and C₁-C₄alkoxy optionally substituted by a halogen atom;
   R^{582c}R^{582d}, R^{582e} and R^{582f} are independently selected from the group consisting of H, halogen, C₁-C₄alkyl optionally substituted by a halogen atom, C₁-C₄alkoxy optionally substituted by a halogen atom, nitro, amino and morpholyl;
   R¹³ is selected from the group consisting of H and C₁-C₄alkyl optionally substituted by a halogen atom;
   X is O; and
   the group 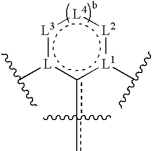
   is selected from the group consisting of phenyl optionally substituted by phenyl optionally substituted by a halogen atom.

In a preferred embodiment of the present invention, the invention provides compounds of formula (I-A) and racemic mixtures, diastereomers and enantiomers thereof: 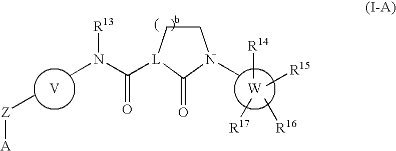
and N-oxides, hydrates, solvates, pharmaceutically acceptable salts, prodrugs and complexes thereof, wherein A, Z, V, W, b, R¹³, R¹⁴, R¹⁵, R¹⁶ and R¹⁷ are as defined in Formula (I), and L is selected from the group consisting of CH, N, C(halogen), C(C≡CH), C(C≡N) and C(NO₂).

According to a preferred embodiment of the present invention, A is selected from the group consisting of

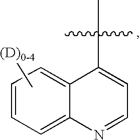

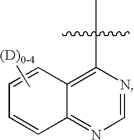

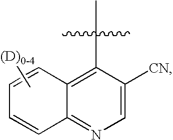

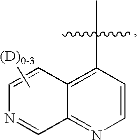

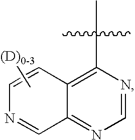

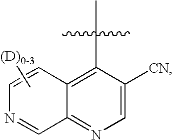

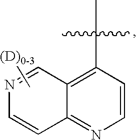

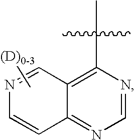

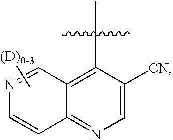

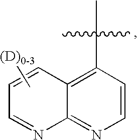

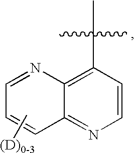

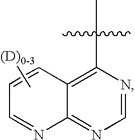

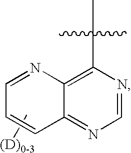

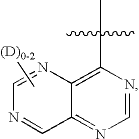

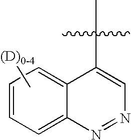

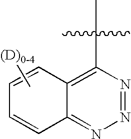

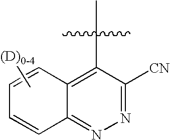

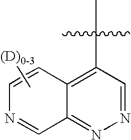

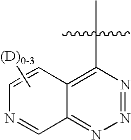

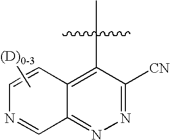

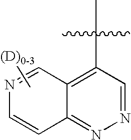

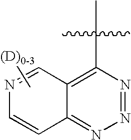

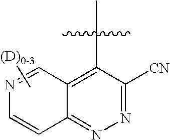

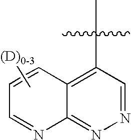

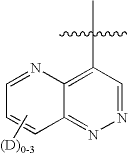

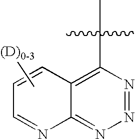

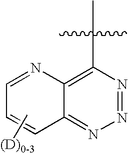

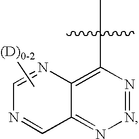

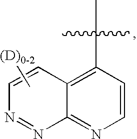

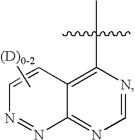

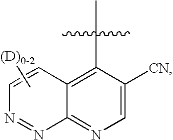

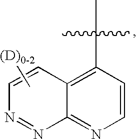

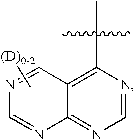

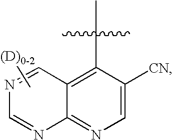

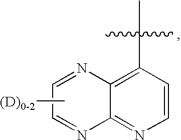

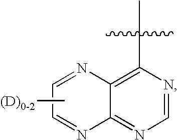

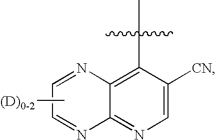

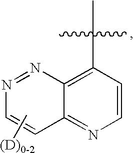

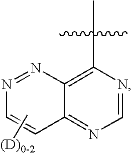

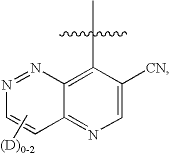

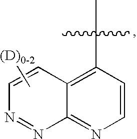

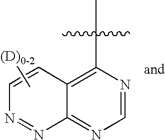

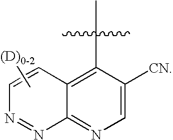

According to a preferred embodiment of the present invention, A is selected from the group consisting of

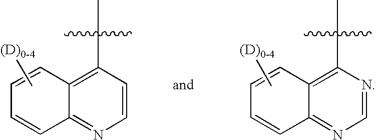

According to another preferred embodiment of the present invention, A is 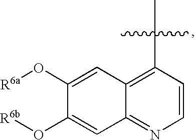
wherein R^{6a} and R^{6b} are as defined above.

According to another preferred embodiment of the present invention, A is 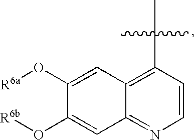
wherein R^{6a} and R^{6b} are independently selected from (C₁-C₆)alkyl and —(CH₂)₀₋₃-heterocycle.

According to another preferred embodiment of the present invention, A is 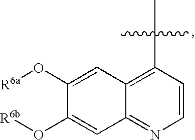
wherein R^{6a} and R^{6b} are independent (C₁-C₆)alkyl groups. According to another preferred embodiment, each D is independently selected from R²⁵⁹, R⁰⁷⁷ and R⁷.

According to another preferred embodiment, each D is independently selected from R⁷.

According to another preferred embodiment, each R²⁵⁹ is independently selected from —X²—R^{d}.

According to another preferred embodiment, X² is O.

According to another preferred embodiment of the present invention A is substituted by 0, 1 or 2 D, preferably 1 or 2 D, more preferably 2 D.

According to another preferred embodiment of the present invention at least one D is —O-M⁴-M³-M²-M¹.

According to another preferred embodiment of the present invention at least one D is H.

According to another preferred embodiment of the present invention, at least one group of D, comprises a chain of at least 3 and preferably at least 4 optionally substituted carbon atoms or heteroatoms such as oxygen, nitrogen or sulphur. Most preferably the chain is substituted by a polar group which assists in solubility.

According to another preferred embodiment of the present invention at least one D is a group X²R^{d}. Preferably in this case, X² is oxygen and R^{d} is selected from group R^{d}(1) or R^{d}(2) below. Particular R^{d} groups are those in group R^{d}(1) below, particularly alkyl, such as methyl, or halogen substituted alkyl, or those in group R^{d}(10) below. In one preferred embodiment, at least one of D is a group —OC₁₋₆alkylR^{dd} and R^{dd} is a heterocyclic ring such as an N-linked morpholine ring such as 3-morpholinopropoxy. In another preferred embodiment, one of D is a group —OC₁₋₆alkylR^{dd} and R^{dd} is a heterocyclic ring such as an N-linked morpholine ring such as 3-morpholinopropoxy.

According to another preferred embodiment of the present invention, each D is independently selected from the group consisting of halogen, cyano, nitro, trifluoromethyl, C₁₋₆alkyl, —NR^{d}R^{e}, wherein R^{d} and R^{e} which may be the same or different, each represents hydrogen or C₁₋₆alkyl), or a group —X²R^{f}. Preferred examples of —X²R^{f} for D include those listed below.

According to another preferred embodiment of the present invention, D is present two times and each are independently selected from methoxy and 3,3,3-trifluoroethoxy.

According to another preferred embodiment of the present invention, each D is independently defined by the group R⁷, wherein R⁷ is selected from the group consisting of —H, halogen, C₁-C₆ alkyl, C₃-C₁₀ cycloalkyl, —C(O)NR⁴²R⁴³, —C(O)(C₆-C₁₀ aryl), —C(O)(heterocyclyl), —C(O)(heteroaryl), —Y—(C₆-C₁₀ aryl), —Y-(5-10 membered heterocyclyl), —Y-(heteroaryl), —S-aryl, —S—C₁-C₆ alkyl, —SO—C₁-C₆ alkyl, —SO₂—C₁-C₆ alkyl, —Y—NR⁴²R⁴³, —SO₂NR⁴²R⁴³, —OR^{6a} and —C(O)OR^{6a}, wherein the aforementioned R⁷ groups other than —H and halogen are optionally substituted.

According to another preferred embodiment of the present invention, each D is independently defined by the group R⁷, wherein R⁷ is selected from the group consisting of —H, —C(O)NR⁴²R⁴³, —Y-(5 to 10 membered heterocyclyl), —Y—(C₆-C₁₀ aryl), —Y-(heteroaryl), —Y—NR⁴²R⁴³, —SO₂NR⁴²R⁴³, —OR^{6a} and —C(O)OR⁴², wherein the aforementioned R⁷ groups other than —H are optionally substituted.

According to another preferred embodiment of the present invention, R^{d} is hydrogen or an alkyl group, optionally substituted with one or more groups selected from functional group, alkenyl, alkynyl, aryl, heterocyclyl, cycloalkyl, cycloalkenyl or cycloalkynyl, any of which may be substituted with a functional group, and where any aryl, heterocyclyl, cycloalkyl, cycloalkenyl, cycloalkynyl groups may also be optionally substituted with hydrocarbyl such as alkyl, alkenyl or alkynyl.

According to another preferred embodiment, R^{d} is an optionally substituted alkoxy.

According to another preferred embodiment of the present invention, R^{d} is selected from one of the following groups:
1) hydrogen or C₁₋₆alkyl which may be unsubstituted or which may be substituted with one or more functional groups, preferably selected from hydroxyl, fluoro and amino,
2) C₁₋₈alkylX³COR^{j}, preferably C₁₋₅alklylX³COR^{j}, wherein said C₁₋₈alkyl or C₁₋₅alky moieties are optionally substituted by one or more functional groups, X³ is —O— or —NR^{k}, (in which R^{k} is selected from the group consisting of H, C₁₋₆alkyl, optionally substituted with a function group, and C₁₋₃alkoxyC₂₋₃alkyl) and R^{j} is selected from the group consisting of C₁₋₆alkyl, —NR^{L}R^{m} and —ORⁿ (wherein R^{L}, R^{m} and Rⁿ, which may be the same or different, are selected from the group consisting of H, alkyl optionally substituted with a functional group and C₁₋₃alkoxyC₂₋₃alkyl);
3) C₁₋₈alkylX⁴R^{o}, preferably C₁₋₅alkylX⁴R^{O}, wherein said C₁₋₈alkyl or C₁₋₅alky moieties are optionally substituted by one or more functional groups (wherein X⁴ is selected from the group consisting of —O—, —S—, —SO—, —SO₂—, —OCO—, —NR^{p}CO—, —CONR^{q}—, —SO₂NR^{s}, —NR^{t}SO₂, and —NR^{u} (wherein R^{p}, R^{q}, R^{s}, R^{t} and R^{u} are independently selected from the group consisting of H, alkyl optionally substituted with a functional group and C₁₋₃alkoxyC₂₋₃alkyl) and Ro is selected from the group consisting of H, hydrocarbyl and a saturated heterocyclic group, wherein the hydroxycarbyl or heterocyclic groups may be optionally substituted by one or more functional groups and the heterocyclic groups may additionally be substituted by a hydrocarbyl group;
4) C₁₋₈alkylX⁵C₁₋₈alkylX 7R^{v}, preferably C₁₋₅alkylX⁵C₁₋₅alkyX⁷R^{v}, wherein said C₁₋₈alkyl or C₁₋₅alkyl moieties are optionally substituted by one or more functional groups (wherein X⁵ and X⁷ which may be the same or different are each selected from the group consisting of —O—, —S—, —SO—, —SO₂—, —NR^{w}CO—, —CONR^{x}—, —SO₂NR^{z}—, —NRₐₐSO₂— and —NR^{bb}— (wherein R^{w}, R^{x}, R^{z}, R^{aa} and R^{bb} are each independently selected from the group consisting of H, alkyl optionally substituted with a functional group and C₁₋₃alkoxyC₂₋₃alkyl) and R^{v} is H or alkyl optionally substituted with a functional group);
5) R^{cc} (wherein R^{cc} is a C₃₋₆cycloalkyl or saturated heterocyclic group (linked via carbon or nitrogen) which cycloalkyl or heterocyclic group may be substituted with one or more functional groups or by a hydrocarbyl or heterocyclyl group which hydrocarbyl or heterocyclyl group may be optionally substituted by one or more functional groups;
6) C₁₋₈alkylR^{cc}, preferably C₁₋₅alkylR^{cc}, wherein said C₁₋₈alkyl or C₁₋₅alkyl moieties are optionally substituted by one or more functional groups;
7) C₂₋₈alkenylR^{cc}, preferably C₂₋₅alkenylR^{cc}, wherein said C₂₋₈alkenyl or C₂₋₅alkenyl moieties are optionally substituted by one or more functional groups;
8) C₂₋₈alkynylR^{cc}, preferably C₂₋₅alkynylR^{cc}, wherein said C₂₋₈alkynyl or C₂₋₅alkynyl moieties are optionally substituted by one or more functional groups;
9) R^{dd} (wherein R^{dd} is selected from the group consisting of a pyridone group, an aryl group, and an aromatic heterocyclic group (linked via carbon or nitrogen) with 1-3 heteroatoms selected from the group consisting of O, N and S, which pyridone, aryl or aromatic hetoercyclic group may be substituted with one or more functional groups or by a hydrocarbyl group optionally substituted by one or more functional groups or heterocyclyl groups, or by a heterocyclyl group optionally substituted with one or more functional groups or hydroxcarbyl groups;
10) C₁₋₈alkylR^{dd}, preferably C₁₋₅alkylR^{dd}, wherein said C₁₋₈alkyl or C₁₋₅alkyl moieties are optionally substituted by one or more functional groups;
11) C₂₋₈alkenylR^{dd}, preferably C₂₋₅alkenylR^{dd}, wherein said C₂₋₈alkenyl or C₂₋₅alkenyl moieties are optionally substituted by one or more functional groups;
12) C₂₋₅alkynylR^{dd}, preferably C₂₋₅alkynylR^{dd}, wherein said C₂₋₈alkynyl or C₂₋₅alkynyl moieties are optionally substituted by one or more functional groups;
13) C₁₋₈alkylX⁸R^{dd}, preferably C₁₋₅alkylX⁸R^{dd}, (wherein X⁸ is selected from the group consisting of —O—, —S—, —SO—, —SO₂—, —NRⁱⁱCO—, —CONR^{jj}—, —SO₂NR^{kk}—, —NR^{LL}SO₂— and —NR^{rr}— (wherein Rⁱⁱ, R^{jj}, R^{kk}, R^{LL} and R^{mm} are each independently selected from the group consisting of H, alkyl optionally substituted with a functional group and C₁₋₃alkoxyC₂₋₃alkyl);
14) C₂₋₈alkenylX R^{dd}, preferably C₂₋₅alkenylX⁹R^{dd}, (wherein X⁹ is selected from the group consisting of —O—, —S—, —SO—, —SO₂—, —NRⁿⁿCO—, —CONR^{oo}—, —SO₂NR^{pp}—, —NR^{qq}S₂— and —NR^{rr}— (wherein Rⁿⁿ, R^{oo}, R^{pp}, R^{qq} and R^{rr} are independently selected from the group consisting of H, alkyl optionally substituted with a functional group and C₁₋₃alkoxyC₂₋₃alkyl);
15) C₂₋₈alkynylX¹⁰R^{dd}, preferably C₂₋₅alkynylX¹⁰R^{dd}, (wherein X¹⁰ is selected from the group consisting of —O—, —S—, —SO—, —SO₂—, —NR^{ss}CO—, —CONR^{tt}—, —SO₂NR^{uu}—, —NR^{vv}SO₂— and and NR^{ww} (wherein R^{ss}, R^{tt}, R^{uu}, R^{vv} and R^{ww} are independently selected from the group consisting of H, alkyl optionally substituted with a functional group and C₁₋₃alkoxyC₂₋₃alkyl);
16) C₁₋₈alkylX C₁₋₈alkylR^{dd}, preferably C₁₋₃alkylX C₁₋₃alkylR^{dd}, (wherein X¹¹ is selected from the group consisting of —O—, —S—, —SO—, —SO₂—, —NR^{xx}CO—, —CONR^{yy}—, —SO₂NR^{zz}—, —NR^{aaa}SO₂— and —NR^{bbb}— (wherein R^{xx}R^{yy}, R^{zz}, R^{aaa} and R^{bbb} are independently selected from the group consisting of H, alkyl optionally substituted with a functional group and C₁₋₃alkoxyC₂₋₃alkyl);
17) C₁₋₈alkylX¹¹C₁₋₈alkylR^{cc}, preferably C₁₋₃alkylX¹¹C₁₋₃alkylRCC;
18) C₂₋₅alkenyl which may be unsubstituted or which may be substituted with one or more functional groups;
19) C₂₋₅alkynyl which may be unsubstituted or which may be substituted with one or more functional groups;
20) —C₁₋₈alkylX¹¹C₁₋₈alkylR^{cc}, preferably —C₁₋₅alkylX¹¹C₁₋₅alkylR^{cc};
21) —C₁₋₈alkynylX¹¹C₁₋₈alkylR^{cc}, preferably —C₁₋₅alkynylX¹¹C₁₋₅alkylR^{cc}; and
22) —C₁-alkylR^{ggg}(C₁₋₈alkyl)₀₋₁(X¹¹)₀₋₁R^{hhh} wherein R^{ggg} is a C₁₋₃alkylene group or a cyclic group selected from divalent cycloalkyl or heterocyclic group, which C₁₋₃alkylene group may be substituted by one or more functional groups and which cyclic group may be substituted by one or more functional groups or by a hydrocarbyl group optionally substituted by one or more functional groups or heterocyclyl groups, or by a heterocyclyl group optionally substituted by one or more functional groups or hydrocarbyl groups; and R^{hhh} is selected from the group consisting of H, C₁₋₃alkyl, and a cyclic group selected from cycloalkyl or heterocyclic group, which C₁₋₃alkylene group may be substituted by one or more functional groups and which cyclic group may be substituted by one or more functional groups or by a hydrocarbyl group optionally substituted by one or more functional groups or heterocyclyl groups, or by a heterocyclyl group optionally substituted by one or more functional groups or hydrocarbyl groups;

According to a preferred embodiment, R⁷ is C₁-C₆alkoxy or —OR^{6a}.

According to another preferred embodiment of the present invention, R⁷ is selected from the group consisting of —(CH₂)ₙ(5 to 10 membered heterocyclyl), —C(O)NR⁴²R⁴³, —SO₂NR⁴²R⁴³, —OR^{6a} and —CO₂R⁴², wherein said R⁷ group —(CH₂)ₙ(5 to 10 membered heterocyclyl) is optionally substituted.

According to another preferred embodiment of the present invention, R⁷ is selected from the group consisting of —(CH₂)ₙ(5 to 10 membered heterocyclyl), OR^{6a} and —C(O)NR⁴²R⁴³.

According to another preferred embodiment of the present invention, R⁷is —C(O)NR⁴²R⁴³, wherein R⁴² and R⁴³ are independently selected from H, (C₁-C₆)alkyl, (C₃-C₁₀)cycloalkyl, —(CH₂)ₙ(C₃-C₁₀ cycloalkyl), —(CH₂)ₙ(C₆-C₁₀ aryl), —(CH₂)ₙ(5 to 10 membered heterocyclyl), —(CH₂)ₙO(CH₂)ᵢOR³⁷—, —(CH₂)ₙOR³⁷, wherein n is an integer from 0 to 6, i is an integer from 2 to 6, and the alkyl, aryl and heterocyclyl moieties of said R⁴² and R⁴³ groups are unsubstituted or substituted with one or more substituents independently selected from R³⁸, or R⁴² and R⁴³ are taken together with the nitrogen to which they are attached to form a C₅-C₈ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₈ azabicyclic, aziridinyl, azetidinyl, pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents, where R⁴² and R⁴³ are not both bonded to the nitrogen directly through an oxygen.

According to another preferred embodiment of the present invention, R⁷ is —C(O)NR⁴²R⁴³, wherein R⁴² and R⁴³ are taken together with the nitrogen to which they are attached to form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉₈ azabicyclic, aziridinyl, azetidinyl, pyrrolidinyl, piperidinyl, piperazinyl, morpholinyl, thiomorpholinyl, isoquinolinyl, or dihydroisoquinolinyl ring are optionally substituted.

According to another preferred embodiment of the present invention, R⁷ is —C(O)NR⁴²R⁴³, wherein R⁴² and R⁴³ are taken together with the nitrogen to which they are attached to form a pyrrolidinyl, piperidinyl, piperazinyl, morpholinyl, thiomorpholinyl, isoquinolinyl, or dihydroisoquinolinyl ring, wherein said pyrrolidinyl, piperidinyl, piperazinyl, morpholinyl, thiomorpholinyl, isoquinolinyl, or dihydroisoquinolinyl ring are optionally substituted.

According to another preferred embodiment of the present invention, R⁷ is —C(O)NR⁴²R⁴³, wherein R⁴² and R⁴³ are taken together with the nitrogen to which they are attached to form a pyrrolidinyl, piperidinyl, piperazinyl, morpholinyl, or thiomorpholinyl ring, wherein said pyrrolidinyl, piperidinyl, piperazinyl, morpholinyl, or thiomorpholinyl rings are optionally substituted.

According to another preferred embodiment of the present invention, R⁷ is —C(O)NR⁴²R⁴³, wherein R⁴² and R⁴³ are taken together with the nitrogen to which they are attached to form a pyrrolidinyl or piperidinyl ring, wherein said pyrrolidinyl or piperidinyl ring are optionally substituted.

According to another preferred embodiment of the present invention, R⁷ is —C(O)NR⁴²R⁴³, wherein R⁴² and R⁴³ are taken together with the nitrogen to which they are attached to form a pyrrolidinyl ring, wherein said pyrrolidinyl ring is optionally substituted.

According to another preferred embodiment of the present invention, R⁷ is —C(O)NR⁴²R⁴³, wherein R⁴² and R⁴³ are taken together with the nitrogen to which they are attached to form a pyrrolidin-1-yl ring, wherein said pyrrolidin-1-yl is optionally substituted.

According to another preferred embodiment of the present invention, R⁷ is —(CH₂)ₙ(5 to 10 membered heterocyclyl) group, wherein said —(CH₂)ₙ(5 to 10 membered heterocyclyl) group is optionally substituted.

According to another preferred embodiment of the present invention, R⁷ is a —(CH₂)ₙ(5-8 membered heterocyclyl) group, wherein said —(CH₂)ₙ(5-8 membered heterocyclyl) group is optionally substituted.

According to another preferred embodiment of the present invention, R⁷ is a —(CH₂)ₙ(5 or 6 membered heterocyclyl) group, wherein said —(CH₂)ₙ(5 or 6 membered heterocyclyl) group is optionally substituted.

According to another preferred embodiment of the present invention, R⁷ is a —(CH₂)ₙ(5 membered heterocyclyl) group, wherein said —(CH₂)ₙ(5 membered heterocyclyl) group is optionally substituted.

According to another preferred embodiment of the present invention, R⁷ is —(CH₂)ₙthiazolyl, wherein n is an integer from 0 to 6, and said —(CH₂)nthiazolyl is optionally substituted.

According to another preferred embodiment of the present invention, R⁷ is a thiazolyl, wherein said thiazolyl is optionally substituted.

According to another preferred embodiment of the present invention, R⁷is an imidazolyl, wherein said imidazolyl is optionally substituted.

According to another preferred embodiment of the present invention, R⁷ is selected from the group consisting of imidazolyl, oxazolyl, oxadiazolyl, isoxazolyl, thiazolyl and thiadiazolyl, wherein the imidazolyl, oxazolyl, oxadiazolyl, isoxazolyl, thiazolyl and thiadiazolyl, is optionally substituted.

According to another preferred embodiment of the present invention, R⁷ is selected from the group consisting of halo, —CO₂H, —CONH₂ and —CSNH₂.

According to another preferred embodiment of the present invention, R⁷ is a heteroaryl group optionally substituted by one or more moiety selected from the group consisting of halo, cyano, nitro, trifluoromethoxy, trofluoromethyl, azido, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —NR³⁶R³⁷, —OR³⁷, —SO₂NR³⁶R³⁹, (C₁-C₆)alkyl, (C₃-C₁₀)cycloalkyl, —(CH₂)ⱼO(CH₂)ᵢNR³⁶R³⁹, —(CH₂)ₙO(CH₂)ᵢOR³⁷, —(CH₂)ₙOR³⁷, —S(O)ⱼ(C₁-C₆ alkyl), —(CH₂)ₙ(C₆-C₁₀ aryl), —(CH₂)ₙ(5 to 10 membered heterocyclyl), —C(O)(CH₂)ₙ(C₆-C₁₀ aryl), —(CH₂)ₙO(CH₂)ⱼ(C₆-C₁₀ aryl), —(CH₂)ₙO(CH₂)ᵢ(5 to 10 membered heterocyclyl), —C(O)(CH₂)ₙ(5 to 10 membered heterocyclyl), —(CH₂)ⱼNR³⁹(CH₂)ᵢNR³⁶R³⁹, —(CH₂)ⱼNR³⁹CH₂C(O)NR³⁶R³⁹, —(CH₂)ⱼNR³⁹(CH₂)ᵢNR³⁷C(O)R⁴⁰, (CH₂)ⱼNR³⁹(CH₂)ₙO(CH₂)ᵢOR³⁷, —(CH₂)ⱼNR³⁹(CH₂)ᵢS(O)ⱼ(C₁-C₆ alkyl), —(CH₂)ⱼNR³⁹, —(CH₂)ₙR³⁶, —SO₂(CH₂)ₙ(C₆-C₁₀ aryl), and —SO₂(CH₂)ₙ(5 to 10 membered heterocyclyl), wherein j is an integer from 0 to 2, n is an integer from 0 to 6, i is an integer from 2 to 6, the —(CH₂)ᵢ— and —(CH₂)ₙ— moieties of the said substituent groups optionally include a carbon-carbon double or triple bond where n is an integer between 2 and 6, and the alkyl, aryl and heterocyclyl moieties of the substituent groups are unsubstituted or substituted with one or more substituents independently selected from halo, cyano, nitro, trifluoromethyl, azido, —OH, —C(O)R⁴⁰, —C(O)OR⁴⁰, —OC(O)R⁴⁰, —OC(O)OR¹⁰, —NR³⁶C(O)R³⁹, —C(O)NR³⁶R³⁹, —(CH₂)NR³⁶R³⁹, (C₁-C₆)alkyl, (C₃-C₁₀)cycloalkyl, —(CH₂)ₙ(C₆-C₁₀ aryl), —(CH₂)ₙ(5 to 10 membered heterocyclyl), —(CH₂)ₙO(CH₂)ᵢOR³⁷, and —(CH₂)ₙOR³⁷, wherein n is an integer from 0 to 6 and i is an integer from 2 to 6, and wherein R³⁶ and R³⁹ are independently selected from the group consisting of H, —OH, (C₁-C₆)alkyl, (C₃-C₁₀)cycloalkyl, —(CH₂)ₙ(C₆-C₁₀ aryl), —(CH₂)ₙ(5 to 10 membered heterocyclyl), —(CH₂)ₙO(CH₂) OR³⁷ and —(CH₂)ₙOR³⁷, wherein n is an integer from 0 to 6 and i is an integer from 2 to 6, and the alkyl, aryl and heterocyclyl moieties of the R³⁶ and R³⁹ groups are unsubstituted or substituted with one or more substituents independently selected from hydroxy, halo, cyano, nitro, trifluoromethyl, azido, —C(O)R⁴⁰, —(CH₂)ₙ(C₆-C₁₀ aryl), —(CH₂)ₙ(5 to 10 membered heterocyclyl), —(CH₂)ₙO(CH₂)ᵢOR³⁷, and —(CH₂)ₙOR³⁷, wherein n is an integer from 0 to 6 and i is an integer from 2 to 6, where when R³⁶ and R³⁹ are both attached to the same nitrogen, then R³⁶ and R³⁹ are not both bonded to the nitrogen directly through an oxygen.

According to another preferred embodiment of the present invention, R⁷ is selected from the group consisting of H, —(C₁-C₆)alkyl, —C(O)NR³⁶R³⁷, —C(O)(C₆-C₁₀ aryl), —(CH₂)ₙ(C₆-C₁₀ aryl) and —(CH₂)ₙ(5 to 10 membered heterocyclyl), wherein the R⁷ groups other than H are optionally substituted. Preferably R⁷ is —(CH₂)ₙ(C₆-C₁₀ aryl) and —(CH₂)ₙ(5 to 10 membered heterocyclyl), optionally substituted, more preferably phenyl or pyridyl, optionally substituted.

According to another preferred embodiment of the present invention, R⁷ is selected from the group consisting of H, —(C₁-C₆)alkyl, —C(O)NR³⁶R³⁷, —C(O)(C₆-C₁₀ aryl), —(CH₂), —(C₆-C₁₀ aryl) and —(CH₂)ₙ(5 to 10 membered heterocyclyl), wherein the R⁷ groups other than H are optionally substituted.

According to another preferred embodiment of the present invention, R⁷ is selected from the group consisting of H, —(C₁-C₆)alkyl, —C(O)NR³⁶R³⁷, —C(O)(C₆-C₁₀ aryl), —(CH₂)ₙ(C₆-C₁₀ aryl) and —(CH₂)ₙ(5 to 10 membered heterocyclyl), wherein the R⁷ groups other than H are optionally substituted by tert-butyl-dimethyl-silanyl and 1 to 3 R³⁸ groups.

According to another preferred embodiment of the present invention, R⁷ is selected from the group consisting of —C(O)NR⁴²R⁴³, —(CH₂)ₙNR⁴²R⁴³, —NR⁴²C(═O)R⁴³, —SO₂R⁴², SO₂NR⁴²R⁴³, —NR³⁷SO₂R⁴², —NR³⁷SO₂NR⁴²R⁴³, —C(═N—OR⁴²)R⁴³, —C(═NR⁴²)R⁴³, —NR³⁷C(═NR⁴²)R⁴³, —C(═NR⁴²)NR³⁷R⁴³, —NR³⁷C(═NR⁴²)NR³⁷R⁴³, —C(O)R⁴², —CO₂R⁴², wherein each R⁴² and R⁴³ is independently selected from the group consisting of H, (C₁-C₆)alkyl, —(CH₂)ₙ(C₃-C₁₀)cycloalkyl), —(CH₂)ₙ(C₆-C₁₀ aryl), —(CH₂)ₙ(5 to 10 membered heterocyclyl), —(CH₂)ₙO(CH₂)ᵢOR³⁷, —(CH₂)ₙOR³⁷, wherein n is an integer from 0 to 6 and i is an integer from 2 to 6, and the alkyl, aryl and heterocyclyl moieties of the foregoing R⁴² and R⁴³ groups are optionally substituted by 1 to 3 substituents independently from R³⁸, or R⁴² and R⁴³ are taken together with the nitrogen to which they are attached to form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents, with the proviso that R⁴² and R⁴³ are not both bonded to the nitrogen directly through an oxygen.

According to another preferred embodiment of the present invention, R⁷ is selected from the group consisting of —C(O)NR⁴²R⁴³, —SO₂R¹², —SO₂NR⁴²R⁴³, —C(═N—OR⁴²)R¹³ and —C(═NR⁴²)R⁴

According to another preferred embodiment of the present invention, R⁷ is —C(O)NR⁴²R⁴³, wherein each R⁴² and R⁴³ is independently selected from the group consisting of H, (C₁-C₆)alkyl, —(CH₂)ₙOR³⁷, wherein n is an integer from 0 to 6 and the alkyl moiety of the foregoing R⁴² and R⁴³ groups are optionally substituted by 1 to 3 substituents independently from halo, cyano, trifluoromethyl, —C(O)R⁴⁰, —NRN³⁷C(O)R⁴¹, —C(O)NR³⁷R⁴¹, —NR³⁷R⁴¹, (C₁-C₆)alkyl, —(CH₂)ₙ(C₆-C₁₀ aryl), —(CH₂)ₙ(5 to 10 membered heterocyclyl), —(CH₂)ₙO(CH₂)ᵢOR³⁷ and —(CH₂)ₙOR₃₇, wherein n is an integer from 0 to 6 and i is an integer from 2 to 6, or R⁴² and R⁴³ are taken together with the nitrogen to which they are attached to form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents, with the proviso that R⁴² and R⁴³ are not both bonded to the nitrogen directly through an oxygen.

According to another preferred embodiment of the present invention, R⁷ is —C(O)NR⁴²R⁴³, wherein R⁴² and R⁴³ are taken together with the nitrogen to which they are attached to form a C₅-C₉ azabicyclic, aziridinyl, azetidinyl, pyrrolidinyl, piperidinyl, piperazinyl or morpholinyl ring, wherein said C₅-C₉ azabicyclic, aziridinyl, azetidinyl, pyrrolidinyl, piperidinyl, piperazinyl or morpholinyl ring are unsubstituted or substituted with 1 to 5 R³⁸ substituents.

According to another preferred embodiment of the present invention, R⁷ is —C(O)NR⁴²R⁴³, wherein R⁴² and R⁴³ are taken together with the nitrogen to which they are attached to form a C₅-C₉ azabicyclic, aziridinyl, azetidinyl or pyrrolidinyl ring, wherein said C₅-C₉ azabicyclic, aziridinyl, azetidinyl or pyrrolidinyl ring are unsubstituted or substituted with 1 to 5 R³⁸ substituents.

According to another preferred embodiment of the present invention, R⁷ is —C(O)NR⁴²R⁴³, wherein R⁴² and R⁴³ are taken together with the nitrogen to which they are attached to form a C₅-C₉ azabicyclic, azetidinyl or pyrrolidinyl ring, wherein said C₅-C₉ azabicyclic, azetidinyl or pyrrolidinyl ring are unsubstituted or substituted with 1 to 5 R³⁸ substituents.

According to another preferred embodiment of the present invention, R⁷ is —C(O)NR⁴²R⁴³, wherein R⁴² and R⁴³ are taken together with the nitrogen to which they are attached to form a C₅-C₉ azabicyclic ring, wherein said C₅-C₉ azabicyclic ring is unsubstituted or substituted with 1 to 5 R³⁸ substituents.

According to another preferred embodiment of the present invention, R⁷ is —C(O)NR⁴²R⁴¹, wherein R⁴² and R⁴³ are taken together with the nitrogen to which they are attached to form a azetidinyl ring, wherein said azetidinyl ring is unsubstituted or substituted with 1 to 5 R³⁸ substituents.

According to another preferred embodiment of the present invention, R⁷ is —C(O)NR⁴²R⁴³, wherein R⁴² and R⁴³ are taken together with the nitrogen to which they are attached to form a pyrrolidinyl ring, wherein said pyrrolidinyl ring is unsubstituted or substituted with 1 to 5 R³⁸ substituents.

According to another preferred embodiment of the present invention, R⁷ is selected from the group consisting of —H, halogen, nitro, azido, —NR^{6a}R^{6b}, —NR^{6a}SO₂R^{6b}, —NR^{6a}C(O)R^{6b}, —OC(O)R^{6b}, —NR^{6a}C(O)OR^{6b}, —OC(O)NR^{6a}R^{6b}, —OR^{6a}, —SR^{6a}, —S(O)R^{6a}, —SO₂R^{6a}, —SO₃R^{6a}, —SO₂NR^{6a}R^{6b}, —COR^{6a}, —CO₂R^{6a}, —CONR^{6a}R^{6b}, —(C₁-C₄)fluoroalkyl, —(C₁-C₄)fluoroalkoxy, —(CZ³Z⁴)ₐCN, and a moiety selected from the group consisting of —(CZ³Z⁴)ₐ-aryl, —(CZ³Z⁴)ₐ-heterocycle, (C₂-C₆)alkynyl, —(CZ³Z⁴)ₐ-(C₃-C₆)cycloalkyl, —(CZ³Z⁴)ₐ-(C₅-C₆)cycloalkenyl, (C₂-C₆) alkenyl and (C¹-C⁶)alkyl, wherein said moiety is optionally substituted with 1 to 3 independently selected Y² groups, where a is 0, 1, 2, or 3, and wherein when a is 2 or 3, the CZ³Z⁴ units may be the same or different; wherein
each R^{6a} and R^{6b} is independently selected from the group consisting of hydrogen and a moiety selected from the group consisting of —(CZ⁵Z⁶)ᵤ-(C₃-C₆)cycloalkyl, —(CZ⁵Z⁶)ᵤ-(C₁-C₆)cycloalkenyl, —(CZ z6)ᵤ-aryl, —(CZ⁵Z⁶)ᵤ-heterocycle, (C₂-C₆)alkenyl, and (C₁-C₆)alkyl, wherein said moiety is optionally substituted with 1 to 3 independently selected Y³ groups, where u is 0, 1, 2, or 3, and wherein when u is 2 or 3, the CZ⁵Z⁶ units may be the same or different, or
R^{6a} and R^{6b} taken together with adjacent atoms form a heterocycle;
each Z³, Z⁴, Z⁵ and Z⁶ is independently selected from the group consisting of H, F and (C₁-C₆)alkyl, or
each Z³ and Z⁴, or Z⁵ and Z⁶ are selected together to form a carbocycle, or
two Z³ groups on adjacent carbon atoms are selected together to optionally form a carbocycle;
each Y² and Y³ is independently selected from the group consisting of halogen, cyano, nitro, tetrazolyl, guanidino, amidino, methylguanidino, azido, —C(O)Z⁷, —OC(O)NH₂, —OC(O) NHZ⁷, —OC(O)NZ⁷Z⁸, —NHC(O)Z⁷, —NHC(O)NH₂, —NHC(O)NHZ⁷, —NHC(O)NZ⁷Z⁸, —C(O)OH, —C(O)OZ⁷, —C(O)NH₂, —C(O)NHZ⁷, —C(O)NZ⁷Z⁸, —P(O)₃H₂, —P(O)₃(Z⁷)₂, —S(O)₃H, —S(O)Z⁷, —S(O)₂Z⁷, —S(O)₃Z⁷, -Z⁷, —OZ⁷, —OH, —NH₂, —NHZ⁷, —NZ⁷Z⁸, —C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxy, —(CZ⁹Z¹⁰)ᵣNH₂, —(CZ⁹Z¹⁰)ᵣNHZ³, —(CZ⁹Z₁₀)ᵣNZ⁷Z⁸, —X⁶(CZ⁹Z¹⁰), —(C₃-C₈)cycloalkyl, —X⁶(CZ⁹Z¹⁰)ᵣ-(C₅-C₈)cycloalkenyl, —X⁶(CZ⁹Z¹⁰)ᵣ-aryl and —X⁶(CZ⁹Z¹⁰)ᵣ-heterocycle, wherein
r is 1, 2, 3 or 4;
X⁶ is selected from the group consisting of O, S, NH, —C(O)—, —C(O)NH—, —C(O)O—, —S(O)—, —S(O)₂— and —S(O)₃—;
Z⁷ and Z⁸ are independently selected from the group consisting of an alkyl of 1 to 12 carbon atoms, an alkenyl of 2 to 12 carbon atoms, an alkynyl of 2 to 12 carbon atoms, a cycloalkyl of 3 to 8 carbon atoms, a cycloalkenyl of 5 to 8 carbon atoms, an aryl of 6 to 14 carbon atoms, a heterocycle of 5 to 14 ring atoms, an aralkyl of 7 to 15 carbon atoms, and a heteroaralkyl of 5 to 14 ring atoms, or
Z⁷ and Z⁸ together may optionally form a heterocycle;
Z⁹ and Z¹⁰ are independently selected from the group consisting of H, F, a (C₁-C₁₂)alkyl, a (C₆-C₁₄)aryl, a (C₅-C₁₄)heteroaryl, a (C₇-C₁₅)aralkyl and a (C₅-C₁₄)heteroaralkyl, or
Z⁹ and Z¹⁰ are taken together form a carbocycle, or
two Z⁹ groups on adjacent carbon atoms are taken together to form a carbocycle; or
any two Y² or Y³ groups attached to adjacent carbon atoms may be taken together to be —O[C(Z⁹)(Z¹⁰)]ᵣO or —O[C(Z⁹)(Z¹⁰)]ᵣ₊₁, or
any two Y² or Y³ groups attached to the same or adjacent carbon atoms may be selected together to form a carbocycle or heterocycle; and wherein
any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C₁-C₄)alkyl, (C₁-C₄)alkoxy and an —N[(C₁-C₄)alkyl][(C₁-C₄)alkyl].

According to another preferred embodiment of the present invention, R⁷ is selected from the group consisting of —H, —Y-(aryl), —Y-(heteroaryl) and C(O)-heterocyclyl, each of which, except for —H, is optionally substituted.

According to another preferred embodiment of the present invention, R⁷ is selected from the group consisting of —H, —Y-(aryl) and —Y-(heteroaryl), each of which, except for —H, is optionally substituted.

According to another preferred embodiment of the present invention, R⁷ is selected from the group consisting of 5-membered aromatic rings containing one or more heteroatoms selected from sulphur, oxygen and nitrogen. Such rings include pyrrole, pyrazole, pyrazolone, imidazole, oxazole, furan, tetrazole, triazole, thiazole, thiophene or thiadiazole, any of which may be optionally substituted. Preferred 5-membered heteroaromatic rings include pyrrole, pyrazole, imidazole, triazole, thiazole, thiophene or thiadiazole.

According to another preferred embodiment of the present invention, R⁷ is selected from the group consisting of

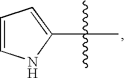

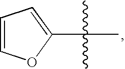

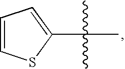

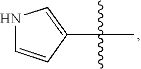

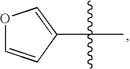

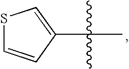

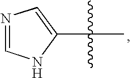

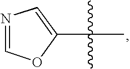

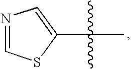

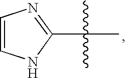

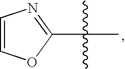

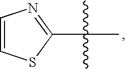

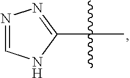

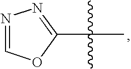

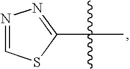

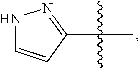

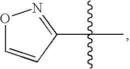

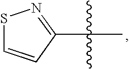

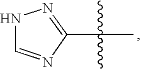

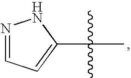

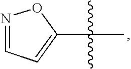

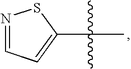

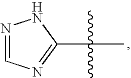

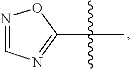

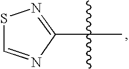

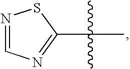

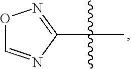

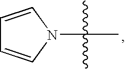

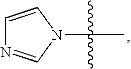

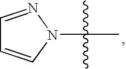

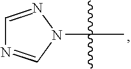

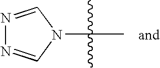

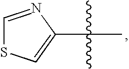

wherein the members of said group are optionally substituted.

According to another preferred embodiment of the present invention, R⁷ is selected from the group consisting of

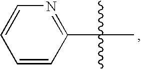

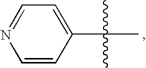

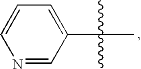

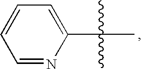

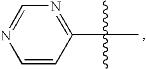

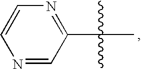

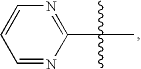

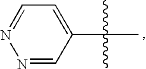

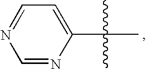

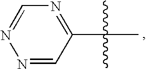

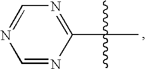

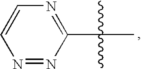

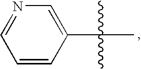

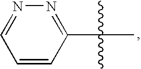

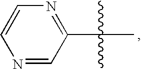

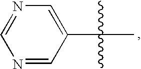

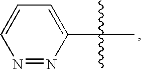

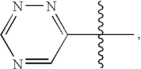

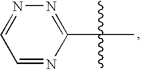

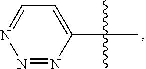

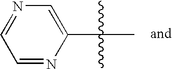

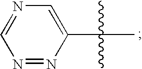

wherein the members of said group are optionally substituted.

According to another preferred embodiment of the present invention, R⁷ is selected from the group consisting of

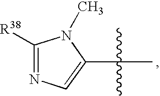

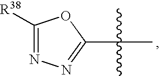

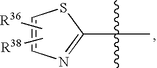

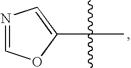

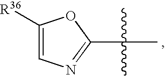

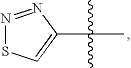

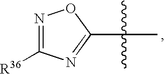

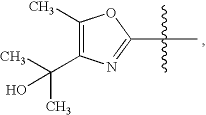

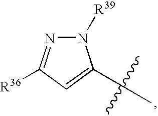

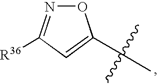

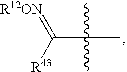

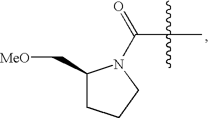

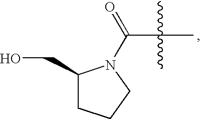

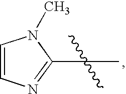

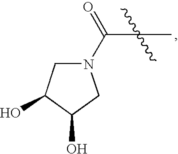

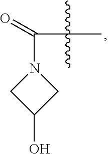

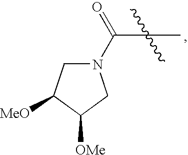

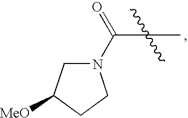

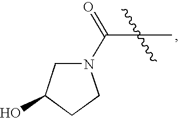

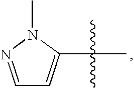

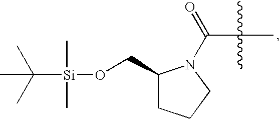

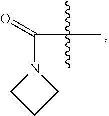

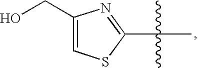

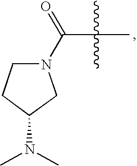

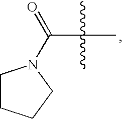

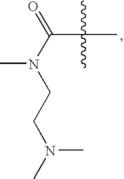

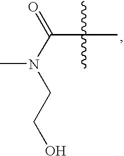

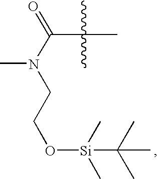

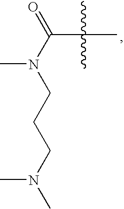

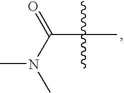

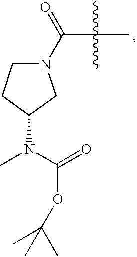

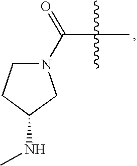

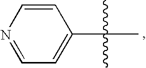

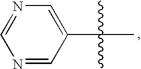

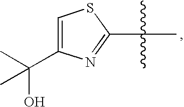

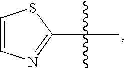

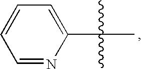

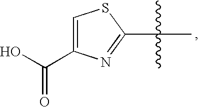

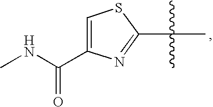

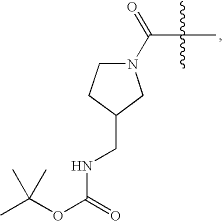

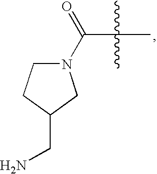

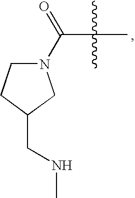

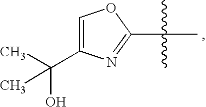

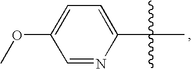

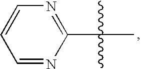

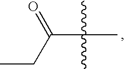

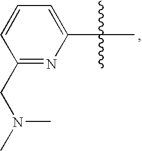

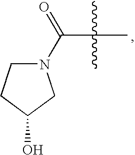

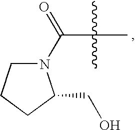

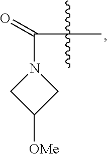

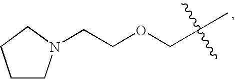

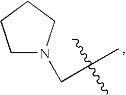

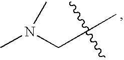

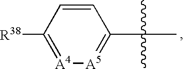

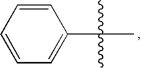

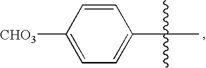

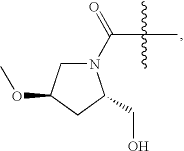

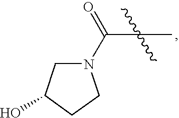

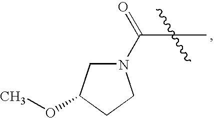

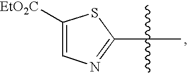

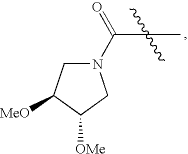

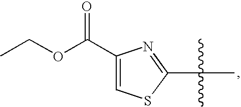

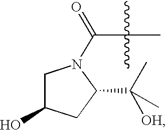

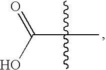

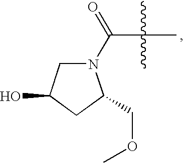

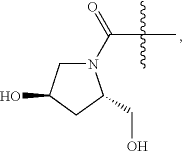

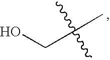

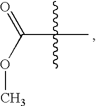

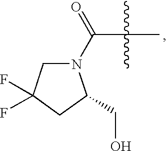

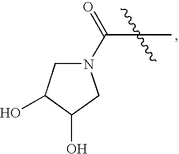

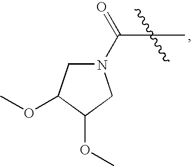

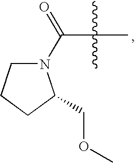

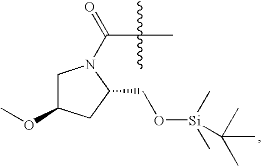

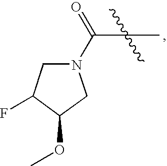

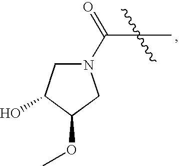

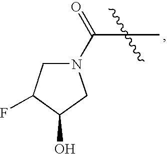

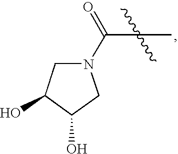

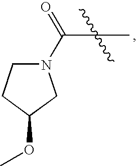

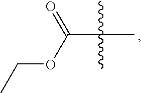

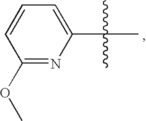

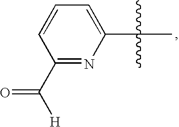

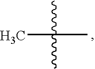

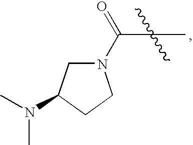

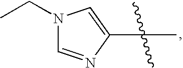

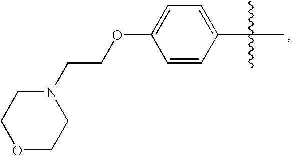

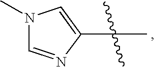

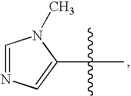

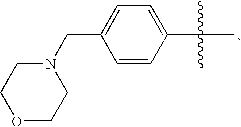

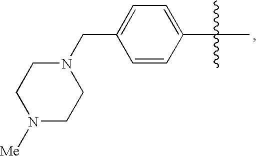

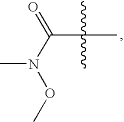

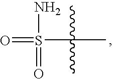

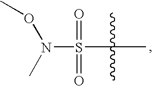

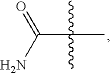

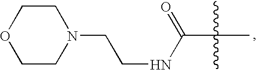

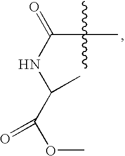

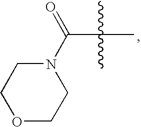

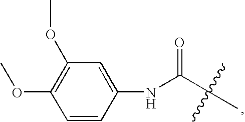

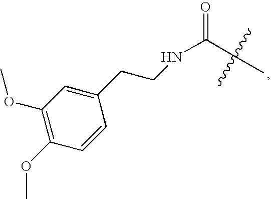

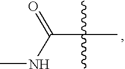

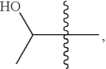

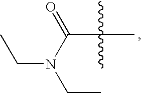

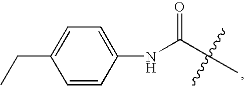

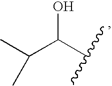

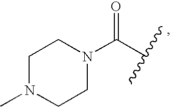

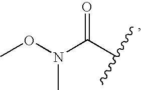

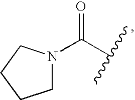

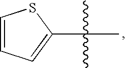

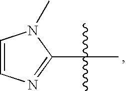

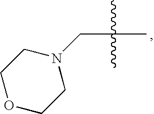

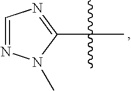

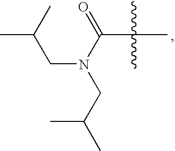

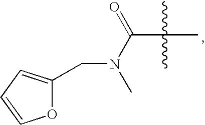

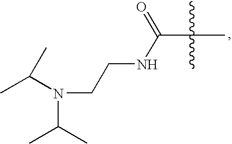

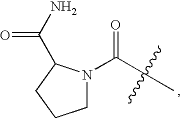

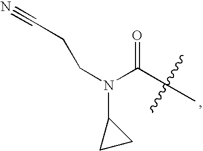

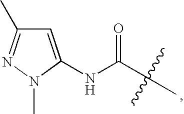

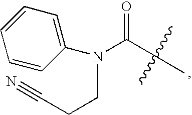

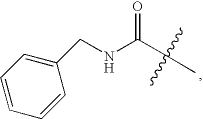

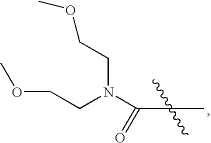

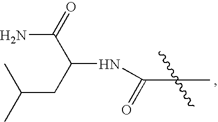

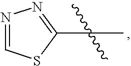

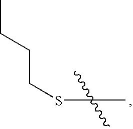

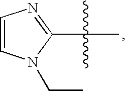

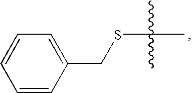

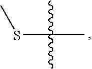

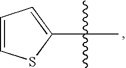

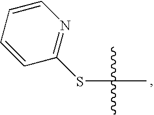

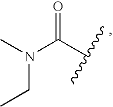

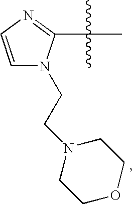

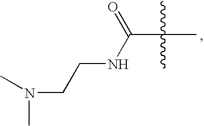

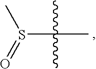

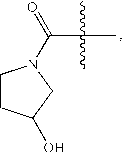

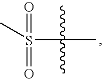

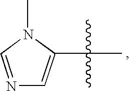

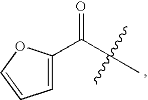

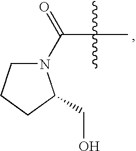

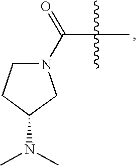

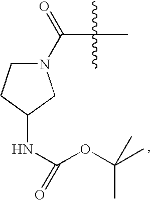

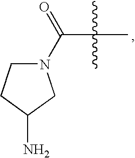

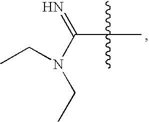

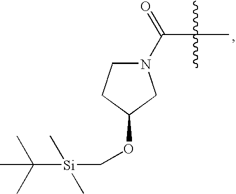

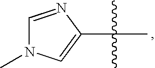

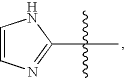

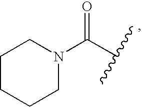

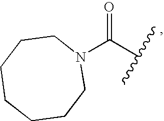

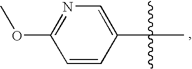

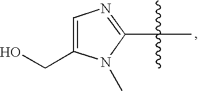

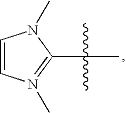

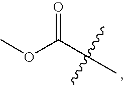

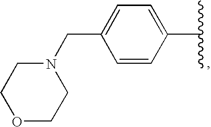

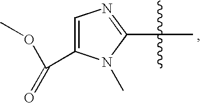

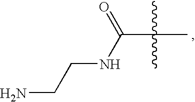

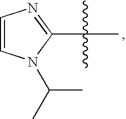

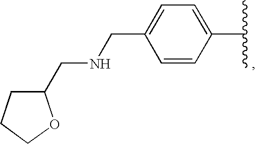

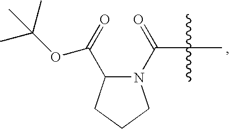

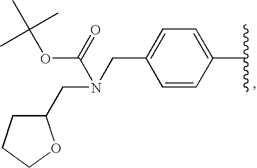

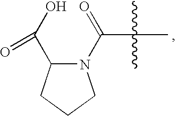

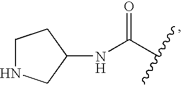

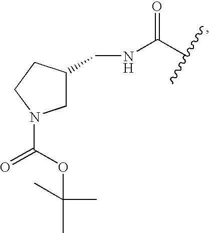

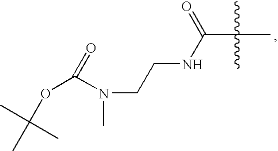

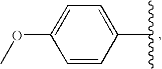

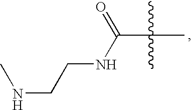

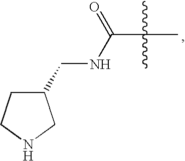

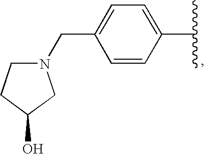

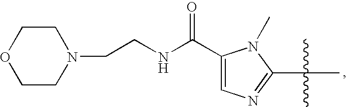

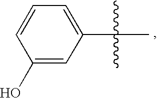

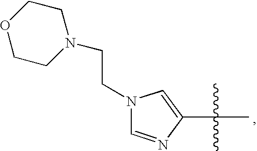

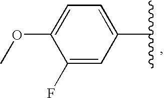

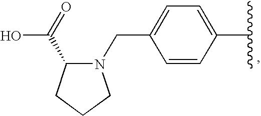

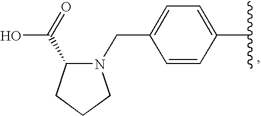

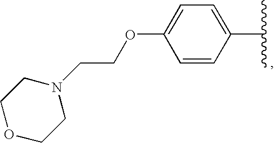

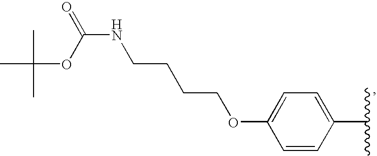

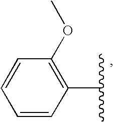

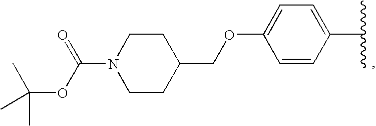

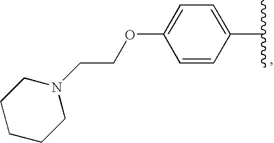

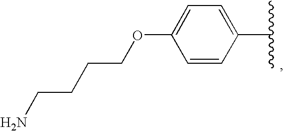

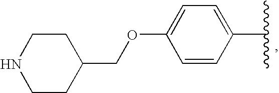

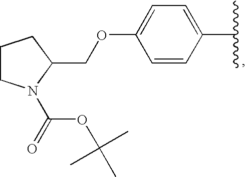

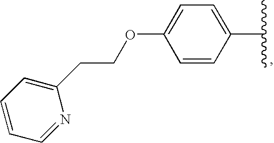

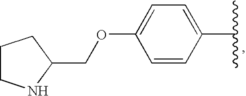

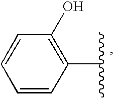

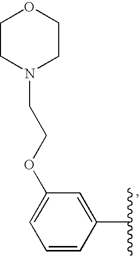

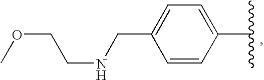

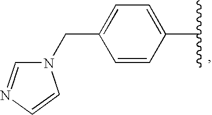

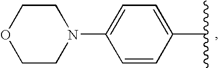

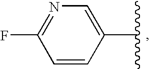

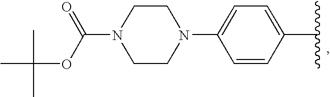

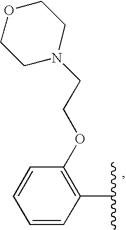

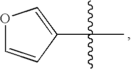

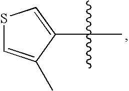

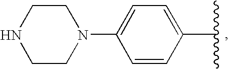

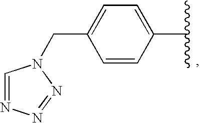

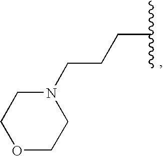

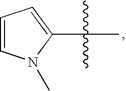

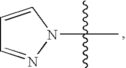

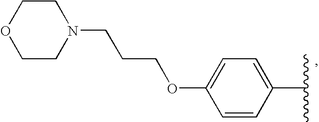

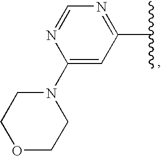

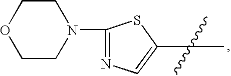

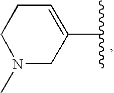

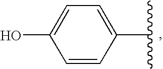

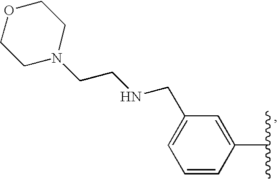

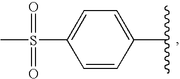

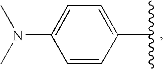

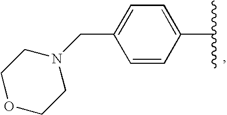

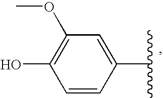

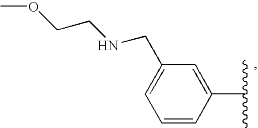

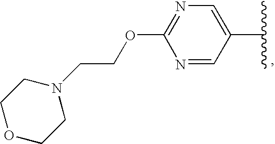

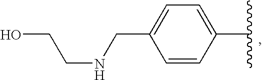

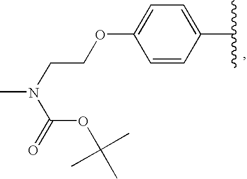

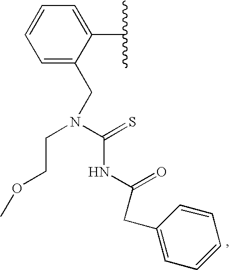

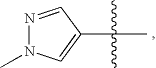

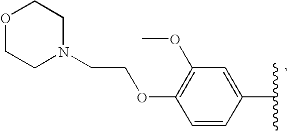

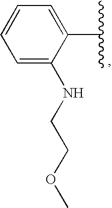

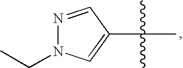

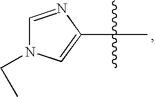

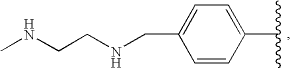

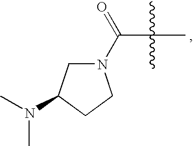

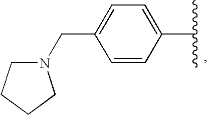

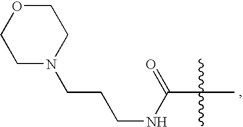

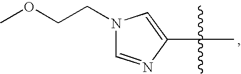

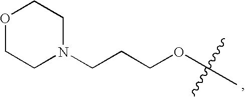

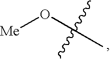

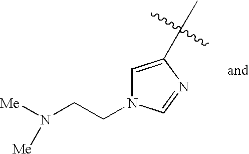

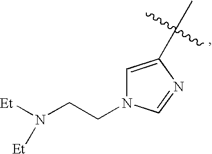

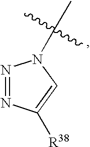

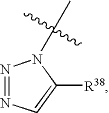

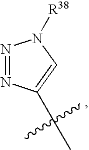

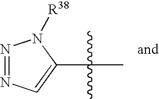

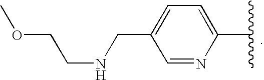

According to another preferred embodiment of the present invention, R⁷ is selected from the group consisting of phenyl and pryidyl, each of which is optionally substituted.

According to another preferred embodiment of the present invention, R⁷ groups other than —H and halogen are optionally substituted by 1 to 5 R³⁸; wherein
each R³⁸ is independently selected from halo, cyano, nitro, trifluoromethoxy, trifluoromethyl, azido, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —NR³⁶R³⁹, —OR³⁷, —SO₂NR³⁶R³⁹, C₁-C₆ alkyl, —(CH₂)ⱼO(CH₂)ᵢNR³⁶R³⁹, —(CH₂)ₙO(CH₂)ᵢOR³⁷, —(CH₂)ₙOR³⁷, —S(O)ⱼ(C₁-C₆ alkyl), —(CH₂)ₙ(C₆-C₁₀ aryl), —(CH₂)ₙ(5-10 membered heterocyclyl); —C(O)(CH₂)ₙ(C₆-C₁₀ aryl), —(CH₂)ₙO(CH₂)ⱼ(C₆-C₁₀ aryl), —(CH₂)ₙO(CH₂)ᵢ(5-10 membered heterocyclyl), —C(O)(CH₂)ₙ(5-10 membered heterocyclyl), —(CH₂)ⱼNR³⁹(CH₂)ᵢNR³⁶R³⁹, —(CH₂)ⱼNR³⁹CH₂C(O)NR³⁶R³⁹, (CH₂)ⱼNR³⁹(CH₂)ᵢNR³⁷C(O)R⁴⁰, —(CH₂)ⱼNR³⁹(CH₂)ₙO(CH₂)ᵢOR³⁷, —(CH₂)ⱼNR³⁹(CH₂)ᵢS(O)ⱼ(C₁-C₆ alkyl), —(CH₂)ⱼNR³⁹(CH₂)R³⁶—SO₂(CH₂)ₙ(C₆-C₁₀ aryl), —SO₂(CH₂)ₙ(5-10 membered heterocyclyl), —(CH₂)ₙNR³⁶R³⁹, —NR³⁷SO₂NR³⁶R³⁹, SO₂R³⁶, C₂-C₆ alkenyl, C₃-C₁₀ cycloalkyl and C₁-C₆ alkylamino, wherein j is an integer ranging from 0 to 2, n is an integer ranging from 0 to 6, i is an integer ranging from 2 to 6, the —(CH₂)ᵢ— and —(CH₂)ₙ— moieties of the foregoing R³⁸ groups optionally include a carbon-carbon double or triple bond where n is an integer between 2 and 6, and the alkyl, aryl and heterocyclyl moieties of the foregoing R³⁸ groups are optionally substituted by one or more substituents independently selected from halo, cyano, nitro, trifluoromethyl, azido, —OH, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —(CH₂)ₙNR³⁶R³⁹, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙO(CH₂)ᵢOR³⁷, and —(CH₂)ₙOR³⁷, wherein n is an integer ranging from 0 to 6 and i is an integer ranging from 2 to 6;
each R³⁶ and R³⁹ is independently selected from the group consisting of H, —OH, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙO(CH₂)ᵢOR³⁷, —(CH₂)ₙCN(CH₂)ₙOR³⁷, —(CH₂)ₙCN(CH₂)ₙR³⁷, and —(CH₂)ₙOR³⁷, wherein n is an integer ranging from 0 to 6 and i is an integer ranging from 2 to 6, and the alkyl, aryl and heterocyclyl moieties of the foregoing R³⁶ and R³⁹ groups are optionally substituted by one or more substituents independently selected from —OH, halo, cyano, nitro, trifluoromethyl, azido, —C(O)R⁴⁰, —C(O)OR⁴⁰, —CO(O)R⁴⁰, —OC(O)OR⁴⁰, —NR³⁷C(O)R⁴¹, —C(O)NR³⁷R⁴¹, —NR³⁷R⁴¹, —C₁-C₆ alkyl, —(CH₂)ₙ(C₆-C₁₀ aryl), —(CH₂)ₙ(5 to 10 membered heterocyclyl), —(CH₂)ₙO(CH₂)ᵢOR³⁷, and —(CH₂)ₙOR³⁷, wherein n is an integer ranging from 0 to 6 and i is an integer ranging from 2 to 6, with the proviso that when R³⁶ and R³⁹ are both attached to the same nitrogen, then R³⁶ and R³⁹ are not both bonded to the nitrogen directly through an oxygen;
each R⁴⁰ is independently selected from H, C₁-C₁₀ alkyl, —(CH₂)ₙ(C₆-C₁₀ aryl), C₃-C₁₀ cycloalkyl, and —(CH₂)ₙ(5-10 membered heterocyclyl), wherein n is an integer ranging from 0 to 6; and
each R³⁷ and R⁴¹ is independently selected from H, OR³⁶, C₁-C₆ alkyl and C₃-C₁₀ cycloalkyl.

According to another preferred embodiment, R^{6a} is C₁-C₆alkyl, optionally substituted with 1 to 3 independently selected Y³ groups.

According to another preferred embodiment, Y³ is —NZ⁷Z⁸.

According to another preferred embodiment, each of Z⁷ and Z⁸ are independently selected from H and an optionally substituted C₁-C₁₂alkyl, preferably an optionally substituted C₁-C₆alkyl.

According to another preferred embodiment of the present invention, each D is independently defined by the group R¹, wherein R¹ is —C≡CH or —C≡C—(CR⁴⁵R⁴⁵)—R⁴⁶; wherein
each R⁴⁵ is independently selected from the group consisting of H, a (C₁-C₆)alkyl and a (C₃-C₈)cycloalkyl;
R⁴⁶ is selected from the group consisting of heterocyclyl, —N(R⁴⁷)—C(O)—N(R⁴⁷)(R⁴⁸), —N(R⁴⁷)—C(S)—N(R⁴⁷)(R⁴⁸), —N(R⁴⁷)—C(O)—OR⁴⁸, —N(R⁴⁷)—C(O)—(CH₂)ₙ—R⁴⁸, —N(R⁴⁷)—SO₂R⁴⁷, —(CH₂)ₙNR⁴⁷R⁴⁸, —(CH₂)ₙOR⁴⁸, —(CH₂)ₙSR⁴⁹, —(CH₂)ₙS(O)R⁴⁹, —(CH₂)ₙS(O)₂R⁴⁹, —OC(O)R⁴⁹, —OC(O)OR⁴⁹, —C(O)NR⁴⁷R⁴⁸, heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, —(CH₂)ₙOR⁵⁰, —(CH₂)ₙC(O)R⁴⁹, —C(O)₂R⁴⁹, —(CH₂)ₙSR⁴⁹, —(CH₂)ₙS(O)R⁴⁹, —(CH₂)ₙS(O)₂R⁴⁹, —(CH₂)ₙR⁴⁹, —(CH₂)ₙCN, aryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, or
R⁴⁷ and R⁴⁸, together with the atom to which they are attached, form a 3-8 membered carbo- or hetero-cyclic ring;
R⁴⁹ is selected from the group consisting of (C₁-C₆)alkyl, (C₃-C₈)cycloalkyl, heterocyclyl(C₁-C₆)alkylene, aryl(C₁-C₆)alkylene wherein the aryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, heteroaryl(C₁-C₆)alkylene wherein the heteroaryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁵⁰ and R⁵¹ are independently selected from the group consisting of H, (C₁-C₆)alkyl, (C₃-C₈)cycloalkyl and —C(O)R⁴⁵, or
R⁵⁰ and R⁵¹, together with the atom to which they are attached, form a 3-8 membered carbo- or hetero-cyclic ring.

According to another preferred embodiment of the present invention,
R⁴⁶ is selected from the group consisting of —N(R⁴⁷)—C(O)—N(R⁴⁷)(R⁴⁸), —N(R⁴⁷)—C(O)—(CH₂)ₙ—R⁴⁸ and —(CH₂)ₙNR⁴⁷R⁴⁸; wherein
R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, —(CH₂)ₙOR⁵⁰, —(CH₂)ₙS(O)₂R⁴⁹ and —(CH₂)ₙCN, or R⁴⁷ and R⁴⁸, together with the atom to which they are attached, form a 3-8 membered carbo- or hetero-cyclic ring; and
R⁵⁰ and R⁵¹ are independently selected from the group consisting of H and (C₁-C₆)alkyl, or R⁵⁰ and R⁵¹, together with the atom to which they are attached, form a 3-8 membered carbo- or hetero-cyclic ring.

According to another preferred embodiment of the present invention, R¹ is selected from the group consisting of

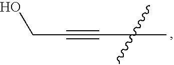

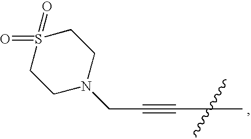

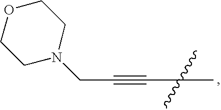

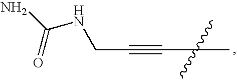

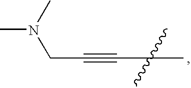

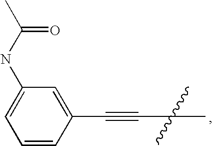

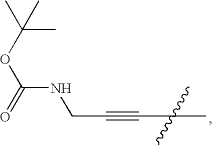

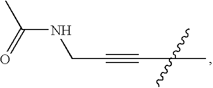

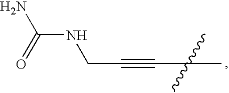

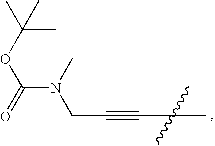

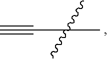

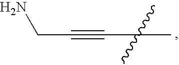

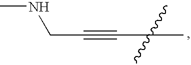

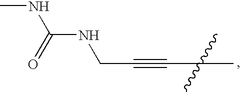

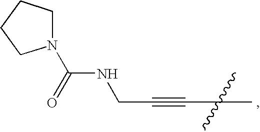

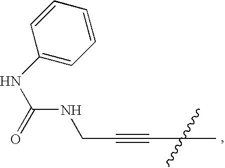

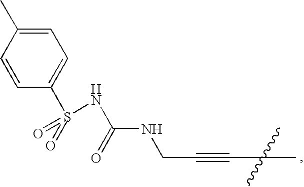

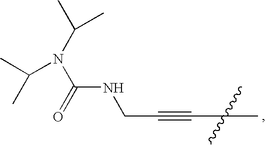

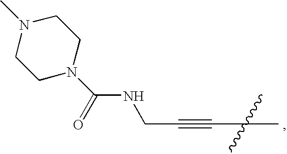

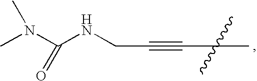

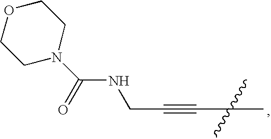

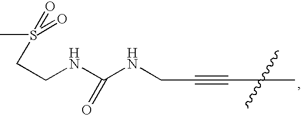

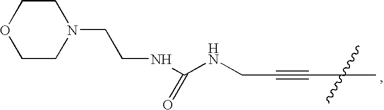

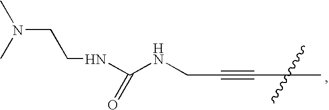

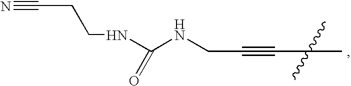

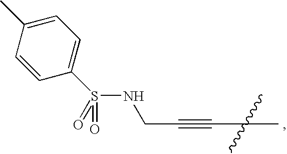

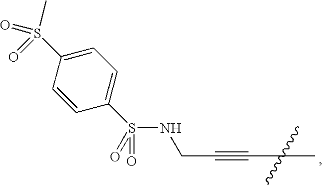

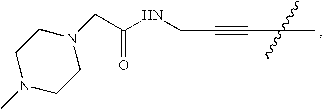

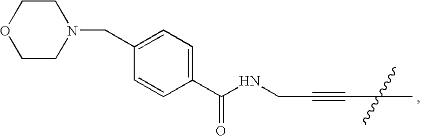

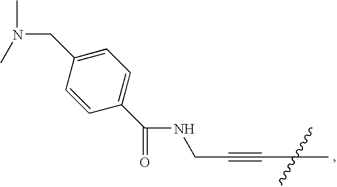

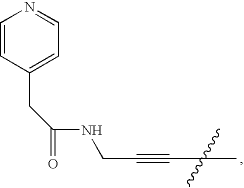

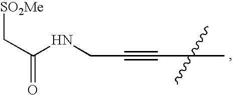

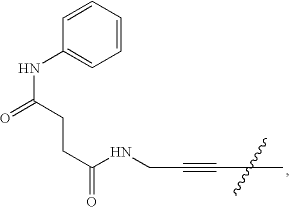

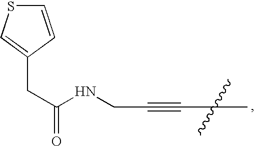

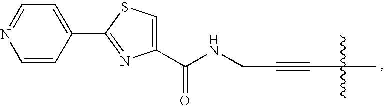

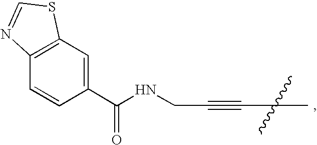

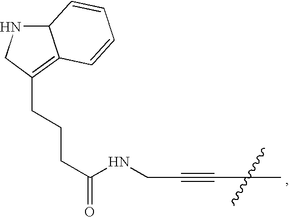

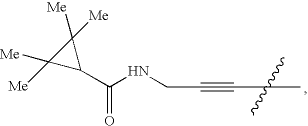

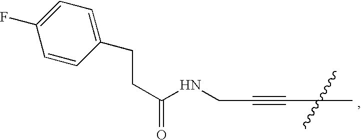

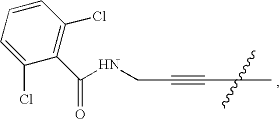

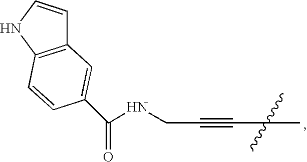

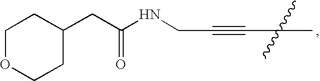

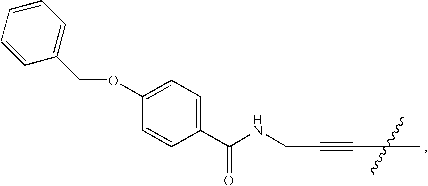

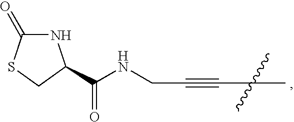

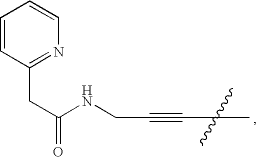

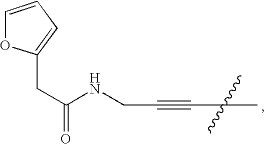

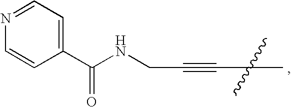

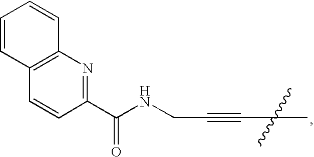

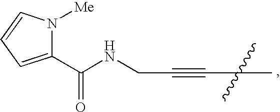

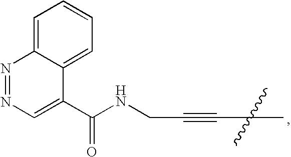

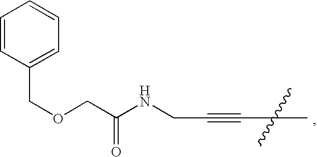

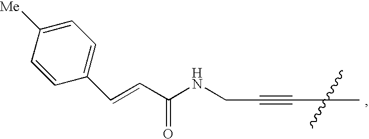

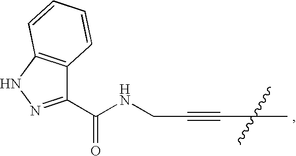

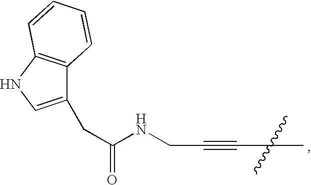

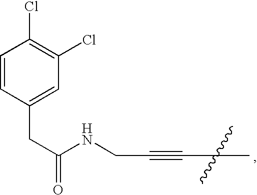

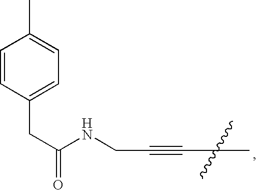

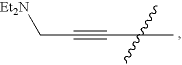

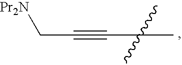

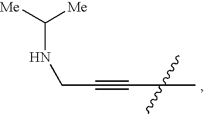

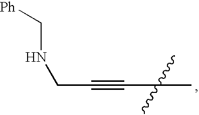

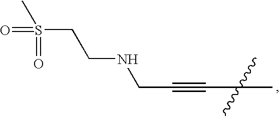

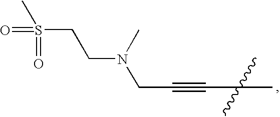

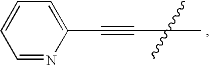

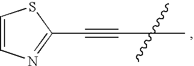

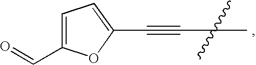

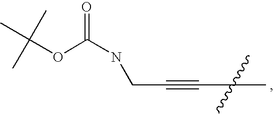

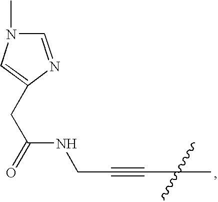

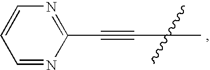

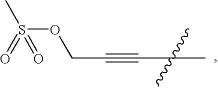

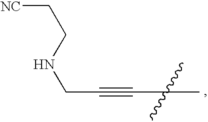

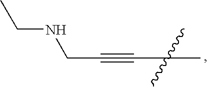

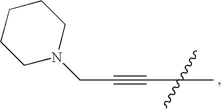

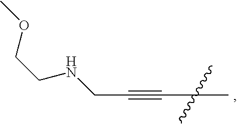

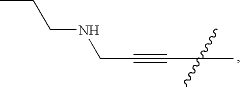

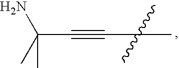

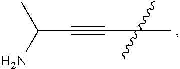

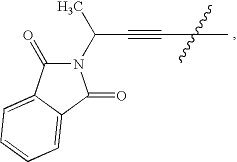

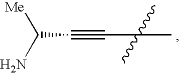

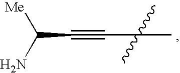

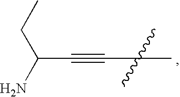

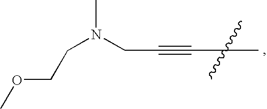

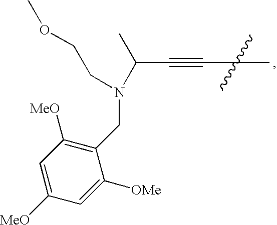

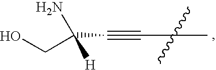

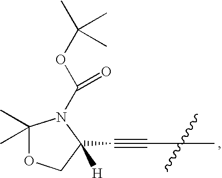

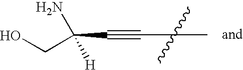

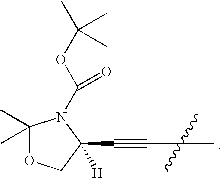

According to another preferred embodiment of the present invention, each D is independently defined by the group R²¹, wherein R²¹ is defined by -(Z¹¹)-(Z¹²)ₘ-(Z¹³)ₘ₁, wherein
Z¹¹ is heterocyclyl, when m and m1 are 0, or heterocyclylene, when either m or m1 are 1;
Z¹² is selected from the group consisting of OC(O), OC(S) and C(O);
Z¹³ is selected from the group consisting of heterocyclyl, aralkyl, N(H)R⁵², (C₁-C₃)alkyl, —OR⁵², halo, S(O)₂R⁵⁶, (C₁-C₃)hydroxyalkyl and (C₁-C₃)haloalkyl;
m is 0 or 1;
m1 is O or I;
R⁵² is selected from the group consisting of H, —(CH₂)_{q}S(O)₂R⁵⁴, —(C₁-C₆)alkyl-NR⁵³R⁵³, (C₁-C₃)alkyl, —(CH₂)_{q}OR⁵³, —C(O)R⁵⁴ and —C(O)OR⁵³;

q is 0, 1, 2, 3 or 4;

each R⁵³ is independently (C₁-C₃)alkyl;

R⁵⁴ is (C₁-C₃)alkyl or N(H)R⁵³; and

R⁵⁶ is selected from the group consisting of NH₂, (C₁-C₃)alkyl and OR⁵².

According to another preferred embodiment of the present invention, Z¹¹ is a heterocyclyl and m and m1 are each 0.

According to another preferred embodiment of the present invention, Z¹¹ is a heterocyclyl and m is 0 and m1 is 0, where the heterocyclyl group is selected from the group consisting of

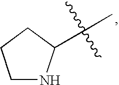

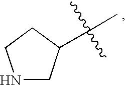

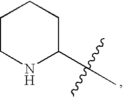

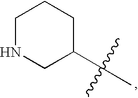

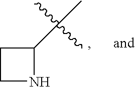

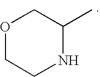

According to another preferred embodiment of the present invention, Z¹¹ is heterocyclylene, Z¹² is OC(O), m is 1, m1 is I and Z¹³ is heterocyclyl.

According to another preferred embodiment of the present invention, Z¹¹ is 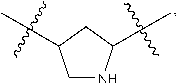
Z¹² is OC(O), and
Z¹³ is 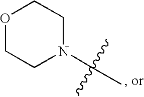
Z¹³ is N(H)R⁵², wherein R⁵² is (C₁-C₃)alkyl.

According to another preferred embodiment of the present invention, Z¹¹ is heterocyclylene, Z¹² is C(O) and m is 1, m1 is 1 and Z¹³ is (C₁-C₃)haloalkyl.

According to another preferred embodiment of the present invention, Z¹¹ is 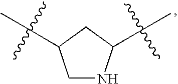
Z¹² is C(O), and
Z¹³ is (C₁-C₃)haloalkyl, preferably —CF₃.

According to another preferred embodiment of the present invention, Z¹¹ is heterocyclylene, m is 0, m1 is 1 and Z¹³ is heterocyclyl.

According to another preferred embodiment of the present invention, Z¹¹ is 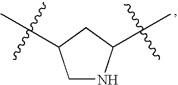
m is 0, and
Z¹³ is 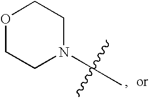
Z¹³ is (C₁-C₃)alkyl, or
Z¹³ is —OH, or
Z¹³ is —OR⁵², wherein R⁵² is (C₁-C₃)alkyl, preferably —CH₃ or
Z¹³ is halo, preferably —F, or
Z¹³ is (C₁-C₃)hydroxyalkyl, preferably —CH₃OH.

According to another preferred embodiment of the present invention, R² is selected from the group consisting of

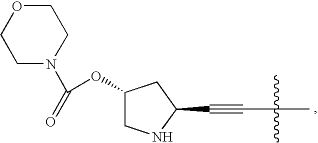

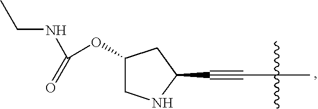

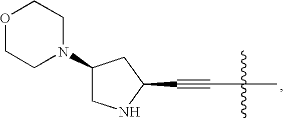

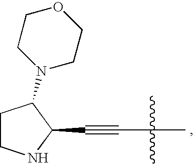

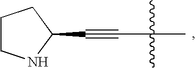

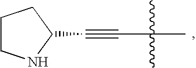

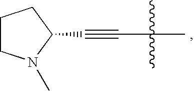

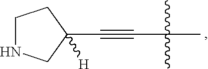

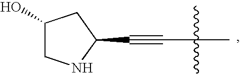

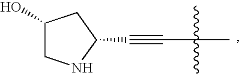

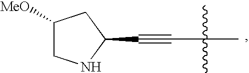

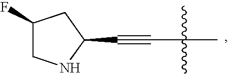

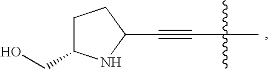

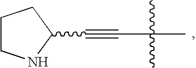

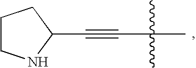

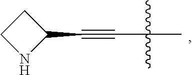

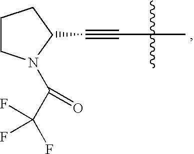

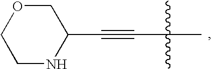

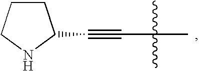

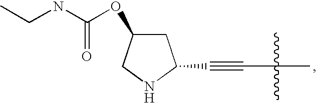

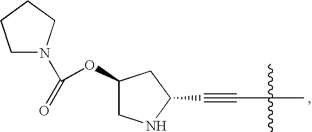

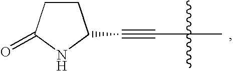

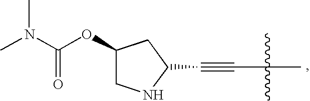

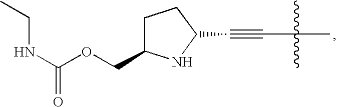

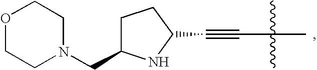

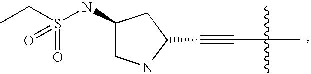

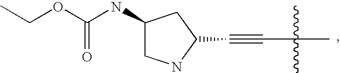

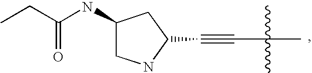

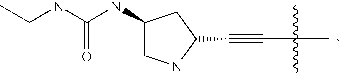

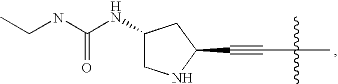

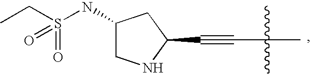

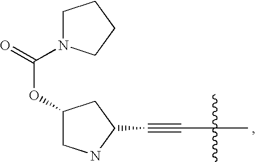

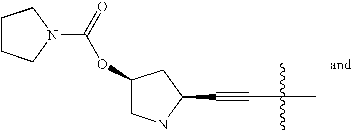

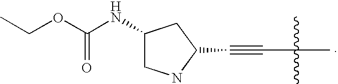

According to another preferred embodiment of the present invention, wherein each D is independently defined by the group R²¹, the heterocyclic or heterocyclyl group is optionally substituted with a substituent selected from the group consisting of (C₁-C₆)alkyl, (C₁-C₆)alkoxy, (C₁-C₆)alkylsufanyl, (C₁-C₆)alkylsulfenyl, (C₁-C₆)alkylsulfonyl, oxo, hydroxyl, mercapto, amino optionally substituted by alkyl, carboxy, carbamoyl optionally substituted by alkyl, alkylcarboxyamide, carboxyamide, aminosulfonyl optionally substituted by alkyl, ureido, arylurea, arylthiourea, alkylurea, cycloalkylurea, sulfonylurea, nitro, cyano, halo, aryl, aralkyl, heteroaryl and (C₁-C₆)perfluoroalkyl. Such a ring may be optionally fused to one or more other “heterocyclic” ring or cycloalkyl ring. Preferred examples of “heterocyclic” moieties include, but are not limited to, tetrahydrofuranyl, pyranyl, 1,4-dioxaneyl, 1,3-dioxanyl, piperidinyl, piperazinyl, 2,4-piperazinedionyl, pyrrolidinyl, pyrrolidinon-2-yl, pyrrolidinon-3-yl, pyrrolidinon-4-yl, pyrrolidinon-5-yl, imidazolidinyl, pyrazolidinyl, morpholinyl, thiomorpholinyl, tetrahydrothiopyranyl, tetrahydrothiophenyl, and the like

According to another preferred embodiment of the present invention, wherein each D is independently defined by the group R²¹, the heterocyclylene group is optionally substituted with substituents selected from the group consisting of (C₁-C₆)alkyl, (C₁-C₆)alkoxy, (C₁-C₆)alkylsufanyl, (C₁-C₆)alkylsulfenyl, (C₁-C₆)alkylsulfonyl, oxo, hydroxyl, mercapto, amino optionally substituted by alkyl, carboxy, carbamoyl optionally substituted by alkyl, alkylcarboxyamide, carboxyamide, aminosulfonyl optionally substituted by alkyl, ureido, arylurea, arylthiourea, alkylurea, cycloalkylurea, sulfonylurea, nitro, cyano, halo and (C₁-C₆)perfluoroalkyl, multiple degrees of substitution being allowed. Such a ring may be optionally fused to one or more benzene rings or to one or more of another “heterocyclic” rings or cycloalkyl rings. Preferred examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, pyrrolidinon-2,3-yl, pyrrolidinon-2,4-yl, pyrrolidinon-2,5-yl, pyrrolidinon-3,4-yl, pyrrolidinon-3,5-yl, pyrrolidinon-4,5-yl, morpholine-2,4-diyl, and the like.

According to another preferred embodiment of the present invention, Z is selected from the group consisting of —O—, —S—, —S(O)₀₋₂ and —NR⁵—, wherein R⁵ is selected from the group consisting of H, an optionally substituted (C₁-C₅)acyl and C₁-C₆ alkyl-O—C(O), wherein C₁-C₆ alkyl is optionally substituted.

According to another preferred embodiment of the present invention, R¹⁴ and R¹⁵ are both H, R¹⁶ is C₂-C₇ alkenyl or C₂-C₆ alkynyl and R¹⁷ is halogen, preferably fluorine.

According to another preferred embodiment, A¹ is a fused 6-membered heteroaryl group, optionally substituted with 0-4 D, preferably 0, 1 or 2 D, more preferably 2 D.

According to another preferred embodiment, A¹ is a fused 6-membered aryl group, optionally substituted with 0-4 D, preferably 0, 1 or 2 D, more preferably 2 D.

According to another preferred embodiment, A² is ═N—, ═CH—, or ═C(CN)—.

According to another preferred embodiment, M¹ is H.

According to another preferred embodiment, M² is a saturated C₃-C₆-monocyclic hydrocarbyl (preferably C₅-C₆, more preferably C₆), optionally containing one or two or three annular heteratoms, the ring being optionally substituted with between zero and four Y² substituents.

According to another preferred embodiment, M³ is absent.

According to another preferred embodiment of the present invention, Z is selected from the group consisting of —O—, —N(H)— and —N(C₁-C₆alkyl).

According to another preferred embodiment of the present invention, Z is —O—.

According to another preferred embodiment of the present invention, Z is —S—.

According to another preferred embodiment of the present invention, V is a 5 to 7 membered aryl or heteroaryl ring system, more preferably a 6 membered ring system, either of which is optionally substituted with 0 to 4 R² groups.

According to another preferred embodiment of the present invention, V is a 5 to 7 membered aryl, more preferably a 6 membered aryl system, wherein said V is optionally substituted with 0 to 4 R² groups.

According to another preferred embodiment of the present invention, V is selected from the group consisting of

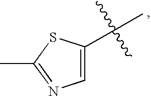

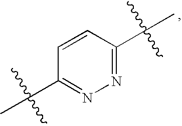

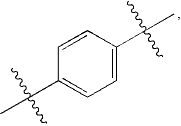

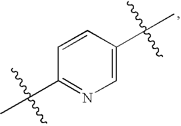

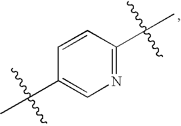

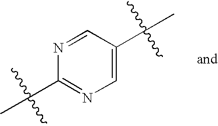

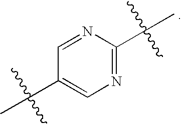

According to another preferred embodiment, V is substituted with one R² group.

According to another preferred embodiment, R² is halo, preferably F.

According to another preferred embodiment of the present invention, V is selected from the group consisting of phenyl, pyrazine, pyridazine, pryimidine and pyridine, wherein each of said phenyl, pyrazine, pyridazine, pryimidine and pyridine is optionally substituted with R¹⁴, R¹⁵, R¹⁶ and R¹⁷.

According to another preferred embodiment of the present invention, V is phenyl, optionally substituted with 0 to 4 R² groups.

According to another preferred embodiment of the present invention, V is phenyl, substituted with between zero and four halo.

According to another preferred embodiment of the present invention, E is —N(R¹³)—.

According to another preferred embodiment of the present invention, E is —NH— or —N(alkyl)-, preferably —NH—.

According to another preferred embodiment of the present invention, X is O or S, more preferably O.

According to another preferred embodiment of the present invention, [symbol] is a double bond and X¹ is O or S, more preferably 0.

According to another preferred embodiment of the present invention, [symbol] is a single bond and X¹ is H, or an optionally substituted alkyl, preferably a C₁alkyl optionally substituted with one, two or three halo, more preferably CF₃.

According to another preferred embodiment of the present invention, L², L³ and L⁴ are each C.

According to another preferred embodiment of the present invention, b is zero and L² and L³ are each —CH₂—.

According to another preferred embodiment of the present invention, L² and L³ are independently —CH₂—, O or N.

According to another preferred embodiment of the present invention, L and L¹ are independently selected from —CH— and —N—.

According to another preferred embodiment of the present invention, L and L¹ are each —N—.

According to another preferred embodiment of the present invention, L is —CH— and L¹-N—.

According to another preferred embodiment of the present invention, the group 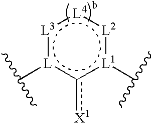
is represented by the group 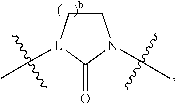
more preferably

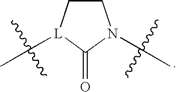

According to another preferred embodiment, L is CH or N.

According to another preferred embodiment of the present invention, W is selected from the group consisting of

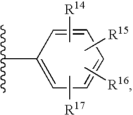

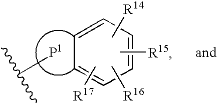

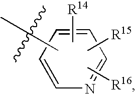

wherein P¹ is a five- to seven-membered ring, including the two shared carbon atoms of the aromatic ring to which P¹ is fused, and wherein P¹ optionally contains between one and three heteroatoms.

According to another preferred embodiment of the present invention, W is selected from the group consisting of phenyl, napthyl, 1,2,3,4-tetrahydronaphthyl, indanyl, benzodioxanyl, benzofuranyl, phenazinyl, phenothiazinyl, phenoxazinyl, tetrahydroisoquinolyl, pyrrolyl, pyrazolyl, pyrazolidinyl, imidazolyl, imidazolinyl, imidazolidinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, thiazolyl, thiazolinyl, thiazolidinyl, isothiazolyl, isothiazolidinyl, indolyl, isoindolyl, indolinyl, isoindolinyl, octahydroindolyl, octahydroisoindolyl, quinolyl, isoquinolyl, benzimidazolyl, thiadiazolyl, benzopyranyl, benzothiazolyl, benzoxazolyl, furyl, thienyl, benzothieliyl, and oxadiazolyl; each optionally substituted.

According to another preferred embodiment of the present invention, W is selected from the group consisting of phenyl, napthyl, 1,2,3,4-tetrahydronaphthyl, indanyl, benzodioxanyl, benzofuranyl, phenazinyl, phenothiazinyl, phenoxazinyl, tetrahydroisoquinolyl, pyrrolyl, pyrazolyl, pyrazolidinyl, imidazolyl, imidazolinyl, imidazolidinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, thiazolyl, thiazolinyl, thiazolidinyl, isothiazolyl, isothiazolidinyl, indolyl, isoindolyl, indolinyl, isoindolinyl, octahydroindolyl, octahydroisoindolyl, quinolyl, isoquinolyl, benzimidazolyl, thiadiazolyl, benzopyranyl, benzothiazolyl, benzoxazolyl, furyl, thienyl, benzothieliyl, and oxadiazolyl; each optionally substituted with one or more of R¹⁴, R¹⁵, R¹⁶ and R¹⁷.

According to another preferred embodiment of the present invention, W is phenyl, optionally substituted.

According to another preferred embodiment of the present invention, W is phenyl, optionally substituted with one or more of R¹⁴, R¹⁵, R¹⁶ and R¹⁷.

According to another preferred embodiment of the present invention, W is substituted by a halogen and either an alkenyl or alkynyl.

According to another preferred embodiment of the present invention, W is phenyl.

According to another preferred embodiment of the present invention, W is phenyl substituted by a halogen and either an alkenyl or alkynyl.

According to another preferred embodiment of the present invention, W is phenyl substituted by a halogen or a C₁-C₆alkoxy, preferably a halogen, more preferably F.

According to another embodiment, W is further selected from alkenyl, preferably C₃ alkenyl.

In a preferred embodiment of the present invention, the invention provides compounds of Formula (I-B) and racemic mixtures, diastereomers and enantiomers thereof: 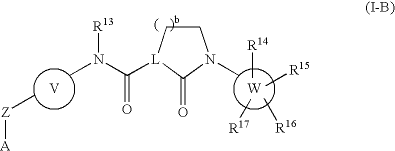
and N-oxides, hydrates, solvates, pharmaceutically acceptable salts, prodrugs and complexes thereof, wherein
A is 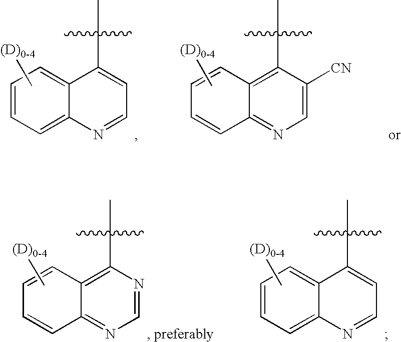
Z is —O—, —S—, —NH— or —N(C₁-C₆alkyl), preferably —O—;
V is phenyl or pyridinyl (each of which is optionally substituted with 0 to 4 R² groups, preferably 1 R² group, more preferably 1 fluorine), preferably phenyl (optionally substituted with 0 to 4 R² groups, preferably 1 R² group, more preferably 1 fluorine);
R¹³ is H or C₁-C₆alkyl, preferably H;
L is —CH—, —N— or —C(halogen)-, preferably, —CH— or —N—;
b is zero;
W is phenyl or pyridinyl, preferably phenyl; and
R¹⁴, R¹⁵, R¹⁶ and R¹⁷ are each independently H, halogen or alkoxy, preferably halogen, more preferably F.

In a preferred embodiment of Formula (I-B), one of R¹⁴, R¹⁵, R¹⁶ and R¹⁷ is halogen or alkoxy (preferably halogen, more preferably F) and the others are H.

In another preferred embodiment of Formula (I-B), each D is independently selected from the group consisting of R²⁵⁹, R⁰⁷⁷ and R⁷.

In another preferred embodiment of Formula (I-B), each D is independently selected from R⁷.

In another preferred embodiment of Formula (I-B), each D is independently selected from the group consisting of H, R⁰⁷⁷, —X²—R^{d}, C₁-C₆alkoxy and —OR^{6a}.

In another preferred embodiment of Formula (I-B), two D are H, and each remaining D is independently selected from the group consisting of R⁰⁷⁷, —X²—R^{d}, C₁-C₆alkoxy and —OR^{6a}.

In another preferred embodiment of Formula (I-B), two D are H and each remaining D is independently selected from the group consisting of alkoxy (preferably methoxy) and —OR^{6a}.

Other preferred embodiments of Formula (I-B) include preferred A, Z, V, R¹³, L, b, W and R¹⁴-R¹⁷ embodiments as described for Formula (I).

According to another embodiment, the invention provides compounds of Formula (II) and racemic mixtures, diastereomers and enantiomers thereof: 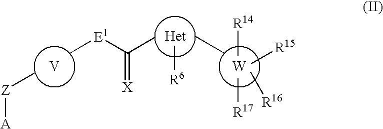
and N-oxides, hydrates, solvates, pharmaceutically acceptable salts, prodrugs and complexes thereof, wherein,
A, Z, V, X, W, R¹⁴, R¹⁵, R¹⁶ and R¹⁷, and preferred embodiments thereof, are as defined for Formula (I); and
E¹ is selected from the group consisting of —CH₂—, —N(R¹³)—, —N(H)—, —N(C₁-C₆alkyl)-, —CH₂N(H)— and —N(H)CH₂—, wherein R¹³ is as defined for Formula (I);
R⁶ is selected from the group consisting of absent, H, halogen, alkyl, alkenyl, alkynyl, CN, alkoxy, NH₂, trihalomethyl, NH(alkyl), di-alkylamino and alkyl-thio, wherein any said alkyl, alkenyl, alkynyl or alkoxy is optionally substituted; and Het is an optionally substituted 5 or 6-membered aryl or heterocyclic.

In a preferred embodiment, E¹ is —NH— or —CH₂—.

In a preferred embodiment, Het is a 5- or 6-membered heteroaryl.

In another preferred embodiment, Het is a 5-membered heteroaryl.

In another preferred embodiment, Het is selected from the group consisting of:

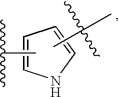

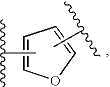

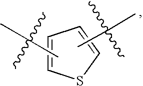

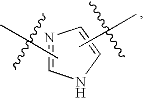

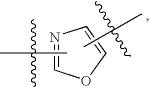

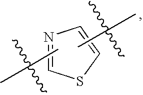

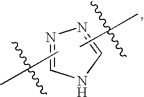

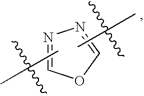

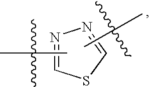

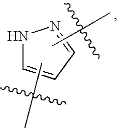

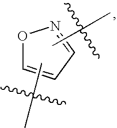

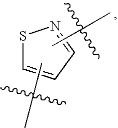

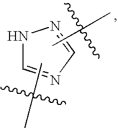

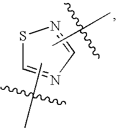

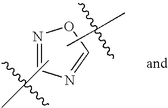

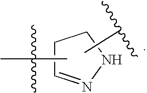

In another preferred embodiment, R⁶ is absent or an optionally substituted alkyl, preferably trihalomethyl, more preferably —CF₃.

Other preferred embodiments of Formula (II) include preferred A, Z, V, X, R¹³, W and R¹⁴-R¹⁷ embodiments as described for Formula (I).

According to another embodiment, the invention provides compounds of Formula (III) and racemic mixtures, diastereomers and enantiomers thereof: 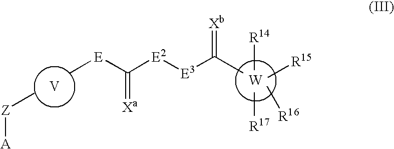
and N-oxides, hydrates, solvates, pharmaceutically acceptable salts, prodrugs and complexes thereof, wherein,
A, Z, V, E, W, R¹⁴, R¹⁵, R¹⁶ and R¹⁷, and preferred embodiments thereof, are as defined for Formula (I); and
X^{a} and X^{b} are independently selected from the group consisting of O, S, N(H), N(alkyl), N(OH), N(O-alkyl), and N(CN); and
E, E² and E³ are each independently selected from the group consisting of —N(R¹³)—, —N(H)—, —N(C₁-C₆alkyl)-, —CH₂N(H)— and —N(H)CH₂—, wherein R¹³ is as defined for Formula (I).

In a preferred embodiment of Formula (III), each of E, E² and E³ are independently selected from —N(R¹³)—.

In a more preferred embodiment at least one of E, E² and E³ is —NH—.

In another preferred embodiment, at least two of E, E² and E³ are —NH—.

In a preferred embodiment of Formula (III), each of E, E² and E³ are —NH—.

In a preferred embodiment of Formula (III), X^{a} and X^{b} are independently selected from O and S.

In a preferred embodiment of Formula (III), at least one of X^{a} and X^{b} are O.

In a preferred embodiment of Formula (III), both of X^{a} and X^{b} are O.

Other preferred embodiments of Formula (III) include preferred A, Z, V, E, W and R¹⁴-R¹⁷ embodiments as described for Formula (I).

According to another embodiment, the invention provides compounds of Formula (IV) and racemic mixtures, diastereomers and enantiomers thereof: 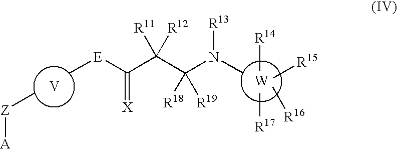
and N-oxides, hydrates, solvates, pharmaceutically acceptable salts, prodrugs and complexes thereof,
and compounds of Formula (V) and racemic mixtures, diatereomers and enantiomers thereof: 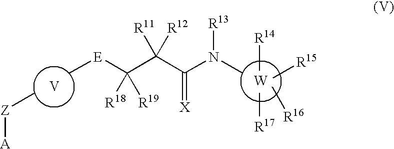
and N-oxides, hydrates, solvates, pharmaceutically acceptable salts, prodrugs and complexes thereof, wherein,
A, Z, V, E, X, W, R¹³, R¹⁴, R¹⁵, R¹⁶ and R¹⁷, and preferred embodiments thereof, are as defined for Formula (I); and
R¹¹ and R¹² are independently selected from the group consisting of H, halogen, —OH, unsubstituted —O—(C₁-C₆alkyl), substituted —O—(C₁-C₆alkyl), unsubstituted —O-(cycloalkyl), substituted —O-(cycloalkyl), unsubstituted —NH(C₁-C₆alkyl), substituted —NH(C₁-C₆alkyl), —NH₂, —SH, unsubstituted —S—(C₁-C₆alkyl), substituted —S—(C₁-C₆alkyl), unsubstituted C₁-C₆alkyl and substituted C₁-C₆alkyl; or
R¹¹ and R¹² taken together with the atom to which they are attached form a C₃-C₇ ring system, wherein said ring system is optionally substituted; or
R¹² and R¹³ taken together with the atoms to which they are attached optionally form a 4 to 8 membered cycloalkyl or heterocyclic ring system, which ring system is optionally substituted; or
R¹³ and R¹⁴ taken together with the atoms to which they are attached optionally form a 4 to 8 membered cycloalkyl or heterocyclic ring system, which ring system is optionally substituted; and
R¹⁸ and R¹⁹ are independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or
R¹⁸ and R¹⁹ together with the atom to which they are attached form a 3 to 6 membered cycloalkyl or heterocycle, each of which is optionally substituted with 1 to 4 halo, preferably F;

According to a preferred embodiment of the present invention, R¹¹ and R¹² are each —H.

According to another preferred embodiment of the present invention, R¹¹, R¹² and R¹³ are each —H.

According to another preferred embodiment of the present invention, one of R¹⁸ and R¹⁹ is —CF₃ and the other is —H.

According to another preferred embodiment of the present invention, X is O, one of R¹⁸ and R¹⁹ is —CF₃ and the other is —H, and R¹¹, R¹² and R¹³ are each —H.

Other preferred embodiments of Formula (IV) and Formula (V) include preferred A, Z, V, E, W and R¹⁴-R¹⁷embodiments as described for Formula (I).

In another embodiment of the present invention, the invention provides compounds of Formula (IV-A) and Formula (V-A) and racemic mixtures, diastereomers and enantiomers thereof: 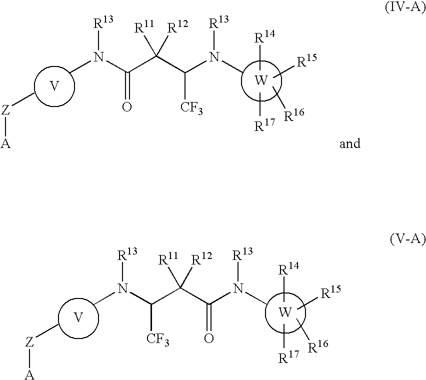
and N-oxides, hydrates, solvates, pharmaceutically acceptable salts, prodrugs and complexes thereof, wherein A, Z, V, W, R¹¹, R¹², R¹³, R¹⁴, R¹⁵, R¹⁶ and R¹⁷, and preferred embodiments thereof, are as defined for Formula (IV) and Formula (V).

In a preferred embodiment of the compounds according to Formula (IV-A) and Formula (V-A), W is phenyl.

Other preferred embodiments of Formula (IV-A) and Formula (V-A) include preferred A, Z, V, W and R¹⁴-R¹⁷ embodiments as described for Formula (I).

Another embodiment of the present invention provides a composition comprising a therapeutically effective amount of a compound, or racemic mixtures, diastereomers and enantiomers thereof, according to any embodiment or preferred embodiment thereof of the present invention, or an N-oxide, hydrate, solvate, pharmaceutically acceptable salt, prodrug or complex thereof, together with a pharmaceutically acceptable carrier, excipient or diluent.

A further aspect of the present invention provides a method of inhibiting receptor type tyrosine kinase signaling, preferably VEGF receptor signaling and HGF receptor signaling, the method comprising contacting the receptor with a compound, or racemic mixtures, diastereomers and enantiomers thereof, according to any embodiment or preferred embodiment thereof of the present invention, or an N-oxide, hydrate, solvate, pharmaceutically acceptable salt, prodrug or complex thereof, or with a composition according to the present invention. Inhibition of receptor type tyrosine kinase activity, preferably VEGF and HGF receptor signaling can be in a cell or a multicellular organism. If in a multicellular organism, the method according to this aspect of the invention comprises administering to the organism a compound, or racemic mixtures, diastereomers and enantiomers thereof, according to any embodiment or preferred embodiment of the present invention, or an N-oxide, hydrate, solvate, pharmaceutically acceptable salt, prodrug or complex thereof, or a composition according to the present invention. Preferably the organism is a mammal, more preferably a human.

Examples of kinases that are inhibited by the compounds and compositions described herein and against which the methods described herein are useful include, but are not limited to, c-Met and KDR.

Depending on the particular condition, or disease, to be treated, additional therapeutic agents, which could be normally administered to treat that condition, may also be present in the compositions of this invention. In other words, compounds of this invention can be administered as the sole pharmaceutical agent or in combination with one or more other additional therapeutic (pharmaceutical) agents where the combination causes no unacceptable adverse effects. This may be of particular relevance for the treatment of hyper-proliferative diseases such as cancer. In this instance, the compound of this invention can be combined with known cytotoxic agents, signal transduction inhibitors, or with other anti-cancer agents, as well as with admixtures and combinations thereof. As used herein, additional therapeutic agents that are normally administered to treat a particular disease, or condition, are known as “appropriate for the disease, or condition, being treated”. As used herein, “additional therapeutic agents” is meant to include chemotherapeutic agents and other anti-proliferative agents.

For example, chemotherapeutic agents or other anti-proliferative agents may be combined with the compounds of this invention to treat proliferative disease or cancer. Examples of chemotherapeutic agents or other anti-proliferative agents include HDAC inhibitors including, but are not limited to, SAHA, MS-275, MGO103, and those described in WO 2006/010264, WO 03/024448, WO 2004/069823, US 2006/0058298, US 2005/0288282, WO 00/71703, WO 01/38322, WO 01/70675, WO 03/006652, WO 2004/035525, WO 2005/030705, WO 2005/092899, and demethylating agents including, but not limited to, 5-aza-dC, Vidaza and Decitabine and those described in U.S. Pat. No. 6,268,137, U.S. Pat. No. 5,578,716, U.S. Pat. No. 5,919,772, U.S. Pat. No. 6,054,439, U.S. Pat. No. 6,184,211, U.S. Pat. No. 6,020,318, U.S. Pat. No. 6,066,625, U.S. Pat. No. 6,506,735, U.S. Pat. No. 6,221,849, U.S. Pat. No. 6,953,783, U.S. Ser. No. 11/393,380 and PCT/US2006/001791.

In another embodiment of the present invention, for example, chemotherapeutic agents or other anti-proliferative agents may be combined with the compounds of this invention to treat proliferative diseases and cancer. Examples of known chemotherapeutic agents include, but are not limited to, for example, other therapies or anticancer agents that may be used in combination with the inventive anticancer agents of the present invention and include surgery, radiotherapy (in but a few examples, gamma-radiation, neutron beam radiotherapy, electron beam radiotherapy, proton therapy, brachytherapy, and systemic radioactive isotopes, to name a few), endocrine therapy, taxanes (taxol, taxotere etc), platinum derivatives, biologic response modifiers (interferons, interleukins, and tumor necrosis factor (TNF), TRAIL receptor targeting agents, to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate, Pemetrexed etc), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), Cell cycle inhibitors (KSP mitotic kinesin inhibitors, CENP-E and CDK inhibitors), enzymes (Asparaginase), and hormones (Tamoxifen, Leuprolide, Flutamide, and Megestrol), Gleevec™, adriamycin, dexamethasone, and cyclophosphamide. Antiangiogenic agents (Avastin and others). Kinase inhibitors (Imatinib (Gleevec), Sutent, Nexavar, Erbitux, Herceptin, Tarceva, Iressa and others). Agents inhibiting or activating cancer pathways such as the mTOR, HIF (hypoxia induced factor) pathways and others. For a more comprehensive discussion of updated cancer therapies see, http://www.nci.nih.gov/, a list of the FDA approved oncology drugs at http://www.fda.gov/cder/cancer/druglistframe.htm, and The Merck Manual, Eighteenth Ed. 2006, the entire contents of which are hereby incorporated by reference.

In another embodiment, the compounds of the present invention can be combined with cytotoxic anti-cancer agents. Examples of such agents can be found in the 13th Edition of the Merck Index (2001) These agents include, by no way of limitation, asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other cytotoxic drugs suitable for use with the compounds of the invention include, but are not limited to, those compounds acknowledged to be used in the treatment of neoplastic diseases, such as those for example in Goodman and Gilman's The Pharmacological Basis of Therapeutics (Ninth Edition, 1996, McGraw-Hill). These agents include, by no way of limitation, aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other cytotoxic anti-cancer agents suitable for use in combination with the compounds of the invention also include newly discovered cytotoxic principles such as oxaliplatin, gemcitabine, capecitabine, epothilone and its natural or synthetic derivatives, temozolomide (Quinn et al., J. Clin. Oncology 2003, 21(4), 646-651), tositumomab (Bexxar), trabedectin (Vidal et al., Proceedings of the American Society for Clinical Oncology 2004, 23, abstract 3181), and the inhibitors of the kinesin spindle protein Eg5 (Wood et al., Curr. Opin. Pharmacol. 2001, 1, 370-377).

In another embodiment, the compounds of the present invention can be combined with other signal transduction inhibitors. Of particular interest are signal transduction inhibitors which target the EGFR family, such as EGFR, HER-2, and HER-4 (Raymond et al., Drugs 2000, 60 (Supp1.1), 15-23; Harari et al., Oncogene 2000, 19 (53), 6102-6114), and their respective ligands. Examples of such agents include, by no way of limitation, antibody therapies such as Herceptin (trastuzumab), Erbitux (cetuximab), and pertuzumab. Examples of such therapies also include, by no way of limitation, small-molecule kinase inhibitors such as ZD-1839/Iressa (Baselga et al., Drugs 2000, 60 (Suppl. 1), 33-40), OSI-774/Tarceva (Pollack et al. J. Pharm. Exp. Ther. 1999, 291(2), 739-748), CI-1033 (Bridges, Curr. Med. Chem. 1999, 6, 825-843), GW-2016 (Lackey et al., 92nd AACR Meeting, New Orleans, Mar. 24-28, 2001, abstract 4582), CP-724,714 (Jani et al., Proceedings of the American Society for Clinical Oncology 2004, 23, abstract 3122), HKI-272 (Rabindran et al., Cancer Res. 2004, 64, 3958-3965), and EKB-569 (Greenberger et al., 11th NCI-EORTC-AACR Symposium on New Drugs in Cancer Therapy, Amsterdam, Nov. 7-10, 2000, abstract 388).

In another embodiment, the compounds of the present invention can be combined with other signal transduction inhibitors targeting receptor kinases of the split-kinase domain families (VEGFR, FGFR, PDGFR, flt-3, c-kit, c-fins, and the like), and their respective ligands. These agents include, by no way of limitation, antibodies such as Avastin (bevacizumab). These agents also include, by no way of limitation, small-molecule inhibitors such as STI-571/Gleevec (Zvelebil, Curr. Opin. Oncol., Endocr. Metab. Invest. Drugs 2000, 2(1), 74-82), PTK-787 (Wood et al., Cancer Res. 2000, 60(8), 2178-2189), SU-11248 (Demetri et al., Proceedings of the American Society for Clinical Oncology 2004, 23, abstract 3001), ZD-6474 (Hennequin et al., 92nd AACR Meeting, New Orleans, Mar. 24-28, 2001, abstract 3152), AG-13736 (Herbst et al., Clin. Cancer Res. 2003, 9, 16 (suppl 1), abstract C253), KRN-951 (Taguchi et al., 95<th> AACR Meeting, Orlando, Fla., 2004, abstract 2575), CP-547,632 (Beebe et al., Cancer Res. 2003, 63, 7301-7309), CP-673,451 (Roberts et al., Proceedings of the American Association of Cancer Research 2004, 45, abstract 3989), CHIR-258 (Lee et al., Proceedings of the American Association of Cancer Research 2004, 45, abstract 2130), MLN-518 (Shen et al., Blood 2003, 102, 11, abstract 476), and AZD-2171 (Hennequin et al., Proceedings of the American Association of Cancer Research 2004, 45, abstract 4539).

In another embodiment, the compounds of the present invention can be combined with inhibitors of the Raf/MEK/ERK transduction pathway (Avruch et al., Recent Prog. Horm. Res. 2001, 56, 127-155), or the PKB (akt) pathway (Lawlor et al., J. Cell Sci. 2001, 114, 2903-2910). These include, by no way of limitation, PD-325901 (Sebolt-Leopold et al., Proceedings of the American Association of Cancer Research 2004, 45, abstract 4003), and ARRY-142886 (Wallace et al., Proceedings of the American Association of Cancer Research 2004, 45, abstract 3891).

In another embodiment, the compounds of the present invention can be combined with inhibitors of histone deacetylase. Examples of such agents include, by no way of limitation, suberoylanilide hydroxamic acid (SAHA), LAQ-824 (Ottmann et al., Proceedings of the American Society for Clinical Oncology 2004, 23, abstract 3024), LBH-589 (Beck et al., Proceedings of the American Society for Clinical Oncology 2004, 23, abstract 3025), MS-275 (Ryan et al., Proceedings of the American Association of Cancer Research 2004, 45, abstract 2452), FR-901228 (Piekarz et al., Proceedings of the American Society for Clinical Oncology 2004, 23, abstract 3028) and MGCDO103 (U.S. Pat. No. 6,897,220).

In another embodiment, the compounds of the present invention can be combined with other anti-cancer agents such as proteasome inhibitors, and m-TOR inhibitors. These include, by no way of limitation, bortezomib (Mackay et al., Proceedings of the American Society for Clinical Oncology 2004, 23, Abstract 3109), and CCI-779 (Wu et al., Proceedings of the American Association of Cancer Research 2004, 45, abstract 3849). The compounds of the present invention can be combined with other anti-cancer agents such as topoisomerase inhibitors, including but not limited to camptothecin.

Those additional agents may be administered separately from the compound-containing composition, as part of a multiple dosage regimen. Alternatively, those agents may be part of a single dosage form, mixed together with the compound of this invention in a single composition. If administered as part of a multiple dosage regimen, the two active agents may be submitted simultaneously, sequentially or within a period of time from one another which would result in the desired activity of the agents.

The amount of both the compound and the additional therapeutic agent (in those compositions which comprise an additional therapeutic agent as described above) that may be combined with the carrier materials to produce a single dosage form will vary depending upon the host treated and the particular mode of administration.

In those compositions which comprise an additional therapeutic agent, that additional therapeutic agent and the compound of this invention may act synergistically.

The data presented herein demonstrate the inhibitory effects of the kinase inhibitors of the invention. These data lead one to reasonably expect that the compounds of the invention are useful not only for inhibition of protein tyrosine kinase activity, or preferably VEGF receptor signaling and HGF receptor signaling, but also as therapeutic agents for the treatment of proliferative diseases, including cancer and tumor growth.

Preferred compounds according to the invention include those described in the examples below. Compounds were named using Chemdraw Ultra version 6.0.2 or version 8.0.3, which are available through Cambridgesoft.com, 100 Cambridge Park Drive, Cambridge, Mass. 02140, Namepro version 5.09, which is available from ACD labs, 90 Adelaide Street West, Toronto, Ontario, M5H, 3V9, Canada, or were derived therefrom.

### Synthetic Schemes and Experimental Procedures

The compounds of the invention can be prepared according to the reaction schemes or the examples illustrated below utilizing methods known to one of ordinary skill in the art. These schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

### General Procedures

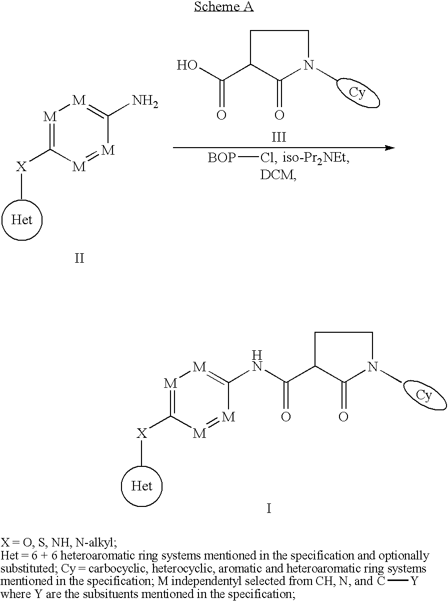

### Synthesis of 2-oxo-1-cyclylpyrrolidine-3-carboxamides (I)

2-Oxo-1-cyclylpyrrolidine-3-carboxamides of a general formula I could be prepared via a coupling reaction between amines II and 2-oxo-1-cyclylpyrrolidine-3-carboxylic acids of a general formula III (scheme A), whereas amines II represent appropriately substituted various scaffolds suitable for the synthesis of kinase inhibitors or other compounds of pharmaceutical interest. Coupling of amines II with the acids III could be achieved in aprotic solvents such as DCM, CHCl₃, toluene, ethylene glycol dimethyl ether, MeCN, DMF, DMSO, THF, dioxane and like, using activating agents used in peptide chemistry and known to the skilled in the art, in the presence of organic bases such as DIPEA, Et₃N, DBU, DMAP, N-methylmorpholine, N-methylpiperidine, and like.

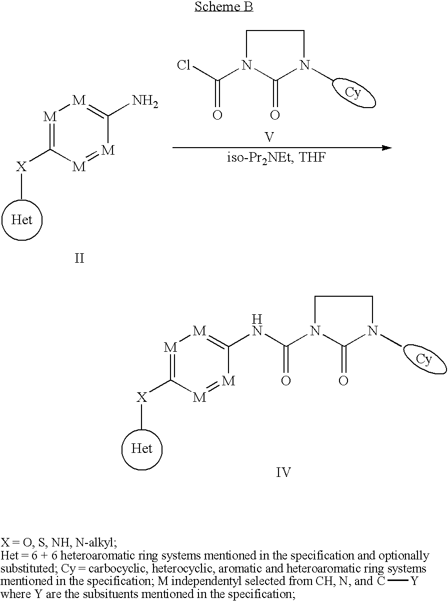

### Synthesis of 2-oxo-3-cyclylimidazolidine-1-carboxamides (IV)

2-Oxo-3-cyclylimidazolidine-1-carboxamides of a general formula IV could be prepared via a condensation reaction between amines II and 2-oxo-3-cyclylimidazolidine-1-carbonyl chlorides of a general formula V (scheme B), whereas amines II represent appropriately substituted various scaffolds suitable for the synthesis of kinase inhibitors or other compounds of pharmaceutical interest. Coupling of amines II with the carbonyl chlorides V could be achieved in aprotic solvents such as DCM, CHCl₃, toluene, ethylene glycol dimethyl ether, MeCN, DMF, DMSO, THF, dioxane and like, in the presence of organic bases such as DIPEA, Et₃N, DBU, DMAP, N-methylmorpholine, N-methylpiperidine, and like.

### PARTICULAR EXAMPLES

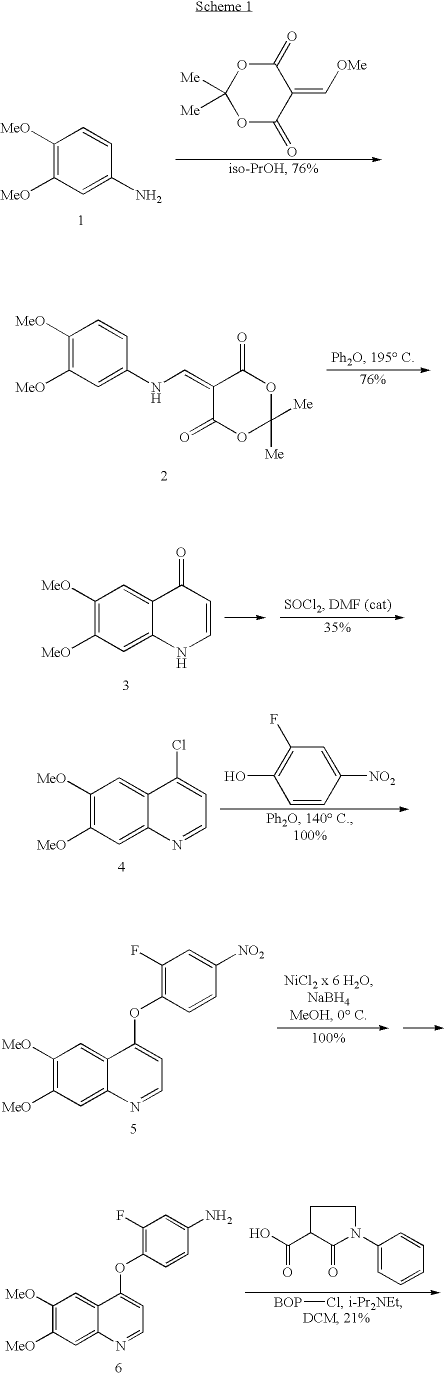

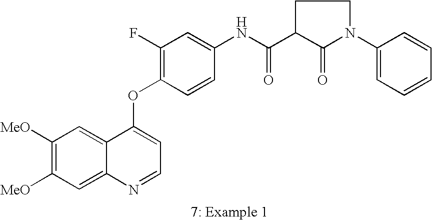

### Example 1

### N-(4-(6,7-Dimethoxyquinolin-4-yloxy)-3-fluorophenyl)-2-oxo-1-phenylpyrrolidine-3-carboxamide (7)

### Step 1: 6,7-Dimethoxyquinolin-4(1H)-one (3)

To a solution of 1 (6.41 g, 41.8 mmol) in iso-PrOH (100 ml) was added 5-(methoxymethylene)-2,2-dimethyl-1,3-dioxane-4,6-dione (7.78 g, 41.8 mmol) (Montatsh. Chem. 1967, 98, 564) and the heterogeneous mixture was heated to reflux for an hour. The mixture was cooled to room temperature and the solid was collected by filtration then washed sequentially with iso-PrOH, iso-PrOH/Et₂O (1:1) and finally with diethyl ether to afford compound 2 (9.8 g, 76% yield) as a white solid which was used immediately in the next step with no additional purification.

A suspension of 2 (9.8 g, 31.9 mmol) in Ph₂O (50 ml) was heated to 195° C. for 30 minutes. The mixture was cooled to room temperature and diluted with Et₂O; a brown precipitate was formed which was collected by filtration and washed with Et₂O, to afford title compound 3 (6.54 g, 76% yield).

LRMS (M+1): 206.2 (100%).

This material was used directly in the next step with no additional purification.

### Step 2: 4-Chloro-6,7-dimethoxyquinoline (4)

A suspension of the quinolone 3 (6.54 g, 31.9 mmol) in SOCl₂ (70 ml) and DMF (cat) was heated to reflux for 1 hr. The reaction mixture was cooled to room temperature and concentrated. The residue was basified with NH₄OH solution and then extracted with DCM. The extract was dried over anhydrous Na₂SO₄, filtered then concentrated. The residue was purified by column chromatography (eluent a gradient of 20% EtOAc/hexane to 100% EtOAc) to afford title compound 4 (2.5 g, 35% yield) as a brown solid.

LRMS (M+1): 224.1 (75%)/226.1 (25%).

### Step 3: 4-(2-Fluoro-4-nitrophenoxy)-6,7-dimethoxyquinoline (5)

A suspension of 4 (791 mg, 3.54 mmol) and 2-fluoro-4-nitrophenol (1.11 g, 7.07 mmol) in Ph₂O (15 ml) was heated to 140° C. for 24 hr. The reaction mixture was cooled to room temperature and diluted with Et₂O. A precipitate formed which was collected by filtration to afford the title compound 5 as a beige solid (1.2 g, 100% yield).

LRMS (M+1): 345.1 (100%).

This material was used directly in the next step with no additional purification.

### Step 4: N-(4-(6,7-Dimethoxyquinolin-4-yloxy)-3-fluorophenyl)-2-oxo-1-phenylpyrrolidine-3-carboxamide (7)

To a solution of 5 (200 mg, 0.58 mmol) in MeOH (10 ml) was added solid NiCl₂×6H₂O (275 mg, 1.16 mmol) at 0° C. followed by NaBH₄ (86 mg, 2.32 mmol) and the reaction mixture was stirred at 0° C. for 30 min, concentrated to dryness and dissolved in 1 M HCl solution. The acidic solution was basified with conc. NH₄OH and extracted with DCM. The organic extract was dried over anhydrous Na₂SO₄ and filtered. The filtrate was concentrated under reduced pressure and the remaining crude amine 6 (182 mg, 100% yield) was used directly in the next step without additional purification.

To a solution of 2-oxo-1-phenylpyrrolidine-3-carboxylic acid (241 mg, 1.16 mmol) in dry DCM (7 ml), at 0° C., was added, BOPCI (294 mg, 1.16 mmol) and the reaction mixture was stirred for 10 minutes. A solution of the amine 6 (182 mg, 0.58 mmol) and iso-Pr₂NEt (448 mg, 3.47 mmol) in dry DCM (7 ml) was then added and the reaction mixture was stirred at room temperature for 2 hrs, concentrated to dryness and partitioned between EtOAc and saturated NaHCO₃ solution. The organic phase was collected, washed twice with saturated NaHCO₃ solution, dried over anhydrous Na₂SO₄ and filtered. The filtrate was concentrated under reduced pressure and the residue was purified by column chromatography (eluent EtOAc), to afford title compound 7 (61 mg, 21% yield) as an off white solid.

¹H NMR (400 MHz, DMSO-d₆) δ (ppm): 10.67 (s, 1H), 8.44 (d, J=5.28 Hz, 1H), 7.90 (dd, J=2.35 and 13.1 Hz, 1H), 7.67-7.35 (m, 6H), 7.14 (m, 1H), 6.45 (dd, J=0.98 and 5.28 Hz, 1H), 3.93 (s, 6H), 3.91 (t, J=1.57 Hz, 1H) 3.31 (s, 4H).

LRMS (M+1): 502.1 (100%).

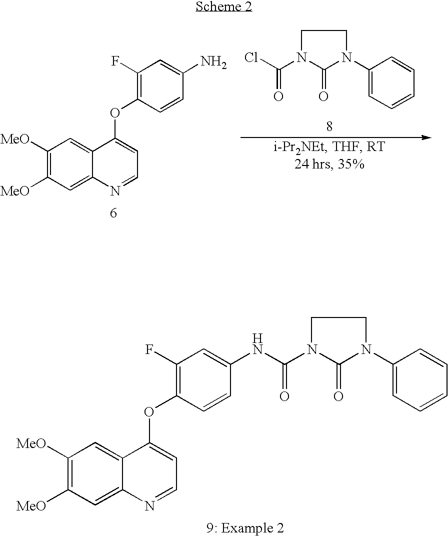

### Example 2

### N-(4-(6,7-Dimethoxyquinolin-4-yloxy)-3-fluorophenyl)-2-oxo-3-phenylimidazolidine-1-carboxamide (9)

J To a solution of amine 6 (182 mg, 0.58 mmol) and iso-Pr₂NEt (448 mg, 3.47 mmol) in dry DCM (7 ml) was added 2-oxo-3-phenylimidazolidine-1-carbonyl chloride (8) (Chem. Abstr.; 88; 6873 and P. Mayer, et al.; J. Med. Chem.; 2000, 43, 3653-3664) (260 mg, 1.16 mmol) and the reaction mixture was stirred at room temperature overnight. The reaction mixture was concentrated to dryness and the residue was partitioned between EtOAc and water. The organic phase was collected, dried over anhydrous Na₂SO₄ and filtered. The filtrate was concentrated under reduced pressure and the remaining solid was purified by column chromatography (eluent EtOAc) to afford title compound 9 as an off white solid (100 mg, 35% yield).

¹H NMR (400 MHz, DMSO-d₆) δ (ppm): 10.55 (s, 1H), 8.46 (d, J=5.28 Hz, 1H), 7.82 (m, 1H), 7.61 (d, J=7.8 Hz, 2H), 7.51 (s, 1H), 7.42 (m, 5H), 7.16 (t, J=7.24 Hz, 1H), 6.45 (d, J=5.09 Hz, 1H), 3.92 (s, 6H), 3.31 (s, 4H).

LRMS (M+1) 503.1 (100%).

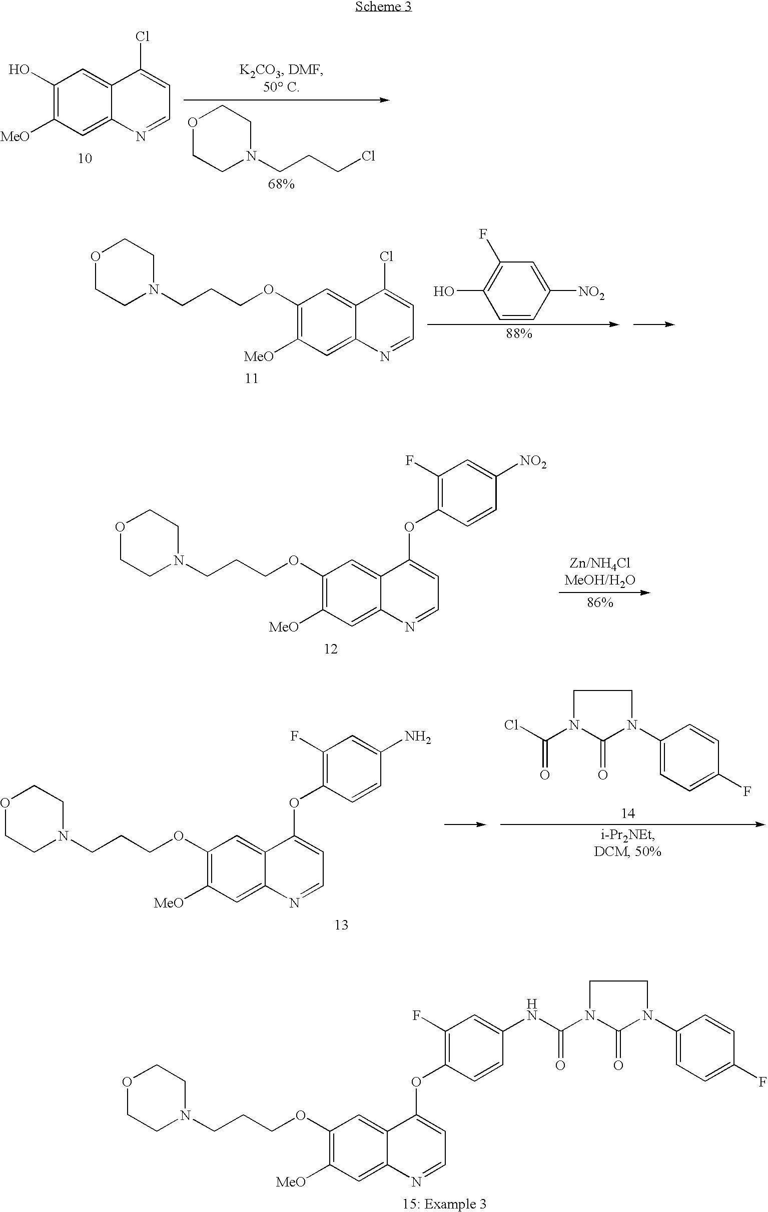

### Example 3

### N-(3-Fluoro-4-(7-methoxy-6-(3-morpholinopropoxy)quinolin-4-yloxy)phenyl)-3-(4-fluorophenyl)-2-oxoimidazolidine-1-carboxamide (15)

### Step 1: 4-(3-(4-Chloro-7-methoxyquinolin-6-yloxy)propyl)morpholine (11)

To a solution of 4-chloro-7-methoxyquinolin-6-ol (10) (1.0 g, 4.77 mmol) (Patent application WO 98/13350) in DMF (20 ml) was added 4-(3-chloropropyl)morpholine (777 mg, 4.77 mmol) and K₂CO₃ (1.97 g, 14.31 mmol). The reaction mixture was heated to 50° C. for 6 hrs, concentrated to 50% of the original volume and partitioned between water and EtOAc. The organic phase was collected, washed with water (3×20 ml), dried over anhydrous Na₂SO₄, filtered and concentrated. The resultant solid was triturated with Et₂O to give 11 as a white solid (1.1 g, 68% yield).

¹H NMR (400 MHz, DMSO-d₆) δ (ppm): 8.60 (d, J=4.70 Hz, 1H), 7.55 (d, J=4.70 Hz, 1H), 7.44 (s, 1H), 7.36 (s, 1H), 4.20 (t, J=6.46 Hz, 2H), 3.95 (s, 3H), 3.57 (t, J=4.70 Hz, 4H), 2.47 (m, 6H), 1.97 (m, 2H).

### Step 2: 4-(3-(4-(2-Fluoro-4-nitrophenoxy)-7-methoxyquinolin-6-yloxy)propyl)morpholine (12)

A mixture of the chloride 11 (993 mg, 2.95 mmol) and 2-fluoro-4-nitrophenol (1.39 g, 8.85 mmol) in Ph₂O (15 ml) were heated to 150° C. for 24 hrs. The reaction mixture was cooled to room temperature, diluted with EtOAc and washed with sat NaHCO₃ solution. The organic phase was collected, dried over anhydrous Na₂SO₄ and filtered. The filtrate was concentrated and the residue was purified by column chromatography (eluent gradient of EtOAc to 10% MeOH in EtOAc), to afford 12 as a pale yellow solid (1.2 g, 88% yield).

¹H NMR (400 MHz, DMSO-d₆) δ (ppm): 8.54 (d, J=5.09 Hz, 1H), 8.43 (dd, J=2.54 and 10.4 Hz, 1H), 8.17 (dd, J=1.37 and 9.0 Hz, 1H), 7.58 (t, J=8.41 Hz, 1H), 7.43 (d, J=4.30 Hz, 1H), 6.75 (d, J=5.09 Hz, 1H), 4.13 (t, J=6.46 Hz, 2H), 3.94 (s, 3H), 3.52 (t, J=4.50 Hz, 4H), 2.48 (m, 2H) 2.41 (t, J=7.04 Hz, 2H), 2.33 (m, 2H), 1.92 (m, 2H).

### Step 3: N-(3-Fluoro-4-(7-methoxy-6-(3-morpholinopropoxy)quinolin-4-yloxy)phenyl)-3-(4-fluorophenyl)-2-oxoimidazolidine-1-carboxamide (15)

To a solution of the nitro compound 12 (1.1 g, 2.40 mmol) in MeOH (20 mL), THF (5 mL) and H₂O (4 mL) was added zinc (1.42 g, 21.6 mmol) and NH₄Cl (128 mg, 2.40 mmol) and the reaction mixture was heated to reflux for 2 hrs. The reaction mixture was cooled to room temperature, filtered and concentrated. The residue was triturated with Et₂O to afford amine 13 as a yellow solid (884 mg, 86% yield) which was used with no additional purification.

To a solution of 13 (200 mg, 0.47 mmol) and iso-Pr₂NEt (362 mg, 2.81 mmol) in dry DCM (10 ml) was added 3-(4-fluorophenyl)-2-oxoimidazolidine-1-carbonyl chloride (14) (Chem. Abstr.; 88; 6873 and P. Mayer, et al.; J. Med. Chem.; 2000, 43, 3653-3664) (228 mg, 9.4 mmol). The reaction mixture was stirred at room temperature overnight, concentrated to dryness and partitioned between EtOAc and water. The organic phase was collected, dried over anhydrous Na₂SO₄ and filtered. The filtrate was evaporated under reduced pressure and the residue was purified by column chromatography (eluent EtOAc), to afford title compound 15 as an off white solid (150 mg, 50% yield).

¹H NMR (400 MHz, DMSO-d₆) δ (ppm): 10.52 (s, 1H), 8.45 (d, J=5.28 Hz, 1H), 7.84 (m, 1H), 7.63 (m, 2H), 7.50 (s, 1H), 7.42 (m, 2H), 7.39 (s, 1H), 7.27 (t, J=8.61 Hz, 2H), 6.44 (d, J=5.09 Hz, 1H), 4.17 (t, J=6.26 Hz, 2H), 3.94 (s, 7H), 3.54 (t, J=4.30 Hz, 4H), 2.48 (m, 6H), 1.95 (m, 2H).

LRMS (M+1): 634.2 (100%).

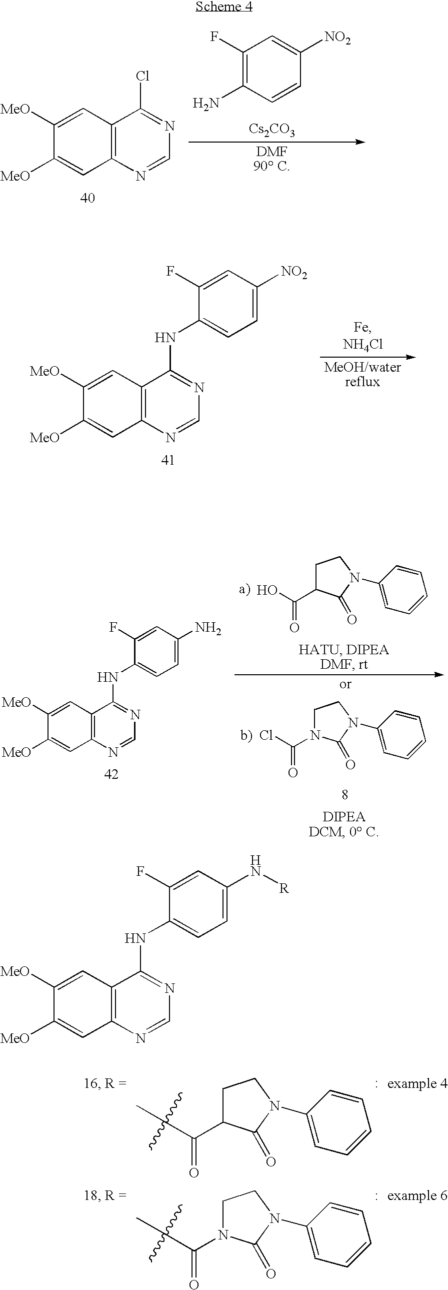

### Example 4

### N-(4-(6,7-dimethoxyquinazolin-4-ylamino)-3-fluorophenyl)-2-oxo-1-phenylpyrrolidine-3-carboxamide (27)

### Step 1. N-(2-fluoro-4-nitrophenyl)-6,7-dimethoxyquinazolin-4-amine (41)

A stirred suspension of 4-chloro-6,7-dimethoxyquinazoline (40) (prepared according to A. J. Bridges et al., J. Med. Chem., 1996, 39, 267) (1.00 g, 4.45 mmol), 2-fluoro-4-nitroaniline (940 mg, 6.02 mmol) and cesium carbonate (3.20 g, 9.82 mmol) in anhydrous DMF (20 mL) was heated at 90° C. overnight under nitrogen. The reaction mixture was allowed to cool to room temperature. The reaction mixture was diluted with AcOEt, successively washed with water and a saturated solution of ammonium chloride, concentrated and triturated with AcOEt/hexanes. After filtration, the cake was adsorbed on silica gel and purified by flash column chromatography on (eluents MeOH/DCM: 2/98→10/90) to afford the title compound 41 (1.06 g, 69% yield) as a yellow-green solid. MS (m/z): 345.0 (M+H).

### Step 2. N¹-(6,7-dimethoxyquinazolin-4-yl)-2-fluorobenzene-1,4-diamine (42)

To a stirred suspension of 41 (1.06 g, 3.079 mmol) in a mixture of methanol/water (70 ml/10 ml) were successively added ammonium chloride (247 mg, 4.62 mmol) and iron powder (776 mg, 13.89 mmol). The reaction mixture was heated to reflux overnight, cooled to room temperature and concentrated. The crude material was diluted with MeOH, suspended with celite then filtered through a celite pad and rinsed with methanol. The filtrate was concentrated and the residue was purified by flash column chromatography (eluents 2% of ammonium hydroxide in MeOH/DCM: 5/95-10/90) and triturated with a mixture of AcOEt/hexanes, to afford the title compound 42 (724 mg, 75% yield) as a pale yellow solid. MS (m/z): 315.0 (M+H).

### Step 3a. N-(4-(6,7-dimethoxyquinazolin-4-ylamino)-3-fluorophenyl)-2-oxo-1-phenyl pyrrolidine-3-carboxamide (16)

To a stirred solution of 42 (150 mg, 0.48 mmol) and 2-oxo-1-phenylpyrrolidine-3-carboxylic acid (147 mg, 0.72 mmol) in anhydrous DMF (5 ml) under nitrogen were added DIPEA (250 μl, 1.43 mmol) and HATU reagent (454 mg, 1.19 mmol), respectively. The reaction mixture was stirred at room temperature overnight, diluted with AcOEt, washed with water and a saturated solution of ammonium chloride, and concentrated. The residue was purified by flash column chromatography on silica gel (eluents 2% of ammonium hydroxide in MeOH/DCM: 5/95) and the product was coprecipitated in AcOEt/hexanes to afford the title compound 16 (165 mg, 69% yield) as a white solid. ¹H NMR (400 MHz, DMSO-d₆) δ (ppm): 10.58 (s, 1H), 9.46 (s, 1H), 8.32 (s, 1H), 7.81 (s, 1H), 7.76 (dd, J=12.7, 2.3 Hz, 1H), 7.71-7.66 (m, 2H), 7.48 (t, J=8.7 Hz, 1H), 7.44-7.38 (m, 3H), 7.21-7.15 (m, 2H), 4.00-3.88 (m, 8H), 3.79 (t, J=8.5 Hz, 1H), 2.50-2.31 (m, 2H). MS (m/z): 502.0 (M+H).

### Example 6

### N-(4-(6,7-dimethoxyquinazolin-4-ylamino)-3-fluorophenyl)-2-oxo-3-phenyl imidazolidine-1-carboxamide (18)

### Step 3b. N-(4-(6,7-dimethoxyquinazolin-4-ylamino)-3-fluorophenyl)-2-oxo-3-phenyl imidazolidine-1-carboxamide (18)

To a stirred solution of 42 (150 mg, 0.48 mmol) and DIPEA (416 μl, 2.39 mmol) in anhydrous DCM (20 mL) at 0° C. under nitrogen was slowly added a solution of 2-oxo-3-phenylimidazolidine-1-carbonyl chloride (8, 7.2 mL, 0.72 mmol, 0.1 M) in THF. The reaction mixture was stirred at 0° C. for 1.5 h, quenched with MeOH and concentrated. The residue was diluted with AcOEt, successively washed with saturated solution of ammonium chloride and water and concentrated. The residue was adsorbed on silica gel and purified by flash column chromatography on silica gel (eluents 2% of ammonium hydroxide in MeOH/DCM: 2/98→5/95). The material obtained was coprecipitated with AcOEt with traces of acetone/hexanes, then triturated with MeOH, and finally triturated with a mixture of MeOH/DMSO/formic acid/water. After filtration, the solid was successively washed with MeOH, water and MeOH, air-dried and dried under high vacuum to afford the title compound 18 (111 mg, 46% yield) as an off-white solid. ¹H NMR (400 MHz, DMSO-d₆) δ (ppm): 10.52 (s, 1H), 9.45 (s, 1H), 8.33 (s, 1H), 7.81 (s, 1H), 7.69 (dd, J=12.5, 2.3 Hz, 1H), 7.66-7.61 (m, 2H), 7.50-7.40 (m, 3H), 7.33 (dd, J=8.5, 2.1 Hz, 1H), 7.21-7.15 (m, 2H), 4.02-3.91 (m, 10H). MS (m/z): 503.0 (M+H).

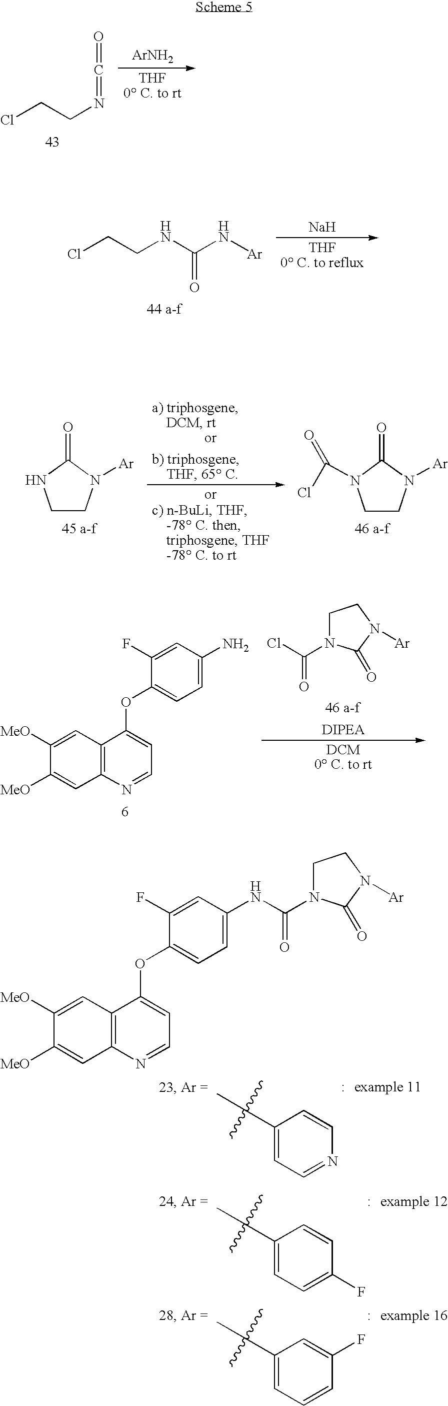

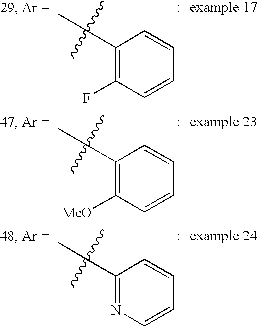

### Example 11

### N-(4-(6,7-dimethoxyquinolin-4-yloxy)-3-fluorophenyl)-2-oxo-3-(pyridin-4-yl)imidazolidine-1-carboxamide (23)

### Step 1. 1-(2-chloroethyl)-3-(pyridin-4-yl)urea (44a)

To a stirred solution of 4-aminopyridine (3.00 g, 31.87 mmol) in anhydrous THF (50 ml) at 0° C. under nitrogen was slowly added 2-chloroethyl isocyanate (43) (3.08 ml, 36.11 mmol). The reaction mixture was stirred at room temperatute overnight whereupon extra isocyanate (1 mL) was added and the reaction mixture was stirred for another day. The reaction mixture was quenched with MeOH then concentrated and coprecipitated with AcOEt/hexanes. After filtration, the solid was rinsed with hexanes and dried under high vacuum to afford the title compound 44a (7.09 g, quantitative yield) as a white fluffy solid. MS (m/z): 200.0 (M+H).

### Step 2. 1-(pyridin-4-yl)imidazolidin-2-one (45a)

To a stirred suspension of NaH (3.55 g, 88.79 mmol, 60% dispersion in oil) in anhydrous THF (75 ml) at 0° C. under nitrogen was slowly added a solution of 44a (7.09 g, 36.11 mmol) in anhydrous THF (50 mL). The reaction mixture was stirred at 0° C. for 30 min, warmed to room temperature for 3.5 h and then heated to reflux for 2.5 h. The reaction was allowed to cool to room temperature and concentrated then quenched by the slow addition of water followed by neutralization to pH 6-7 with 3N HCl and finally washed twice with DCM. The aqueous phase was collected, concentrated, adsorbed onto silica gel then purified by flash column chromatography (eluents 2% of ammonium hydroxide in MeOH/DCM: 10/90w 15/85) and triturated with a mixture of AcOEt with traces of DCM/hexanes to afford the title compound 45a (3.00 g, 52% yield) as a white crystalline solid. MS (m/z): 164.1 (M+H).

### Step 3. 2-oxo-3-(pyridin-4-yl)imidazolidine-1-carbonyl chloride (46a)

To a stirred solution of 45a (326 mg, 2.0 mmol) in anhydrous DCM (60 mL) at room temperature under nitrogen was added triphosgene (445 mg, 1.5 mmol). The reaction mixture (suspension) was stirred at room temperature for 3 h, and used in the next step without any further purification.

### Step 4. N-(4-(6,7-dimethoxyquinolin-4-yloxy)-3-fluorophenyl)-2-oxo-3-(pyridin-4-yl) imidazolidine-1-carboxamide (23)

To a stirred solution at 0° C. under nitrogen of 6 (200 mg, 0.64 mmol) and DIPEA (554 μl, 3.18 mmol) in anhydrous DCM (20 mL) was slowly added a suspension of 46a (25 ml, 0.95 mmol). The reaction mixture was stirred at room temperature overnight then quenched by addition of MeOH and concentrated. The residue was purified by flash column chromatography (eluents MeOH/DCM: 05/95) and the obtained product was coprecipitated with AcOEt/hexanes to afford the title compound 23 (25 mg, 8% yield) as an off-white solid. ¹H NMR (400 MHz, DMSO-d₆) δ (ppm): 10.42 (s, 1H), 8.54 (d, J=6.7 Hz, 2H), 8.49 (d, J=5.3 Hz, 1H), 7.86 (dd, J=12.3, 2.3 Hz, 1H), 7.65 (d, J=6.5 Hz, 2H), 7.53 (s, 1H), 7.51-7.43 (m, 2H), 7.41 (s, 1H), 6.47 (d, J=4.5 Hz, 1H), 3.99-3.93 (m, 10H). MS (m/z): 504.2 (M+H).

Examples 12, 17 and 23 (compounds 24, 29 and 47) were prepared in four steps from the appropriate substituted aniline type compounds (Scheme 5) similarly to compound 28 (example 16).

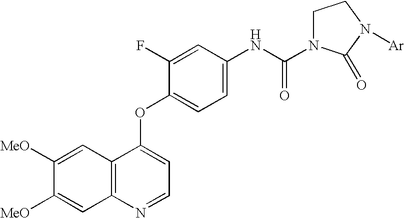

| Cpd | Ex. | Ar | Name | Characterization |
|---|---|---|---|---|
| 24 | 12 | 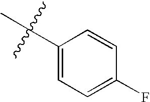 | N-(4-(6,7- dimethoxyquinolin- 4-yloxy)-3- fluorophenyl)-3- (4-fluorophenyl)- 2- oxoimidazolidine- 1-carboxamide | ¹H NMR (400 MHz, DMSO-d₆) δ(ppm): 10.54 (s, 1H), 8.48 (d, J = 5.3 Hz, 1H), 7.85 (d, J = 12.1 Hz, 1H), 7.70-7.61 (m, 2H), 7.53 (s, 1H), 7.48- 7.42 (m, 2H), 7.41 (s, 1H), 7.29 (t, J = |
| # 8.8 Hz, 2H), 6.47 (d, J = 5.3 Hz, 1H), 4.01-3.90 (m, 10H). MS (m/z): 521.2 (M + H). | | | | |
| 29 | 17 | 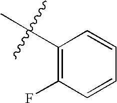 | N-(4-(6,7- dimethoxyquinolin- 4-yloxy)-3- fluorophenyl)-3- (2-fluorophenyl)- 2- oxoimidazolidine- 1-carboxamide | ¹H NMR (400 MHz, DMSO-d₆) δ(ppm): 10.50 (s, 1H), 8.49 (d, J = 5.3 Hz, 1H), 7.86 (dd, J = 12.5, 2.3 Hz, 1H), 7.61 (dt, J = 11.9, 2.2 Hz, 1H), 7.53 (s, 1H), 7.51-7.41 (m, 5H), 7.05- |
| # 6.98 (m, 1H), 6.47 (dd, J = 5.2, 1.1 Hz, 1H), 4.02-3.91 (m, 10H). MS (m/z): 521.1 (M + H). | | | | |

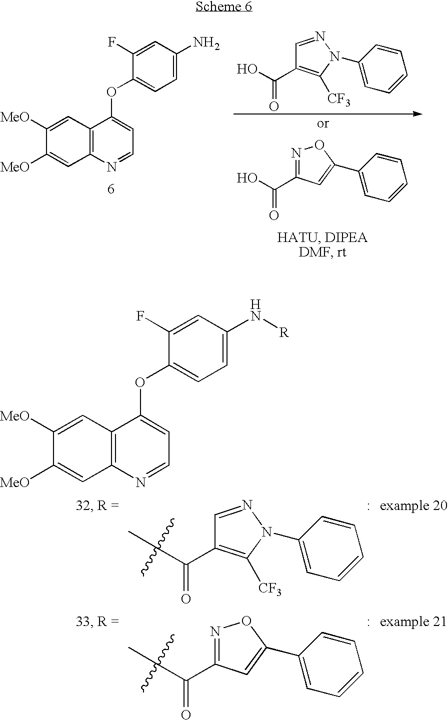

Examples 20 and 21 (compounds 32 and 33) were prepared in one step from 6 and the appropriate aryl-heteroarylcarboxylic acid (Scheme 6) similarly to compound 16 (example 4, step 3a, Scheme 4).

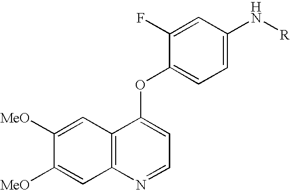

Characterization of compounds 32 and 33 (examples 20 and 21)

| Cpd | Ex. | R | Name | Characterization |
|---|---|---|---|---|
| 32 | 20 | 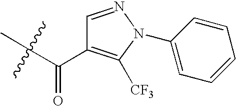 | N-(4-(6,7- dimethoxyquinolin- 4-yloxy)-3- fluorophenyl)-1- phenyl-5- (trifluoromethyl)- 1H-pyrazole-4- carboxamide | ¹H NMR (400 MHz, DMSO-d₆) □(ppm): 10.90 (s, 1H), 8.49 (d, J = 5.3 Hz, 1H), 8.36 (s, 1H), 7.96 (dd, J =12.8, 2.4 Hz, 1H), 7.66-7.53 (m, 7H), 7.50 (t, J = 8.9 Hz, 1H), 7.42 (s, 1H), |
| # 6.49 (dd, J = 5.2, 1.1 Hz, 1H), 3.96 and 3.95 (2s, 2x3H). MS (m/z): 553.1 (M + H). | | | | |
| 33 | 21 | 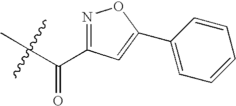 | N-(4-(6,7- dimethoxyquinolin- 4-yloxy)-3- fluorophenyl)-5- phenylisoxazole- 3-carboxamide | ¹H NMR (400 MHz, DMSO-d₆) □(ppm): 11.17 (s, 1H), 8.49 (d, J = 5.1 Hz, 1H), 8.04 (dd, J = 12.9, 2.3 Hz, 1H), 8.02-7.97 (m, 2H), 7.82-7.76 (m, 1H), 7.63-7.48 (m, 6H), 7.42 (s, 1H), 6.49 (dd, J = 5.3, 1.0 Hz, 1H), 3.96 (s, |
| # 6H). MS (m/z): 521.1 (M + 1). | | | | |

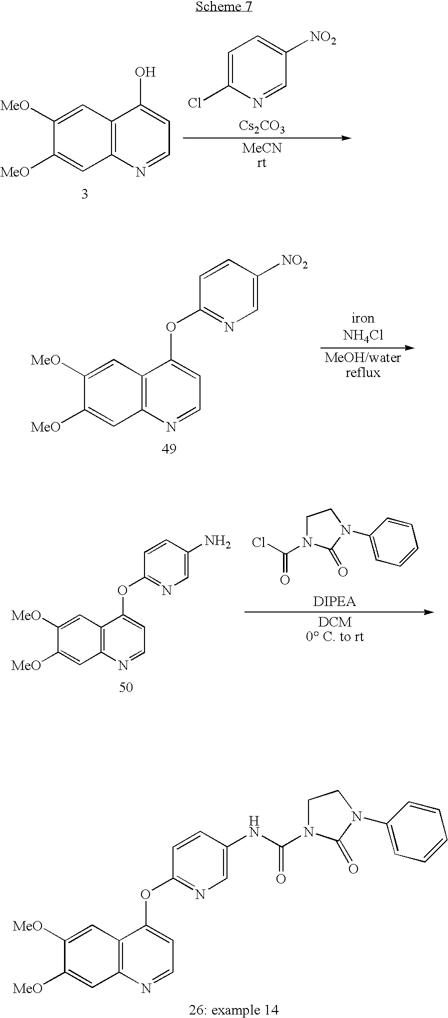

### Example 14

### N-(6-(6,7-dimethoxyquinolin-4-yloxy)pyridin-3-yl)-2-oxo-3-phenylimidazolidine-1-carboxamide (26)

### Step 1. 6,7-dimethoxy-4-(5-nitropyridin-2-yloxy)quinoline (49)

A suspension of 3 (1.00 g, 4.87 mmol), 2-chloro-5-nitropyridine (1.00 g, 6.31 mmol) and cesium carbonate (2.02 g, 6.20 mmol) in anhydrous acetonitrile (110 mL) was stirred for two days under nitrogen. The reaction mixture was concentrated, diluted with AcOEt, and successively washed with a saturated solution of NaHCO₃, a saturated solution of NH₄Cl and brine. The aqueous layer was extracted with AcOEt. The combined organic layer was concentrated, adsorbed on silica gel and purified twice by flash column chromatography (eluents MeOH/DCM: 02/98→05/95) and triturated in a mixture of AcOEt/hexanes to afford the title compound 49 (420 mg, 26% yield) as an orange fluffy solid. MS (m/z): 328.2 (M+H).

### Step 2. 6-(6,7-dimethoxyquinolin-4-yloxy)pyridin-3-amine (50)

To a stirred suspension of 49 (420 mg, 3.08 mmol) in a mixture of methanol/water (20 mL/5 mL) were successively added ammonium chloride (103 mg, 1.93 mmol) and iron powder (215 mg, 3.85 mmol). The reaction mixture was heated to reflux for 6 h then cooled to room temperature, filtered, and concentrated. The residue was adsorbed on silica gel, purified by flash column chromatography (eluents MeOH/DCM: 5/95w 10/90) and coprecipitated in AcOEt with traces of DCM/hexanes to afford the title compound 50 (241 mg, 63% yield) as a yellow solid. MS (m/z): 298.2 (M+H).

### Step 3. N-(6-(6,7-dimethoxyquinolin-4-yloxy)pyridin-3-yl)-2-oxo-3-phenyl imidazolidine-1-carboxamide (26)

The title compound 26 (example 14) was obtained in one step from 50 and 2-oxo-3-phenylimidazolidine-1-carbonyl chloride (8) as a white solid in 89% yield following the same procedure as in scheme 2, example 2, compound 9. ¹H NMR (400 MHz, DMSO-d₆) δ (ppm): 10.43 (s, 1H), 8.57 (d, J=5.1 Hz, 1H), 8.45 (d, J=2.9 Hz, 1H), 8.22 (dd, J=8.8, 2.7 Hz, 1H), 7.63 (d, J=7.8 Hz, 2H), 7.43 (t, J=7.8 Hz, 2H), 7.42 (s, 1H), 7.37 (s, 1H), 7.33 (d, J=8.8 Hz, 1H), 7.17 (t, J=7.3 Hz, 1H), 6.86 (d, J=5.1 Hz, 1H), 4.01-3.86 (m, 10H). MS (m/z): 486.3 (M+H).

### Example 16

### N-(4-(6,7-dimethoxyquinolin-4-yloxy)-3-fluorophenyl)-3-(3-fluorophenyl)-2-oxoimidazolidine-1-carboxamide (28)

### Step 1. 1-(2-chloroethyl)-3-(3-fluorophenyl)urea (44c)

To a stirred solution of 3-fluoroaniline (3.00 g, 27.00 mmol) in anhydrous THF (50 mL) at 0° C. under nitrogen was slowly added 2-chloroethyl isocyanate (43) (2.61 ml, 30.60 mmol). The reaction mixture was stirred at room temperature overnight. Extra isocyanate (1 mL) was added and the reaction mixture was stirred for another day, concentrated then diluted a bit with a mixture of Et₂O/AcOEt and coprecipitated by addition of hexanes. The suspension was shaken, filtered and then the collected solid was washed with additional hexane to afford compound 44c (5.35 g, 92 yield) as a beige fluffy solid. MS (m/z): 217.0 (M+H).

### Step 2. 1-(3-fluorophenyl)imidazolidin-2-one (45c)

To a stirred suspension of NaH (2.48 g, 61.91 mmol, 60% dispersion in oil) in anhydrous THF (50 mL) at 0° C. under nitrogen was slowly added a solution of 44c (5.35 g, 24.76 mmol) in anhydrous THF (50 mL). The reaction mixture was stirred at 0° C. for 45 min, warmed to room temperature for 30 min and then heated to reflux for 3 h. The reaction mixture was allowed to cool to room temperature, concentrated then quenched by the slow addition of water and the pH was adjusted to pH 6-7 with 3N HCl. The mixture was shaken for 2 h and the solid was collected by filtration, washed with water and hexanes and dried under high vacuum to afford the title compound 45c (4.03 g, 90% yield) as a pale brown solid. MS (m/z): 181.1 (M+H).

### Step 3. 3-(3-fluorophenyl)-2-oxoimidazolidine-1-carbonyl chloride (46c)

To a stirred solution of 45c (360 mg, 2.0 mmol) in anhydrous THF (20 ml) at room temperature under nitrogen was added triphosgene (208 mg, 0.70 mmol). The reaction mixture was heated at 65° C. for 3 h then was allowed to cool to room temperature, and the solution (or suspension) was used in the next step without any further purification.

### Step 4. N-(4-(6,7-dimethoxyquinolin-4-yloxy)-3-fluorophenyl)-3-(3-fluorophenyl)-2-oxoimidazolidine-1-carboxamide (28)

To a stirred solution of 6 (200 mg, 0.64 mmol) and DIPEA (333 μl, 1.91 mmol) in anhydrous DCM (20 ml) at 0° C. under nitrogen was slowly added a solution of 46c (15 ml, 1.5 mmol). The reaction mixture was stirred at room temperature overnight then quenched by addition of MeOH and concentrated. The residue was purified by flash column chromatography on silica gel (eluents MeOH/DCM: 02/98→05/95) followed by trituration with a minimum of MeOH to afford the title compound 28 (240 mg, 72% yield) as an off-white solid. ¹H NMR (400 MHz, DMSO-d₆) δ (ppm): 10.49 (s, 1H), 8.48 (d, J=5.3 Hz, 1H), 7.86 (dd, J=13.0, 2.1 Hz, 1H), 7.61 (dt, J=11.9, 2.2 Hz, 1H), 7.53 (s, 1H), 7.51-7.39 (m, 5H), 7.02 (td, J=8.2, 2.3 Hz, 1H), 6.47 (d, J=5.1 Hz, 1H), 4.02-3.91 (m, 10H). MS (m/z): 521.2 (M+H).

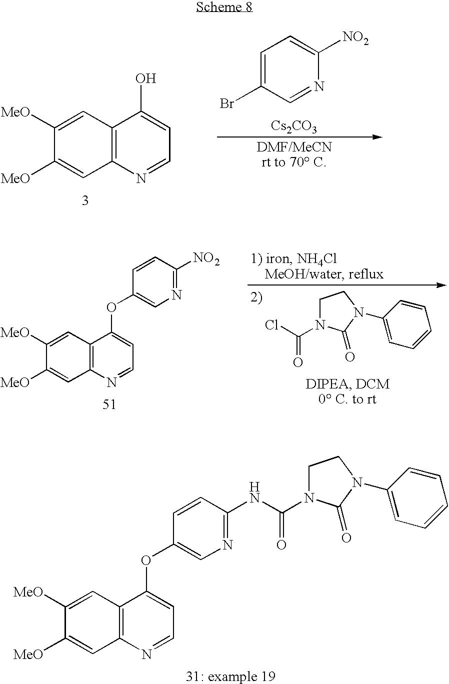

### Example 19

### N-(5-(6,7-dimethoxyquinolin-4-yloxy)pyridin-2-yl)-2-oxo-3-phenylimidazolidine-1-carboxamide (31)

### Step 1. 6,7-dimethoxy-4-(6-nitropyridin-3-yloxy)quinoline (51)

A stirred suspension of 3 (500 mg, 2.44 mmol), 5-bromo-2-nitropyridine (694 mg, 3.42 mmol) and cesium carbonate (1.19 g, 3.66 mmol) in a mixture of anhydrous DMF/acetonitrile (20 mL/20 mL) under nitrogen was heated at 70° C. for two days. The reaction was allowed to cool to room temperature and then concentrated, diluted with AcOEt and successively washed with water, a saturated solution of NH₄Cl, water and brine, and concentrated. The residue was purified twice by flash column chromatography on silica gel (eluents MeOH/DCM: 02/98→05/95) and triturated with MeOH to afford the title compound 51 (190 mg, 24% yield) as a yellow solid. MS (m/z): 328.1 (M+H).

### Step 2. N-(5-(6,7-dimethoxyquinolin-4-yloxy)pyridin-2-yl)-2-oxo-3-phenyl imidazolidine-1-carboxamide (31)

The title compound 31 (example 19) was obtained in two steps from 51 as a white fluffy solid following the same procedure as in example 14, steps 2 and 3 (scheme 7). ¹H NMR (400 MHz, DMSO-d₆) δ (ppm): 10.99 (s, 1H), 8.50 (d, J=5.3 Hz, 1H), 8.36 (dd, J=2.9, 0.6 Hz, 1H), 8.16 (dd, J=9.1, 0.7 Hz, 1H), 7.85 (dd, J=9.0, 2.9 Hz, 1H), 7.67-7.61 (m, 2H), 7.54 (s, 1H), 7.47-7.41 (m, 2H), 7.41 (s, 1H), 7.19 (t, J=7.4 Hz, 1H), 6.54 (d, J=5.1 Hz, 1H), 4.01-3.93 (m, 10H). MS (m/z): 486.2 (M+H).

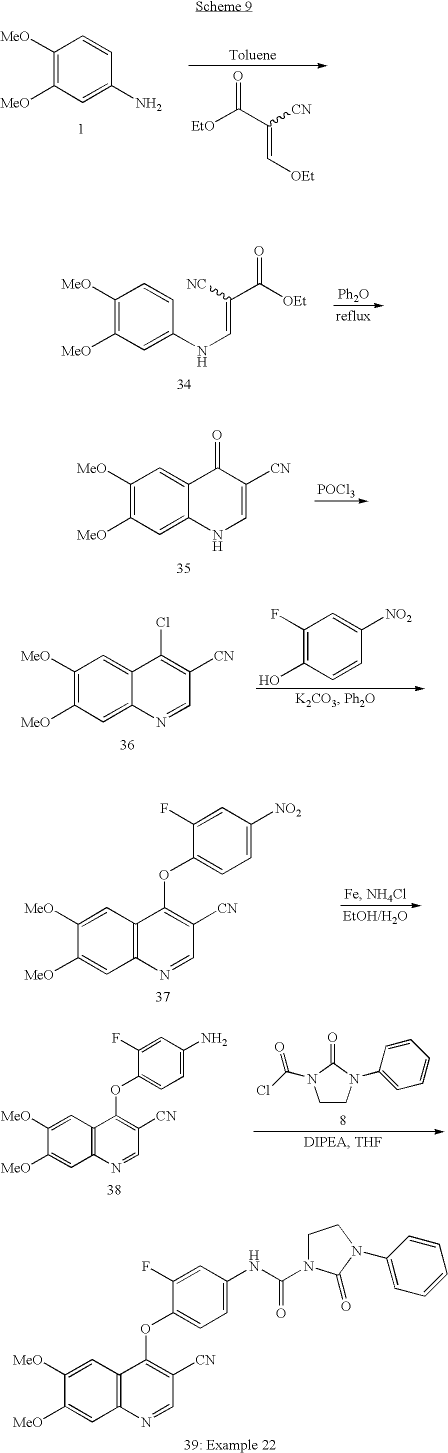

### Example 22

### N-(4-(3-cyano-6,7-dimethoxyquinolin-4-yloxy)-3-fluorophenyl)-2-oxo-3-phenylimidazolidine-1-carboxamide (39)

### Step 1: ethyl 2-cyano-3-(3,4-dimethoxyphenylamino)acrylate (34)

A reaction mixture of 3,4-dimethoxyaniline (1) (6.13 g, 40 mmol) and ethyl 2-cyano-3-ethoxyacrylate (Aldrich) (6.77 g, 40 mmol) in toluene was stirred at 100° C. for 1 h and at 125° C. for 15 min. The solvent was removed under reduced pressure, and the residue was recrystallized from EtOAc to give the title compound 34 (10.33 g, 94% yield) as a yellowish solid. MS (m/z): 277.1 (M+H).

### Step 2: 6,7-dimethoxy-4-oxo-1,4-dihydroquinoline-3-carbonitrile (35)

A reaction mixture of 34 (10.33 g, 37.4 mmol) in diphenylether (125 mL) was heated to reflux for 4 hours before cooled down to room temperature. The residue was triturated with hexane and diethyl ether to afford the desired title compound 35 (1.44 g, 16% yield) as beige solid. MS (m/z): 231.0 (M+H).

### Step 3: 4-chloro-6,7-dimethoxyquinoline-3-carbonitrile (36)

A reaction mixture of 35 (1.4 g, 6.08 mmol) in POCl₃ (3 mL) was heated to reflux for 2 hours before cooled down and poured into ice/DCM with stirring. K₂CO₃ was added to adjust to pH™9, the mixture was filtered through a pad of Celite, and the organic phase was separated, dried and concentrated to give the title compound 36 (960 mg, 64% yield) as solid. MS (m/z): 249.0 (M+H).

### Step 4: 4-(2-fluoro-4-nitrophenoxy)-6,7-dimethoxyquinoline-3-carbonitrile (37)

A reaction mixture of quinoline 36 (836 mg, 3.36 mmol), 2-fluoro-4-nitrophenol (1056 mg, 6.72 mmol) and potassium carbonate (929 mg, 6.72 mmol) in Ph₂O (13 mL) was stirred at 120° C. for another 2 hours before cooling to room temperature. The reaction mixture was diluted with EtOAc, washed with brine. The organic phase was dried with Na₂SO₄, filtered and concentrated to give the title compound 37 (460 mg, 37% yield). MS (m/z): 370 (M+H).

### Step 5: 4-(4-amino-2-fluorophenoxy)-6,7-dimethoxyquinoline-3-carbonitrile (38)

The reaction mixture of nitro compound 37 (460 mg, 1.246 mmol), iron powder (591 mg, 10.59 mmol) and ammonium chloride (57.3 mg, 1.071 mmol) in ethanol (12.00 mL)/water (6 mL) was heated to 90° C. for 30 min before cooling to room temperature and filtered through a pad of Celite to give the title compound 38 (100 mg, 24% yield) as brown solid. MS (m/z)=340.0 (M+1).

### Step 6: N-(4-(3-cyano-6,7-dimethoxyquinolin-4-yloxy)-3-fluorophenyl)-2-oxo-3-phenylimidazolidine-1-carboxamide (39)

The reaction mixture of aniline 38 (100 mg, 0.295 mmol), 2-oxo-3-phenylimidazolidine-1-carbonyl chloride (8, 135 mg, 0.601 mmol) and DIPEA (0.309 mL, 1.768 mmol) in THF (3 mL) was stirred at room temperature for 1 h. The reaction mixture was concentrated, and the residue was diluted with EtOAc, and collected by filtration. The solid was triturated with EtOAc, to give the title compound 39 (120 mg, 0.227 mmol, 77% yield) as beige solid. ¹H NMR (400 MHz, DMSO-d₆) δ (ppm): 10.53 (s, 1H), 8.82 (s, 1H), 7.82 (d, J=13.3 Hz, 1H), 7.62-7.60 (m, 2H), 7.52 (s, 1H), 7.47 (s, 1H), 7.41 (t, J=7.3 Hz, 2H), 7.35-7.27 (m, 2H), 7.16 (t, J=7.2 Hz, 1H), 3.99 (s, 3H), 3.49 (s, 3H), 3.90 (s, 4H). MS (m/z): 528.2 (M+H).

### Example 24

### N-(4-(6,7-dimethoxyquinolin-4-yloxy)-3-fluorophenyl)-2-oxo-3-(pyridin-2-yl)imidazolidine-1-carboxamide (48)

### Step 1. 1-(2-chloroethyl)-3-(pyridin-2-yl)urea (44f)

To a stirred solution of 2-aminopyridine (2.00 g, 21.25 mmol) in anhydrous THF (40 ml) at 0° C. under nitrogen was slowly added 2-chloroethyl isocyanate (43) (2.80 ml, 31.87 mmol). The reaction mixture was stirred at room temperatute overnight, concentrated, and dried under high vacuum to afford the title compound 44f (crude material) as a white solid. MS (m/z): 200.1 (M+H).

### Step 2. 1-(pyridin-2-yl)imidazolidin-2-one (45f)

To a stirred suspension NaH (1.28 g, 53.13 mmol, 60% dispersion in oil) in anhydrous THF (50 mL) at 0° C. under nitrogen was slowly added a solution of 44f (crude material) in anhydrous THF (25 mL). The reaction mixture was warmed to room temperature over 15 min and then heated to reflux for 2 h. The reaction was allowed to cool to room temperature then poured into a mixture of ice/water, and shaken for 1 h. After extraction with DCM, the combined organic layer was dried over anhydrous magnesium sulfate, filtered, and concentrated. The crude solid was triturated with a mixture of AcOEt/hexanes for 1 h, collected by filtration, rinsed with hexanes, and dried under high vacuum to afford the title compound 45f (3.02 g, 87% yield over two steps) as a white crystalline solid. MS (m/z): 164.1 (M+H).

### Step 3. 2-oxo-3-(pyridin-2-yl)imidazolidine-1-carbonyl chloride (46f)

To a stirred solution of 45f (500 mg, 3.06 mmol) in anhydrous THF (20 mL) at −78° C. under nitrogen was slowly added a solution of n-BuLi (1.47 mL, 3.68 mmol, 2.5 M in hexanes). After 1 h triphosgene (318 mg, 1.07 mmol) was added at −78° C., and the reaction mixture was stirred for 30 min. The reaction mixture was allowed to warm to room temperature over 40 min, and then stirred at room temperature for an additional 1.5 h. The resulting suspension was used in the next step without any further purification.

### Step 4. N-(4-(6,7-dimethoxyquinolin-4-yloxy)-3-fluorophenyl)-2-oxo-3-(pyridin-2-yl) imidazolidine-1-carboxamide (48)

The title compound 48 (example 24) was obtained in one step from 6 and 46f as a white solid following the same procedure as in example 9, (scheme 2). ¹H NMR (400 MHz, DMSO-d₆) δ (ppm): 10.49 (s, 1H), 8.48 (d, J=5.3 Hz, 1H), 8.42 (ddd, J=4.9, 1.9, 0.9 Hz, 1H), 8.18 (dt, J=8.4, 0.9 Hz, 1H), 7.91-7.82 (m, 2H), 7.54 (s, 1H), 7.51-7.42 (m, 2H), 7.41 (s, 1H), 7.19 (ddd, J=7.2, 4.9, 1.0 Hz, 1H), 6.47 (dd, J=5.2, 1.1 Hz, 1H), 4.12-4.04 (m, 2H), 3.99-3.89 (m, 8H). MS (m/z): 504.3 (M+H).

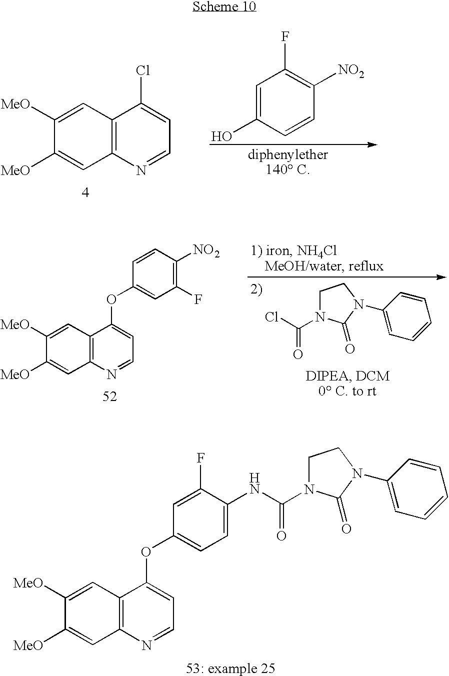

### Example 25

### N-(4-(6,7-dimethoxyquinolin-4-yloxy)-2-fluorophenyl)-2-oxo-3-phenylimidazolidine-1-carboxamide (53)

### Step 1. 4-(3-fluoro-4-nitrophenoxy)-6,7-dimethoxyquinoline (52)

A stirred suspension of 4 (500 mg, 2.24 mmol) and 3-fluoro-4-nitrophenol (862 mg, 5.49 mmol) in diphenylether (10 mL) was heated at 140° C. for one day. The reaction mixture was allowed to cool to room temperature, then purified by flash column chromatography on silica gel (eluents MeOH/DCM: 0/100-05/95) and triturated with Et₂O to afford the title compound 52 (857 mg, contaminated with the phenol starting material) as a pale yellow solid. MS (m/z): 345.2 (M+H).

### Step 2. N-(4-(6,7-dimethoxyquinolin-4-yloxy)-2-fluorophenyl)-2-oxo-3-phenyl imidazolidine-1-carboxamide (53)

The title compound 53 (example 25) was obtained in two steps from 52 as a white solid in 53% yield over two steps following the same procedure as in example 14, steps 2 and 3 (scheme 7). ¹H NMR (400 MHz, DMSO-d₆) δ (ppm): 10.72 (d, J=2.5 Hz, 1H), 8.50 (d, J=5.1 Hz, 1H), 8.27 (t, J=9.1 Hz, 1H), 7.64-7.60 (m, 2H), 7.49 (s, 1H), 7.47-7.41 (m, 3H), 7.41 (s, 1H), 7.22-7.12 (m, 2H), 6.57 (d, J=5.3 Hz, 1H), 4.02-3.91 (m, 10H). MS (m/z): 503.3 (M+H).

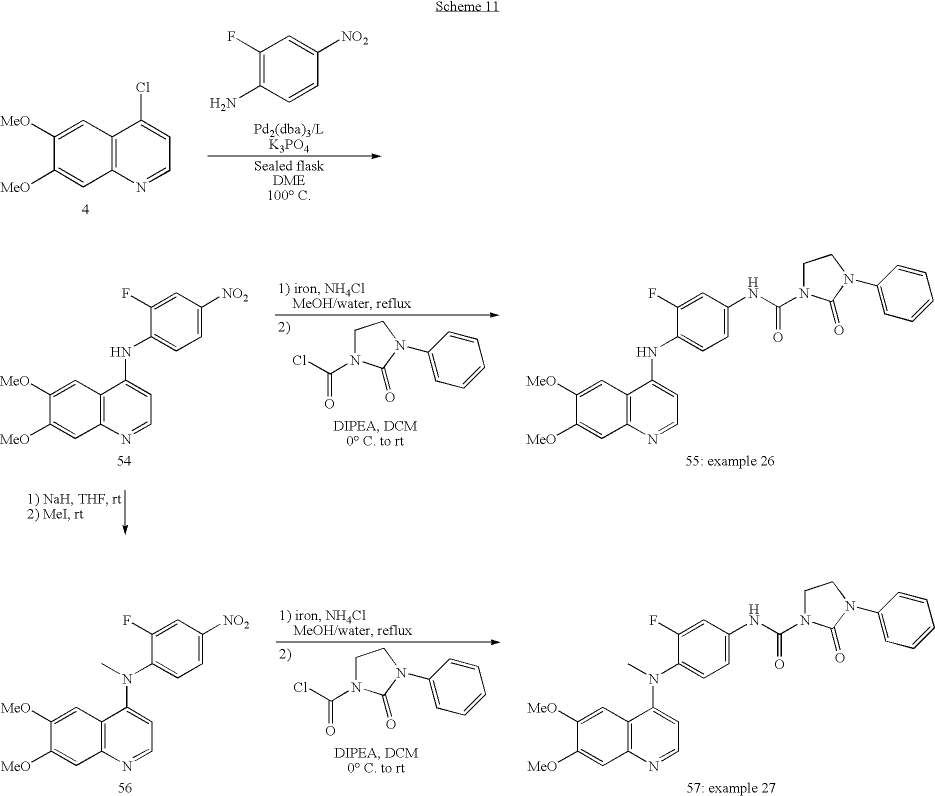

### Example 26

### N-(4-(6,7-dimethoxyquinolin-4-ylamino)-3-fluorophenyl)-2-oxo-3-phenylimidazolidine-1-carboxamide (55)

### Step 1. N-(2-fluoro-4-nitrophenyl)-6,7-dimethoxyquinolin-4-amine (54)

To a degassed mixture of Pd₂(dba)₃ (102 mg, 0.11 mmol), (2-biphenyl)dicyclohexylphosphine (78 mg, 0.22 mmol) and K₃PO₄ (807 mg, 3.80 mmol) in a under nitrogen were added DME (20 mL), 4-chloro-6,7-dimethoxyquinoline (4) (500 mg, 2.24 mmol) and 2-fluoro-4-nitroaniline (523 mg, 3.35 mmol), respectively. The reaction mixture was degassed again, stirred for 15 min at room temperature and heated at 100° C. in a sealed flask for 17 h. The reaction mixture was allowed to cool to room temperature then diluted with AcOEt and successively washed with water, a saturated solution of NaHCO₃ and a saturated solutionof NH₄Cl. The combined aqueous layers were extracted with AcOEt. The extract and the original AcOEt phase were combined, concentrated, adsorbed on silica gel, purified by flash column chromatography (eluents MeOH/DCM: 5/95-10/90, then 2% of ammonium hydroxide in MeOH/DCM: 10/90→15/85) and triturated with AcOEt with traces of DCM/hexanes, to afford the title compound 54 (748 mg, 97% yield) as a dark red-brown solid. MS (m/z): 344.2 (M+H).

### Step 2. N-(4-(6,7-dimethoxyquinolin-4-ylamino)-3-fluorophenyl)-2-oxo-3-phenyl imidazolidine-1-carboxamide (55)

The title compound 55 (example 18) was obtained in two steps from 54 as an off-white fluffy solid in 58% yield [over two steps] by following the same procedures as in example 14, steps 2 and 3 (scheme 7), but by treating the concentrated reaction mixture at the final step with 2% of ammonium hydroxide in MeOH/DCM overnight before the column purification step. ¹H NMR (400 MHz, DMSO-d₆) δ (ppm): 10.53 (s, 1H), 8.55 (s, 1H), 8.23 (d, J=5.3 Hz, 1H), 7.75 (dd, J=12.9, 2.0 Hz, 1H), 7.70 (s, 1H), 7.67-7.61 (m, 2H), 7.47-7.35 (m, 4H), 7.24 (s, 1H), 7.18 (tt, J=7.4, 1.0 Hz, 1H), 6.27-6.21 (m, 1H), 4.02-3.92 (m, 4H), 3.93 (s, 3H), 3.90 (s, 3H). MS (m/z): 502.3 (M+H).

### Example 27

### N-(4-((6,7-dimethoxyquinolin-4-yl)(methyl)amino)-3-fluorophenyl)-2-oxo-3-phenyl imidazolidine-1-carboxamide (57)

### Step 1. N-(2-fluoro-4-nitrophenyl)-6,7-dimethoxy-N-methylquinolin-4-amine (56)

To a stirred suspension of NaH (65 mg, 1.62 mmol, 60% dispersion in oil) in anhydrous DMF (2 mL) at room temperature under nitrogen was slowly added a solution of 54 (370 mg, 1.08 mmol) in anhydrous DMF (1 ml). The reaction mixture was stirred for 15 min, and MeI (81 μl, 1.29 mmol) was added. After 2 h, the reaction mixture was quenched by a slow addition of MeOH followed by water. The resulting suspension was shaken for 30 min, the solid material was collected by filtration, rinsed with water, air-dried, and dried under high vacuum to afford the title compound 56 (340 mg, 88% yield) as a dark red powder. MS (m/z): 164.1 (M+H).

### Step 2. N-(4-((6,7-dimethoxyquinolin-4-yl)(methyl)amino)-3-fluorophenyl)-2-oxo-3-phenyl imidazolidine-1-carboxamide (57)

The title compound 57 (example 19) was obtained in two steps from 56 as a pale yellow solid in 7% yield [over two steps] following the same procedures as in example 14, steps 2 and 3 (scheme 7), but by treating the concentrated reaction mixture at the final step with 2% of ammonium hydroxide solution in MeOH/DCM for few hours before the chromatography purification step. ¹H NMR (400 MHz, DMSO-d₆) δ (ppm): 10.52 (s, 1H), 8.62 (d, J=5.9 Hz, 1H), 8.13 (s, 0.3H, formate), 7.70 (dd, J=13.3, 2.1 Hz, 1H), 7.62 (d, J=7.6 Hz, 2H), 7.42 (t, J=8.0 Hz, 2H), 7.38-7.25 (m, 3H), 7.21-7.13 (m, 2H), 6.70 (s, 1H), 4.00-3.88 (m, 4H), 3.90 (s, 3H), 3.47 (s, 3H), 3.39 (s, 3H). MS (m/z): 516.3 (M+H).

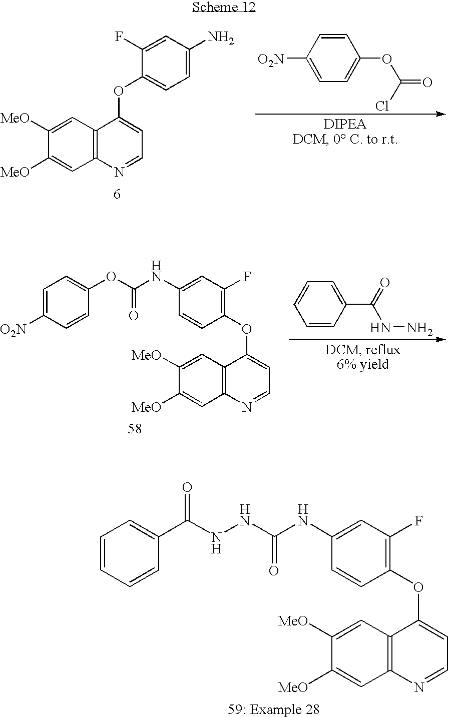

### Example 28

### 2-Benzoyl-N-(4-(6,7-dimethoxyquinolin-4-yloxy)-3-fluorophenyl)hydrazinecarboxamide (59)

### Step 1. 4-nitrophenyl 4-(6,7-dimethoxyquinolin-4-yloxy)-3-fluorophenylcarbamate (58)

4-Nitrophenyl chloroformate (0.257 g, 1.273 mmol) was added to a solution of compound 6 (0.2 g, 0.636 mmol) and DIPEA (0.244 ml, 1.400 mmol) in DCM (6.36 ml) at 0° C. The mixture was stirred at 0° C. for 5 h, warmed gradually to room temperature and stirred overnight. The title compound [MS (m/z): 480.2 (M+H)]. was not isolated from the reaction mixture which was used in the next step as is.

### Step 2. 2-benzoyl-N-(4-(6,7-dimethoxyquinolin-4-yloxy)-3-fluorophenyl)hydrazinecarboxamide (59)

Benzoic hydrazide (0.260 g, 1.908 mmol) was added to a solution of compound 58 (˜0.293 g, 0.087 mmol) and the mixture was heated to reflux over night. The reaction mixture was concentrated under reduced pressure, the residue was dissolved in MeOH and purified by preparative HPLC (column: Luna C18 (2), 5 cm ID; gradient: 60% MeOH to 95% MeOH in water, 60 min) affording compound 59 (0.02 g, 6.40% yield) as cream-colored solid. ¹H-NMR (400 MHz, DMSO-d₆) δ (ppm): 10.35 (s, 1H), 9.26 (br, 1H), 8.47 (d, J=5.2 Hz, 1H), 8.42 (s, 1H), 7.94 (d, J=7.2 Hz, 2H), 7.76 (d, J=13.7 Hz, 1H), 7.62-7.58 (m, 1H), 7.54-7.5 (m, 3H), 7.42-7.37 (m, 3H), 6.45 (d, J=5.2 Hz, 1H), 3.95 (s, 6H). MS (m/z): 477.2 (M+H).

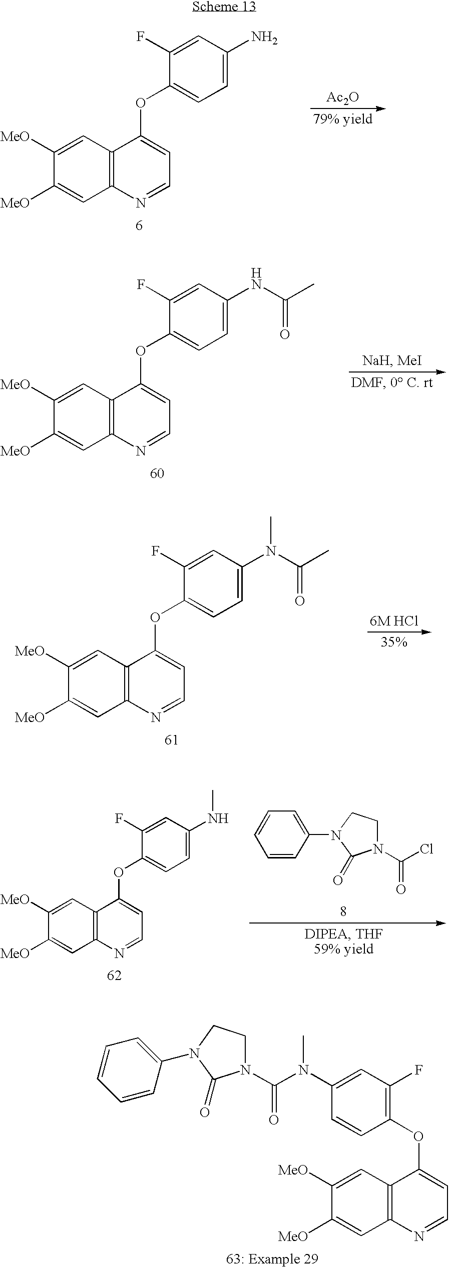

### Example 29

### N-(4-(6,7-dimethoxyquinolin-4-yloxy)-3-fluorophenyl)-N-methyl-2-oxo-3-phenylimidazolidine-1-carboxamide (63)

### Step 1. N-(4-(6,7-dimethoxyquinolin-4-yloxy)-3-fluorophenyl)acetamide (60)

Compound 6 (0.15 g, 0.477 mmol) was dissolved in acetic anhydride (2.386 ml) and the reaction mixture was stirred at room temperature overnight. MeOH was added and the mixture was stirred for an additional 1 h. The reaction mixture was diluted with DCM, washed with 2N NaOH, icy water then dried over anhydrous sodium sulfate, filtered and concentrated under reduced pressure. The residue was purified by flash chromatography (Biotage, Si 12+M column, gradient: 5% MeOH in DCM, 5 CV; 3% to 10%, 2CV and 10% 5CV in DCM) affording compound 60 (0.1193 g, 70% yield) as a white solid. MS (m/z): 357.2 (M+H). The crude material was used in the next step without further purification.

### Step 2. N-(4-(6,7-dimethoxyquinolin-4-yloxy)-3-fluorophenyl)-N-methylacetamide (61)

Sodium hydride (0.057 g, 1.431 mmol) was added to a solution of 60 (0.170 g, 0.477 mmol) in DMF (4.77 ml) at 0° C. and the mixture was stirred for 1 h. MeI (0.030 ml, 0.477 mmol) was added to the reaction mixture which was stirred for an additional hour at ambient temperature, diluted with EtOAc, washed with water and concentrated under reduced pressure to afford title compound 61 (MS (m/z): 371.2 (M+H)) which was used in the next step without further purification.

### Step 3. 4-(6,7-dimethoxyquinolin-4-yloxy)-3-fluoro-N-methylaniline (62)

Crude compound 61 (˜0.163 g, 0.44 mmol) was dissolved in 6M HCl (10 mL) and the mixture was heated to reflux for 1 h. The reaction mixture was cooled to room temperature, diluted with water and washed with EtOAc. The aqueous solution was basified to pH˜11 by addition of 2N NaOH, extracted with EtOAc and the organic extract was washed with water and brine, dried over anhydrous sodium sulfate and concentrated under reduced pressure. The residue was purified by flash chromatography (Biotage, Si 12+M column, gradient: 2% (5CV), 2% to 5% (2CV) and 5% (5CV) MeOH in DCM) affording compound 62 (0.0498 g, 34.5% yield) as white solid. MS (m/z): 329.2 (M+H).

### Step 4. N-(4-(6,7-dimethoxyquinolin-4-yloxy)-3-fluorophenyl)-N-methyl-2-oxo-3-phenylimidazolidine-1-carboxamide (63)

Compound 8 (0.036 g, 0.159 mmol) was added to a solution of compound 62 (0.0498 g, 0.152 mmol) and DIPEA (0.032 mL, 0.183 mmol) in THF (1.517 mL) and the mixture was stirred at room temperature for 1 h then concentrated under reduced pressure. The residue was dissolved in DMSO and purified by preparative HPLC (column: Luna C18 (2), 2.2 cm ID; gradient: 30% MeOH to 95% MeOH in water, 45 min) affording compound 63 (0.047 g, 59% yield) as pink solid. ¹H-NMR (400 MHz, DMSO-d₆) δ (ppm): 8.26 (d, J=5.3 Hz, 1H), 7.60 (dd, J=12.1, 2.3 Hz, 1H), 7.50-7.46 (m, 3H), 7.42-7.3 (m, 5H), 7.1 (t, J=7.4 Hz, 1H), 6.25 (dd, J=4.5, 0.8 Hz, 1H), 3.94 (s, 3H), 3.91 (s, 3H), 3.86 (br, 4H), 3.39 (s, 3H). MS (m/z): 517.3 (M+H).

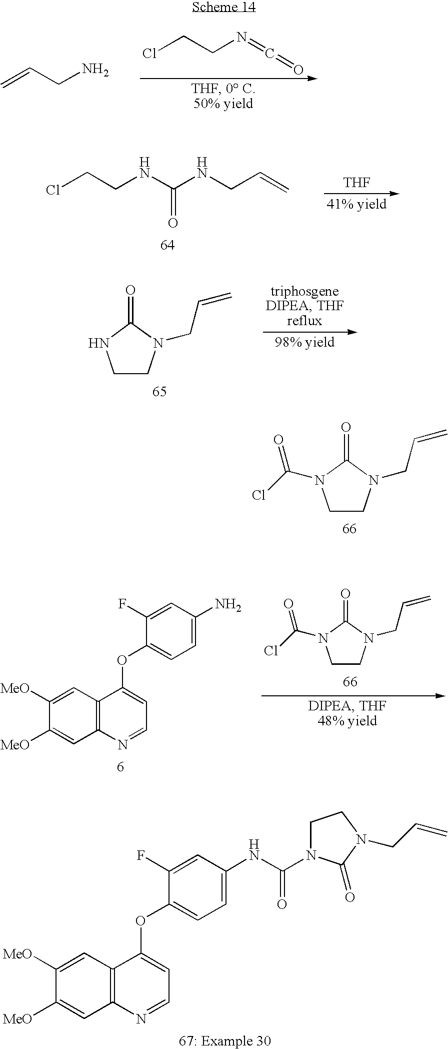

### Example 30

### 3-allyl-N-(4-(6,7-dimethoxyquinolin-4-yloxy)-3-fluorophenyl)-2-oxoimidazolidine-1-carboxamide (56)

### Step 1. 1-allyl-3-(2-chloroethyl)urea (64)

2-Chloroethyl isocyanate (3.13 ml, 36.7 mmol) was added dropwise at 0° C. to a solution of allylamine (2.5 mL, 33.3 mmol) in THF (33.3 mL) and the mixture was stirred for 2 h. The reaction mixture was concentrated under reduced pressure and the residue triturated with ether and filtered affording compound 64 (2.70 g, 49% yield) as white solid. MS (m/z): 163.2 (M+H).

### Step 2. 1-allylimidazolidin-2-one (65)

Sodium hydride (0.995 g, 24.89 mmol) was carefully added to a solution of compound 64 (2.70 g, 16.59 mmol) in THF (16.59 ml) at room temperature and the suspension was stirred for 3 h. The reaction mixture was diluted with EtOAc and extracted with water. The organic layer was concentrated under reduced pressure affording compound 65 (0.86 g, 41% yield) as transparent oil. MS (m/z): 127.2 (M+H).

### Step 3. 3-allyl-2-oxoimidazolidine-1-carbonyl chloride (66)

Triphosgene (1.02 g, 3.42 mmol) was carefully added (highly exothermic reaction) to a solution of 65 (0.86 g, 6.85 mmol) and DIPEA (1.196 ml, 6.85 mmol) in THF (68.5 ml). The solution was heated to reflux for 15 min then concentrated under reduced pressure. The residue was dissolved in DCM and washed with water and IN ammonium chloride solution, dried over anhydrous sodium sulfate, filtered and concentrated under reduced pressure affording compound 66 (1.27 g, 98% yield) as light brown syrup that was used in the next step without further purification. MS (m/z): 189.1 (M+H).

### Step 4. 3-allyl-N-(4-(6,7-dimethoxyquinolin-4-yloxy)-3-fluorophenyl)-2-oxoimidazolidine-1-carboxamide (67)

Compound 66 (0.090 g, 0.477 mmol) was added to a solution of compound 6 (0.15 g, 0.477 mmol) and DIPEA (0.100 ml, 0.573 mmol) in THF (4.77 ml) and the mixture was stirred at room temperature overnight. The reaction mixture was concentrated under reduced pressure and purified by flash chromatography [Biotage, Si 12+S column, gradient: pure EtOAc (5CV), pure EtOAc to 2% MeOH (2CV)] and pure EtOAc to 2% MeOH (5CV) affording compound 67 (0.109 g, 48% yield) as white foam. ¹H-NMR (400 MHz, DMSO-d₆) δ (ppm): 10.59 (s, 1H), 8.47 (d, J=5.3 Hz, 1H), 7.80 (dd, J=12.9, 2.2 Hz, 1H), 7.53 (s, 1H), 7.44-7.35 (m, 3H), 6.45 (d, J=5.0 Hz, 1H), 5.86-5.76 (m, 1H), 5.30-5.21 (m, 2H), 3.95 (s, 6H), 3.87-3.81 (m, 4H), 3.45-3.41 (m, 2H). MS (m/z): 467.2 (M+H).

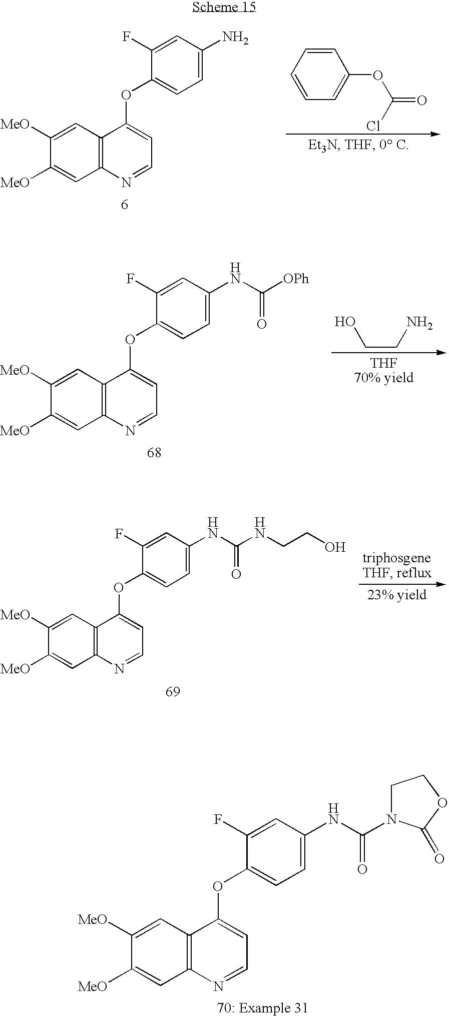

### Example 31

### N-(4-(6,7-dimethoxyquinolin-4-yloxy)-3-fluorophenyl)-2-oxooxazolidine-3-carboxamide (70)

### Step 1. phenyl 4-(6,7-dimethoxyquinolin-4-yloxy)-3-fluorophenylcarbamate (68)

A solution of compound 6 (0.065 g, 0.162 mmol), DIPEA (0.057 ml, 0.324 mmol) and triphosgene (0.024 g, 0.081 mmol) in THF (1.619 ml) was heated to reflux overnight. The suspension was concentrated under reduced pressure and the residuewas purified by flash chromatography (Biotage, Si 12+S column, gradient: 2% (5CV), 2% to 5% (2CV) and 5% (10CV) MeOH in DCM) to afford compound 68 (0.016 g, 23% yield) as white solid. ¹H NMR (400 MHz, DMSO-d₆) δ (ppm): 10.02 (s, 1H), 8.47 (m, 2H), 7.79 (dd, J=11.7, 2.1 Hz, 1H), 7.51-7.39 (m, 3H), 6.45 (dd, J=5.3, 0.8 Hz, 1H), 4.45 (dd, J=8.4, 7.8 Hz, 2H), 3.99 (dd, J=8.4, 7.8 Hz, 2H), 3.93 (s, 6H). MS (m/z): 428.2 (M+H).

### Step 2. 1-(4-(6,7-dimethoxyquinolin-4-yloxy)-3-fluorophenyl)-3-(2-hydroxyethyl)urea (69)

Ethanolamine (0.058 ml, 0.954 mmol) was added to a solution of compound 68 (0.207 g, 0.477 mmol) in THF (4.77 ml) and the mixture was stirred at room temperature overnight. The reaction mixture was diluted with EtOAc then water and aqueous sodium bicarbonate was added and the mixture was stirred for 10 mins. The resultant suspension was filtered and the collected solid was washed with water and dried under reduced pressure affording compound 69 (0.1349 g, 71% yield) as white solid. MS (m/z): 402.2 (M+H).

### Step 3. N-(4-(6,7-dimethoxyquinolin-4-yloxy)-3-fluorophenyl)-2-oxooxazolidine-3-carboxamide (70)

A solution of compound 69 (0.065 g, 0.162 mmol), DIPEA (0.057 mL, 0.324 mmol) and triphosgene (0.024 g, 0.081 mmol) in THF (1.619 mL) was heated to reflux overnight. The suspension was concentrated under reduced pressure and the residue was purified by flash chromatography (Biotage, Si 12+S column, gradient: 2% (5CV), 2% to 5% (2CV) and 5% (10CV) MeOH in DCM) to afford compound 70 (0.016 g, 23% yield) as white solid. ¹H NMR (400 MHz, DMSO-d₆) δ (ppm): 10.02 (s, 1H), 8.47 (m, 2H), 7.79 (dd, J=11.7, 2.1 Hz, 1H), 7.51-7.39 (m, 3H), 6.45 (dd, J=5.3, 0.8 Hz, 1H), 4.45 (dd, J=8.4, 7.8 Hz, 2H), 3.99 (dd, J=8.4, 7.8 Hz, 2H), 3.93 (s, 6H). MS (m/z): 428.2 (M+H).

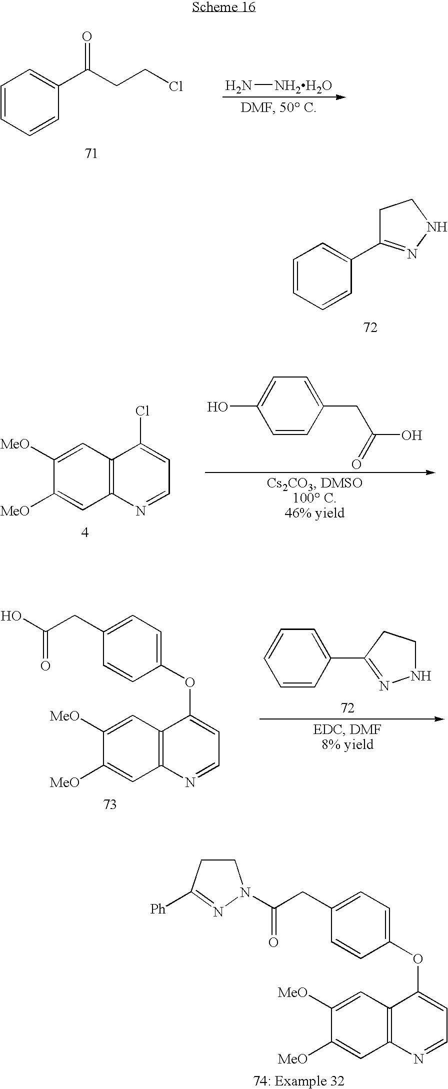

### Example 32

### 2-(4-(6,7-dimethoxyquinolin-4-yloxy)phenyl)-1-(3-phenyl-4,5-dihydro-1H-pyrazol-1-yl)ethanone (74)

### Step 1. 3-phenyl-4,5-dihydro-1H-pyrazole (72)

Hydrazine hydrate (0.098 mL, 1.995 mmol) was added to a solution of 3-chloropropiophenone (0.160 g, 0.95 mmol) in DMF (1.900 mL) and the mixture was stirred at 50° C. for 1 h. The reaction mixture was used in the step 3 without isolating the title compound 72. MS (m/z): 147.1 (M+H).

### Step 2. 2-(4-(6,7-dimethoxyquinolin-4-yloxy)phenyl)acetic acid (73)

A suspension of cesium carbonate (0.728 g, 2.236 mmol) in DMSO (2.236 mL) was heated at 100° C. under atmosphere of nitrogen for 20 mins. 4-hydroxyphenyacetic acid (0.136 g, 0.894 mmol) was added and the mixture was stirred for an additional 30 mins followed by the addition of compound 4 (0.200 g, 0.894 mmol). The reaction mixture was stirred at 100° C. for a further 2 h. The reaction mixture was diluted with water, washed with EtOAc then acidified (pH˜4) by addition of 2N HCl and filtered. The precipitate was dried overnight under vacuum to afford crude compound 73 (0.140 g, 46% yield) as a brown solid that was used in the next step without further purification. MS (m/z): 340.2 (M+H).

### Step 3. 2-(4-(6,7-dimethoxyquinolin-4-yloxy)phenyl)-1-(3-phenyl-4,5-dihydro-1H-pyrazol-1-yl)ethanone (74)

A solution of compound 73 (0.078 g, 0.530 mmol), compound 72 (0.078 g, 0.530 mmol) and EDC (0.112 g, 0.583 mmol) in DMF (1.061 ml) was stirred overnight at room temperature. The reaction mixture diluted with EtOAc, washed with water, dried over anhydrous sodium sulfate and concentrated under reduced pressure. The crude product was purified by flash chromatography (Biotage, Si 25+S column, gradient: 3%, 10 CV; 3% to 5%, 2CV and 5% 10 CV MeOH in DCM) affording compound 74 (0.010 g, 8% yield) as a cream-colored solid. ¹H NMR (400 MHz, DMSO-d₆) δ (ppm): 8.46 (d, J=5.2 Hz, 1H), 7.82-7.80 (m, 2H), 7.51-7.45 (m, 6H), 7.39 (s, 1H), 7.22-7.20 (m, 2H), 6.46 (d, J=5.2 Hz, 1H), 4.09 (s, 2H), 3.97-3.91 (m, 2H), 3.94 (s, 3H), 3.91 (s, 3H), 3.34-3.29 (m, 2H). MS (m/z): 546.2 (M+H).

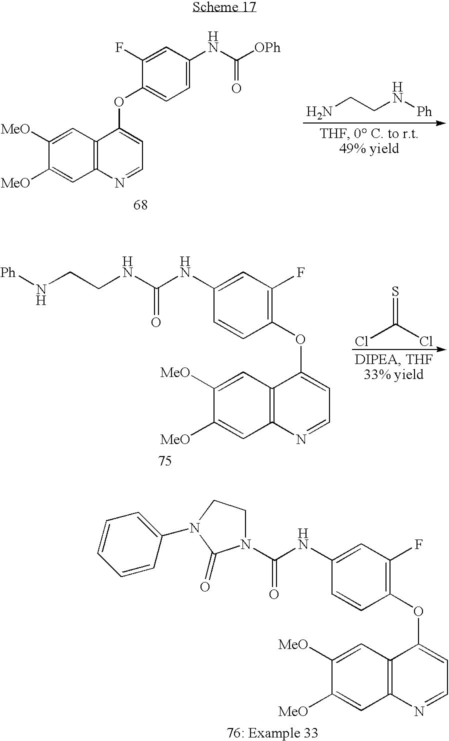

### Example 33

### N-(4-(6,7-dimethoxyquinolin-4-yloxy)-3-fluorophenyl)-3-phenyl-2-thioxoimidazolidine-1-carboxamide (76)

### Step 1. 1-(4-(6,7-dimethoxyquinolin-4-yloxy)-3-fluorophenyl)-3-(2-(phenylamino)ethyl)urea (75)

N-phenylethylenediamine (0.062 ml, 0.477 mmol) was added to a solution of compound 68 (0.207 g, 0.477 mmol) in THF (4.77 mL) at 0° C. and the mixture was stirred overnight. The reaction mixture was diluted with EtOAc then water and aqueous sodium bicarbonate was added and the mixture was stirred. The resultant suspension was filtered and the solid thus collected was washed with additional water and EtOAc then dried under vacuum to afford compound 75 (0.111 g, 49% yield) as white solid. MS (m/z): 477.2 (M+H).

### Step 2. N-(4-(6,7-dimethoxyquinolin-4-yloxy)-3-fluorophenyl)-3-phenyl-2-thioxoimidazolidine-1-carboxamide (76)

Thiophosgene (0.026 mL, 0.346 mmol) was added to a suspension of compound 75 (0.1098 g, 0.230 mmol) and DIPEA (0.121 mL, 0.691 mmol) in THF (2.304 mL) and the reaction mixture was stirred overnight at room temperature. The mixture was diluted with DCM, washed with saturated ammonium chloride solution, dried over anhydrous sodium sulfate and concentrated under reduced pressure. The residue was purified by flash chromatography [Biotage, Si 25+S column, gradient: 3% MeOH in DCM (10CV), 3% MeOH to 5% in DCM (2CV) and 5% MeOH in DCM (10CV)]; the product thus obtained was crushed with MeOH and filtered affording compound 65 (0.033 g, 6% yield) as white solid. ¹H NMR (400 MHz, DMSO-d₆) δ (ppm): 12.54 (br, 1H), 8.48 (d, J=5.3 Hz, 1H), 7.82 (dd, J=12.7, 2.5 Hz, 1H), 7.53-7.36 (m, 9H), 6.49-6.48 (m, 1H), 4.26-4.22 (m, 2H), 4.13-4.09 (m, 2H). MS (m/z): 519.2 (M+H).

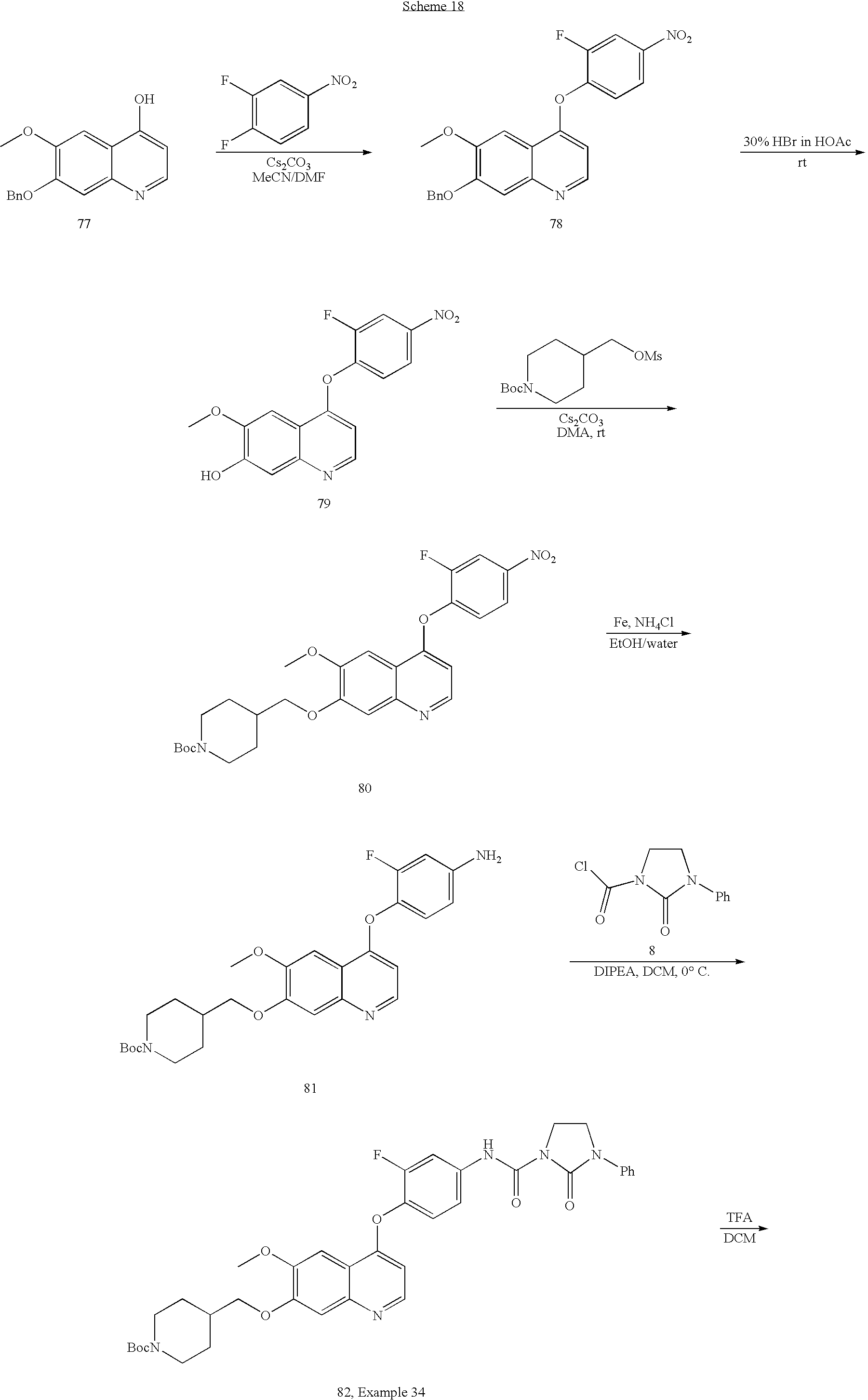

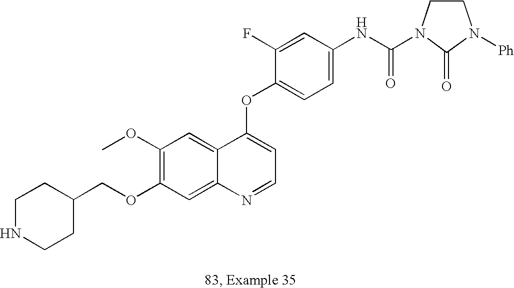

### Example 34

### tert-butyl 4-((4-(2-fluoro-4-(2-oxo-3-phenylimidazolidine-1-carboxamido)phenoxy)-6-methoxyquinolin-7-yloxy)methyl)piperidine-1-carboxylate (82)

### Step 1. 7-(benzyloxy)-4-(2-fluoro-4-nitrophenoxy)-6-methoxyquinoline (78)

To a suspension of compound 77 (WO 2005/030140; 0.76 g, 2.7 mmol) in N,N-dimethylformamide (10 mL) and acetonitrile (10 mL) was added cesium carbonate (1.76 g, 5.4 mmol) followed by 1,2-difluoro-4-nitrobenzene (0.33 mL, 2.97 mmol). The reaction mixture was stirred at room temperature for 3 h and acetonitrile was removed under reduced pressure. The residue was diluted with ethyl acetate and the resultant suspension was successively washed with water, a saturated aqueous solution of sodium bicarbonate and a saturated aqueous solution of sodium chloride, dried over anhydrous sodium sulfate and evaporated under reduced pressure. The residue was purified by column chromatography (eluent a gradient of 45% EtOAc/hexane to 80% EtOAc/hexane) to afford title compound 78 (0.67 g, 32% yield) as a pale yellow solid.

### Step 2. 4-(2-fluoro-4-nitrophenoxy)-6-methoxyquinolin-7-ol (79)

A solution of compound 78 (3.00 g, 7.14 mmol) in 33% HBr in acetic acid (32 ml) was stirred overnight at room temperature. The reaction mixture was then diluted with ether and the solid suspension was collected by filtration and dried under high vacuum to yield the acetic acid salt of compound 79 (2.12 g, 76% yield) as a pale green solid.

### Step 3. tert-butyl 4-((4-(2-fluoro-4-nitrophenoxy)-6-methoxyquinolin-7-yloxy)methyl)piperidine-1-carboxylate (80)

To a solution of compound 79 (2.12 g, 6.42 mmol) in dimethylacetamide (32 ml) was added cesium carbonate (6.28 g, 19.3 mmol) followed by tert-butyl 4-((methylsulfonyloxy)methyl)piperidine-1-carboxylate (2.07 g, 7.06 mmol). The reaction mixture was stirred at room temperature overnight, diluted with water and the solid suspension thus formed was collected by filtration and dried under vacuum. The solid was purified by column chromatography (eluent 50:50 dichloromethane/ethyl acetate) to afford title compound 80 (1.49 g) as pale brown solid. The material was still impure and was used in the next step without additional purification.

### Step 4. tert-butyl 4-((4-(4-amino-2-fluorophenoxy)-6-methoxyquinolin-7-yloxy)methyl)piperidine-1-carboxylate (81)

To a solution of compound 80 (0.50 g, 0.95 mmol) in ethanol (8 mL) and water (4 mL) were added iron (0.254 g, 4.55 mmol) and ammonium chloride (0.123 g, 2.26 mmol). The reaction mixture was heated to reflux for 2 h, diluted with methanol and the solid suspension was filtered off. The filtrate was concentrated and the residue was purified by Gilson (eluent gradient 35% to 57% methanol/dichloromethane) to afford title compound 81 (0.25 g, 52% yield).

### Step 5. tert-butyl 4-((4-(2-fluoro-4-(2-oxo-3-phenylimidazolidine-1-carboxamido)phenoxy)-6-methoxyquinolin-7-yloxy)methyl)piperidine-1-carboxylate (82)

Starting from compound 81 (0.22 g, 0.44 mmol) and following a similar procedure to the one described for compound 9 (scheme 2, example 2), title compound 82 (0.16 g, 54% yield) was obtained as a white solid. ¹H NMR (400 MHz, DMSO-d₆) δ (ppm): 10.6 (s, 1H), 8.46 (d, J=5.3 Hz, 1H), 7.84 (m, 1H), 7.61-7.63 (m, 2H), 7.51 (s, 1H), 7.38-7.46 (m, 5H), 7.17 (m, 1H), 6.44 (dd, J=1.0, 5.3 Hz, 1H), 3.90-4.03 (m, 11H), 2.77 (bs, 2H), 2.03 (bs, 1H), 1.77 (bd, J=12.3 Hz, 2H), 1.39 (s, 9H), 1.20 (m, 2H). MS (m/z): 686.3 (M+H).

### Example 35

### N-(3-fluoro-4-(6-methoxy-7-(piperidin-4-ylmethoxy)quinolin-4-yloxy)phenyl)-2-oxo-3-phenylimidazolidine-1-carboxamide (83)

### Step 1: N-(3-fluoro-4-(6-methoxy-7-(piperidin-4-ylmethoxy)quinolin-4-yloxy)phenyl)-2-oxo-3-phenylimidazolidine-1-carboxamide (83)

To a solution of compound 82 (0.162, 0.24 mmol) in dichloromethane (0.37 mL) was added trifluoroacetic acid (0.37 mL, 4.8 mmol). The reaction mixture was stirred at room temperature for 5 h. The solvent was removed under reduced pressure, the residue was triturated with ethyl ether, and dried under high vacuum to afford the bis-trifluoroacetic acid salt of compound 83 (0.177 g, 91% yield) as a white solid. ¹H NMR (400 MHz, DMSO-d₆) δ (ppm): 10.6 (s, 1H), 8.70 (d, J=6.0 Hz, 1H), 8.61 (bd, J=10.6 Hz, 1H), 8.30 (bm, 1H), 7.90 (m, 1H), 7.63-7.68 (m, 2H), 7.56-7.62 (m, 2H), 7.50-7.52 (m, 2H), 7.40-7.44 (m, 2H), 7.15-7.19 (m, 1H), 6.79 (d, J=5.7 Hz, 1H), 4.11 (d, J=6.3 Hz, 2H), 4.00 (s, 3H), 3.91-3.97 (m, 4H), 3.34 (m, 2H), 2.95 (m, 2H), 2.20 (bs, 1H), 1.97 (bd, J=12.7 Hz, 2H), 1.52 (m, 2H). MS (m/z): 586.4 (M+H).

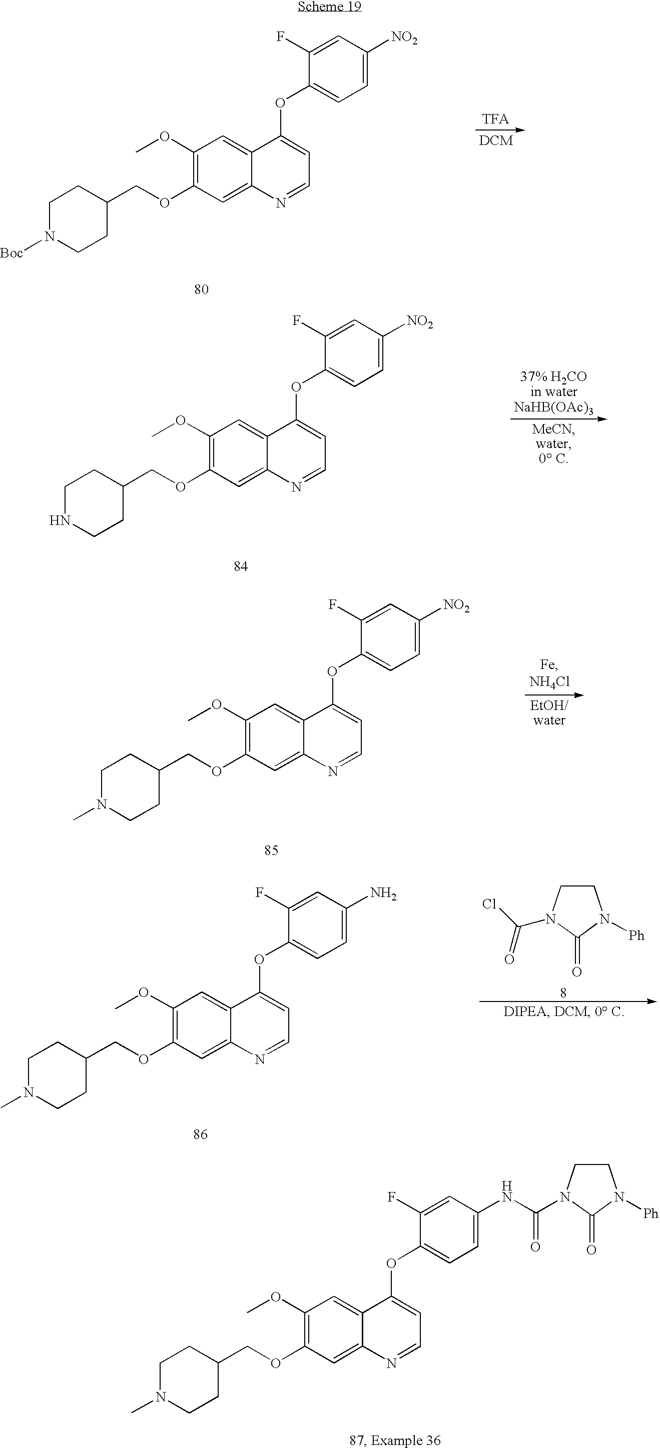

### Example 36

### N-(3-fluoro-4-(6-methoxy-7-((1-methylpiperidin-4-yl)methoxy)quinolin-4-yloxy)phenyl)-2-oxo-3-phenylimidazolidine-1-carboxamide (87)

### Step 1. 4-(2-fluoro-4-nitrophenoxy)-6-methoxy-7-(piperidin-4-ylmethoxy)quinoline (84)

To a solution of compound 80 (0.45 g, 0.85 mmol) in dichloromethane (0.22 mL) was added trifluoroacetic acid (0.22 mL, 2.8 mmol). The reaction mixture was stirred at room temperature for 2 h. The solvent was removed under reduced pressure and the residue was dried under high vacuum then used directly for next step.

### Step 2. 4-(2-fluoro-4-nitrophenoxy)-6-methoxy-7-((1-methylpiperidin-4-yl)methoxy)quinoline (85)

To a solution of compound 84 (0.36 g, 0.85 mmol) in acetonitrile (1 mL) and water (1 mL) at 0° C. was added formaldehyde (37% solution in water) (0.51 mL) and the mixture was stirred 30 min. Sodium triacetoxyborohydride (0.901 g, 4.25 mmol) was slowly added in small portions and stirring was continued for 1 h at 0° C. A 1N sodium hydroxide solution was added until pH 10 was reached and the aqueous solution was extracted twice with dichloromethane. The combined organic extracts were washed with a saturated aqueous solution of sodium chloride, dried over anhydrous sodium sulfate and the solvent was removed under reduced pressure. The residue was purified by Gilson (eluent gradient 20% to 45% (methanol+2% formic acid)/(dichloromethane+2% formic acid)) to afford title compound 85 (0.19 g, 51%).

### Step 3. 3-fluoro-4-(6-methoxy-7-((1-methylpiperidin-4-yl)methoxy)quinolin-4-yloxy)aniline (86)

Starting from compound 85 and following the same procedure as described for compound 81 (example 35, step 4), compound 86 was obtained in one step.

### Step 4. N-(3-fluoro-4-(6-methoxy-7-((1-methylpiperidin-4-yl)methoxy)quinolin-4-yloxy)phenyl)-2-oxo-3-phenylimidazolidine-1-carboxamide (87)

Starting from compound 86 and following the same procedure as described for compound 82 (example 34, step 5), compound 87 was obtained in one step as a white solid. ¹H NMR (400 MHz, DMSO-d₆) δ (ppm): 8.43 (bs, 2H), 7.81 (m, 1H), 7.63 (m, 3H), 7.40 (dd, J=8.6, 7.4 Hz, 2H), 7.34 (m, 3H), 7.18 (dd, J=7.4, 7.4 Hz, 1H), 6.51 (d, J=4.7 Hz, 1H), 4.11 (d, J=5.7 Hz, 2H), 4.00 (s, 7H), 3.48 (d, J=11.9 Hz, 2H), 2.95 (dd, J=11.7, 11.7 Hz, 2H), 2.80 (s, 3H), 2.15-2.28 (bm, 3H), 1.76 (bm, 2H). MS (m/z): 600.3 (M+H).

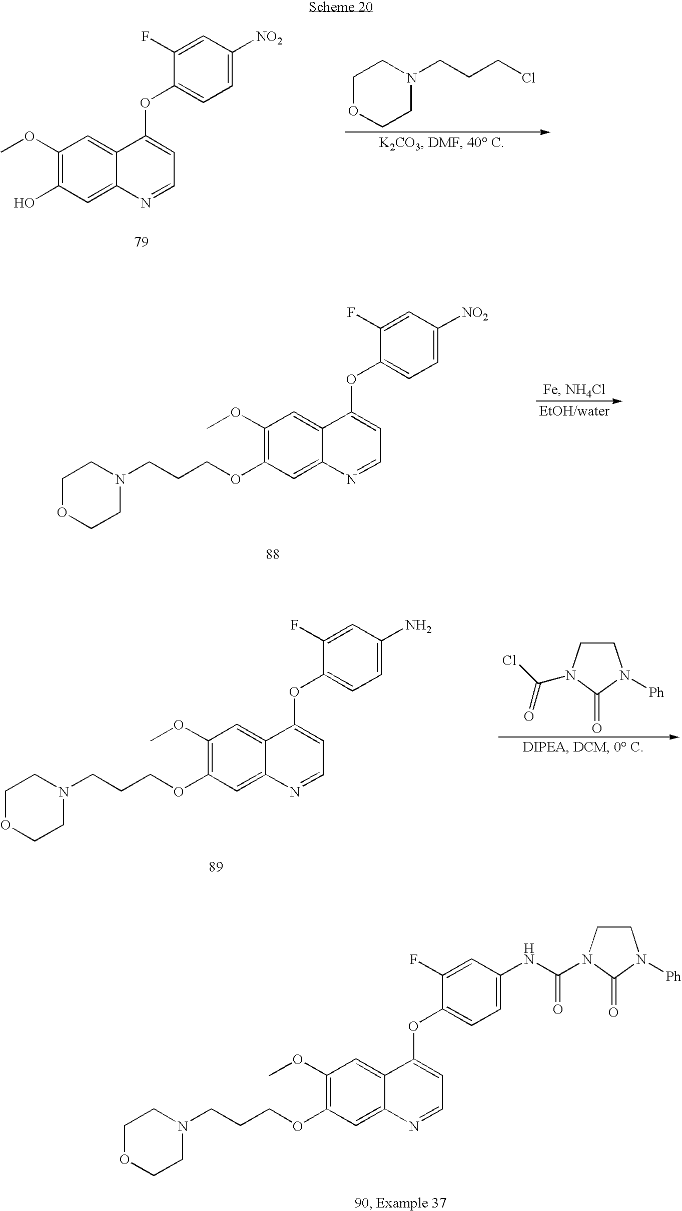

### Example 37

### N-(3-fluoro-4-(6-methoxy-7-(3-morpholinopropoxy)quinolin-4-yloxy)phenyl)-2-oxo-3-phenylimidazolidine-1-carboxamide (90)

### Step 1. 4-(3-(4-(2-fluoro-4-nitrophenoxy)-6-methoxyquinolin-7-yloxy)propyl)morpholine (88)

To a solution of compound 79 (0.500 g, 1.51 mmol) in dry N,N-dimethylformamide (7.5 mL) was added potassium carbonate (1.05 g, 3.78 mmol) followed by 4-(3-chloropropyl)morpholine (0.92 g, 5.6 mmol) and the mixture was stirred at 40° C. for 20 h. The reaction mixture was cooled, cold water was added and the aqueous phase was extracted three times with ethyl acetate. The combined organic layers were washed with a saturated aqueous solution of sodium chloride, dried over anhydrous sodium sulfate and the solvent was removed under reduced pressure. The crude product-pale brown oil (1.5 g) was used directly for next step without further purification.

### Step 2. 3-fluoro-4-(6-methoxy-7-(3-morpholinopropoxy)quinolin-4-yloxy)aniline (89)

Starting from compound 88 and following the same procedure as described for compound 81 (example 35, step 4), compound 89 was obtained in one step as a pale yellow solid which was used in the next step with no additional purification.

### Step 3. N-(3-fluoro-4-(6-methoxy-7-(3-morpholinopropoxy)quinolin-4-yloxy)phenyl)-2-oxo-3-phenylimidazolidine-1-carboxamide (90)

Starting from compound 89 and following the same procedure as described for compound 82 (example 34, step 5), compound 90 was obtained in one step as a pale yellow solid. ¹H NMR (400 MHz, DMSO-d₆) δ (ppm): 10.56 (s, 1H), 8.46 (d, J=5.3 Hz, 1H), 7.83 (m, 1H), 7.62 (m, 2H), 7.51 (s, 1H), 7.41 (m, 5H), 7.17 (m, 1H), 6.44 (dd, J=5.2, 1.1 Hz, 1H), 4.19 (t, J=6.4 Hz, 2H), 3.95 (m, 7H), 3.57 (m, 4H), 2.45 (at almost overlapped by DMSO signal), 2.38 (bs, 4H), 1.97 (m, 2H). MS (m/z): 616.3 (M+H).

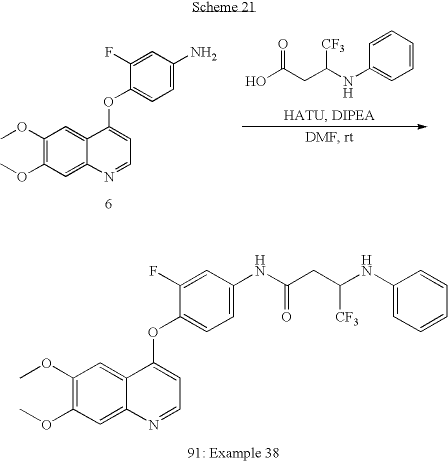

### Example 38

### N-(4-(6,7-dimethoxyquinolin-4-yloxy)-3-fluorophenyl)-4,4,4-trifluoro-3-(phenylamino)butanamide (91)

### Step 1: N-(4-(6,7-dimethoxyquinolin-4-yloxy)-3-fluorophenyl)-4,4,4-trifluoro-3-(phenylamino)butanamide (91)

To a solution of compound 6 (60 mg, 0.19 mmol) in N,N-dimethylformamide (3 mL) was added 4,4,4-trifluoro-3-(phenylamino)butanoic acid (156 mg, 0.66 mmol) (US 2007/0004675 A1, scheme 76, compound 327) followed by N,N-diisopropylethylamine (0.35 mL, 2.0 mmol) and HATU reagent (0.65 g, 1.72 mmol). The reaction mixture was stirred at room temperature for 2 days then diluted with ethyl acetate and washed with a saturated aqueous solution of sodium bicarbonate and a saturated aqueous solution of ammonium chloride. The organic phase was collected dried over anhydrous sodium sulfate and the solvent was removed under reduced pressure. The residue was purified by column chromatography (eluent EtOAc) followed by trituration with methanol/water to afford title compound 91 (16 mg, 16% yield) as a white solid. ¹H NMR (400 MHz, DMSO-d₆) δ (ppm): 10.47 (s, 1H), 8.45 (d, J=5.3 Hz, 1H), 7.81 (dd, J=12.8, 2.4 Hz, 1H), 7.51 (s, 1H), 7.45-7.35 (m, 3H), 7.11 (t, J=8.0 Hz, 2H), 6.75 (d, J=8.0 Hz, 2H), 6.62 (t, J=7.2 Hz, 1H), 6.42 (d, J=4.8 Hz, 1H), 6.12 (d, J=9.2 Hz, 1H), 4.76-4.65 (m, 1H), 3.94 (s, 3H), 3.94 (s, 3H), 2.93 (dd, J=16.0, 4.0 Hz, 1H), 2.78 (dd, J=16.0, 9.6 Hz, 1H). MS (m/z): 530.2 (M+H).

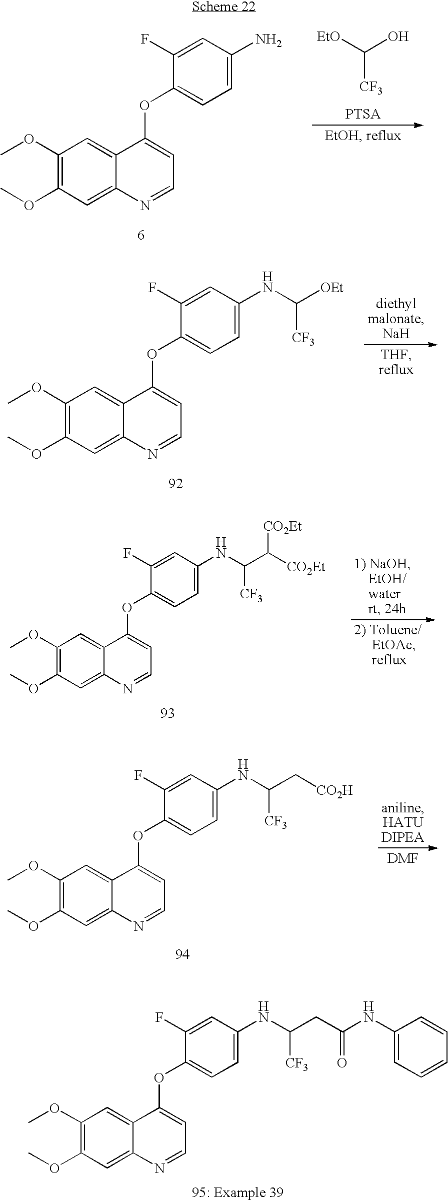

### Example 39

### 3-(4-(6,7-dimethoxyquinolin-4-yloxy)-3-fluorophenylamino)-4,4,4-trifluoro-N-phenylbutanamide (84)

### Step 1. 4-(6,7-dimethoxyquinolin-4-yloxy)-N-(1-ethoxy-2,2,2-trifluoroethyl)-3-fluoroaniline (92)

A mixture of compound 6 (100 mg, 0.24 mmol), trifluoroacetaldehyde ethyl hemiacetal (86 μL, 0.73 mmol) and recrystallized 4-toluenesulfonic acid monohydrate (51 mg, 0.27 mmol) in ethanol (25 mL) was heated to reflux for 24 h. The reaction mixture was concentrated and the residue was dissolved in ethyl acetate, washed with a saturated aqueous solution of sodium bicarbonate, dried over anhydrous sodium sulfate, filtered and the solvent was removed under reduced pressure. The crude material was used directly for next step without further purification. MS (m/z): 441.2 (M+H).

### Step 2. diethyl 2-(1-(4-(6,7-dimethoxyquinolin-4-yloxy)-3-fluorophenylamino)-2,2,2-trifluoroethyl)malonate (93)

To a solution of compound 92 (107 mg, 0.24 mmol) and diethyl malonate (41 μL, 0.27 mmol) in anhydrous tetrahydrofuran (10 mL) under nitrogen was added sodium hydride (60% in oil, 22 mg, 0.54 mmol). The mixture was heated to reflux for 2 h then cooled to room temperature and diluted with EtOAc and water before acidification to pH 3 using a 1N HCl solution. The organic layer was separated and the aqueous layer extracted twice with EtOAc. The extracts and original organic layer were combined, dried over sodium sulfate and the solvents were removed under reduced pressure. The residue was purified by Biotage (Si 12M, gradient: MeOH in dichloromethane 0% to 60%) to afford title compound 93 (70 mg, 54% yield) as a brown oil. MS (m/z): 555.2 (M+H).

### Step 3. 3-(4-(6,7-dimethoxyquinolin-4-yloxy)-3-fluorophenylamino)-4,4,4-trifluorobutanoic acid (94)

A solution of compound 93 (70 mg, 0.13 mmol) and sodium hydroxide (50 mg, 1.3 mmol) in water (0.3 mL) and ethanol (1.5 mL) was stirred at room temperature for 48 h. The solvents were removed under reduced pressure and the residue was dissolved in water (10 mL). The solution was acidified to pH 4 with a 3N HCl solution and extracted twice with ethyl acetate. The combined organic layers were dried over anhydrous sodium sulfate and the solvent was removed under reduced pressure. The residue was suspended in ethyl acetate (2 mL) and anhydrous toluene (30 mL) then heated to reflux for 1 h with continuous stirring before removing the solvent under reduced pressure. The residue was purified by column chromatography on silica gel (eluent MeOH-dichloromethane, 30:70) to afford title compound 94 (20 mg, 35% yield). MS (m/z): 455.2 (M+H).

### Step 4. 3-(4-(6,7-dimethoxyquinolin-4-yloxy)-3-fluorophenylamino)-4,4,4-trifluoro-N-phenylbutanamide (95)

To a stirred solution of compound 94 (20 mg, 0.044 mmol), aniline (6 μL, 0.066 mmol) and N,N-diisopropylethylamine (27 μL, 0.154 mmol) in dry N,N-dimethylformamide (2 mL) at room temperature was added HATU reagent (50 mg, 0.132 mmol). The mixture was stirred at room temperature for 48 h. A saturated aqueous solution of ammonium chloride and ethyl acetate were added and the layers were separated. The organic layer was successively washed with a saturated aqueous solution of sodium bicarbonate and brine, dried over anhydrous sodium sulfate then filtered and the solvent was removed under reduced pressure. The residue was purified by Biotage (Si 12M, gradient: MeOH in dichloromethane 0% to 20%) to afford title compound 95 (1.5 mg, 7% yield) as a beige solid. ¹H NMR (400 MHz, DMSO-d₆) δ (ppm): 10.11 (s, 1H), 8.44 (d, J=5.2 Hz, 1H), 7.62-7.54 (m, 2H), 7.50 (s, 1H), 7.38 (s, 1H), 7.31 (t, J=8.0 Hz, 2H), 7.18 (t, J=9.2 Hz, 1H), 7.05 (t, J=7.6 Hz, 1H), 6.86 (dd, J=13.6, 2.8 Hz, 1H), 6.68 (dd, J=9.2, 2.4 Hz, 1H), 6.54 (d, J=9.6 Hz, 1H), 6.36 (d, J=5.2 Hz, 1H), 4.85-4.73 (m, 1H), 3.94 (s, 6H), 2.92 (dd, J=15.6, 3.6 Hz, 1H), 2.76 (dd, J=15.6, 9.6 Hz, 1H). MS (m/z): 530.2 (M+H).

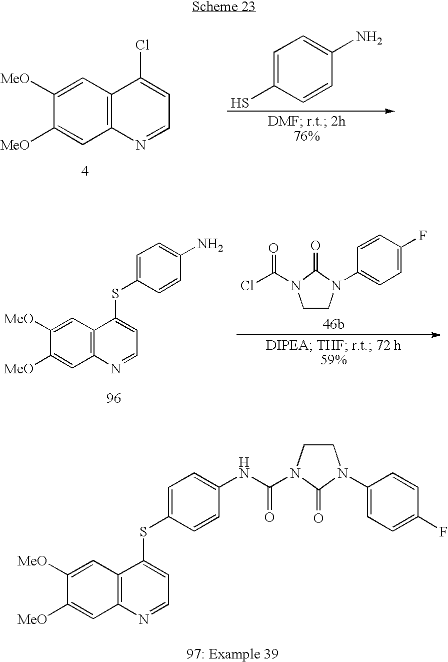

### Example 40

### N-(4-(6,7-Dimethoxyquinolin-4-ylthio)phenyl)-3-(4-fluorophenyl)-2-oxoimidazolidine-1-carboxamide, (97)

### Step 1: 4-(6,7-dimethoxyquinolin-4-ylthio)aniline (96)

To chloroquinoline 4 (0.24 g, 1.1 mmol) in dry DMF was added 4-aminothiophenol (0.30 g, 2.4 mmol) and the resulting mixture was stirred at r.t. for 2 h. The mixture was partitioned between ethyl acetate and 1M NaOH(aq). The organic phase was washed with water and brine, dried (MgSO₄), filtered and concentrated. The residue was purified by silica gel chromatography (ethyl acetate) to afford 96 (0.25 g, 76%) as a colorless solid. ¹H NMR (400 MHz, DMSO-d₆) δ (ppm): 8.36 (d, J=4.9 Hz, 1H), 7.35 (s, 1H), 7.27 (s, 1H), 7.25 (d, J=7.4 Hz, 2H), 6.70 (d, J=7.4 Hz, 2H), 6.51 (d, J=4.9 Hz, 1H), 5.70 (s, 2H), 3.93 (s, 3H), 3.92 (s, 3H). MS (m/z): 313.1 (M+H).

### Step 2: N-(4-(6,7-Dimethoxyquinolin-4-ylthio)phenyl)-3-(4-fluorophenyl)-2-oxoimidazolidine-1-carboxamide (97)

To a suspension of aniline 96 (0.11 g, 0.36 mmol) and DIPEA (0.25 mL, 1.4 mmol) in dry THF (30 mL) was added a suspension of freshly prepared carbamyl chloride 46b (0.12M in THF, 4.0 mL, 0.48 mmol) and the resulting mixture was stirred at r.t. for 72 h. The reaction was concentrated and the residue was purified by silica gel chromatography (5% methanol/ethyl acetate). The residue was triturated with methanol to yield 97 (0.110 g, 59%). ¹H NMR (400 MHz, DMSO-d₆) δ (ppm): 10.54 (s, 1H), 8.41 (d, J=4.9 Hz, 1H), 7.73-7.70 (m, 2H), 7.65-7.60 (m, 2H), 7.59-7.56 (m, 2H), 7.37 (s, 1H), 7.30 (s, 1H), 7.29-7.24 (m, 2H), 6.65 (d, J=4.9 Hz, 1H), 3.93 (s, 4H), 3.92 (s, 3H), 3.91 (s, 3H). MS (m/z): 519.2 (M+H).

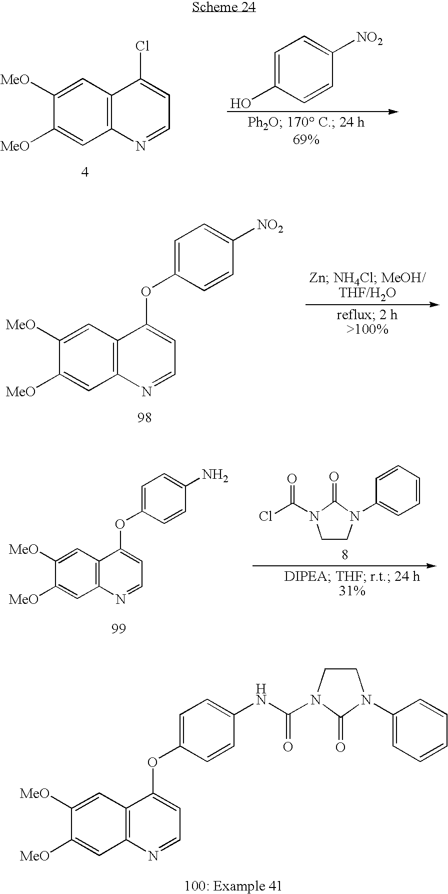

### Example 41

### N-(4-(6,7-dimethoxyquinolin-4-yloxy)phenyl)-2-oxo-3-phenylimidazolidine-1-carboxamide (100)

### Step 1: 6,7-dimethoxy-4-(4-nitrophenoxy)quinoline (98)

To chloroquinoline 4 (0.24 g, 1.1 mmol) in diphenyl ether (20 mL) was added 4-nitrophenol (0.30 g, 2.2 mmol) and the resulting mixture was heated to 170° C. for 24 h. The mixture was partitioned between ethyl acetate and 1M NaOH_{(aq)}. The organic phase was collected, washed with water and brine, dried (MgSO₄), filtered and concentrated. The residue was purified by flash column chromatography (90% ethyl acetate/hexanes-ethyl acetate) to afford 98 (0.25 g, 69%) as a colorless solid. ¹H NMR (400 MHz, DMSO-d₆) δ (ppm): 8.61 (d, J=5.1 Hz, 1H), 8.36-8.32 (m, 2H), 7.46-7.42 (m, 3H), 7.37 (s, 1H), 6.87 (d, J=5.1 Hz, 1H), 3.96 (s, 3H), 3.88 (s, 3H). MS (m/z): 327.1 (M+H).

### Step 2: 4-(6,7-dimethoxyquinolin-4-yloxy)aniline (99)

To 98 (0.25 g, 0.77 mmol) in 1:1 MeOH/THF (50 mL) was added Zn dust (0.55 g, 8.4 mmol) and ammonium chloride (0.085 g, 1.6 mmol) in water (5 mL). The resulting mixture was heated to reflux for 2 h, then filtered through celite and concentrated. The residue was dissolved in dichloromethane, washed with water, brine, dried (MgSO₄), filtered and concentrated to provide crude 99 (0.25 g, >100%) which was used without further purification. MS (m/z): 297.1 (M+H).

### Step 3: N-(4-(6,7-dimethoxyquinolin-4-yloxy)phenyl)-2-oxo-3-phenylimidazolidine-1-carboxamide (100)

To a suspension of aniline 99 (0.13 g, 0.44 mmol) and DIPEA (0.7 mL, 4 mmol) in dry THF (30 mL) was added a suspension of freshly prepared carbamyl chloride 8 (0.1M in THF, 6 mL, 0.6 mmol) and the resulting mixture was stirred at r.t. for 24 h. The reaction mixture was concentrated and the residue was purified by silica gel chromatography (4-8% methanol/ethyl acetate). The residue was triturated with ethyl acetate and methanol to yield 100 (0.065 g, 31%). ¹H NMR (400 MHz, DMSO-d₆) δ (ppm): 10.44 (s, 1H), 8.46 (d, J=5.3 Hz, 1H), 7.69-7.64 (m, 2H), 7.63-7.60 (m, 2H), 7.50 (s, 1H), 7.43-7.39 (m, 2H), 7.38 (s, 1H), 7.27-7.22 (m, 2H), 7.18-7.13 (m, 1H), 6.45 (d, J=5.1 Hz, 1H), 3.96-3.93 (m, 4H), 3.93 (s, 3H), 3.92 (s, 3H). MS (m/z): 485.3 (M+H).

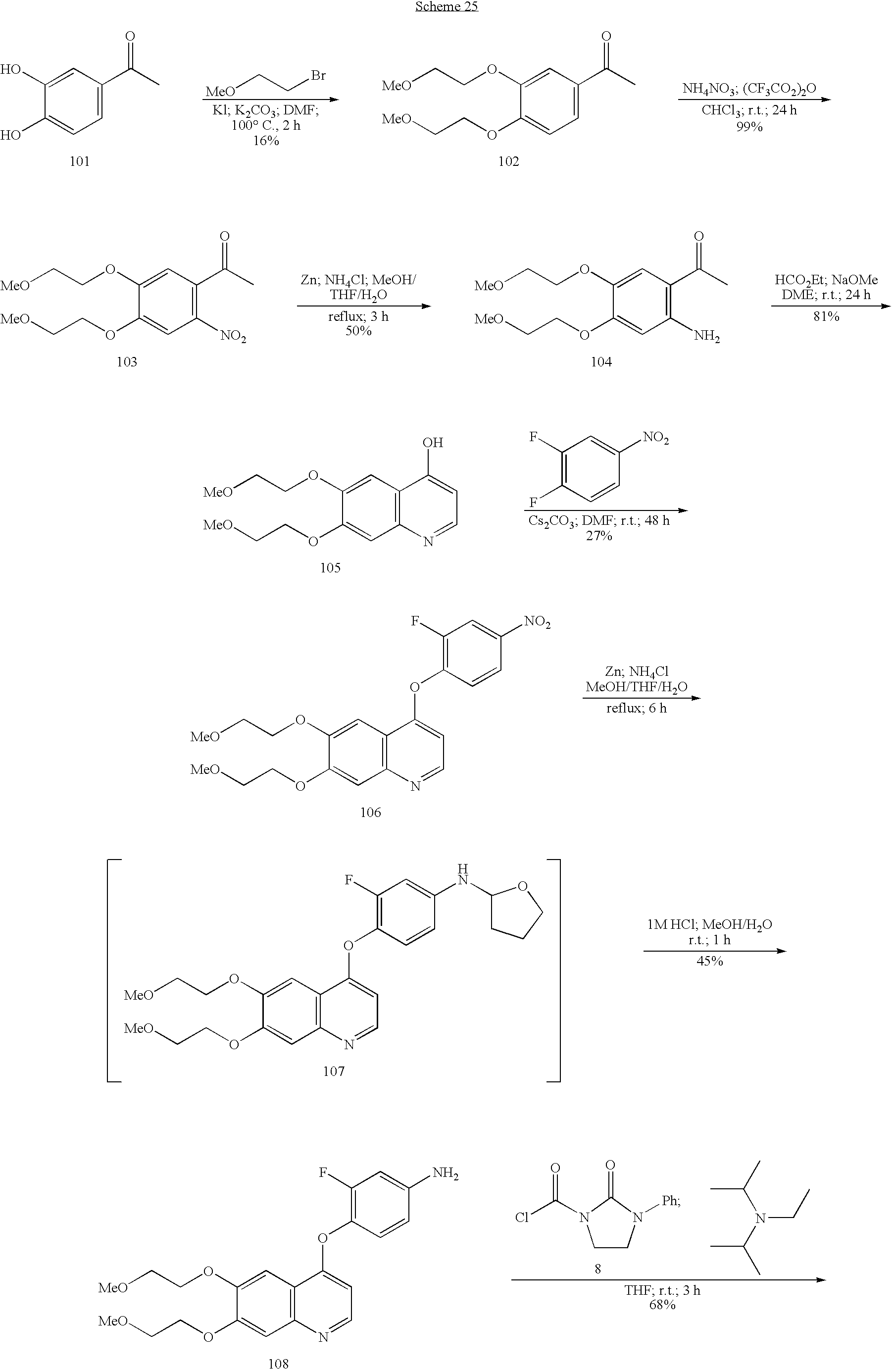

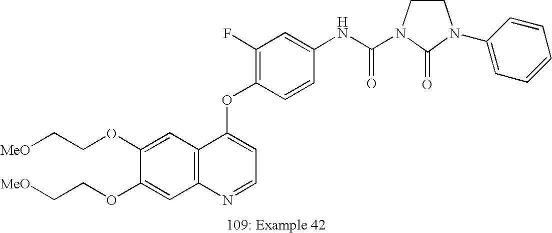

### Example 42

### N-(4-(6,7-Bis(2-methoxyethoxy)quinolin-4-yloxy)-3-fluorophenyl)-2-oxo-3-phenylimidazolidine-1-carboxamide (109)

### Step 1: 1-(3,4-bis(2-methoxyethoxy)phenyl)ethanone (102)

3,4-Dihydroxyacetophenone 101 (5.01 g, 32.9 mmol), bromoethyl methyl ether (10 mL, 106 mmol), potassium iodide (9.6 g, 58 mmol) and potassium carbonate (12.5 g, 90.4 mmol) were dissolved in DMF (50 mL), and the mixture was heated to 100° C. for 2 h. The recation mixture was cooled and partitioned between diethyl ether and water. The organic phase was washed with water, 1M NaOH, 1M HCl, and brine, dried (MgSO₄), filtered and concentrated. Silica gel chromatography (80% ethyl acetate/hexanes) gave 102 (1.37 g, 16%). ¹H NMR (400 MHz, DMSO-d₆) δ (ppm): 7.57-7.54 (m, 2H), 6.92 (d, J=8.6 Hz, 1H), 4.23-4.19 (m, 4H), 3.82-3.77 (m, 4H), 3.46 (s, 6H), 2.55 (s, 3H). MS (m/z): 269.2 (M+H).

### Step 2: 1-(4,5-bis(2-methoxyethoxy)-2-nitrophenyl)ethanone (103)

To ketone 102 (1.37 g, 5.11 mmol) in chloroform (50 mL) was added ammonium nitrate (0.47 g, 5.9 mmol) and trifluoroacetic anhydride (1.5 mL, 11 mmol) and the mixture was stirred for 24 h. The reaction was quenched with saturated sodium bicarbonate solution (1.0 mL) and the organic phase was washed with saturated sodium bicarbonate, water, brine, dried (MgSO₄), filtered and concentrated to provide crude 103 (1.58 g, 99%) which was used without further purification. MS (m/z): 314.2 (M+H).

### Step 3: 1-(2-amino-4,5-bis(2-methoxyethoxy)phenyl)ethanone (104)

To nitro ketone 103 (1.12 g, 3.57 mmol) in 1:1 MeOH/THF (75 mL) was added Zn dust (2.0 g, 31 mmol) and ammonium chloride (0.45 g, 8.4 mmol) in water (5 mL). The resulting mixture was heated to reflux for 3 h, then cooled, filtered through celite and concentrated. The residue was partitioned between ethyl acetate and water then washed with 1M NaOH. It was then extracted with 1M HCl, and the aqueous acidic layer was neutralized with 1M NaOH. This neutralized aqueous layer was extracted with ethyl acetate, and the organic phase was washed with water, dried (MgSO₄), filtered and concentrated. The crude product was run through a short plug of silica (eluent ethyl acetate) to provide 104 (0.51 g, 50%). ¹H NMR (400 MHz, DMSO-d₆) δ (ppm): 7.17 (s, 1H), 7.05 (s, 2H), 6.29 (s, 1H), 4.05-4.02 (m, 2H), 3.99-3.96 (m, 2H), 3.67-3.64 (m, 2H), 3.60-3.57 (m, 2H), 3.31 (s, 3H), 3.29 (s, 3H), 2.39 (s, 3H). MS (m/z): 284.2 (M+H).

### Step 4: 6,7-bis(2-methoxyethoxy)quinolin-4-ol (105)

To aniline 104 (0.51 g, 1.8 mmol) in DME (50 mL) was added sodium methoxide (0.30 g, 5.5 mmol) and the mixture was stirred for 30 min. Ethyl formate (1.0 mL, 12 mmol) was added and the mixture was heated to reflux. After 18 h, the reaction was not complete, so more sodium methoxide (0.55 g, 1.9 mmol) and ethyl formate (1.0 mL, 12 mmol) were added and heating was continued for 6 h. The reaction mixture was quenched with saturated ammonium chloride solution (2 mL) and concentrated. The residue was triturated with 3:1 ethyl acetate/methanol, the insoluble salts were removed by filtration, and the filtrate was concentrated. The residue was purified by silica gel chromatography (20%-40% methanol/ethyl acetate) to provide 105 (0.43 g, 81%). MS (m/z): 294.2 (M+H).

### Step 5: 4-(2-fluoro-4-nitrophenoxy)-6,7-bis(2-methoxyethoxy)quinoline (106)

Hydroxyquinoline 105 (0.89 g, 3.0 mmol), 3,4-difluoronitrobenzene (1.0 mL, 8.8 mmol) and cesium carbonate (2.5 g, 7.7 mmol) were dissolved in DMF (10 mL). The mixture was stirred at r.t. for 48 h, then it was partitioned between ethyl acetate and water, the organic phase was washed with water, brine, dried (MgSO₄), filtered and concentrated. Silica gel chromatography of the residue (5% methanol/ethyl acetate) gave 106 (0.35 g, 27%). ¹H NMR (400 MHz, DMSO-d₆) δ (ppm): 8.57 (d, J=5.1 Hz, 1H), 8.46 (dd, J=10.6, 2.7 Hz, 1H), 8.21-8.17 (m, 1H), 7.60 (t, J=9.0 Hz, 1H), 7.49 (s, 1H), 7.48 (s, 1H), 6.79 (d, J=5.1 Hz, 1H), 4.32-4.30 (m, 2H), 4.27-4.24 (m, 2H), 3.78-3.75 (m, 2H), 3.73-3.71 (m, 2H), 3.36 (s, 3H), 3.33 (s, 3H). MS (m/z): 433.2 (M+H).

### Step 6 and 7: 4-(6,7-bis(2-methoxyethoxy)quinolin-4-yloxy)-3-fluoroaniline (108)

To 106 (0.33 g, 0.0.75 mmol) in 1:1 MeOH/THF (50 mL) was added Zn dust (0.61 g, 9.3 mmol) and ammonium chloride (0.11 g, 2.1 mmol) in water (5 mL). The resulting mixture was heated to reflux for 2 h, then filtered through celite and concentrated. The residue was dissolved in dichloromethane, washed with water, brine, dried (MgSO₄), filtered and concentrated. Silica gel chromatography (ethyl acetate-5% methanol/ethyl acetate) gave protected amine 107 (MS=473.3 (M+H)), which was suspended in methanol (10 mL) and 1M HCl (10 mL). This was stirred at r.t. for 1 h then concentrated. The residue was made alkaline with 3M NaOH, and extracted with ether. This organic phase was washed with water, brine, dried (MgSO₄), filtered and concentrated to yield crude aniline 108 (0.14 g, 45%). ¹H NMR (400 MHz, DMSO-d₆) δ (ppm): 8.39 (d, J=5.6 Hz, 1H), 7.66 (s, 1H), 7.36 (s, 1H), 7.03 (t, J=8.8 Hz, 1H), 6.61 (dd, J=12.5, 2.5 Hz, 1H), 6.56 (dd, J=8.6, 1.2 Hz, 1H), 6.46 (dd, J=5.5, 1.2, 1H), 4.32-4.30 (m, 4H), 3.88-3.84 (m, 4H), 3.48 (s, 3H), 3.47 (s, 3H). MS (m/z): 403.2 (M+H).

### Step 8: N-(4-(6,7-Bis(2-methoxyethoxy)quinolin-4-yloxy)-3-fluorophenyl)-2-oxo-3-phenylimidazolidine-1-carboxamide (109)

To a suspension of aniline 108 (0.13 g, 0.33 mmol) and DIPEA (0.35 mL, 0.26 g, 6.1 mmol) in dry THF (30 mL) was added a suspension of freshly prepared carbamyl chloride 8 (0.07M in THF, 6.0 mL, 0.42 mmol) and the resulting mixture was stirred at r.t. for 3 h. The reaction mixture concentrated and the residue was purified by silica gel chromatography (ethyl acetate—5% methanol/ethyl acetate) to give 109 (0.132 g, 68%). ¹H NMR (400 MHz, DMSO-d₆) δ (ppm): 10.55 (s, 1H), 8.46 (d, J=5.1 Hz, 1H), 7.85-7.81 (m, 1H), 7.63-7.60 (m, 2H), 7.55 (s, 1H), 7.44 (s, 1H), 7.42-7.39 (m, 4H), 7.19-7.15 (s, 1H), 6.45 (d, J=5.2 Hz, 1H), 4.29-4.25 (m, 4H), 3.97-3.93 (m, 4H), 3.76-3.72 (m, 4H), 3.35 (s, 3H), 3.33 (s, 3H). MS (m/z): 591.3 (M+H).

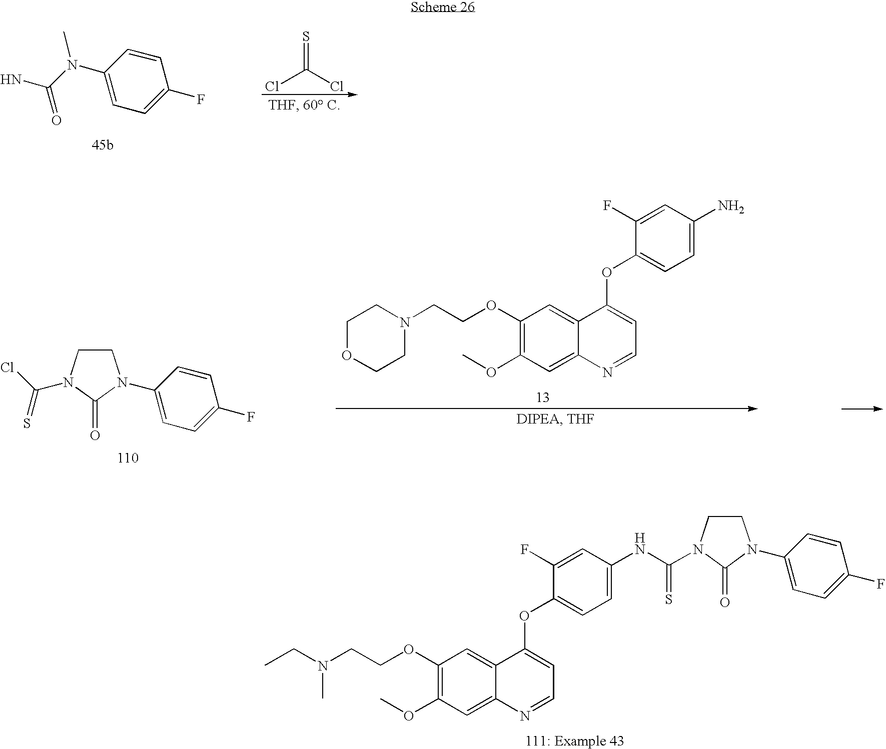

### N-(3-fluoro-4-(7-methoxy-6-(2-morpholinoethoxy)quinolin-4-yloxy)phenyl)-3-(4-fluorophenyl)-2-oxoimidazolidine-1-carbothioamide (112)

### Step 1: 3-(4-fluorophenyl)-2-oxoimidazolidine-1-carbothioyl chloride (110)

To a solution of 45b (227 mg, 1.40 mmol) in THF (14 ml) was added thiophosgene (161 mg, 1 eq, 1.40 mmol) and the reaction mixture was heated to reflux for 4 hours. The mixture was cooled to RT, concentrated and the product was used without additional purification (assumed quantitative).

### Step 2: N-(3-fluoro-4-(7-methoxy-6-(2-morpholinoethoxy)quinolin-4-yloxy)phenyl)-3-(4-fluorophenyl)-2-oxoimidazolidine-1-carbothioamide (111)

To a solution of the aniline 13 (200 mg, 0.47 mmol) in THF (10 ml) was added Hunig's base (361 mg, 6 eq, 2.82 mmol) and 110 (363 mg, 3 eq, 1.41 mmol) and the reaction mixture was stirred at RT for 2 hours. The mixture was adsorbed onto silica gel and purified using column chromatography (5% MeOH in EtOAc) then using the Gilson (35% MeOH in water to 80% MeOH in water, 45 mins, Aquasil C18) to afford 111 (20 mg, 7% yield) as a white solid. ¹H NMR (400 MHz, DMSO-d₆) δ (ppm): 12.29 (s, 1H), 8.48 (d, J=5.3 Hz, 1H), 8.01 (d, J=11.8, 1.2 Hz, 1H), 7.65-7.62 (m, 2H), 7.52-7.54 (m, 3H), 7.39 (s, 1H), 7.30-7.26 (m, 2H), 6.48 (dd, J=5.1, 0.8 Hz, 1H), 4.24-4.16 (m, 4H), 3.97-3.94 (m, 5H), 3.54 (t, J=4.5 Hz, 2H), 2.44 (t, J=7.0 Hz, 3H), 2.36 (s, br, 3H), 1.95 (m, 2H). MS (m/z): 650.3 (M+H).

### Pharmaceutical Compositions

In one embodiment, the invention provides pharmaceutical compositions comprising an inhibitor of VEGF receptor signaling and HGF receptor signaling according to the invention and a pharmaceutically acceptable carrier, excipient, or diluent. Compositions of the invention may be formulated by any method well known in the art and may be prepared for administration by any route, including, without limitation, parenteral, oral, sublingual, transdermal, topical, intranasal, intratracheal, or intrarectal. In certain preferred embodiments, compositions of the invention are administered intravenously in a hospital setting. In certain other preferred embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salt(s)” refers to salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

Inhibition of VEGF Receptor Signaling and HGF Receptor Signaling

In another embodiment the invention provides a method of inhibiting VEGF receptor signaling and HGF receptor signaling in a cell, comprising contacting a cell in which inhibition of VEGF receptor signaling and HGF receptor signaling is desired with an inhibitor of VEGF receptor signaling and HGF receptor signaling according to the invention. Because compounds of the invention inhibit VEGF receptor signaling and HGF receptor signaling, they are useful research tools for in vitro study of the role of VEGF receptor signaling and HGF receptor signaling in biological processes.

Preferably, the method according to this embodiment of the invention causes an inhibition of cell proliferation of the contacted cells. The phrase “inhibiting cell proliferation” is used to denote an ability of an inhibitor of VEGF receptor signaling and HGF receptor signaling to retard the growth of cells contacted with the inhibitor as compared to cells not contacted. An assessment of cell proliferation can be made by counting contacted and non-contacted cells using a Coulter Cell Counter (Coulter, Miami, Fla.) or a hemacytometer. Where the cells are in a solid growth (e.g., a solid tumor or organ), such an assessment of cell proliferation can be made by measuring the growth with calipers and comparing the size of the growth of contacted cells with non-contacted cells.

Preferably, growth of cells contacted with the inhibitor is retarded by at least 50% as compared to growth of non-contacted cells. More preferably, cell proliferation is inhibited by 100% (i.e., the contacted cells do not increase in number). Most preferably, the phrase “inhibiting cell proliferation” includes a reduction in the number or size of contacted cells, as compared to non-contacted cells. Thus, an inhibitor of VEGF receptor signaling and HGF receptor signaling according to the invention that inhibits cell proliferation in a contacted cell may induce the contacted cell to undergo growth retardation, to undergo growth arrest, to undergo programmed cell death (i.e., to apoptose), or to undergo necrotic cell death.

In some preferred embodiments, the contacted cell is a neoplastic cell. The term “neoplastic cell” is used to denote a cell that shows aberrant cell growth. Preferably, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastasis in vivo and that may recur after attempted removal. The term “tumorigenesis” is used to denote the induction of cell proliferation that leads to the development of a neoplastic growth.

In some preferred embodiments, the contacted cell is in an animal. Thus, the invention provides a method for treating a cell proliferative disease or condition in an animal, comprising administering to an animal in need of such treatment a therapeutically effective amount of a VEGF receptor signaling and HGF receptor signaling inhibitor of the invention. Preferably, the animal is a mammal, more preferably a domesticated mammal. Most preferably, the animal is a human.

The term “cell proliferative disease or condition” is meant to refer to any condition characterized by aberrant cell growth, preferably abnormally increased cellular proliferation. Examples of such cell proliferative diseases or conditions amenable to inhibition and treatment include, but are not limited to, cancer. Examples of particular types of cancer include, but are not limited to, breat cancer, lung cancer, colon cancer, rectal cancer, bladder cancer, leukemia and renal cancer. In particularly preferred embodiments, the invention provides a method for inhibiting neoplastic cell proliferation in an animal comprising administering to an animal having at least one neoplastic cell present in its body a therapeutically effective amount of a VEGF receptor signaling and HGF receptor signaling inhibitor of the invention.

### ASSAY EXAMPLES

### Assay Example 1

### Inhibition of c-met and VEGF Activity

The following protocols were used to assay the compounds of the invention.

In Vitro Receptor Tyrosine Kinase Assays (c-Met/HGF receptor and VEGF receptor KDR)

These tests measure the ability of compounds to inhibit the enzymatic activity of recombinant human c-Met/HGF receptor and VEGF receptor enzymatic activity.

A 1.3-kb cDNA corresponding to the intracellular domain of c-Met or c-Met IC (Genbank accession number NP000236-1 amino acid 1078 to 1337) is cloned into the BamHI/XhoI sites of the pBlueBacHis2A vector (Invitrogen) for the production of a histidine-tagged version of that enzyme. This constuct is used to generate recombinant baculovirus using the Bac-N-Blue™ system according to the manufacturer's instructions (Invitrogen).

The c-Met IC protein is expressed in Hi-5 cells (Trichoplusia Ni) upon infection with recombinant baculovirus construct. Briefly, Hi-5 cells grown in suspension and maintained in serum-free medium (Sf900 II supplemented with gentamycin) at a cell density of about 2×10⁶ cells/ml are infected with the above-mentioned viruses at a multiplicity of infection (MOI) of 0.2 during 72 hours at 27° C. with agitation at 120 rpm on a rotary shaker. Infected cells are harvested by centrifugation at 398 g for 15 min. Cell pellets are frozen at −80° C. until purification is performed.

All steps described in cell extraction and purification are performed at 4° C. Frozen Hi-5 cell pellets infected with the C-Met IC recombinant baculovirus are thawed and gently resuspended in Buffer A (20 mM Tris pH 8.0, 10% glycerol, 1 μg/ml pepstatin, 2 μg/ml Aprotinin and leupeptin, 50 μg/ml PMSF, 50 μg/ml TLCK and 10 μM E64, 0.5 mM DTT and 1 mM Levamisole) using 3 ml of buffer per gram of cells. The suspension is Dounce homogenized after which it is centrifuged at 22500 g, 30 min., 4° C. The supernatant (cell extract) is used as starting material for purification of c-Met IC.

The supernatant is loaded onto a QsepharoseFF column (Amersham Biosciences) equilibrated with Buffer B (20 mM Tris pH 8.0, 10% glycerol) supplemented with 0.05M NaCl. Following a ten column volume (CV) wash with equilibration buffer, bound proteins are eluted with a 5 CV salt linear gradient spanning from 0.05 to 1M NaCl in Buffer B. Typically, the conductivity of selected fractions rank between 6.5 and 37 mS/cm. This Qsepharose eluate has an estimated NaCl concentration of 0.33M and is supplemented with a 5M NaCl solution in order to increase NaCl concentration at 0.5M and also with a 5M Imidazole (pH 8.0) solution to achieve a final imidazole concentration of 15 mM. This material is loaded onto a H isTrap affinity column (GE Healthcare) equilibrated with Buffer C (50 mM NaPO₄ pH 8.0, 0.5M NaCl, 10% glycerol) supplemented with 15 mM imidazole. After a 10 CV wash with equilibration buffer and an 8 CV wash with buffer C+40 mM imidazole, bound proteins are eluted with an 8 CV linear gradient (15 to 500 mM) of imidazole in buffer C. C-Met IC enriched fractions from this chromatography step are pooled based on SDS-PAGE analysis. This pool of enzyme undergoes buffer exchange using PD-11 column (GE Healthcare) against buffer D (25 mM HEPES pH 7.5, 0.1M NaCl, 10% glycerol and 2 mM P-mercaptoethanol). Final C-Met IC protein preparations concentrations are about 0.5 mg/ml with purity approximating 80%. Purified c-Met IC protein stocks are supplemented with BSA at 1 mg/ml, aliquoted and frozen at −80° C. prior to use in enzymatic assay.

In the case of VEGF receptor KDR a 1.6-kb cDNA corresponding to the catalytic domain of VEGFR2 or KDR (Genbank accession number AF035121 amino acid 806 to 1356) is cloned into the Pst I site of the pDEST20 Gateway vector (Invitrogen) for the production of a GST-tagged version of that enzyme. This constuct is used to generate recombinant baculovirus using the Bac-to-Bac system according to the manucfacturer's instructions (Invitrogen).

The GST-VEGFR2₈₀₆₋₁₃₅₆ protein is expressed in Sf9 cells (Spodoptera frugiperda) upon infection with recombinant baculovirus construct. Briefly, Sf9 cells grown in suspension and maintained in serum-free medium (Sf900 II supplemented with gentamycin) at a cell density of about 2×10⁶ cells/ml are infected with the above-mentioned viruses at a multiplicity of infection (MOI) of 0.1 during 72 hours at 27° C. with agitation at 120 rpm on a rotary shaker. Infected cells are harvested by centrifugation at 398 g for 15 min. Cell pellets are frozen at −80° C. until purification is performed.

All steps described in cell extraction and purification are performed at 4° C. Frozen Sf9 cell pellets infected with the GST-VEGFR2₈₀₆₋₁₃₅₆ recombinant baculovirus are thawed and gently resuspended in Buffer A (PBS pH 7.3 supplemented with 1 μg/ml pepstatin, 2 μg/ml Aprotinin and leupeptin, 50 μg/ml PMSF, 50 μg/ml TLCK and 10 μM E64 and 0.5 mM DTT) using 3 ml of buffer per gram of cells. Suspension is Dounce homogenized and 1% Triton X-100 is added to the homogenate after which it is centrifuged at 22500 g, 30 min., 4° C. The supernatant (cell extract) is used as starting material for purification of GST-VEGFR2₈₀₆₋₁₃₅₆.

The supernatant is loaded onto a GST-agarose column (Sigma) equilibrated with PBS pH 7.3. Following a four column volume (CV) wash with PBS pH 7.3+1% Triton X-100 and 4 CV wash with buffer B (50 mM Tris pH 8.0, 20% glycerol and 100 mM NaCl), bound proteins are step eluted with 5 CV of buffer B supplemented with 5 mM DTT and 15 mM glutathion. GST-VEGFR2₈₀₆₋₁₃₅₆ enriched fractions from this chromatography step are pooled based on U.V. trace i.e. fractions with high O.D.₂₈₀. Final GST-VEGFR2₈₀₆₋₁₃₅₆ protein preparations concentrations are about 0.7 mg/ml with purity approximating 70%. Purified GST-VEGFR2₈₀₆₋₁₃₅₆ protein stocks are aliquoted and frozen at −80° C. prior to use in enzymatic assay.

Inhibition of c-Met/HGF receptor and VEGFR/KDR is measured in a DELFIA™ assay (Perkin Elmer). The substrate poly(Glu4, Tyr) is immobilized onto black high-binding polystyrene 96-well plates. The coated plates are washed and stored at 4° C. During the assay, enzymes are pre-incubated with inhibitor and Mg-ATP on ice in polypropylene 96-well plates for 4 minutes, and then transferred to the coated plates. The subsequent kinase reaction takes place at 30° C. for 10-30 minutes. ATP concentrations in the assay are 10 uM for C-Met (5× the Kₘ) and 0.6 uM for VEGFR/KDR (2× the Kₘ). Enzyme concentration is 25 nM (C-Met) or 5 nM (VEGFR/KDR). After incubation, the kinase reactions are quenched with EDTA and the plates are washed. Phosphorylated product is detected by incubation with Europium-labeled anti-phosphotyrosine MoAb. After washing the plates, bound MoAb is detected by time-resolved fluorescence in a Gemini SpectraMax reader (Molecular Devices). Compounds are evaluated over a range of concentrations and IC₅₀'s (concentration of compounds giving 50% inhibition of enzymatic activity) are determined.

C-Met Phosphorylation Cell-Based Assay

This test measures the ability of compounds to inhibit HGF stimulated auto-phosphorylation of the c-Met/HGF receptor itself in a whole cell system.

MNNGHOS cell line expressing TPR-MET fusion protein are purchased from ATCC. The TPR-MET is the product of a chromosomal translocation placing the TPR locus on chromosome I upstream of the MET gene on chromosome 7 encoding for its cytoplasmic region catalytic domain. Dimerization of the Mᵣ 65,000 TPR-Met oncoprotein through a leucine zipper motif encoded by the TPR portion leads to constitutive activation of the met kinase. Constitutive autophosphorylation occurs on residues Tyr361/365/366 of TPR-Met. These residues are homologous to Tyr1230/1234/1235 of MET which become phosphorylated upon dimerization of the receptor upon HGF binding.

Inhibitor of c-Met formulated as 30 mM stocks in DMSO. For MNNGHOS treatments, cells, compounds are added to tissue culture media at indicated doses for 3 hours prior to cell lysis. Cells are lysed in ice-cold lysis buffer containing 50 mM HEPES (pH 7.5), 150 mM NaCl, 1.5 mM MgCl₂, 10% glycerol, 1% Triton X-100, 1 mM 4-(2-Aminoethyl)benzenesulfonyl fluoride hydrochloride, 200 μM sodium orthovanadate, 1 mM sodium fluoride, 10 μg/ml of leupeptin, 10 μg/ml of aprotinin/ml, 1 ug/ml of pepstatin and 50 ug/ml Na-p-Tosyl-L-lysine chloromethyl ketone hydrochloride.

Lysate are separated on 5-20% PAGE-SDS and immunoblots are performed using Immobilon P polyvinylidene difluoride membranes (Amersham) according to the manufacturer's instructions for handling. The blots are washed in Tris-buffered saline with 0.1% Tween 20 detergent (TBST). Tyr361/365/366 of TPR-Met are detected with polyclonal rabbit antibodies against tyrosine phosphorylated Met (Biosource International) and secondary antibodies anti-rabbit-horseradish peroxidase (Sigma) by chemiluminescence assays (Amersham, ECL) performed according to the manufacturer's instructions and followed by film exposure. Signal is quantitated by densitometry on Alpha-Imager. IC₅₀ values, as shown in Table 2, are defined as the dose required to obtain 50% inhibition of the maximal HGF stimulated phosphorylated c-Met levels.

TABLE 2
Biological profile of selected compounds

| Ex | Cpd | |
|---|---|---|
| No. | No. | Structure |
| 1 | 7 | 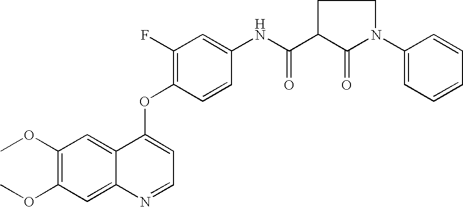 |
| 2 | 9 | 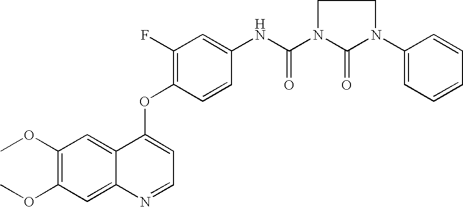 |
| 3 | 15 | 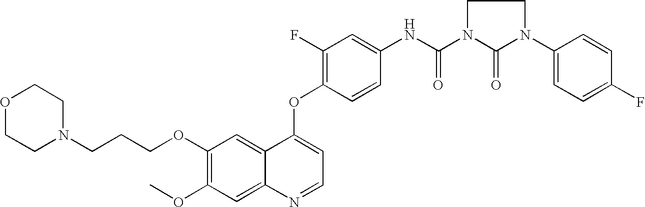 |
| 4 | 16 | 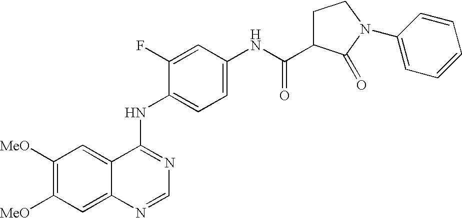 |
| 5 | 17 | 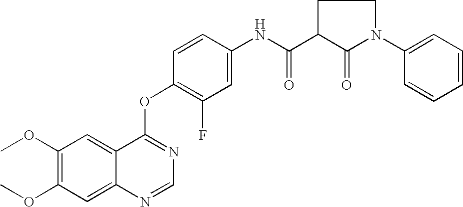 |
| 6 | 18 | 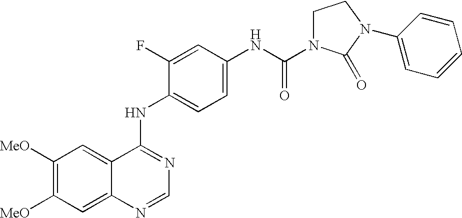 |
| 7 | 19 | 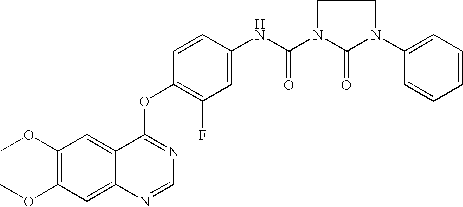 |
| 9 | 21 | 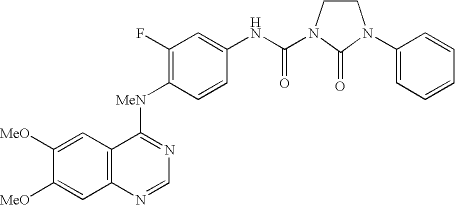 |
| 10 | 22 | 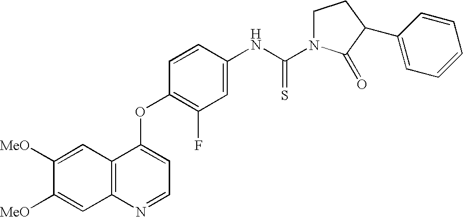 |
| 11 | 23 | 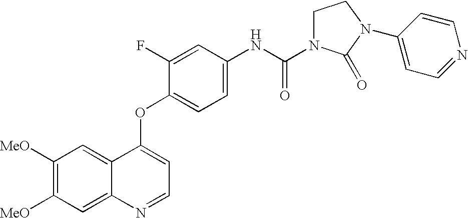 |
| 12 | 24 | 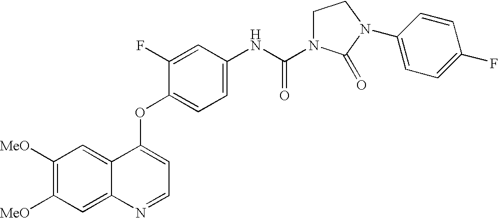 |
| 13 | 25 | 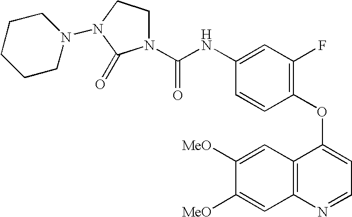 |
| 14 | 26 | 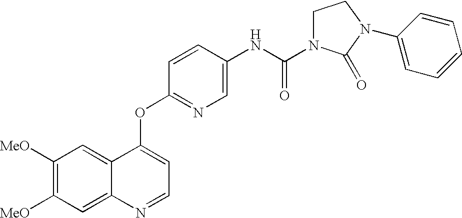 |
| 15 | 27 | 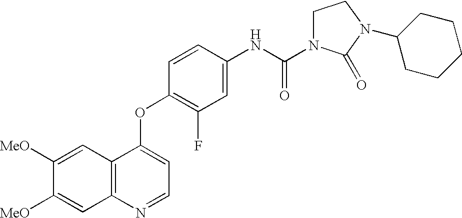 |
| 16 | 28 | 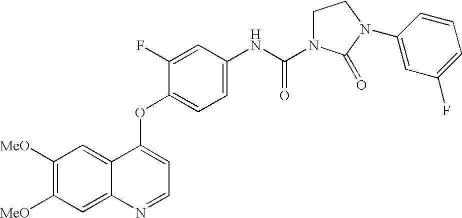 |
| 17 | 29 | 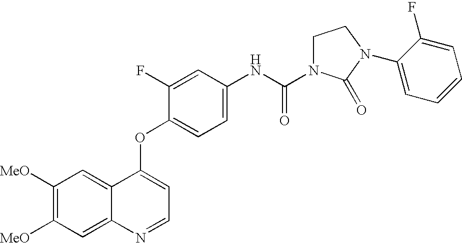 |
| 18 | 30 | 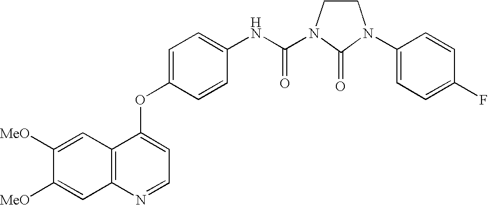 |
| 19 | 31 | 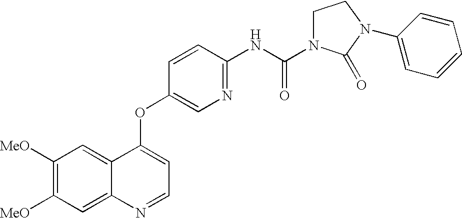 |
| 20 | 32 | 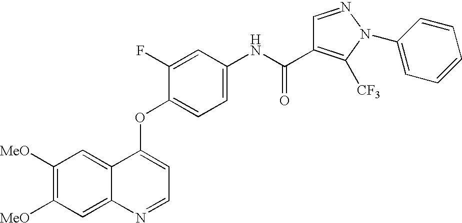 |
| 21 | 33 | 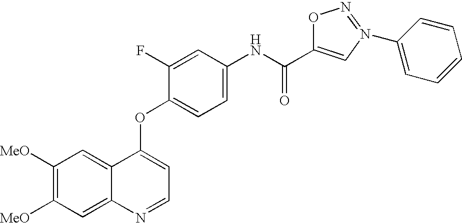 |
| 22 | 39 | 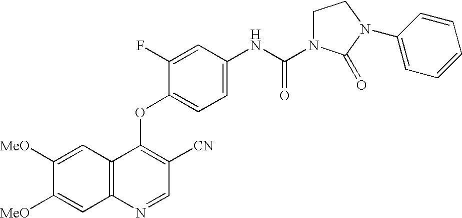 |
| 23 | 47 | 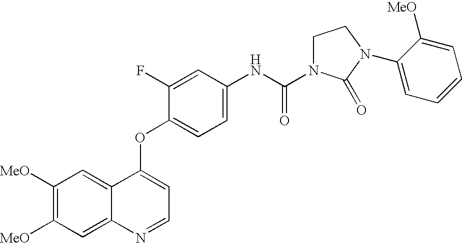 |
| 24 | 48 | 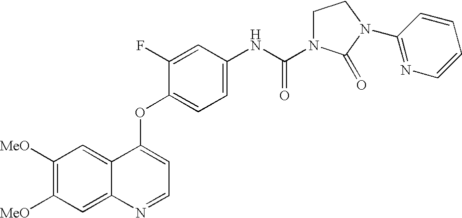 |
| 25 | | 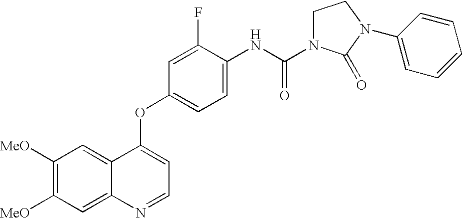 |
| 26 | 55 | 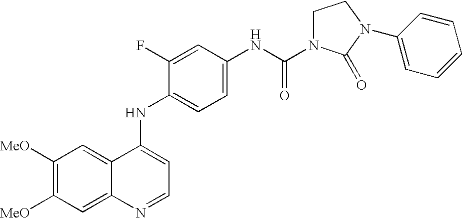 |
| 27 | 57 | 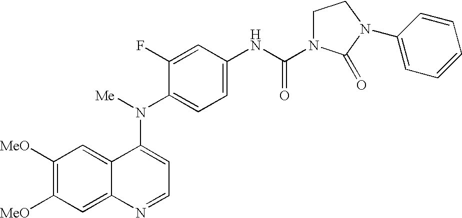 |
| 28 | 59 | 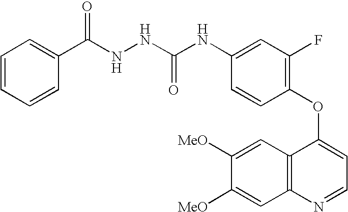 |
| 29 | 63 | 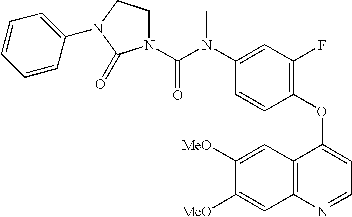 |
| 30 | 67 | 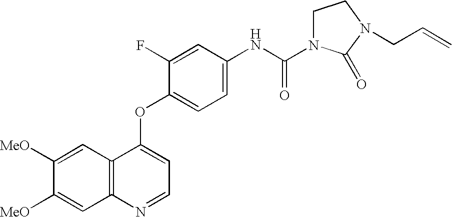 |
| 31 | 70 | 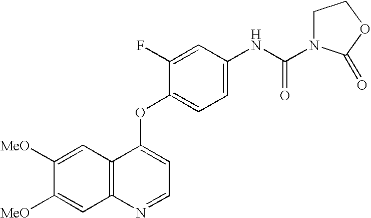 |
| 32 | 74 | 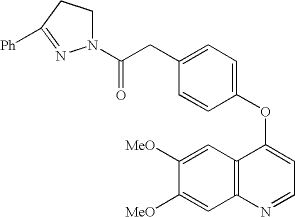 |
| 33 | 76 | 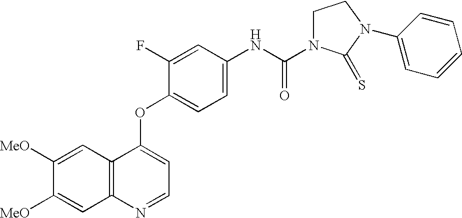 |
| 34 | 82 | 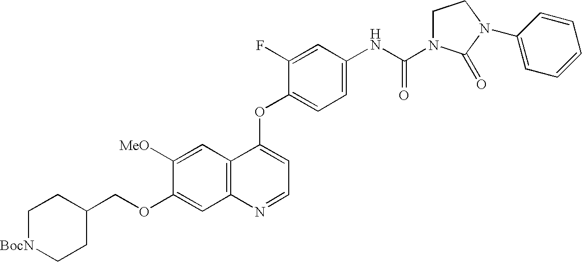 |
| 35 | 83 | 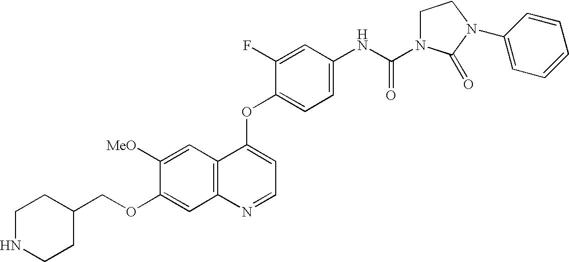 |
| 36 | 87 | 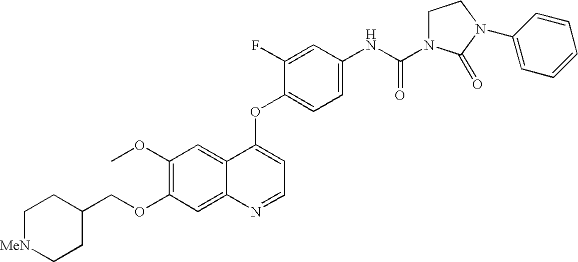 |
| 37 | 90 | 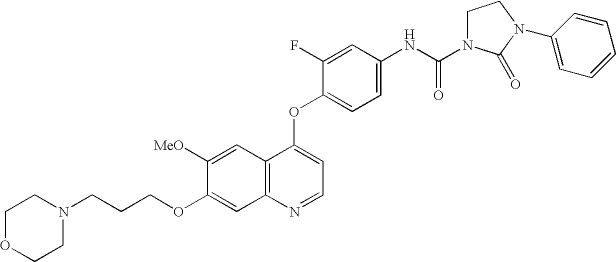 |
| 38 | 91 | 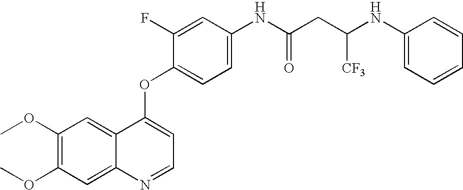 |
| 39 | 95 | 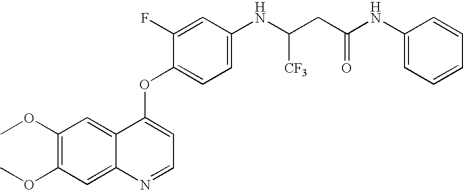 |
| 40 | 97 | 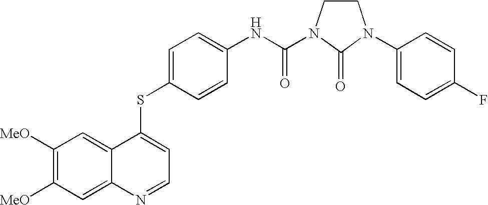 |
| 41 | 100 | 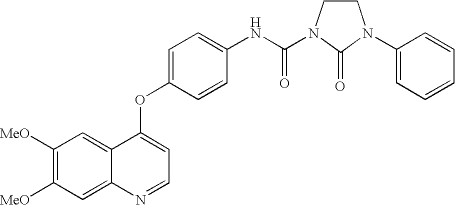 |
| 42 | 109 | 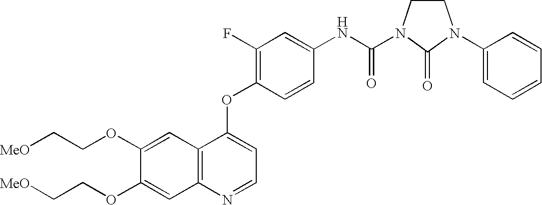 |
| 43 | 111 | 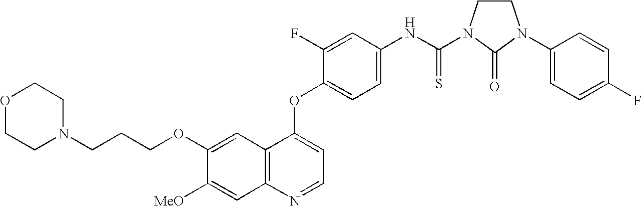 |

| | | | | | Potency in cell-based | |
|---|---|---|---|---|---|---|
| | | | | | assays | |
| | | | Potency in | | A549 | |
| | | | enzyme | | wound | DU145 |
| | | | assays | | healing | scattering |
| Ex | Cpd | | Cmet | VEGF | inhibition | inhibition |
| No. | No. | Characterization | (μM) | (μM) | (μM) | (μM) |
| 1 | 7 | ¹H NMR (400 MHz, DMSO-d₆) δ (ppm): | A | a | d | d |
| | | 10.67 (s, 1H), 8.44 (d, J = 5.28 Hz, 1H), 7.90 | | | | |
| | | (dd, J = 2.35 and 13.1 Hz, 1H), 7.67-7.35 | | | | |
| | | (m, 6H), 7.14 (m, 1H), 6.45 (dd, J = 0.98 and | | | | |
| | | 5.28 Hz, 1H), 3.93 (s, 6H), 3.91 (t, J = 1.57 | | | | |
| | | Hz, 1H) 3.31 (s, 4H). | | | | |
| | | LRMS: 501.1 (calc) 502.1 (found) | | | | |
| 2 | 9 | ¹H NMR (400 MHz, DMSO-d₆) δ (ppm): | A | a | b | 0.4 |
| | | 10.55 (s, 1H), 8.46 (d, J = 5.28 Hz, 1H), 7.82 | | | | |
| | | (m, 1H), 7.61 (d, J = 7.8 Hz, 2H), 7.51 (s, | | | | |
| | | 1H), 7.42 (m, 5H), 7.16 (t, J = 7.24 Hz, 1H), | | | | |
| | | 6.45 (d, J = 5.09 Hz, 1H), 3.92 (s, 6H), 3.31 | | | | |
| | | (s, 4H). | | | | |
| | | LRMS: 502.5 (calc) 503.0 (found) | | | | |
| 3 | 15 | ¹H NMR (400 MHz, DMSO-d₆) δ (ppm): | A | a | b | a |
| | | 10.52 (s, 1H), 8.45 (d, J = 5.28 Hz, 1H), 7.84 | | | | |
| | | (m, 1H), 7.63 (m, 2H), 7.50 (s, 1H), 7.42 (m, | | | | |
| | | 2H), 7.39 (s, 1H), 7.27 (t, J = 8.61 Hz, 2H), | | | | |
| | | 6.44 (d, J = 5.09 Hz, 1H), 4.17 (t, J = 6.26 | | | | |
| | | Hz, 2H), 3.94 (s, 7H), 3.54 (t, J = 4.30 Hz, 4 | | | | |
| | | H), 2.48 (m, 6H), 1.95 (m, 2H). | | | | |
| | | LRMS(ESI): 633.64 (calc.), 634.2 (found) | | | | |
| | | (MH)+ | | | | |
| 4 | 16 | 1H NMR (400 MHz, DMSO-d₆) δ (ppm): | D | d | e | e |
| | | 10.58 (s, 1H), 9.46 (s, 1H), 8.32 (s, 1H), 7.81 | | | | |
| | | (s, 1H), 7.76 (dd, J = 12.7, 2.3 Hz, 1H), 7.71- | | | | |
| | | 7.66 (m, 2H), 7.48 (t, J = 8.7 Hz, 1H), 7.44- | | | | |
| | | 7.38 (m, 3H), 7.21-7.15 (m, 2H), 4.00-3.88 | | | | |
| | | (m, 8H), 3.79 (t, J = 8.5 Hz, 1H), 2.50-2.31 | | | | |
| | | (m, 2H). | | | | |
| | | LRMS: 501.51 (calc) 502.0 (found) | | | | |
| 5 | 17 | 1H NMR (400 MHz, DMSO-d₆) δ (ppm): | B | d | e | e |
| | | 10.63 (s, 1H), 8.53 (s, 1H), 7.83 (d, 12.2 Hz), | | | | |
| | | 7.66 (d, 2H, 8.8 Hz), 7.56 (s, 1H), 7.35-7.45 | | | | |
| | | (m, 5H), 7.16 (t, 1H, J = 7.4 Hz), 3.97 (s, 3H), | | | | |
| | | 3.96 (s, 3H), 3.85-3.95 (m, 2H), 3.77 (t, 1H, | | | | |
| | | J = 8.2 Hz), 2.3-2.5 (m, 2H) | | | | |
| | | LRMS: 502.1 (calc) 503.0 (found) | | | | |
| 6 | 18 | 1H NMR (400 MHz, DMSO-d₆) δ (ppm): | C | d | d | e |
| | | 10.52 (s, 1H), 9.45 (s, 1H), 8.33 (s, 1H), 7.81 | | | | |
| | | (s, 1H), 7.69 (dd, J = 12.5, 2.3 Hz, 1H), 7.66- | | | | |
| | | 7.61 (m, 2H), 7.50-7.40 (m, 3H), 7.33 (dd, J = | | | | |
| | | 8.5, 2.1 Hz, 1H), 7.21-7.15 (m, 2H), 4.02- | | | | |
| | | 3.91 (m, 10H). | | | | |
| | | LRMS: 502.5 (calc) 503.0 (found) | | | | |
| 7 | 19 | 1H NMR (400 MHz, CDCl₃) δ (ppm): 10.55 | A | a | e | e |
| | | (s, 1H), 8.65 (s, 1H), 7.72 (d, 1H, J = 12.4 Hz), | | | | |
| | | 7.54 (s, 1H), 7.51 (d, 2H, J = 7.6 Hz), 7.40 (t, | | | | |
| | | 2H, J = 7.4 Hz), 7.24 (m, 2H), 7.17 (t, 1H, | | | | |
| | | J = 7.4 Hz), 4.07 (m, 2H), 4.05 (s, 3H), 4.04 | | | | |
| | | (s, 3H), 3.92 (m, 2H) | | | | |
| | | LRMS: 503.1 (calc) 504.0 (found) | | | | |
| 9 | 21 | 1H NMR (400 MHz, DMSO-d₆) δ (ppm): | D | d | b | d |
| | | 10.52 (s, 1H); 8.61 (s, 1H); 7.67 (dd, J = 12.9, | | | | |
| | | 2.1, 1H); 7.63-7.61 (m, 2H); 7.48-7.38 (m, | | | | |
| | | 4H); 7.17-7.13 (m, 2H); 6.40 (s, 1H); 3.96- | | | | |
| | | 3.88 (m, 4H); 3.85 (s, 3H); 3.46 (s, 3H); 3.27 | | | | |
| | | (s, 3H). | | | | |
| | | LRMS: 517.2 (calc) 517.1 (found) | | | | |
| 10 | 22 | 1H NMR (400 MHz, DMSO-d6) δ (ppm): | A | a | a | b |
| | | 12.35 (s, 1H), 8.51 (d, J = 5.3 Hz, 1H), 8.04 (dd, | | | | |
| | | J = 2 Hz, J = 12.3 Hz, 1H), 7.64 (dd, J = 8.8 Hz, | | | | |
| | | J = 1 Hz, 2H), 7.55-7.42 (m, 6H), 7.21 (t, | | | | |
| | | J = 7.3 Hz, 1H), 6.51 (dd, J = 1 Hz, J = 5.3 Hz, | | | | |
| | | 1H), 4.24 (dd, J = 7.2 Hz, J = 9.8 Hz, 2H), 4.0-3.95 | | | | |
| | | (m, 8H) | | | | |
| | | LRMS: (M + 1) 519.57 (calc) (M + 1) 519.1 | | | | |
| | | (found) | | | | |
| 11 | 23 | 1H NMR (400 MHz, DMSO-d6) δ (ppm): | A | b | e | e |
| | | 10.42 (s, 1H), 8.54 (d, J = 6.7 Hz, 2H), 8.49 | | | | |
| | | (d, J = 5.3 Hz, 1H), 7.86 (dd, J = 12.3, 2.3 | | | | |
| | | Hz, 1H), 7.65 (d, J = 6.5 Hz, 2H), 7.53 (s, | | | | |
| | | 1H), 7.51-7.43 (m, 2H), 7.41 (s, 1H), 6.47 (d, | | | | |
| | | J = 4.5 Hz, 1H), 3.99-3.93 (m, 10H). | | | | |
| | | LRMS: 503.48 (calc) 504.2 (found) | | | | |
| 12 | 24 | 1H NMR (400 MHz, DMSO-d6) δ (ppm): | A | a | b | a |
| | | 10.54 (s, 1H), 8.48 (d, J = 5.3 Hz, 1H), 7.85 | | | | |
| | | (d, J = 12.1 Hz, 1H), 7.70-7.61 (m, 2H), 7.53 | | | | |
| | | (s, 1H), 7.48-7.42 (m, 2H), 7.41 (s, 1H), 7.29 | | | | |
| | | (t, J = 8.8 Hz, 2H), 6.47 (d, J = 5.3 Hz, 1H), | | | | |
| | | 4.01-3.90 (m, 10H). | | | | |
| | | LRMS: 520.48 (calc) 521.2 (found) | | | | |
| 13 | 25 | 1H NMR (400 MHz, DMSO-d6) δ (ppm): | A | b | d | b |
| | | 10.55 (s, 1H), 8.48 (d, J = 5.4 Hz, 1H), 7.8 (dd, | | | | |
| | | J = 2.5Hz, 13.1 Hz, 1H), 7.53 (s, 1H), 7.45- | | | | |
| | | 7.35 (m, 3H), 6.45 (d, J = 5.3 Hz, 1H), 3.95 (s, | | | | |
| | | 6H), 3.76 (dd, J = 7.8 Hz, 8.4 Hz, 2H), 3.54 (dd, | | | | |
| | | J = 8.4 Hz, 7.8 Hz, 2H), 3.34-2.87 (m, 4H), | | | | |
| | | 1.61-1.57 (m, 4H), 1.35 (m, 2H) | | | | |
| | | LRMS: (M + 1) 510.41 (calc) (M + 1) 510.3 | | | | |
| | | (found) | | | | |
| 14 | 26 | 1H NMR (400 MHz, DMSO-d6) δ (ppm): | A | d | d | d |
| | | 10.43 (s, 1H), 8.57 (d, J = 5.1 Hz, 1H), 8.45 | | | | |
| | | (d, J = 2.9 Hz, 1H), 8.22 (dd, J = 8.8, 2.7 Hz, | | | | |
| | | 1H), 7.63 (d, J = 7.8 Hz, 2H), 7.43 (t, J = 7.8 | | | | |
| | | Hz, 2H), 7.42 (s, 1H), 7.37 (s, 1H), 7.33 (d, J = | | | | |
| | | 8.8 Hz, 1H), 7.17 (t, J = 7.3 Hz, 1H), 6.86 | | | | |
| | | (d, J = 5.1 Hz, 1H), 4.01-3.86 (m, 10H). | | | | |
| | | LRMS: 485.49 (calc) 486.3 (found) | | | | |
| 15 | 27 | 1H NMR (400 MHz, DMSO-d6) δ (ppm): | A | a | d | d |
| | | 10.6 (s, 1H), 8.48 (d, J = 5.1 Hz, 1H), 7.79 (dd, | | | | |
| | | J = 12.9 Hz, J = 2.2 Hz, 1H), 7.52 (s, 1H), 7.42 (s, | | | | |
| | | J = 9.8 Hz, J = 8.8 Hz, 1H), 7.41 (s, 1H), 7.35 (dd, | | | | |
| | | J = 8.8 Hz, 2.2 Hz, 1H), 6.45 (d, J = 5.1 Hz, 1H), | | | | |
| | | 3.94 (s, 6H), 3.8 (m, 2H), 3.68-3.58 (m, 1H), | | | | |
| | | 3.44 (m, 2H), 1.79-1.54 (m, 5H), 1.5-1.22 (m, | | | | |
| | | 4H), 1.5-1.05 (m, 1H) | | | | |
| | | LRMS: (M + 1) 509.55 (calc) (M + 1) 509.2 | | | | |
| | | (found) | | | | |
| 16 | 28 | 1H NMR (400 MHz, DMSO-d6) δ (ppm): | A | a | d | d |
| | | 10.49 (s, 1H), 8.48 (d, J = 5.3 Hz, 1H), 7.86 | | | | |
| | | (dd, J = 13.0, 2.1 Hz, 1H), 7.61 (dt, J = 11.9, | | | | |
| | | 2.2 Hz, 1H), 7.53 (s, 1H), 7.51-7.39 (m, 5H), | | | | |
| | | 7.02 (td, J = 8.2, 2.3 Hz, 1H), 6.47 (d, J = 5.1 | | | | |
| | | Hz, 1H), 4.02-3.91 (m, 10H). | | | | |
| | | LRMS: 520.48 (calc) 521.2 (found) | | | | |
| 17 | 29 | 1H NMR (400 MHz, DMSO-d6) δ (ppm): | A | a | d | d |
| | | 10.50 (s, 1H), 8.49 (d, J = 5.3 Hz, 1H), 7.86 | | | | |
| | | (dd, J = 12.5, 2.3 Hz, 1H), 7.61 (dt, J = 11.9, | | | | |
| | | 2.2 Hz, 1H), 7.53 (s, 1H), 7.51-7.41 (m, 5H), | | | | |
| | | 7.05-6.98 (m, 1H), 6.47 (dd, J = 5.2, 1.1 Hz, | | | | |
| | | 1H), 4.02-3.91 (m, 10H). | | | | |
| | | LRMS: 520.48 (calc) 521.1 (found) | | | | |
| 18 | 30 | 1H NMR (400 MHz, DMSO-d6) δ (ppm): | A | a | d | b |
| | | 10.41 (s, 1H); 8.46 (d, J = 5.3, 1H); 7.67-7.61 | | | | |
| | | (m, 4H); 7.49 (s, 1H); 7.38 (s, 1H); 7.29-7.22 | | | | |
| | | (m, 4H); 6.45 (d, J = 5.1, 1H)3.95-3.90 (m, | | | | |
| | | 4H); 3.93 (s, 3H); 391 (s, 3H). | | | | |
| | | LRMS: 503.5 (calc) 503.2 (found) | | | | |
| 19 | 31 | 1H NMR (400 MHz, DMSO-d6) δ (ppm): | A | a | b | b |
| | | 10.99 (s, 1H), 8.50 (d, J = 5.3 Hz, 1H), 8.36 | | | | |
| | | (dd, J = 2.9, 0.6 Hz, 1H), 8.16(dd, J = 9.1, | | | | |
| | | 0.7 Hz, 1H), 7.85 (dd, J = 9.0, 2.9 Hz, 1H), | | | | |
| | | 7.67-7.61 (m, 2H), 7.54 (s, 1H), 7.47-7.41 | | | | |
| | | (m, 2H), 7.41 (s ,1H), 7.19 (t, J = 7.4 Hz, | | | | |
| | | 1H), 6.54 (d, J = 5.1 Hz, 1H), 4.01-3.93 (m, | | | | |
| | | 10H). | | | | |
| | | LRMS(ESI): (calc.) 485.49 (found) 486.2 | | | | |
| | | (MH)+ | | | | |
| 20 | 32 | 1H NMR (400 MHz, DMSO-d6) δ (ppm): | A | d | d | e |
| | | 10.90 (s, 1H), 8.49 (d, J = 5.3 Hz, 1H), 8.36 | | | | |
| | | (s, 1H), 7.96 (dd, J = 12.8, 2.4 Hz, 1H), 7.66- | | | | |
| | | 7.53 (m, 7H), 7.50 (t, J = 8.9 Hz, 1H), 7.42 | | | | |
| | | (s, 1H), 6.49 (dd, J = 5.2, 1.1 Hz, 1H), 3.96 | | | | |
| | | and 3.95 (2s, 2x3H). | | | | |
| | | LRMS: 552.48 (calc) 553.1 (found) | | | | |
| 21 | 33 | 1H NMR (400 MHz, DMSO-d6) δ (ppm): | C | d | e | e |
| | | 11.17 (s, 1H), 8.49 (d, J = 5.1 Hz, 1H), 8.04 | | | | |
| | | (dd, J = 12.9, 2.3 Hz, 1H), 8.02-7.97 (m, 2H), | | | | |
| | | 7.82-7.76 (m, 1H), 7.63-7.48 (m, 6H), 7.42 | | | | |
| | | (s, 1H), 6.49 (dd, J = 5.3, 1.0 Hz, 1H), 3.96 | | | | |
| | | (s, 6H). | | | | |
| | | LRMS(ESI): (calc) 485.49 (found) 486.2 | | | | |
| | | (MH)+ | | | | |
| 22 | 39 | 1H NMR (400 MHz, DMSO-d6) δ (ppm): | d | d | d | d |
| | | 10.53 (s, 1H), 8.82 (s, 1H), 7.82 (d, J = 13.3 | | | | |
| | | Hz, 1H), 7.62-7.60 (m, 2H), 7.52 (s, 1H), | | | | |
| | | 7.47 (s, 1H), 7.41 (t, J = 7.3 Hz, 2H), 7.35- | | | | |
| | | 7.27 (m, 2H), 7.16 (t, J = 7.2 Hz, 1H), 3.99 | | | | |
| | | (s, 3H), 3.49 (s, 3H), 3.90 (s, 4H). MS (m/z): | | | | |
| | | 528.2 (M + H). | | | | |
| 23 | 47 | 1H NMR (400 MHz, DMSO-d6) δ (ppm): | a | a | d | d |
| | | 10.58 (s, 1H), 8.48 (d, J = 5.3 Hz, 1H), 7.82 | | | | |
| | | (dd, J = 13.0, 2.2 Hz, 1H), 7.53 (s, 1H), 7.47- | | | | |
| | | 7.37 (m, 5H), 7.17 (dd, J = 8.7, 1.3 Hz, 1H), | | | | |
| | | 7.03 (td, J = 7.6, 1.4 Hz, 1H), 6.46 (dd, J = | | | | |
| | | 5.2, 1.1 Hz, 1H), 4.02-3.93 (m, 8H), 3.85- | | | | |
| | | 3.78 (m, 5H). | | | | |
| | | MS (m/z): 533.3 (M + H). | | | | |
| 24 | 48 | 1H NMR (400 MHz, DMSO-d6) δ (ppm): | a | a | d | e |
| | | 10.49 (s, 1H), 8.48 (d, J = 5.3 Hz, 1H), 8.42 | | | | |
| | | (ddd, J = 4.9, 1.9, 0.9 Hz, 1H), 8.18 (dt, J = | | | | |
| | | 8.4, 0.9 Hz, 1H), 7.91-7.82 (m, 2H), 7.54 (s, | | | | |
| | | 1H), 7.51-7.42 (m, 2H), 7.41 (s, 1H), 7.19 | | | | |
| | | (ddd, J = 7.2, 4.9, 1.0 Hz, 1H), 6.47 (dd, J = | | | | |
| | | 5.2, 1.1 Hz, 1H), 4.12-4.04 (m, 2H), 3.99- | | | | |
| | | 3.89 (m, 8H). | | | | |
| | | MS (m/z): 504.3 (M + H). | | | | |
| 25 | | ¹H NMR (400 MHz, DMSO-d₆) δ (ppm): | a | c | d | d |
| | | 10.72 (d, J = 2.5 Hz, 1H), 8.50 (d, J = 5.1 Hz, | | | | |
| | | 1H), 8.27 (t, J = 9.1 Hz, 1H), 7.64-7.60 (m, | | | | |
| | | 2H), 7.49 (s, 1H), 7.47-7.41 (m, 3H), 7.41 (s, | | | | |
| | | 1H), 7.22-7.12 (m, 2H), 6.57 (d, J = 5.3 Hz, | | | | |
| | | 1H), 4.02-3.91 (m, 10H). | | | | |
| | | MS (m/z): 503.3 (M + H). | | | | |
| 26 | 55 | ¹H NMR (400 MHz, DMSO-d₆) δ (ppm): | a | d | d | d |
| | | 10.53 (s, 1H), 8.55 (s, 1H), 8.23 (d, J = 5.3 | | | | |
| | | Hz, 1H), 7.75 (dd, J = 12.9, 2.0 Hz, 1H), 7.70 | | | | |
| | | (s, 1H), 7.67-7.61 (m, 2H), 7.47-7.35 (m, | | | | |
| | | 4H), 7.24 (s, 1H), 7.18 (tt, J = 7.4, 1.0 Hz, | | | | |
| | | 1H), 6.27-6.21 (m, 1H), 4.02-3.92 (m, 4H), | | | | |
| | | 3.93 (s, 3H), 3.90 (s, 3H). | | | | |
| | | MS (m/z): 502.3 (M + H). | | | | |
| 27 | 57 | ¹H NMR (400 MHz, DMSO-d₆) δ (ppm): | b | d | d | d |
| | | 10.52 (s, 1H), 8.62 (d, J = 5.9 Hz, 1H), 8.13 | | | | |
| | | (s, 0.3H, formate), 7.70 (dd, J = 13.3, 2.1 Hz, | | | | |
| | | 1H), 7.62 (d, J = 7.6 Hz, 2H), 7.42 (t, J = 8.0 | | | | |
| | | Hz, 2H), 7.38-7.25 (m, 3H), 7.21-7.13 (m, | | | | |
| | | 2H), 6.70 (s, 1H), 4.00-3.88 (m, 4H), 3.90 (s, | | | | |
| | | 3H), 3.47 (s, 3H), 3.39 (s, 3H). | | | | |
| | | MS (m/z): 516.3 (M + H). | | | | |
| 28 | 59 | ¹H NMR (400 MHz, DMSO-d₆) δ (ppm): | c | a | d | d |
| | | 10.35 (s, 1H), 9.26 (br, 1H), 8.47 (d, J = 5.2 | | | | |
| | | Hz, 1H), 8.42 (s, 1H), 7.94 (d, J = 7.2 Hz, | | | | |
| | | 2H), 7.76 (d, J = 13.7 Hz, 1H), 7.62-7.58 (m, | | | | |
| | | 1H), 7.54-7.5 (m, 3H), 7.42-7.37 (m, 3H), | | | | |
| | | 6.45 (d, J = 5.2 Hz, 1H), 3.95 (s, 6H). | | | | |
| | | MS (m/z): 477.2 (M + H). | | | | |
| 29 | 63 | ¹H NMR (400 MHz, DMSO-d₆) δ (ppm): | d | b | d | d |
| | | 8.26 (d, J = 5.3 Hz, 1H), 7.60 (dd, J = 12.1, | | | | |
| | | 2.3 Hz, 1H), 7.50-7.46 (m, 3H), 7.42-7.3 (m, | | | | |
| | | 5H), 7.1 (t, J = 7.4 Hz, 1H), 6.25 (dd, J = 4.5, | | | | |
| | | 0.8 Hz, 1H), 3.94 (s, 3H), 3.91 (s, 3H), 3.86 | | | | |
| | | (br, 4H), 3.39 (s, 3H) | | | | |
| | | MS (m/z): 517.3 (M + H). | | | | |
| 30 | 67 | ¹H NMR (400 MHz, DMSO-d₆) δ (ppm): | a | c | e | e |
| | | 10.59 (s, 1H), 8.47 (d, J = 5.3 Hz, 1H), 7.80 | | | | |
| | | (dd, J = 2.2 Hz, J = 12.9 Hz, 1H), 7.53 (s, 1H), | | | | |
| | | 7.44-7.35 (m, 3H), 6.45 (d, J = 5.0 Hz, 1H), | | | | |
| | | 5.86-5.76 (m, 1H), 5.30-5.21 (m, 2H), 3.95 | | | | |
| | | (s, 6H), 3.87-3.81 (m, 4H), 3.45-3.41 (m, | | | | |
| | | 2H) | | | | |
| | | MS (m/z): 467.2 (M + H). | | | | |
| 31 | 70 | ¹H NMR (400 MHz, DMSO-d₆) δ (ppm): | d | d | e | e |
| | | 10.02 (s, 1H), 8.47 (m, 2H), 7.79 (dd, J = | | | | |
| | | 11.7, 2.1 Hz, 1H), 7.51-7.39 (m, 3H), 6.45 | | | | |
| | | (dd, J = 5.3, 0.8 Hz, 1H), 4.45 (dd, J = 8.4, | | | | |
| | | 7.8 Hz, 2H), 3.99 (dd, J = 8.4, 7.8 Hz, 2H), | | | | |
| | | 3.93 (s, 6H) | | | | |
| | | MS (m/z): 428.2 (M + H). | | | | |
| 32 | 74 | ¹H NMR (400 MHz, DMSO-d₆) δ (ppm): | d | d | — | — |
| | | 8.46 (d, J = 5.2 Hz, 1H), 7.82-7.80 (m, 2H), | | | | |
| | | 7.51-7.45 (m, 6H), 7.39 (s, 1H), 7.22-7.20 | | | | |
| | | (m, 2H), 6.46 (d, J = 5.2 Hz, 1H), 4.09 (s, | | | | |
| | | 2H), 3.97-3.91 (m, 2H), 3.94 (s, 3H), 3.91 (s, | | | | |
| | | 3H), 3.34-3.29 (m, 2H) | | | | |
| | | MS (m/z): 546.2 (M + H). | | | | |
| 33 | 76 | ¹H NMR (400 MHz, DMSO-d₆) δ (ppm): | — | d | — | e |
| | | 12.54 (br, 1H), 8.48 (d, J = 5.3 Hz, 1H), 7.82 | | | | |
| | | (dd, J = 12.7, 2.5 Hz, 1H), 7.53-7.36 (m, 9H), | | | | |
| | | 6.49-6.48 (m, 1H), 4.26-4.22 (m, 2H), 4.13- | | | | |
| | | 4.09 (m, 2H) | | | | |
| | | MS (m/z): 519.2 (M + H). | | | | |
| 34 | 82 | ¹H NMR (400 MHz, DMSO-d₆) δ (ppm): | b | a | d | d |
| | | 10.6 (s, 1H), 8.46 (d, J = 5.3 Hz, 1H), 7.84 | | | | |
| | | (m, 1H), 7.61-7.63 (m, 2H), 7.51 (s, 1H), | | | | |
| | | 7.38-7.46 (m, 5H), 7.17 (m, 1H), 6.44 (dd, J = | | | | |
| | | 1.0, 5.3 Hz, 1H), 3.90-4.03 (m, 11H), 2.77 | | | | |
| | | (bs, 2H), 2.03 (bs, 1H), 1.77 (bd, J = 12.3 Hz, | | | | |
| | | 2H), 1.39 (s, 9H), 1.20 (m, 2H). | | | | |
| | | MS (m/z): 686.0 (M + H). | | | | |
| 35 | 83 | ¹H NMR (400 MHz, DMSO-d₆) δ (ppm): | a | a | b | b |
| | | 10.6 (s, 1H), 8.70 (d, J = 6.0 Hz, 1H), 8.61 | | | | |
| | | (bd, J = 10.6 Hz, 1H), 8.30 (bm, 1H), 7.90 | | | | |
| | | (m, 1H), 7.63-7.68 (m, 2H), 7.56-7.62 (m, | | | | |
| | | 2H), 7.50-7.52 (m, 2H), 7.40-7.44 (m, 2H), | | | | |
| | | 7.15-7.19 (m, 1H), 6.79 (d, J = 5.7 Hz, 1H), | | | | |
| | | 4.11 (d, J = 6.3 Hz, 2H), 4.00 (s, 3H), 3.91- | | | | |
| | | 3.97 (m, 4H), 3.34 (m, 2H), 2.95 (m, 2H), | | | | |
| | | 2.20 (bs, 1H), 1.97 (bd, J = 12.7 Hz, 2H), 1.52 | | | | |
| | | (m, 2H). | | | | |
| | | MS (m/z): 586.0 (M + H). | | | | |
| 36 | 87 | ¹H NMR (400 MHz, DMSO-d₆) δ (ppm): | a | a | b | b |
| | | 8.43 (bs, 2H), 7.81 (m, 1H), 7.63 (m, 3H), | | | | |
| | | 7.40 (dd, J = 7.4, 8.6 Hz, 2H), 7.34 (m, 3H), | | | | |
| | | 7.18 (dd, J = 7.4, 7.4 Hz, 1H), 6.51 (d, J = 4.7 | | | | |
| | | Hz, 1H), 4.11 (d, J = 5.7 Hz, 2H), 4.00 (s, | | | | |
| | | 7H), 3.48 (d, J = 11.9 Hz, 2H), 2.95 (dd, J = | | | | |
| | | 11.7, 11.7 Hz, 2H), 2.80 (s, 3H), 2.15-2.28 | | | | |
| | | (bm, 3H), 1.76 (bm, 2H). | | | | |
| | | MS (m/z): 600.0 (M + H). | | | | |
| 37 | 90 | ¹H NMR (400 MHz, DMSO-d₆) δ (ppm): | a | a | a | a |
| | | 8.46 (d, J = 5.3 Hz, 1H), 7.83 (m, 1H), 7.62 | | | | |
| | | (m, 2H), 7.51 (s, 1H), 7.41 (m, 5H), 7.17 (m, | | | | |
| | | 1H), 6.44 (dd, J = 1.1, 5.2 Hz, 1H), 4.19 (t, J = | | | | |
| | | 6.4 Hz, 2H), 3.95 (m, 7H), 3.57 (m, 4H), | | | | |
| | | 2.45 (a t almost overlapped by DMSO | | | | |
| | | signal), 2.38 (bs, 4H), 1.97 (m, 2H). | | | | |
| | | MS (m/z): 616 (M + H). | | | | |
| 38 | 91 | ¹H NMR (400 MHz, DMSO-d₆) δ (ppm): | C | b | d | d |
| | | 10.47 (s, 1H), 8.45 (d, J = 5.3 Hz, 1H), 7.81 | | | | |
| | | (dd, J = 12.8, 2.4 Hz, 1H), 7.51 (s, 1H), 7.45- | | | | |
| | | 7.35 (m, 3H), 7.11 (t, J = 8.0 Hz, 2H), 6.75 | | | | |
| | | (d, J = 8.0 Hz, 2H), 6.62 (t, J = 7.2 Hz, 1H), | | | | |
| | | 6.42 (d, J = 4.8 Hz, 1H), 6.12 (d, J = 9.2 Hz, | | | | |
| | | 1H), 4.76-4.65 (m, 1H), 3.94 (s, 3H), 3.94 (s, | | | | |
| | | 3H), 2.93 (dd, J = 16.0, 4.0 Hz, 1H), 2.78 | | | | |
| | | (dd, J = 16.0, 9.6 Hz, 1H). MS (m/z): 530.2 | | | | |
| | | (M + H). | | | | |
| 39 | 95 | ¹H NMR (400 MHz, DMSO-d₆) δ (ppm): | C | a | d | d |
| | | 10.11 (s, 1H), 8.44 (d, J = 5.2 Hz, 1H), 7.62- | | | | |
| | | 7.54 (m, 2H), 7.50 (s, 1H), 7.38 (s, 1H), 7.31 | | | | |
| | | (t, J = 8.0 Hz, 2H), 7.18 (t, J = 9.2 Hz, 1H), | | | | |
| | | 7.05 (t, J = 7.6 Hz, 1H), 6.86 (dd, J = 13.6, | | | | |
| | | 2.8 Hz, 1H), 6.68 (dd, J = 9.2, 2.4 Hz, 1H), | | | | |
| | | 6.54 (d, J = 9.6 Hz, 1H), 6.36 (d, J = 5.2 Hz, | | | | |
| | | 1H), 4.85-4.73 (m, 1H), 3.94 (s, 6H), 2.92 | | | | |
| | | (dd, J = 15.6, 3.6 Hz, 1H), 2.76 (dd, J = 15.6, | | | | |
| | | 9.6 Hz, 1H). MS (m/z): 530.2 (M + H). | | | | |
| 40 | 97 | ¹H NMR (400 MHz, DMSO-d₆) δ (ppm): | a | a | a | a |
| | | 10.54 (s, 1H), 8.41 (d, J = 4.9, 1H), 7.73-7.70 | | | | |
| | | (m, 2H), 7.65-7.60 (m, 2H), 7.59-7.56 (m, | | | | |
| | | 2H), 7.37 (s, 1H), 7.30 (s, 1H). | | | | |
| | | MS (m/z): 519.2 (M + H). | | | | |
| 41 | 100 | ¹H NMR (400 MHz, DMSO-d₆) δ (ppm): | a | a | a | b |
| | | 10.44 (s, 1H), 8.46 (d, J = 5.3 Hz, 1H), 7.69- | | | | |
| | | 7.64 (m, 2H), 7.63-7.60 (m, 2H), 7.50 (s, | | | | |
| | | 1H), 7.43-7.39 (m, 2H), 7.38 (s, 1H), 7.27- | | | | |
| | | 7.22 (m, 2H), 7.18-7.13 (m, 1H), 6.45 (d, J = | | | | |
| | | 5.1 Hz, 1H), 3.96-3.93 (m, 4H), 3.93 (s, 3H), | | | | |
| | | 3.92 (s, 3H). | | | | |
| | | MS (m/z): 485.3 (M + H). | | | | |
| 42 | 109 | ¹H NMR (400 MHz, DMSO-d₆) δ (ppm): | a | a | a | a |
| | | 10.55 (s, 1H), 8.46 (d, J = 5.1 Hz, 1H), 7.85- | | | | |
| | | 7.81 (m, 1H), 7.63-7.60 (m, 2H), 7.55 (s, | | | | |
| | | 1H), 7.44 (s, 1H), 7.42-7.39 (m, 4H), 7.19- | | | | |
| | | 7.15 (s, 1H), 6.45 (d, J = 5.2 Hz, 1H), 4.29- | | | | |
| | | 4.25 (m, 4H), 3.97-3.93 (m, 4H), 3.76-3.72 | | | | |
| | | (m, 4H), 3.35 (s, 3H), 3.33 (s, 3H). | | | | |
| | | MS (m/z): 591.3 (M + H). | | | | |
| 43 | 111 | ¹H NMR (400 MHz, DMSO-d₆) δ (ppm): | a | a | d | B |
| | | 12.29 (s, 1H), 8.48 (d, J = 5.3 Hz, 1H), 8.01 | | | | |
| | | (d, J = 11.8, 1.2 Hz, 1H), 7.65-7.62 (m, 2H), | | | | |
| | | 7.52-7.54 (m, 3H), 7.39 (s, 1H), 7.30-7.26 | | | | |
| | | (m, 2H), 6.48 (dd, J = 5.1, 0.8 Hz, 1H), 4.24- | | | | |
| | | 4.16 (m, 4H), 3.97-3.94 (m, 5H), 3.54 (t, J = | | | | |
| | | 4.5 Hz, 2H), 2.44 (t, J = 7.0 Hz, 3H), 2.36 (s, | | | | |
| | | br, 3H), 1.95 (m, 2H). | | | | |
| | | MS (m/z): 650.3 (M + H). | | | | |

In Vivo Solid Tumor Disease Model

This test measures the capacity of compounds to inhibit solid tumor growth.

Tumor xenografts are established in the flank of female athymic CDl mice (Charles River Inc.), by subcutaneous injection of 1X10⁶ cells/mouse. Once established, tumors are then serially passaged s.c. in nude mice hosts. Tumor fragments from these host animals are used in subsequent compound evaluation experiments. For compound evaluation experiments female nude mice weighing approximately 20 g are implanted s.c. by surgical implantation with tumor fragments of 30 mg from donor tumors. When the tumors are approximately 100 mm³ in size (˜7-10 days following implantation), the animals are randomized and separated into treatment and control groups. Each group contains 6-8 tumor-bearing mice, each of which is ear-tagged and followed individually throughout the experiment.

Mice are weighed and tumor measurements are taken by calipers three times weekly, starting on Day 1. These tumor measurements are converted to tumor volume by the well-known formula (L+W/4)³ 4/3π. The experiment is terminated when the control tumors reach a size of approximately 1500 mm³. In this model, the change in mean tumor volume for a compound treated group/the change in mean tumor volume of the control group (non-treated or vehicle treated)×100 (T/C) is subtracted from 100 to give the percent tumor growth inhibition (% TGI) for each test compound. In addition to tumor volumes, body weight of animals is monitored twice weekly for up to 3 weeks.

Compound 9 (Example 2) was evaluated in vivo in a MNNGHOS tumor xenograft model in mice. The compound was dosed orally at 40 mg/kg in a mixture PEG 400/0.1 N HCl in saline (40:60). The compound caused full regression of the tumors (112% tumor growth inhibition).

1. A compound of the formula (I) and racemic mixtures, diastereomers and enantiomers thereof: 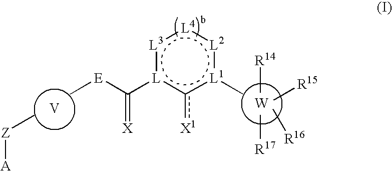
and N-oxides, hydrates, solvates, pharmaceutically acceptable salts, prodrugs and complexes thereof, wherein, 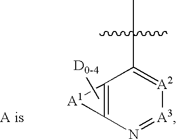
wherein
A¹ represents a fused 6-membered aryl or heteroaryl group;
A2 and A³ are independently selected from N and CR¹⁰⁷;
R¹⁰⁷ is selected from the group consisting of hydrogen, halogen, C₁-C₁₂alkyl, C₂-C₁₂alkenyl, C₂-C₁₂alkynyl, C₃-C₁₂cycloalkyl, C₆-C₁₂aryl, 3-12 membered heteroalicyclic, 5-12 membered heteroaryl, —S(O)R¹⁰⁸—SO₂NR¹⁰⁸R¹⁰⁹, —S(O)₂OR¹⁰⁸, —NO₂, —NR¹⁰⁸R¹⁰⁹, —(CR¹¹⁰R¹¹¹)₀₋₄OR¹⁰⁸—CN, —C(O)R¹⁰⁸, —OC(O)R¹⁰⁸, —O(CR110R111)₀₋₄R^{10s}, —NR¹⁰⁸C(O)R¹⁰⁹, —(CR¹¹⁰R111)₀₋₄C(O)OR¹⁰⁸, —(CR¹¹⁰R¹¹¹)₀₋₄NR¹⁰⁸R¹⁰⁹, —C(═NR¹¹⁰)NR¹⁰⁸R¹⁰⁹, —NR¹⁰⁸C(O)NR¹⁰⁹R¹¹⁰, —NR¹⁰⁸S(O) ₁₋₂R¹⁰⁹ and —C(O)NR¹⁰⁸R¹⁰⁹, wherein each hydrogen of which is optionally substituted by an R¹¹⁷ group;
each R¹⁰⁸, R¹⁰⁹, R¹¹⁰ and R¹¹¹, which may be the same or different, is independently selected from hydrogen, halogen, C₁-C₁₂alkyl, C₂-C₁₂alkenyl, C₂-C₁₂alkynyl, C₃-C₁₂cycloalkyl, C₆-C12aryl, 3-12 membered heteroalicyclic and 5-12 membered heteroaryl, or any two of R¹⁰⁸, R¹⁰⁹, R¹¹⁰ and R¹¹¹ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from N, O and S, or any two of R¹⁰⁸, R¹⁰⁹, R¹¹⁰ and R¹¹¹ . . . bound to the same carbon atom may be combined to form a C₃-C₁₂ cycloalkyl, C₆-C₁₂ aryl, 3-12 membered heteroalicyclic or 5-12 membered heteroaryl group, and each hydrogen of R¹⁰⁸, R¹⁰⁹, R¹¹⁰ and R¹¹¹ is optionally substituted by from 1 to 6 R¹¹⁷ groups;
each R¹¹⁷, which may be the same or different, is independently selected from halogen, C₁-C₁₂ alkyl, C₂-C₁₂ alkenyl, C₂-C₁₂ alkynyl, C₃-C₁₂ cycloalkyl, C₆-C₁₂ aryl, 3-12 membered heteroalicyclic, 5-12 membered heteroaryl, —CN, —O—C₁-C₁₂ alkyl, —O—(CH₂)₀₋₄C₃-C₁₂ cycloalkyl, —O—(CH₂)₀₋₄C₆-C₁₂ aryl, —O—(CH₂)₀₋₄(3-12 membered heteroalicyclic) and —O—(CH₂)₀₋₄(5 to 12 membered heteroaryl), —C(O)R¹⁹, —C(O)OR¹⁹ and —C(O)NR¹¹⁹R¹²⁰, and each hydrogen in R¹¹⁷ is optionally substituted by an R¹¹⁸ group;
each R¹¹⁸, which may be the same or different, is independently selected from hydrogen, halogen, C₁-C₁₂ alkyl, C₁-C₁₂ alkoxy, C₃-C₁₂ cycloalkyl, C₆-C₁₂ aryl, 3-12 membered heteroalicyclic, 5-12 membered heteroaryl, —O—C₁-C₁₂ alkyl, —O—(CH₂)₀₋₄C₃-C₁₂ cycloalkyl, —O—(CH₂)₀₋₄C₆-C₁₂ aryl, —O—(CH₂)₀₋₄(3-12 membered heteroalicyclic), —O—(CH₂)ₒ₄(5-12 membered heteroaryl) and —CN, and each hydrogen in R¹¹⁸ is optionally substituted by a group selected from halogen, —OH, —CN, —C₁-C₁₂alkyl which may be partially or fully halogenated, —O—C₁-C₁₂ alkyl which may be partially or fully halogenated, —CO, —SO, —SO₂ and —SO₃H;
each R¹⁹ and R¹²⁰, which may be the same or different, is independently selected from hydrogen, halogen, C₁-C₁₂ alkyl, C₁-C₁₂ alkoxy, C₃-C₁₂ cycloalkyl, C₆-C₁₂ aryl, 3-12 membered heteroalicyclic and 5-12 membered heteroaryl, and each R¹⁹ and R¹²⁰ is optionally substituted by a group selected from halogen, —OH, —CN, —C₁-C₁₂ alkyl which may be partially or fully halogenated, —O—C₁-C₁₂ alkyl which may be partially or fully halogenated and SO₃H, or R¹¹⁹ and R¹²⁰, taken together with the nitrogen atom to which they are attached, may form a 3-12 membered heteroalicyclic ring optionally substituted by from 1 to 6 R¹¹⁸ groups;
each D is independently selected from the group consisting of R²⁵⁹, R⁰⁷⁷, R⁷, R¹ and R²¹, wherein
R²⁵⁹ is selected from the group consisting of H, halogen, cyano, nitro, C₁-C₃alkylsulphanyl, —N(OH)H, —N(OH)C₁-C₃alkyl, trifluoromethyl, C₁₋₆alkyl, —NR^{b}R^{c} (wherein R^{b} and R^{c}, which may be the same or different, each represent hydrogen or C₁₋₆alkyl), and —X²R^{d};
wherein X² is selected from the group consisting of a direct bond, —O—, —CH₂—, —OC(O)—, carbonyl, —S—, —SO—, —SO₂—, —NR^{e}C(O)—, —C(O)NR^{f}—, —SO₂NR^{g}—, NR^{h}SO₂— and —NRⁱ—;
wherein R^{e}, R^{f}, R^{g}, R^{h} and Rⁱ are independently selected from the group consisting of H, C₁₋₆alkyl, hydroxyC₁₋₄alkyl, and C₁₋₃alkoxyC₂₋₃alkyl; and
R^{d} is selected from the group consisting of H, optionally substituted hydrocarbyl, optionally substituted heterocyclyl and optionally substituted alkoxy;
R⁰⁷⁷ is —O-M⁴-M³-M²-Ml, wherein
M¹ is H, C₁-C₈alkyl-L 202-L 2- optionally substituted by Y², G²⁰⁰(CH₂)₀₋₃—, and R²⁵³(R²⁵⁴)N(CH₂)₀₋₃—, wherein
G²⁰⁰ is a saturated five- to seven-membered heterocyclyl or heteroaryl containing one or two annular heteroatoms and optionally substituted with between one and three Y² substitutents;
L²¹¹ is —C(O)— or —SO₂—;
L²⁰² is a direct bond, —O— or —NH—;
R²⁵³ and R²⁵⁴ are independently C₁-C₃alkyl optionally substituted with between one and three Y² substituents;
M² is a saturated or mono- or poly-unsaturated C₃-C₁₄ mono- or fused-polycyclic hydrocarbyl optionally containing one or two or three annular heteroatoms per ring and optionally substituted with between zero and four Y² substituents;
M³ is —NH—, —N(optionally substituted lower alkyl)-, —O—, or absent;
M⁴ is —CH₂—, —CH₂—CH₂—, —CH₂CH₂CH₂—, or absent;
R⁷ is selected from the group consisting of —H, halogen, nitro, azido, C₁-C₆ alkyl, trifluoromethyl, C₃-C₁₀ cycloalkyl, (C₁-C₆)alkoxy, —C(O)NR⁴²R⁴³, —Y—NR⁴²R⁴³, —NR⁴²C(═O)R¹³, —SO₂R⁴², —SO₂NR¹²R⁴³, —NR³⁷SO₂R⁴², —NR³⁷SO₂NR⁴²R⁴³, —C(═N—OR⁴²)R⁴³, —C(═NR⁴²)R⁴³, —NR³⁷C(═NR⁴²)R⁴³, —C(═NR⁴²)NR¹⁷R⁴³, —NR³⁷C(═NR⁴²)NR³⁷R⁴³, —C(O)R⁴², —CO₂R⁴², —C(O)(heterocyclyl), —C(O)(C₆-C₁₀ aryl), —C(O)(heteroaryl), —Y—(C₆-C₁₀ aryl), —Y-(heteroaryl), —Y-(5-10 membered heterocyclyl), —NR^{6a}R^{6b}, —NR^{6a}SO₂R^{6b}, NR^{6a}C(O)R^{6b}, —OC(O)R^{6b}, NR^{6a}C(O)OR^{6b}, OC(O)NR^{6a}R^{6b} —OR^{6a}, SR^{6a}, S(O)R^{6a}, SO₂R^{6a}, SO₃R^{6a}, SO₂NR^{6a}R^{6b}, —SO₂NR⁴²R⁴³, —COR^{6a}, —CO₂R^{6a}, —CONR^{6a}R^{6b}, —(C₁-C₄)fluoroalkyl, —(C₁-C₄)fluoroalkoxy, —(CZ³Z⁴)ₐCN, wherein a is an integer ranging from 0 to 6, and the aforementioned R⁷ groups other than —H and halogen are optionally substituted, or R⁷ is a moiety selected from the group consisting of —(CZ³Z⁴)ₐ-aryl, —(CZ³Z⁴)ₐ-heterocycle, (C₂-C₆)alkynyl, —(CZ³Z⁴)ₐ-(C₃-C₆)cycloalkyl, —(CZ³Z⁴)ₐ-(C₅-C₆)cycloalkenyl, (C₂-C₆) alkenyl and (C₁-C₆)alkyl, wherein said moiety is optionally substituted with 1 to 3 independently selected Y² groups, where a is 0, 1, 2, or 3, and wherein when a is 2 or 3, the CZ³Z⁴ units may be the same or different; wherein
each R⁴² and R⁴³ is independently selected from the group consisting of H, C₁-C₆ alkyl, —Y⁴—(C₃-C₁₀ cycloalkyl), —Y⁴—(C₆-C₁₀ aryl), —Y⁴—(C₆-C₁₀ heteroaryl), —Y⁴-(5-10 membered heterocyclyl), —Y⁴—O—Y¹—OR³⁷, —Y⁴—CO₂—R³⁷, and —Y⁴—OR³⁷, wherein the alkyl, cycloalkyl, aryl, heteroaryl and heterocyclyl moieties of the foregoing R⁴² and R⁴³ groups are optionally substituted by 1 or more substituents independently selected from R⁴⁴; or
R⁴² and R⁴³ taken together with the nitrogen to which they are attached form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are optionally substituted by 1 to 5 R⁴⁴ substituents, with the proviso that R⁴² and R⁴³ are not both bonded to the nitrogen directly through an oxygen;
Y is a bond or —(C(R^{y})(H))ₜ—, wherein t is an integer from 1 to 6; and
R^{y} at each occurrence is independently selected from the group consisting of H and C₁-C₆ alkyl, wherein the C₁-C₆ alkyl is optionally substituted;
Y⁴ is a bond or is —(C(R³⁷)(H))ₙ, wherein n is an integer ranging from 1 to 6;
R³⁷ is selected from H, OR³⁶, C₁-C₆ alkyl and C₃-C₁₀ cycloalkyl;
Y¹ is —(C(R³⁷)(H))₁₋₆;
each R⁴⁴ is independently selected from the group consisting of halo, cyano, nitro, trifluoromethoxy, trifluoromethyl, azido, —C(O)R⁴⁰, —C(O)OR⁴⁰, —OC(O)R⁴⁰, —OC(O)OR⁴⁰—NR³⁶C(O)R³⁹, —C(O)NR³⁶R³⁹, —NR³⁶R³⁹, —OR³⁷, —SO₂NR³⁶R³⁹, —SO₂R³⁶, —NR³⁶SO₂R³⁹, NR³⁶SO₂NR³⁷R⁴¹, C₁-C₆ alkyl, C₂-C₆ alkenyl, C₂-C₆ alkynyl, C₃-C₁₀ cycloalkyl, —C₁-C₆ alkylamino, —(CH₂)ⱼO(CH₂)ᵢNR³⁶R³⁹, —(CH₂)ₙO(CH₂)ᵢOR³⁷, —(CH₂)ₙOR³⁷, —S(O)ⱼ(C₁-C₆ alkyl), —(CH₂)ₙ(C₆-C₁₀ aryl), —(CH₂)ₙ(5-10 membered heterocyclyl), —C(O)(CH₂)ₙ(C₆-C₁₀ aryl), —(CH₂)ₙO(CH₂)ⱼ(C₆-C₁₀ aryl), —(CH₂)ₙO(CH₂)ᵢ(5 to 10 membered heterocyclyl), —C(O)(CH₂)ₙ(5 to 10 membered heterocyclyl), —(CH₂)ⱼNR³⁹(CH₂)ᵢNR³⁶R³⁹,
(CH₂)ⱼNR³⁹CH₂C(O)NR³⁶R³⁹—(CH₂)ⱼNR³⁹(CH₂)ᵢNR³⁷C(O)R⁴⁰,
(CH₂)ⱼNR³⁹(CH₂)ₙO(CH₂)ᵢOR³⁷—(CH₂)ⱼNR³⁹(CH₂)ᵢS(O)ⱼ(C₁-C₆ alkyl),
(CH₂)ⱼNR³⁹(CH₂)ₙR³⁶, —SO₂(CH₂)ₙ(C₆-C₁₀ aryl), and —SO₂(CH₂)ₙ(5 to 10 membered heterocyclyl) wherein, j is an integer from 0 to 2, n is an integer from 0 to 6 and i is an integer ranging from 2 to 6, the —(CH₂)ᵢ— and —(CH₂)ₙ₁— moieties of the foregoing R⁴⁴ groups optionally include a carbon-carbon double or triple bond wherein n is an integer from 2 to 6, and the alkyl, aryl and heterocyclyl moieties of the foregoing R⁴⁴ groups are optionally substituted by 1 or more substituents independently selected from the group consisting of halo, cyano, nitro, trifluoromethyl, azido, —OH, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —(CH₂)ₙNR³⁶R³⁹5-SO₂R³⁶, —SO₂NR³⁶R³⁹, C₁-C₆ alkyl, C₃-C₁₀ cycloalkyl, —(CH₂)ₙ(C₆-C₁₀ aryl), —(CH₂)ₙ(5 to 10 membered heterocyclyl), —(CH₂)ₙO(CH₂)ᵢOR³⁷ and —(CH₂)ₙOR³⁷, wherein n is an integer from 0 to 6 and i is an integer from 2 to 6;
each R³⁶ and R³⁹ is independently selected from the group consisting of H, —OH, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙO(CH₂)ᵢOR³⁷, —(CH₂)ₙCN(CH₂)ₙOR³⁷, —(CH₂)ₙCN(CH₂)ₙR³⁷, and —(CH₂)ₙOR³⁷, wherein n is an integer ranging from 0 to 6 and i is an integer ranging from 2 to 6, and the alkyl, aryl and heterocyclyl moieties of the foregoing R³⁶ and R³⁹ groups are optionally substituted by one or more substituents independently selected from —OH, halo, cyano, nitro, trifluoromethyl, azido, —C(O)R⁴⁰, —C(O)OR⁴⁰, —CO(O)R⁴⁰, —OC(O)OR⁴⁰, —NR³⁷C(O)R⁴¹, —C(O)NR³⁷R⁴¹, —NR³⁷R⁴¹, —C₁-C₆ alkyl, —(CH₂)ₙ(C₆-C₁₀ aryl), —(CH₂)ₙ(5 to 10 membered heterocyclyl), —(CH₂)ₙO(CH₂)ₙOR³⁷, and —(CH₂)ₙOR³⁷, wherein n is an integer ranging from 0 to 6 and i is an integer ranging from 2 to 6, with the proviso that when R³⁶ and R³⁹ are both attached to the same nitrogen, then R³⁶ and R³⁹ are not both bonded to the nitrogen directly through an oxygen;
each R⁴⁰ is independently selected from H, C₁-C₁₀ alkyl, —(CH₂)ₙ(C₆-C₁₀ aryl), C₃-C₁₀ cycloalkyl, and —(CH₂)ₙ(5-10 membered heterocyclyl), wherein n is an integer ranging from 0 to 6;
each R³⁷ and R⁴¹ is independently selected from H, OR³⁶, C₁-C₆ alkyl and C₃-C₁₀ cycloalkyl;
each R^{6a} and R^{6b} is independently selected from the group consisting of hydrogen and a moiety selected from the group consisting of —(CZ⁵Z⁶)-(C₃-C₆)cycloalkyl, —(CZ⁵Z⁶)ᵤ-(C₅-C₆)cycloalkenyl, —(CZ⁵Z⁶)ᵤ-aryl, —(CZ⁵Z⁶)ᵤ-heterocycle, alkoxy, (C₂-C₆)alkenyl, and (C₁-C₆)alkyl, wherein said moiety is optionally substituted with 1 to 3 independently selected Y³ groups, where u is 0, 1, 2, or 3, and wherein when u is 2 or 3, the CZ⁵Z⁶ units may be the same or different, or
R^{6a} and R^{6b} taken together with adjacent atoms form a heterocycle;
each Z³, Z⁴, Z⁵ and Z⁶ is independently selected from the group consisting of H, F and (C₁-C₆)alkyl, or
each Z³ and Z⁴, or Z⁵ and Z⁶ are selected together to form a carbocycle, or
two Z³ groups on adjacent carbon atoms are selected together to optionally form a carbocycle;
each Y² and Y³ is independently selected from the group consisting of H, halogen, trihalomethyl, cyano, nitro, tetrazolyl, guanidino, amidino, methylguanidino, azido, alkoxy, —C(O)Z⁷, —OC(O)NH₂, —OC(O)NHZ⁷, —OC(O)NZ⁷Z⁸, —N(Z⁷)C(O)Z⁷, —N(Z⁷)CO₂Z⁷, —NHC(O)NH₂, —NHC(O)NHZ⁷, —NHC(O)NZ⁷Z⁸, —C(O)OH, —C(O)OZ⁷, —C(O)NH₂, —C(O)NHZ⁷, —C(O)NZ⁷Z⁸, —P(O)₃H₂, —P(O)₃(Z⁷)₂, —S(O) ₃H, —SZ⁷, —S(O)Z⁷, —S(O)₂Z⁷, —S(O)₃Z⁷, —S(O)₂—NZ⁷Z⁸, —N(Z⁷)SO₂Z⁸, -Z⁷, —OZ⁷, —OH, —NH₂, —NHZ⁷, —NZ⁷Z⁸, —C(═NR²²⁵)N(Z⁷)Z⁸, —C(═NR²²⁵)Z⁸, C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxy, —(CZ⁹Z¹⁰)ᵣNH₂, —(CZ⁹Z¹⁰)ᵣNHZ³, —(CZ⁹Z¹⁰)ᵣNZ⁷Z⁸, —X⁶(CZ⁹Z¹⁰)ᵣ-(C₃-C₈)cycloalkyl, —X⁶(CZ⁹Z¹⁰)ᵣ-(C₅-C₈)cycloalkenyl, —X⁶(CZ⁹Z¹⁰)ᵣ-aryl and —X⁶(CZ⁹Z¹⁰)ᵣ-heterocycle, each of which is optionally substituted, wherein
R²²⁵ is selected from the group consisting of H, CN, NO₂, —OZ⁷, —S(O)₀₋₂Z⁸, —CO₂Z⁷ optionally substituted lower alkyl, optionally substituted lower alkenyl and optionally substituted lower alkynyl;
r is 1, 2, 3 or 4;
X⁶ is selected from the group consisting of O, S, NH, —C(O)—, —C(O)NH—, —C(O)O—, —S(O)—, —S(O)₂— and —S(O)₃—;
Z⁷ and Z⁸ are independently selected from the group consisting of H, an alkyl of 1 to 12 carbon atoms, an alkenyl of 2 to 12 carbon atoms, an alkynyl of 2 to 12 carbon atoms, a cycloalkyl of 3 to 8 carbon atoms, a heterocycloalkyl of 3 to 8 carbon atoms, a cycloalkenyl of 5 to 8 carbon atoms, an aryl of 6 to 14 carbon atoms, a heterocycle of 5 to 14 ring atoms, an aralkyl of 7 to 15 carbon atoms, and a heteroaralkyl of 5 to 14 ring atoms, each of which is optionally substituted, or
Z⁷ and Z⁸ together may optionally form a heterocycle;
Z⁹ and Z¹⁰ are independently selected from the group consisting of H, F, a (C₁-C₁₂)alkyl, a (C₆-C₁₄)aryl, a (C₅-C₁₄)heteroaryl, a (C₇-C₁₅)aralkyl and a (C₅-C₁₄)heteroaralkyl, or
Z⁹ and Z¹⁰ are taken together form a carbocycle, or
two Z⁹ groups on adjacent carbon atoms are taken together to form a carbocycle; or
any two Y² or Y³ groups attached to adjacent carbon atoms may be taken together to be —O[C(Z⁹)(Z¹⁰)]ᵣO or —O[C(Z⁹)(Z¹⁰)]ᵣ₊₁, or
any two Y² or Y³ groups attached to the same or adjacent carbon atoms may be selected together to form a carbocycle or heterocycle; and wherein
any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C₁-C₄)alkyl, (C₁-C₄)alkoxy and an —N[(C₁-C₄)alkyl][(C₁-C₄)alkyl];
R¹ is —C≡CH or —C≡C—(CR⁴⁵R¹⁵)ₙ—R⁴⁶; n is an integer from 0 to 6;
each R⁴⁵ is independently selected from the group consisting of H, a (C₁-C₆)alkyl and a (C₃-C₈)cycloalkyl;
R⁴⁶ is selected from the group consisting of heterocyclyl, —N(R⁴⁷)—C(O)—N(R⁴⁷)(R⁴⁸), —N(R⁴⁷)—C(S)—N(R⁴⁷)(R⁴⁸), —N(R⁴⁷)—C(O)—OR⁴⁸, —N(R⁴⁷)—C(O)—(CH₂), —R⁴⁸, —N(R⁴⁷)—SO₂R⁴⁷, —(CH₂)ₙNR⁴⁷R⁴⁸, —(CH₂)ₙOR⁴⁸, —(CH₂)ₙSR⁴⁹, —(CH₂)ₙS(O)R⁴⁹, —(CH₂)ₙS(O)₂R⁴⁹, —OC(O)R⁴⁹, —OC(O)OR⁴⁹, —C(O)NR⁴⁷R⁴⁸, heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, —(CH₂)ₙOR⁵⁰(CH₂)ₙC(O)R⁴⁹—C(O)₂R⁴⁹, —(CH₂)ₙSR⁴⁹, —(CH₂)ₙS(O)R⁴⁹, —(CH₂)ₙS(O)₂R⁴⁹, —(CH₂)ₙR⁴⁹, —(CH₂)ₙCN, aryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, or
R⁴⁷ and R⁴⁸, together with the atom to which they are attached, form a 3-8 membered carbo- or hetero-cyclic ring;
R⁴⁹ is selected from the group consisting of (C₁-C₆)alkyl, (C₃-C₈)cycloalkyl, heterocyclyl(C₁-C₆)alkylene, aryl(C₁-C₆)alkylene wherein the aryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, heteroaryl(C₁-C₆)alkylene wherein the heteroaryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁵⁰ and R⁵¹ are independently selected from the group consisting of H, (C₁-C₆)alkyl, (C₃-C₈)cycloalkyl and —C(O)R⁴⁵, or
R⁵⁰ and R⁵¹, together with the atom to which they are attached, form a 3-8 membered carbo- or hetero-cyclic ring; and
R²¹ is the group defined by -(Z₁₁)-(Z¹²)ₘ-(Z¹³)ₘ₁, wherein
Z¹¹ is heterocyclyl, when m and m1 are 0, or heterocyclylene, when either m or m1 are 1,
Z¹² is selected from the group consisting of OC(O), OC(S) and C(O);
Z¹³ is selected from the group consisting of heterocyclyl, aralkyl, N(H)R⁵², (C₁-C₃)alkyl, —OR⁵², halo, S(O)₂R⁵⁶, (C₁-C₃)hydroxyalkyl and (C₁-C₃)haloalkyl;
m is 0 or 1;
m1 is 0 or 1;
R⁵² is selected from the group consisting of H, —(CH₂)_{q}S(O)₂R⁵⁴, —(C₁-C₆) alkyl-NR⁵³R⁵³ (C₁-C₃)alkyl, —(CH₂)_{q}OR⁵³, —C(O)R⁵⁴ and —C(O)OR⁵³;
q is 0, 1, 2, 3 or 4;
each R⁵³ is independently (C₁-C₃)alkyl;
R⁵⁴ is (C₁-C₃)alkyl or N(H)R⁵³;
R⁵⁶ is selected from the group consisting of NH₂, (C₁-C₃)alkyl and OR⁵²;
V is a 5 to 7 membered cycloalkyl, aryl, heterocylic or heteroaryl ring system, any of which is optionally substituted with 0 to 4 R² groups;
R² at each occurrence is independently selected from the group consisting of R¹⁰⁷, —H, halogen, trihalomethyl, —O-trihalomethyl, —CN, —NO₂, —NH₂, —OR³, —NR³R⁴, —S(O)₀₋₂R³, S(O)₂NR³R³, —C(O)OR³, —C(O)NR³R³, —N(R³)SO₂R³, —N(R³)C(O)R³, —N(R³)CO₂R³,
C(O)R³, C₁-C₄ alkoxy, C₁-C₄ alkylthio, —O(CH₂)naryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, alkylamino, —CH₂(CH₂)₀₋₄-T², wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and
C₂-C₆ alkynyl are optionally substituted;
each R³ is independently selected from the group consisting of —H and R⁴;
R⁴ is selected from the group consisting of a (C₁-C₆)alkyl, an aryl, a lower arylalkyl, a heterocyclyl and a lower heterocyclylalkyl, each of which is optionally substituted, or
R³ and R⁴, taken together with a common nitrogen to which they are attached, form an optionally substituted five- to seven-membered heterocyclyl, the optionally substituted five- to seven-membered heterocyclyl optionally containing at least one additional annular heteroatom selected from the group consisting of N, O, S and P;
Z is selected from the group consisting of —O—, —S—, —S(O)—, S(O)₂—, —CH₂—, NBn, —NR⁵—, —OCH₂—, and —N(R⁵)CH₂—, wherein R⁵ is selected from the group consisting of H, C₁-C₆ alkyl, an optionally substituted (C₁-C₈)acyl and C₁-C₆ alkyl-O—C(O), wherein C₁-C₆ alkyl is optionally substituted;
E is selected from the group consisting of —O—, —N(R¹³)—, —N(H)—, —N(C₁-C₆alkyl)-, —CH₂N(H)— and —N(H)CH₂—;
X is selected from the group consisting of O, S, NH, N-alkyl, N—OH, N—O-alkyl, and NCN; [symbol] is a single or double bond;
X¹ is selected from the group consisting of O, S, CH₂, N—CN, N—O-alkyl, NH and N(C₁-C₆alkyl) when [symbol] when is a double bond, wherein any alkyl is optionally substituted, or
X¹ is selected from the group consisting of H, halogen, alkyl, alkenyl, alkynyl, CN, alkoxy, NH₂, trihalomethyl, NH(alkyl) and alkyl-thio, when is a single bond, wherein any said alkyl, alkenyl, alkynyl or alkoxy is optionally substituted;
L and L¹ are independently selected from the group consisting of CH, C, N, C(halogen) and C(C₁-C₆alkyl);
or
L¹ is O and W is absent;
L² and L are independently selected from the group consisting of CH, CH₂, N, NH, O, S, —C(O)— and —C(S)—, —C(NH)— and —C(N—C₁-C₆alkyl)-;
L⁴ is selected from the group consisting of absent, CH, CH₂, N, NH, O, S, —C(O)—, —C(S)—, —C(NH)— and —C(N—C₁-C₆alkyl)-;
the group 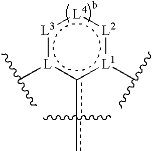
is aromatic or non-aromatic, provided that two 0 are not adjacent to each other;
and wherein any group 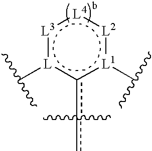
which comprises a CH₂ group which is attached to 2 carbon atoms or a CH₃ group which is attached to a carbon atom may optionally bear on each said CH₂ or CH₃ group a substituent selected from the group consisting of hydroxy, amino, C₁₋₆alkoxy, N—C₁₋₆alkylamino, N,N—(C₁₋₆alkyl)₂-amino and heterocyclyl;
b is 0-5, preferably 0-4, more preferably 0-1, more preferably 0; and
W is a five- to ten-membered cycloalkyl, aryl, heterocylic or heteroaryl ring system, which is optionally substituted;
R¹³ is selected from the group consisting of H, C₁-C₆alkyl, substituted C₁-C₆alkyl, cycloalkyl, substituted cycloalkyl, OH, unsubstituted —O—(C₁-C₆alkyl) and substituted —O—(C₁-C₆alkyl);
and
R¹⁴, R¹⁵, R¹⁶ and R¹⁷ are independently selected from the group consisting of —H, halogen, trihalomethyl, —O-trihalomethyl, —CN, —NO₂, —NH₂, —OR³, —OCF₃, —NR³R⁴, —S(O)₀₋₂R³, —S(O)₂NR³R³, —C(O)OR³, —C(O)NR³R³, —N(R³)SO₂R³, —N(R³)C(O)R³, —N(R³)C(O)OR³, —C(O)R³, —C(O)SR³, C₁-C₆ alkoxy, C₁-C₄ alkylthio, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², carboxy, C₁₋₆alkoxycarbonyl, carbamoyl, N—C₁₋₆alkylcarbamoyl, N—(C₁₋₆alkyl)₂-carbamoyl, C₂₋₆alkanoyl, amino, N—C₁₋₆alkylamino, N,N—(C₁₋₆alkyl)₂-amino, an optionally substituted C₁₋₄ alkylcarbonyl, C₁₋₄ alkoxy, an amino optionally substituted by C₁₋₄ alkyl optionally substituted by C₁₋₄ alkoxy and a saturated or unsaturated three- to seven-membered carboxyclic or heterocyclic group, wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted;
with the proviso that Formula (I) excludes those compounds wherein
A is 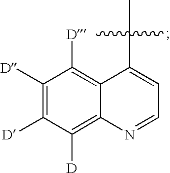
D, D′, D″ and D′″ are independently selected from R²⁵⁹;
Z is NH;
V is an optionally substituted 6-membered aromatic ring containing at least one nitrogen atom,
E is —N(H)— or —CH₂N(H)—;
X is O;
X¹ is selected from the group consisting of halogen, alkyl, alkoxy, amino, alkylamino and one or two oxo, thioxo;
the group 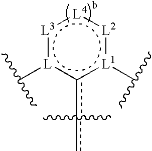
is aryl, heteroaryl or heterocyclyl, optionally substituted with one or more groups selected from halogen and C₁-C₆alkyl, and wherein said heterocyclyl is optionally substituted with one or two oxo or thioxo substituents, and wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituent selected from the group consisting of hydroxy, amino, C₁₋₆alkoxy, N—C₁₋₆alkylamino, N,N—(C₁₋₆alkyl)₂amino and heterocyclyl; and
W is selected from the group consisting of aryl, heteroaryl and heterocyclyl, each optionally substituted with one or more groups selected from hydroxy, halo, C₁₋₆alkyl, C₁₋₆alkoxy, carboxy, C₁₋₆alkoxycarbonyl, carbamoyl, N—C₁₋₆alkylcarbamoyl, N—(C₁₋₆alkyl)₂-carbamoyl, C₂₋₆alkanoyl, amino, N—C₁₋₆alkylamino and N,N—(C₁₋₆alkyl)₂-amino, and wherein said heterocyclyl is optionally substituted with one or two oxo or thioxo substituents, wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituents selected from hydroxyl, amino, C₁₋₆alkoxy, N—C₁₋₆alkylamino, N,N—(C₁₋₆alkyl)₂-amino and heterocyclyl.
with the proviso that Formula (I) excludes those compounds wherein
A is 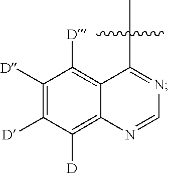
D, D′, D″ and D′″ are independently selected from R²⁵⁹;
Z is selected from the group consisting of O, S, S(O), S(O)₂, NH and N(C₁₋₆alkyl);
V is a group selected from: 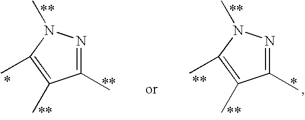
wherein * indicates the point of attachment to the group Z of Formula I and ** indicates optional points of attachment to the group E of Formula I;
E is —N(H)— or —CH₂N(H)—;
X is O or NCN;
X¹ is selected from the group consisting of halogen, alkyl, alkoxy, amino, alkylamino and one or two oxo, thioxo;
the group 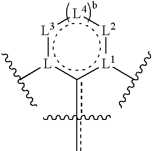
is aryl, heteroaryl or heterocyclyl, optionally substituted with one or more groups selected from halogen and C₁-C₆alkyl, and wherein said heterocyclyl is optionally substituted with one or two oxo or thioxo substituents, and wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituent selected from the group consisting of hydroxy, amino, C₁₋₆alkoxy, N—C₁₋₆alkylamino, N,N—(C₁₋₆alkyl)₂amino and heterocyclyl; and
W is selected from the group consisting of aryl, heteroaryl and heterocyclyl, each optionally substituted with one or more groups selected from hydroxy, halo, C₁₋₆alkyl, C₁₋₆alkoxy, carboxy, C₁₋₆alkoxycarbonyl, carbamoyl, N—C₁₋₆alkylcarbamoyl, N—(C₁₋₆alkyl)₂-carbamoyl, C₂₋₆alkanoyl, amino, N—C₁₋₆alkylamino and N,N—(C₁₋₆alkyl)₂amino, and wherein said heterocyclyl is optionally substituted with one or two oxo or thioxo substituents, wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituents selected from hydroxyl, amino, C₁₋₆alkoxy, N—C₁₋₆alkylamino, N,N—(C₁₋₆alkyl)₂amino and heterocyclyl.
with the proviso that Formula (I) excludes those compounds wherein
A is 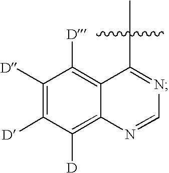
D, D′, D″ and D′″ are independently selected from R²⁵⁹;
Z is selected from the group consisting of O, S, S(O), S(O)₂, NH and N(C₁₋₆alkyl);
V is an optionally substituted 5-membered heteroaromatic ring;
E is —N(H)— or —CH₂N(H)—;
X is O or NCN;
the group 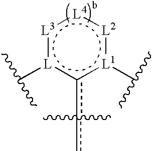
is aryl, heteroaryl or heterocyclyl, optionally substituted with one or more groups selected from halogen and C₁-C₆alkyl, and wherein said heterocyclyl is optionally substituted with one or two oxo or thioxo substituents, and wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituent selected from the group consisting of hydroxy, amino, C₁₋₆alkoxy, N—C₁₋₆alkylamino, N,N—(C₁₋₆alkyl)₂amino and heterocyclyl; and
W is selected from the group consisting of aryl, heteroaryl and heterocyclyl, each optionally substituted with one or more groups selected from hydroxy, halo, C₁₋₆alkyl, C₁₋₆alkoxy, carboxy, C₁₋₆alkoxycarbonyl, carbamoyl, N—C₁₋₆alkylcarbamoyl, N—(C₁₋₆alkyl)₂carbamoyl, C₂₋₆alkanoyl, amino, N—C₁₋₆alkylamino and N,N—(C₁₋₆alkyl)₂amino, and wherein said heterocyclyl is optionally substituted with one or two oxo or thioxo substituents, wherein any of said aryl, heteroaryl or heterocyclyl which comprises a CH₂ group which is attached to 2 carbon atoms or a CH₃ group which is attached to a carbon atom may optionally bear on each said CH₂ or CH₃ group a substituents selected from hydroxyl, amino, C₁₋₆alkoxy, N—C1-6alkylamino, N,N—(C₁₋₆alkyl)₂amino and heterocyclyl;
with the proviso that Formula (I) excludes those compounds wherein
A is 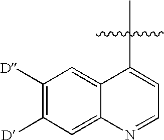
D′ and D″ are each independently OH or —O—C₁-C₅ alkyl, wherein D′ and D″ optionally together form C₁-C₃alkylene;
Z is selected from the group consisting of O and S;
V is a phenyl group;
E is —N(H)— or —N(C₁-C₄alkyl)-;
X is selected from the group consisting of O, S, NH, NCN, N(C₁-C₅alkyl) and N—O—C₁-C₅alkyl;
the group 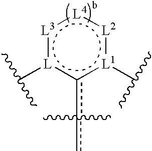
is C₃-C₁₀cycloalkyl, phenyl, furyl, thienyl, 5- or 6-membered heteroaryl having 1 or 2 nitrogen atoms and optionally having another heteroatom selected from the group consisting of nitrogen, oxygen and sulfur atoms, wherein each of said C₃-C₁₀cycloalkyl, phenyl, furyl, thienyl and 5- or 6-membered heteroaryl is optionally substituted by halogen or C₁-C₅alkyl; and
W is selected from the group consisting of C₃-C₁₀cycloalkyl, phenyl, substituted phenyl, phenoxy, substituted phenoxy, phenylthio, substituted phenylthio, phenyl(C₁-Calkyl), substituted phenyl(C₁-C₄alkyl), pyridyl, pyrazinyl, pryimidinyl, pyridazinyl, pyrrolidinyl, piperidinyl, piperazinyl, homopyperazinyl, morpholinyl, quinolyl, quinazolinyl, benzoyl and substituted benzoly;
with the proviso that Formula (I) excludes those compounds wherein
A is 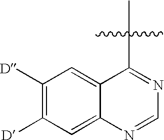
D′ and D″ are each independently OH or —O—C₁-C₅ alkyl, wherein D′ and D″ optionally together form C₁-C₃alkylene;
Z is selected from the group consisting of O, S and CH₂;
V is a phenyl group;
E is —N(H)— or —N(C₁-C₄alkyl)-;
X is selected from the group consisting of O, S, NH, NCN, N(C₁-C₅alkyl) and N—O—C₁-C₅alkyl;
the group 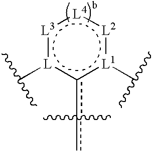
is C₃-C₁₀cycloalkyl, phenyl, furyl, thienyl, 5- or 6-membered heteroaryl having 1 or 2 nitrogen atoms and optionally having another heteroatom selected from the group consisting of nitrogen, oxygen and sulfur atoms, wherein each of said C₃-C₁₀cycloalkyl, phenyl, furyl, thienyl and 5- or 6-membered heteroaryl is optionally substituted by halogen or C₁-C₅alkyl; and
W is selected from the group consisting of C₃-C₁₀cycloalkyl, phenyl, substituted phenyl, phenoxy, substituted phenoxy, phenylthio, substituted phenylthio, phenyl(C1-Calkyl), substituted phenyl(C₁-C₄alkyl), pyridyl, pyrazinyl, pryimidinyl, pyridazinyl, pyrrolidinyl, piperidinyl, piperazinyl, homopyperazinyl, morpholinyl, quinolyl, quinazolinyl, benzoyl and substituted benzoly;
with the proviso that Formula (I) excludes those compounds wherein
A is 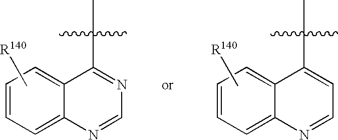
R¹⁴⁰ is selected from the group consisting of H, halogen, —OR^{140a}, —NO₂, —NH₂, —NR^{140a}R^{140b}, and optionally substituted lower alkyl, alkenyl, alkynyl or cycloalkyl, wherein
R^{140a} is H or R^{140b}
R^{140b} is selected from the group consisting of optionally substituted lower alkyl, alkenyl, alkynyl or cycloalkyl, optionally substituted aryl, optionally substituted lower arylalkyl, optionally substituted heterocyclyl, optionally substituted heteroaryl, optionally substituted heterocyclylalkyl and optionally substituted lower heteroarylalkyl, or
R^{140a} and R^{140b}, when taken together with a common nitrogen to which they are attached, form an optionally substituted five- to seven-membered heterocyclyl or optionally substituted five- to seven-membered heteroaryl, said optionally substituted five- to seven-membered heterocyclyl or optionally substituted five- to seven-membered heteroaryl optionally containing at least one additional annular heteroatom selected from N, O and S;
Z is selected from the group consisting of —S(O)₀₋₂, —O— and —N(H)— and —N(lower alkyl, alkenyl, alkynyl or cycloalkyl)-;
V is selected from the group consisting of 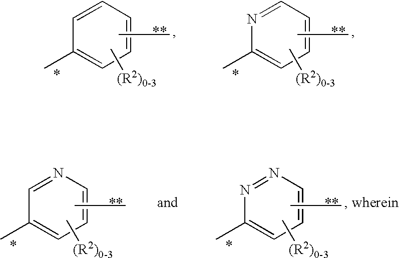
R² is selected from the group consisting of H, halogen, trihalomethyl, —CN, —NO₂, —NH₂, —OR^{140a}, —NR^{140a}R^{140b} —S(O)₀₋₂R^{140a}, —SO₂NR^{140a}R^{140b}, —CO₂R^{140a}, —C(O)NR^{140a}R^{140b}, —N(R^{140a})SO₂R^{140a}, —N(R^{140a})C(O)R^{140a}, —N(R^{140a})CO₂R^{140a}, —C(O)R^{140a} and optionally substituted lower alkyl, alkenyl, alkynyl or cycloalkyl,;
E is selected from the group consisting of —N(H)—, —N(lower alkyl, alkenyl, alkynyl or cycloalkyl,)-;
X is O;
the group 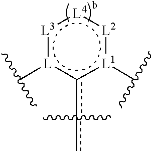
is an optionally substituted five- to seven-membered heterocyclyl or heteroaryl, said optionally substituted five- to seven-membered heterocyclyl or heteroaryl optionally containing at least one additional annular heteroatom selected from N, O, S; and
X¹ is O, S and CH₂;
with the proviso that Formula (I) excludes those compounds wherein
A is 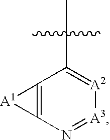
wherein
wherein each hydrogen in A¹ is optionally substituted by an R¹⁰⁶ group, wherein
R¹⁰⁶ is selected from the group consisting of halogen, C₁-C₁₂alkyl, C₂-C₁₂alkenyl, C₂-C₁₂alkynyl, C₃-C₁₂cycloalkyl, C₆-C₁₂aryl, 3-12 membered heteroalicyclic, 5-12 membered heteroaryl, —S(O)₀₋₂R¹⁰⁸, —SO₂NR¹⁰⁸R¹⁰⁹, —S(O)₂OR¹⁰⁸, —NO₂, —NR¹⁰⁸R¹⁰⁹, —(CR¹¹⁰R¹¹¹)₀₋₄OR¹⁰⁸, —CN, —C(O)R¹⁰⁸, —OC(O)R¹⁰⁸, —O(CR¹¹⁰R¹¹¹)₀₋₄R¹⁰⁸, —NR¹⁰⁸C(O)R¹⁰⁹, —(CR¹¹⁰R¹¹¹)₀₋₄C(O)OR¹⁰⁸, —(CR¹¹⁰R¹¹¹)₀₋₄NR¹⁰⁸R¹⁰⁹, —C(═NR¹¹⁰)NR¹⁰⁸R¹⁰⁹, —NR¹⁰⁸C(O)NR¹⁰⁹R¹¹⁰, —NR¹⁰⁸S(O)₁₋₂R¹⁰⁹ and —C(O)NR¹⁰⁸R¹⁰⁹, and each hydrogen in R¹⁰⁶ is optionally substituted by an R¹¹⁷ group;
Z is O;
V is 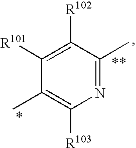
wherein * indicates the point of attachment to the group Z of Formula I and ** indicates point of attachment to the group E of Formula I,
R^{10l}, R¹⁰² and R¹⁰³, which may be the same or different, are each independently selected from R¹⁰⁷.
E is —N(H)—;
X is O or S;
X¹ is selected from the group consisting of alkyl, alkenyl, alkynyl, CN, alkoxy and halogen; and
R⁴, R⁵, R¹⁶ and R¹⁷ are independently selected from the group consisting of R¹¹⁸;
with the proviso that Formula (I) excludes those compounds wherein
A is 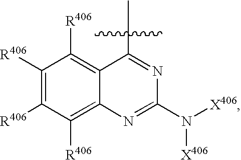
wherein
each R⁴⁰⁶ is independently selected from the group consisting of hydrogen, —F, —Cl, —Br, —I, —OH, —SH, —NO₂, —CN, —OR^{406r}, —SR^{406d}, —S(O)R^{406d}, —S(O)₂R^{406d}, —NR^{406b}, R^{406c}, C(O)R^{406a}, —C(O)OR^{406e} and an optionally substituted aliphatic, alicyclic, heteroaliphatic, heterocyclic, aromatic, heteroaromatic or acyl moiety, and any two R⁴⁰⁶, together with the carbons to which they are bound, may represent a fused 5-9 membered alicyclic, heterocyclic, aromatic or heteroaromatic ring;
R^{406r} is selected from the group consisting of an optionally substituted aliphatic, alicyclic heteroaliphatic, heterocyclic, aromatic, heteroaromatic or acyl moiety;
R^{406a} is selected from the group consisting of hydrogen or an optionally substituted aliphatic, alicyclic, heteroaliphatic, heterocyclic, aromatic or heteroaromatic moiety;
R^{406b} is selected from the group consisting of hydrogen, —OH, —SO₂R^{406d}, or an optionally substituted aliphatic, alicyclic, heteroaliphatic, heterocyclic, aromatic, heteroaromatic or acyl moiety;
R^{406c}, is selected from the group consisting of hydrogen, —OH, —SO₂R^{406d}, or an optionally substituted aliphatic, alicyclic, heteroaliphatic, heterocyclic, aromatic, heteroaromatic or acyl moiety;
R^{406d} is selected from the group consisting of hydrogen, —N(R^{406e})₂, or an optionally substituted aliphatic, alicyclic, heteroaliphatic, heterocyclic, aromatic or heteroaromatic moiety; and
R^{406d} is hydrogen or an optionally substituted aliphatic moiety;
Z is NH, N (optionally substituted Bn), N (optionally substituted alkyl) or N (optionally substituted acyl);
E is —N(H)— or —N(optionally substituted alkyl);
X is O or S; and
X¹ is selected from the group consisting of alkyl, alkenyl, alkynyl, alkoxy, halogen, cyano, trifluoromethyl, and alkyl-thio;
with the proviso that Formula (I) excludes those compounds wherein
A-Z-V- is 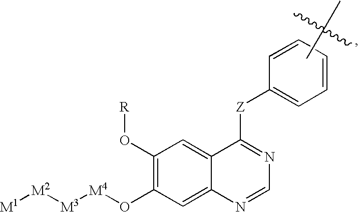
wherein
M¹, M², M³ and M⁴ are as defined above;
R is C₁-C₃alkyl optionally substituted with between one and three groups independently selected from the group consisting of H, halogen, trihalomethyl, —OZ⁷, —NZ⁷Z⁸, —SZ⁷, —S(O)Z⁷, —S(O)₂Z⁷, —S(O)₂—NZ⁷Z⁸, —C(O)OZ⁷, —C(O)NZ⁷Z⁸, —C(═NR²²⁵)N(Z⁷)Z⁸, —C(═NR²²⁵)Z⁸, —N(Z⁷)SO₂Z⁸, —N(Z⁷)C(O)Z⁷, —N(Z⁷)CO₂Z⁷, —C(O)Z⁷, optionally substituted alkoxy, optionally substituted lower alkyl, optionally substituted aryl, optionally substituted lowe arylalkyl, optionally substituted heterocyclyl, and optionally substituted lower heterocyclylalkyl; or each of said groups, when taken together on the same carbon are oxo; or two of said groups, when taken together with a common carbon to which they are attached, form an optionally substituted three- to seven-membered spirocyclyl, said optionally substituted three- to seven-membered spirocyclyl containing at least one additional heteroatom selected from N, O, S and P;
Z is selected from the group consisting of —OCH₂—, —O—, —S(O)₀₋₂—, —N(H)CH₂—, —N(C₁-C₆ alkyl)CH₂—, —NH— and —N(C₁-C₆ alkyl)-;
R¹³ is selected from the group consisting of H, optionally substituted C₁-C₆alkyl;
X is O; and
the group 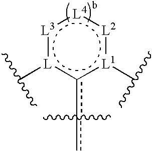
is an optionally substituted aryl, an optionally substituted lower arylalkyl, optionally substituted heterocyclyl and an optionally substituted lower heterocyclylalkyl;
with the proviso that Formula (I) excludes those compounds wherein
A-Z-V- is 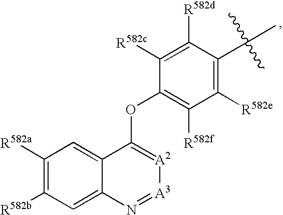
wherein
A² and A³ are independently selected from CH and N;
R^{582a} and R^{582b} are independently selected from the group consisting of H and C₁-C₄alkoxy optionally substituted by a halogen atom;
R⁵⁸²c, R^{582d}, R^{582e} and R^{582f} are independently selected from the group consisting of H, halogen, C₁-C₄alkyl optionally substituted by a halogen atom, C₁-C₄alkoxy optionally substituted by a halogen atom, nitro, amino and morpholyl;
R¹³ is selected from the group consisting of H and C₁-C₄alkyl optionally substituted by a halogen atom;
X is O; and
the group 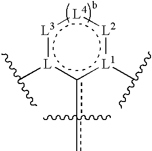
is selected from the group consisting of phenyl optionally substituted by phenyl optionally substituted by a halogen atom.

2. The compound according to claim 1, wherein A is selected from the group consisting of

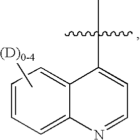

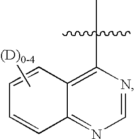

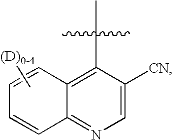

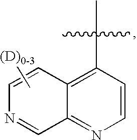

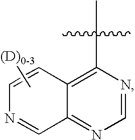

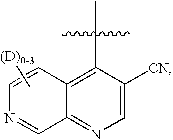

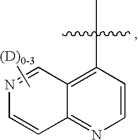

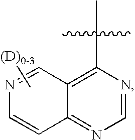

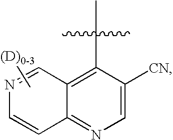

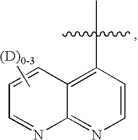

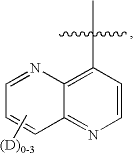

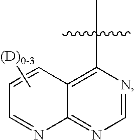

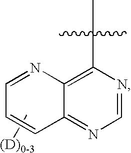

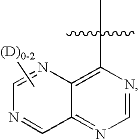

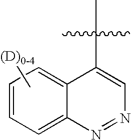

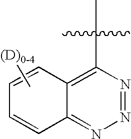

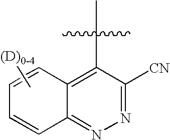

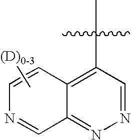

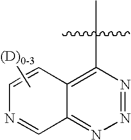

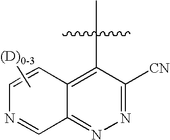

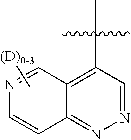

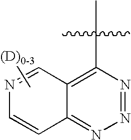

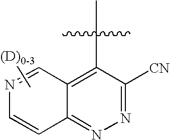

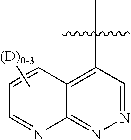

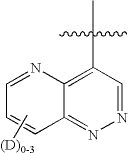

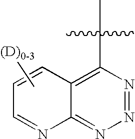

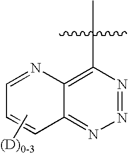

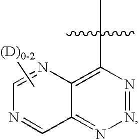

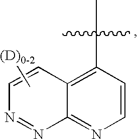

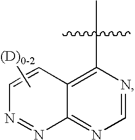

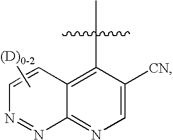

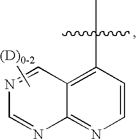

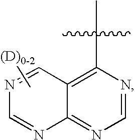

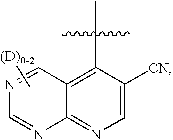

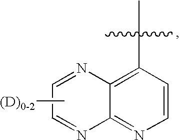

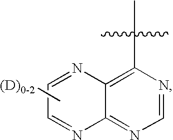

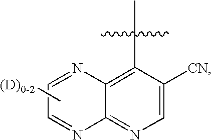

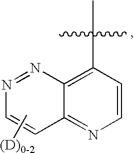

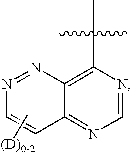

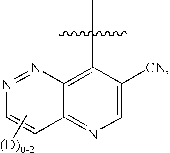

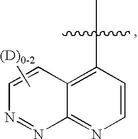

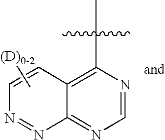

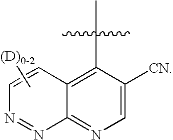

3. The compound according to claim 1, wherein A is substituted by 1 or 2 D groups.

4. The compound according to claim 1, wherein each D is independently defined by the group R⁷, wherein R⁷ is selected from the group consisting of —H, halogen, C₁-C₆ alkyl C₃-C₁₀ cycloalkyl, —C(O)NR⁴²R⁴³, —C(O)(C₆-C₁₀ aryl), —C(O)(heterocyclyl), —C(O)(heteroaryl), —Y—(C₆-C₁₀ aryl), —Y-(5-10 membered heterocyclyl), —Y-(heteroaryl), —S-aryl, —S—C₁-C₆ alkyl, —SO—C₁-C₆ alkyl, —SO₂—C₁-C₆ alkyl, —Y—NR⁴²R⁴³, —SO₂NR⁴²R⁴³, —OR^{6a} and —C(O)OR^{6a}, wherein the aforementioned R⁷ groups other than —H and halogen are optionally substituted.

5. The compound according to claim 1, wherein R⁷ is selected from the group consisting of —(CH₂)ₙ(5 to 10 membered heterocyclyl), OR^{6a} and —C(O)NR⁴²R⁴³.

6. The compound according to claim 6, wherein R^{6a} is C₁-C₆alkyl, optionally substituted with 1 to 3 independently selected Y³ groups.

7. The compound according to claim 6, whereinY³ is —NZ⁷Z⁸.

8. The compound according to claim 7, wherein each of Z⁷ and Z⁸ is independently selected from H and an optionally substituted C₁-C₁₂alkyl.

9. The compound according to claim 1, wherein Z is —O—.

10. The compound according to claim 1, wherein V is a 6 membered aryl or heteroaryl ring system, wherein said V is optionally substituted with 0 to 4 R² groups.

11. The compound according to claim 1, wherein E is —N(R¹³)—.

12. The compound according to claim 1, wherein X is O.

13. The compound according to claim 1, wherein [symbol] is a double bond and X¹ is O.

14. The compound according to claim 1, wherein [symbol] is a single bond and X¹ is H, or an optionally substituted alkyl.

15. The compound according to claim 1, wherein L and L¹ are independently selected from —CH— and —N—.

16. The compound according to claim 1, wherein the group 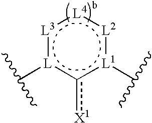
is represented by the group

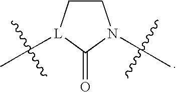

17. The compound according to claim 1, wherein W is phenyl.

18. The compound according to claim 1, wherein W is phenyl and one of R¹⁴, R¹⁵, R¹⁶ and R¹⁷ is a halogen or a C₁-C₆alkoxy, and the other three are H.

19. A compound of the formula (I-A) and racemic mixtures, diastereomers and enantiomers thereof: 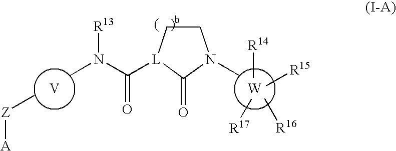
and N-oxides, hydrates, solvates, pharmaceutically acceptable salts, prodrugs and complexes thereof, wherein A, Z, V, W, b, R¹³, R¹⁴, R¹⁵, R¹⁶ and R¹⁷ are as defined in claim 1,
and L is selected from the group consisting of CH, N, C(halogen), C(C≡CH), C(C≡N) and C(NO₂).

20. A compound of the Formula (I-B) and racemic mixtures, diastereomers and enantiomers thereof: 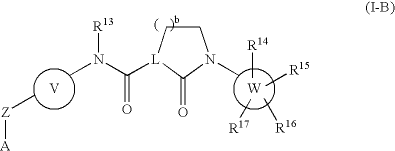
and N-oxides, hydrates, solvates, pharmaceutically acceptable salts, prodrugs and complexes thereof, wherein
A is 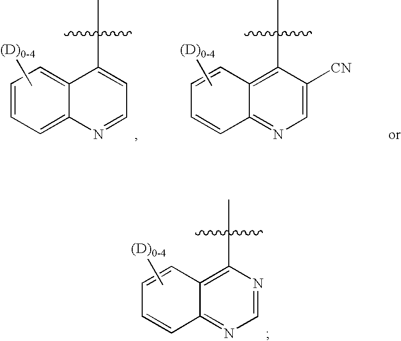
Z is —O—, —S—, —NH— or —N(C₁-C₆alkyl);
V is phenyl or pyridinyl, each of which is optionally substituted with 0 to 4 R² groups;
R¹³ is H or C₁-C₆alkyl;
L is —CH—, —N— or —C(halogen)-;
b is zero;
W is phenyl or pyridinyl; and
R¹⁴, R¹⁵, R¹⁶ and R¹⁷ are each independently H, halogen or alkoxy.

21. The compound according to claim 20, wherein A is substituted by two D, each independently selected from the group consisting of R⁰⁷⁷, X²—R^{d}, C₁-C₆alkoxy and —OR^{6a}.

22. The compound according to claim 21, wherein each D is independently alkoxy or —OR^{6a}.

23. A compound of Formula (II) and racemic mixtures, diastereomers and enantiomers thereof: 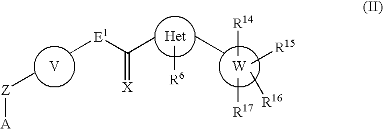
and N-oxides, hydrates, solvates, pharmaceutically acceptable salts, prodrugs and complexes thereof, wherein,
E¹ is selected from the group consisting of —CH₂—, —N(R¹³)—, —N(H)—, —N(C₁-C₆alkyl)-, —CH₂N(H)— and —N(H)CH₂—, and
A, Z, V, X, W, R¹³, R¹⁴R¹⁵, R¹⁶ and R¹⁷ are as defined in claim 1; and
R⁶ is selected from the group consisting of absent, H, halogen, alkyl, alkenyl, alkynyl, CN, alkoxy, NH₂, trihalomethyl, NH(alkyl), di-alkylamino and alkyl-thio, wherein any said alkyl, alkenyl, alkynyl or alkoxy is optionally substituted; and
Het is an optionally substituted 5 or 6-membered aryl or heterocyclic.

24. The compound according to claim 23, wherein E¹ is —NH— or —CH₂—.

25. The compound according to claim 23, wherein Het is a 5-membered heteroaryl.

26. The compound according to claim 23, wherein R⁶ is absent or an optionally substituted alkyl.

27. A compound of Formula (III) and racemic mixtures, diastereomers and enantiomers thereof: 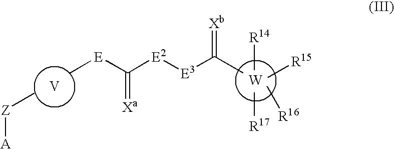
and N-oxides, hydrates, solvates, pharmaceutically acceptable salts, prodrugs and complexes thereof, wherein,
X^{a} and X^{b} are independently selected from the group consisting of O, S, N(H), N(alkyl), N(OH), N(O-alkyl), and N(CN);
E, E and E³ are each independently selected from the group consisting of —N(R¹³)—, —N(H)—, —N(C₁-C₆alkyl)-, —CH₂N(H)— and —N(H)CH₂—; and
A, Z, V, E, W, R¹³, R¹⁴, R¹⁵, R¹⁶ and R¹⁷ are as defined in claim 1.

28. The compound according to claim 27, wherein each of E, E² and E³ are independently selected from —N(R¹³)—.

29. The compound according to claim 27, wherein each of E, E² and E³ are —NH—.

30. The compound according to claim 27, wherein X^{a} and X^{b} are independently selected from O and S.

31. A compound of Formula (IV) and racemic mixtures, diastereomers and enantiomers thereof: 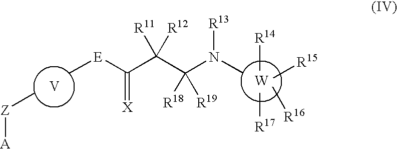
and N-oxides, hydrates, solvates, pharmaceutically acceptable salts, prodrugs and complexes thereof,
or
a compound of Formula (V) and racemic mixtures, diastereomers and enantiomers thereof: 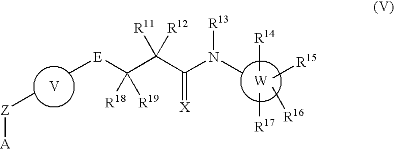
and N-oxides, hydrates, solvates, pharmaceutically acceptable salts, prodrugs and complexes thereof.
wherein,
A, Z, V, E, X, W, R¹³, R¹⁴, R¹⁵, R¹⁶ and R¹⁷are as defined in claim 1; and
R¹¹ and R¹² are independently selected from the group consisting of H, halogen, —OH, unsubstituted —O—(C₁-C₆alkyl), substituted —O—(C₁-C₆alkyl), unsubstituted —O-(cycloalkyl), substituted —O-(cycloalkyl), unsubstituted —NH(C₁-C₆alkyl), substituted —NH(C₁-C₆alkyl), —NH₂, —SH, unsubstituted —S—(C₁-C₆alkyl), substituted —S—(C₁-C₆alkyl), unsubstituted C₁-C₆alkyl and substituted C₁-C₆alkyl; or
R¹¹ and R¹² taken together with the atom to which they are attached form a C₃-C₇ ring system, wherein said ring system is optionally substituted; or
R¹² and R¹³ taken together with the atoms to which they are attached optionally form a 4 to 8 membered cycloalkyl or heterocyclic ring system, which ring system is optionally substituted; or
R¹³ and R¹⁴ taken together with the atoms to which they are attached optionally form a 4 to 8 membered cycloalkyl or heterocyclic ring system, which ring system is optionally substituted; and
R¹⁸ and R¹⁹ are independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or
R¹⁸ and R¹⁹ together with the atom to which they are attached form a 3 to 6 membered cycloalkyl or heterocycle, each of which is optionally substituted with 1 to 4 halo.

32. The compound according to claim 31 wherein R¹¹ and R¹² are each —H.

33. The compound according to claim 31, wherein R¹¹, R¹² and R¹³ are each —H.

34. The compound according to claim 31, wherein one of R¹⁸ and R¹⁹ is —CF₃ and the other is —H.

35. The compound according to claim 31, wherein X is O, one of R¹⁸ and R¹⁹ is —CF₃ and the other is —H, and R¹¹, R¹² and R¹³ are each —H.

36. A compound of the Formula (IV-A) and racemic mixtures, diastereomers and enantiomers thereof: 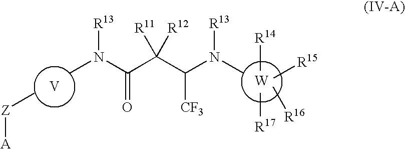
and N-oxides, hydrates, solvates, pharmaceutically acceptable salts, prodrugs and complexes thereof,
or
a compound of Formula (V-A) and racemic mixtures, diastereomers and enantiomers thereof: 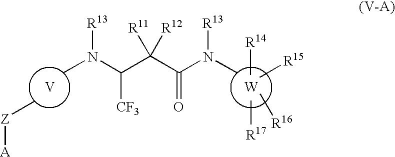
and N-oxides, hydrates, solvates, pharmaceutically acceptable salts, prodrugs and complexes thereof,
wherein A, Z, V, W, R¹¹, R¹², R¹³, R¹⁴, R¹⁵, R¹⁶ and R¹⁷ are as defined in claim 31.

37. The compound according to claim 36, wherein W is phenyl.

38. A pharmaceutical composition comprising a compound according to claim 1 and a pharmaceutically acceptable carrier.

39. The composition according to claim 38, further comprising an additional therapeutic agent.

40. A method of inhibiting kinase activity, the method comprising contacting the kinase with a kinase inhibiting amount of a compound according to claim 1 or a composition thereof.

41. A method of inhibiting kinase activity in a cell, the method comprising contacting the cell with a kinase inhibiting amount of a compound according to claim 1 or a composition thereof.

42. A method of inhibiting proliferative activity of a cell, the method comprising contacting the cell with an effective proliferative inhibiting amount of a compound according to claim 1 or a composition thereof.

43. The method of claim 42, further comprising contacting the cell with an additional therapeutic agent.

44. A method of treating a cell proliferative disease in a patient, the method comprising administering to the patient in need of such treatment an effective therapeutical amount of a compound according to claim 1 or a composition thereof.

45. The method of claim 44, further comprising administering an additional therapeutic agent.

46. A method of inhibiting tumor growth in a patient, the method comprising administering to the patient in need there of an effective therapeutical amount of a compound according to claim 1 or a composition thereof.

47. The method of claim 46, further comprising administering an additional therapeutic agent.
